(19) United States
(12) Patent Application Publication
(10) Pub. No.: US 2023/0089247 A1
(43) Pub. Date: Mar. 23, 2023

(54) NOVEL COMPOUNDS SUITABLE FOR THE TREATMENT OF DYSLIPIDEMIA

(71) Applicant: ZYDUS LIFESCIENCES LIMITED, Ahmedabad, Gujarat (IN)

(72) Inventors: Rajiv SHARMA, Ahmedabad, Gujarat (IN); Sanjay KUMAR, Ahmedabad, Gujarat (IN); Harikishore PINGALI, Ahmedabad, Gujarat (IN); Suresh POLA, Ahmedabad, Gujarat (IN); Pankaj MAKADIA, Ahmedabad, Gujarat (IN); Prashant DESHMUKH, Ahmedabad, Gujarat (IN); Pandurang ZAWARE, Ahmedabad, Gujarat (IN); Kiran SHAH, Ahmedabad, Gujarat (IN)

(21) Appl. No.: 17/788,611

(22) PCT Filed: Dec. 24, 2020
(86) PCT No.: PCT/IB2020/062444

§ 371 Date: Jun. 23, 2022

(30) Foreign Application Priority Data
Dec. 24, 2019 (IN) 201921053637

(51) Int. Cl. C07D 405/10; C07D 487/04; C07D 401/10; C07D 409/10; C07D 413/14; C07D 471/10; C07D 405/14

(52) U.S. Cl. CPC C07D 405/10; C07D 487/04; C07D 401/10; C07D 409/10; C07D 413/14; C07D 471/10; C07D 405/14

(57) ABSTRACT

The present invention relates to compounds of the general formula (I), their tautomeric forms, their stereoisomers, their pharmaceutically acceptable salts, pharmaceutical compositions containing them, methods for their preparation, use of these compounds in medicine and the intermediates involved in their preparation The present invention is directed towards compounds which can be useful in treating diseases such as Hyperlipidemia and also have a beneficial effect on lowering cholesterol.

### FIELD OF INVENTION

The present invention relates to compounds of the general formula (I), their tautomeric forms, their stereoisomers, their pharmaceutically acceptable salts, pharmaceutical compositions containing them, methods for their preparation, use of these compounds in medicine and the intermediates involved in their preparation.

The present invention is directed towards compounds which can be used to treat diseases such as hyperlipidemia and also have a beneficial effect on cholesterol.

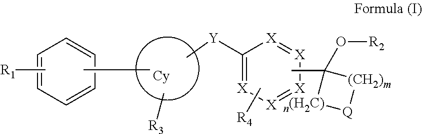

The compounds of the general formula (I) lower or modulate triglyceride levels and/or cholesterol levels and/or low-density lipoproteins (LDL) and raises the high-density lipoproteins (HDL) plasma levels, lower blood glucose and hence are useful in combating different medical conditions, where such lowering of LDL (and/or raising of HDL) is beneficial. Thus, it could be used in the treatment and/or prophylaxis of obesity, hyperlipidemia, hypercholesteremia, hypertension, atherosclerotic disease events, vascular restenosis, diabetes and many other related conditions.

The compounds of general formula (I) are useful to prevent or reduce the risk of developing atherosclerosis, which leads to diseases and conditions such as artereosclerotic cardiovascular diseases, stroke, coronary heart diseases, cerebrovascular diseases, peripheral vessel diseases and related disorders.

These compounds of general formula (I) are useful for the treatment and/or prophylaxis of metabolic disorders loosely defined as Syndrome X. The characteristic features of Syndrome X include initial insulin resistance followed by hyperinsulinemia, dyslipidemia and impaired glucose tolerance. The glucose intolerance can lead to non-insulin dependent diabetes mellitus (NIDDM, Type 2 diabetes), which is characterized by hyperglycemia, which if not controlled may lead to diabetic complications or metabolic disorders caused by insulin resistance. Diabetes is no longer considered to be associated only with glucose metabolism, but it affects anatomical and physiological parameters, the intensity of which vary depending upon stages/duration and severity of the diabetic state. The compounds of this invention are also useful in prevention, halting or slowing progression or reducing the risk of the above mentioned disorders along with the resulting secondary diseases such as cardiovascular diseases, like arteriosclerosis, atherosclerosis; diabetic retinopathy, diabetic neuropathy and renal disease including diabetic nephropathy, glomerulonephritis, glomerular sclerosis, nephrotic syndrome, hypertensive nephrosclerosis and end stage renal diseases, like microalbuminuria and albuminuria, which may be result of hyperglycemia or hyperinsulinemia.

The compounds of the present invention can be useful as aldose reductase inhibitors; for improving cognitive functions in dementia, and in the treatment and/or prophylaxis of disorders such as psoriasis, polycystic ovarian syndrome (PCOS), cancer, osteoporosis, leptin resistance, inflammation and inflammatory bowel diseases, wound healing, xanthoma, pancreatitis, myotonic dystrophy, endothelial cell dysfunction and hyperlipidemia.

### BACKGROUND OF THE INVENTION

Higher LDL cholesterol levels in the plasma increase cardiovascular risk and reduction in the levels of LDL would decrease CVD risk by a comparable percentage (PNAS, 2009, 106, 9546-9547). Clearance of LDL cholesterol from plasma is through the action of LDL receptors in the liver and LDL receptors are cell surface glycoproteins that bind to apolipoprotein B100 (apoB100) on LDL particles with high affinity and mediate their endocytic uptake (Journal of Biological Chemistry, 2009, 284, 10561-10570). Defect in hepatic cholesterol clearance and elevated levels of plasma LDL cholesterol that result from the mutations cause familial hypercholesterolemia. Such mutations are identified in the human LDL receptor and later in apolipoprotein-B (Nature Structural and Molecular Biology, 2007, 14, 413-419). Recently, mutations within certain subtypes of the pro-protein convertasesubtilisin/gene such as the subtype nine (hereinafter “the gene”) were found to represent a third class of mutations associated with autosomal dominant hypercholesterolemia (ADH). The discovery, etiology and functions of this subtype gene is discussed in details in Nature Genetics, 2003, 34, 154-156, Trends in Biochemical Sciences, 2008, 33, 426-434 etc. Several missense mutations (S127R, D129G, F216L, D374H, D374Y) are associated with hypercholesterolemia and premature atherosclerosis (J Lipid Res. 2008, 49, 1333-1343). Loss-of-function mutations (R46L, L253F, A433T) lead to elevated receptor abundance, enhancing clearance of LDL cholesterol from the circulation and reducing cardiovascular risk (Nature Structural and Molecular Biology, 2007, 14, 413-419).

Detailed molecular mechanisms explaining the association of LDLR and the particular subtype gene and LDLR degradation is not very clear (Drug News Perspectives, 2008, 21, 323-330). Because of inhibition of LDLR recycling, number of LDL receptors on the cell surface are decreased and this increases plasma LDL levels (PNAS, 2009, 106, 9546-9547).

Various approaches for inhibiting this particular subtype gene are reported, including gene silencing by siRNA or antisense oligonucleotides, mAb disrupting protein-protein interactions or by peptides; all the above-mentioned strategies have shown lowering of LDL cholesterol which may be effective therapy for treating hypercholesterolemia (Biochemical Journal, 2009, 419, 577-584; PNAS, 2008, 105, 11915-11920; Journal of Lipid Research, 2007, 48, 763-767; PNAS, 2009, 106, 9820-9825). However, very little success has been reported in trying to inhibit this subtype gene by using small molecules. Such small molecule inhibitors have their obvious clinical and therapeutic benefits over the other known approaches as discussed above. We herein disclose novel small molecules which have shown to inhibit this particular gene in in-vitro studies and therefore provide an alternate beneficial approach for treating patients in need of such therapy.

We have disclosed earlier compounds which inhibits this particular gene in patent applications no. WO2015107541, WO2014192023, WO2012090220, WO2014002105. Inhibitors of this gene have been disclosed by few companies in application no WO2014150395, WO2014150326, WO 2014151936, WO2016021706, WO2016055901, WO2017222953, WO2017034990, WO2017034997, WO2017034994, WO2018165718, WO2018053517, WO2018057409, WO2020110009, WO2020150473, WO2020150474, WO 2020028723.

### EMBODIMENTS OF THE INVENTION

The main objective of the present invention is to provide novel heterocyclic derivatives represented by the general formula (I), their tautomeric forms, their stereoisomers, their pharmaceutically acceptable salts, and pharmaceutical compositions containing them or their mixtures thereof.

In an embodiment of the present invention is provided a process for the preparation of novel heterocyclic derivatives represented by the general formula (I), their tautomeric forms, their stereoisomers, their pharmaceutically acceptable salts.

In a further embodiment of the present invention is provided pharmaceutical compositions containing compounds of the general formula (I), their tautomeric forms, their stereoisomers, their pharmaceutically acceptable salts, or their mixtures in combination with suitable pharmaceutical excipients such as carriers, solvents, diluents and other media normally employed in preparing such compositions.

In a further embodiment of the present invention is provided a method for treatment of diseases such as dyslipidemia, hyperlipidemia etc. by providing therapeutically effective amount of the compounds of formula (I) or their pharmaceutically acceptable salts or their suitable pharmaceutical compositions.

The above and other embodiments are described in details hereinafter.

### DETAILED DESCRIPTION OF THE INVENTION

Accordingly, the present invention relates to compounds of the general formula (I),

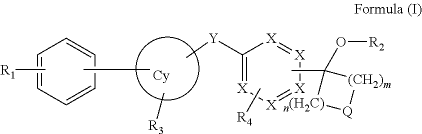

their tautomeric forms, their stereoisomers, their pharmaceutically acceptable salts, and pharmaceutical compositions containing them wherein
‘Cy’ represents heterocyclic groups selected from saturated or partially unsaturated or unsaturated, monocyclic or bicyclic or spirocyclic groups containing 0-4 heteroatoms selected from O, N or S.
‘Y’ at each occurrence independently represents either a bond, or may be selected from O, S(O)O, CO, (C₁-C₃)alkyl, C(O)NR₅, NR₅ or SO₂NR₅; wherein R₅ represents H, (C₁-C₆)alkyl, (C₃-C₆)cycloalkyl;
‘Q’ represents O, S(O)ₒ or NR₇ wherein R₇ represents H, (C₁-C₆)alkyl, (C₃-C₆)cycloalkyl, acyl, —C(O)OR₆, wherein R₆ represents (C₁-C₆) linear or branched alkyl;
‘o’ represents integers from 0-2;
‘m’ and ‘n’ represents integers from 0-4;
‘X’ at each occurrence independently represents either C or N;

R₁ hydrogen, halo or substituted or unsubstituted groups selected from alkyl, haloalkyl, perhaloalkyl, alkoxy, haloalkoxy, perhaloalkoxy, cycloalkyl, alkoxy, cycloalkoxy, aryl, aryloxy, aralkyl, aralkoxy, heterocyclyl, heterocyclylalkyl, heterocyclooxy, heterocyclylalkoxy, heterocyclylalkoxyacyl, carboxylic acid and its derivatives such as esters and amides, carbonylamino, hydroxyalkyl, alkoxyalkyl, aryloxyalkyl, aralkoxyalkyl, alkylthio, thioalkyl, alkylsulfonyl, hydroxyl, sulfonic acid and its derivatives;

R₂ represents hydrogen, or substituted or unsubstituted groups selected from alkyl, haloalkyl, perhaloalkyl, cycloalkyl, deuterated alkyl, alkynyl, alkenyl, aryl, aralkyl, heterocyclyl, heterocyclylalkyl, heterocyclylalkoxyacyl, Acyl, carbonylamino, hydroxyalkyl, alkoxyalkyl, aryloxyalkyl, aralkoxyalkyl, alkylsulfonyl, alkylthioalkyl, alkylsulfinylalkyl, alkylsulfonylalkyl, alkylcarboxylic acid;

R₃ and R₄ independently represents hydrogen, halo, cyano, hydroxy or substituted or unsubstituted groups selected from alkyl, haloalkyl, perhaloalkyl, alkoxy, haloalkoxy, perhaloalkoxy, cycloalkyl, alkoxy, cycloalkoxy, aryl, aryloxy, aralkyl, aralkoxy, heterocyclyl, heterocyclylalkyl, heterocyclooxy, heterocyclylalkoxy, heterocyclylalkoxyacyl, carboxylic acid and its derivatives such as esters and amides, carbonylamino, hydroxyalkyl, alkoxyalkyl, aryloxyalkyl, aralkoxyalkyl, alkylthio, thioalkyl, alkylsulfonyl, hydroxyl, sulfonic acid and its derivatives.

When ‘Cy’ is substituted, the substituents at each occurrence may be independently selected from hydroxyl, oxo, halo, thiol, nitro, amino, cyano, formyl, or substituted or unsubstituted groups selected from amidino, alkyl, haloalkyl, perhaloalkyl, alkoxy, haloalkoxy, perhaloalkoxy, alkenyl, alkynyl, cycloalkyl, cycloalkenyl, bicycloalkyl, bicycloalkenyl, alkoxy, alkenoxy, cycloalkoxy, aryl, aryloxy, aralkyl, aralkoxy, heterocyclyl, heterocyclylalkyl, heterocyclooxy, heterocyclylalkoxy, heterocyclylalkoxyacyl, acyl, acycloxy, acylamino, monosubstituted or disubstituted amino, arylamino, aralkylamino, carboxylic acid and its derivatives such as esters and amides, carbonylamino, hydroxyalkyl, aminoalkyl, alkoxyalkyl, aryloxyalkyl, aralkoxyalkyl, alkylthio, thioalkyl, cycloalkylthio, arylthio, heterocyclylthio, alkylsulfinyl, cycloalkylsulfinyl, arylsulfinyl, heterocyclylsulfinyl, alkylsulfonyl, cycloalkylsulfonyl, arylsulfonyl, heterocyclylsulfonyl, alkylsulfonylamino, cycloalkylsulfonylamino, arylsulfonylamino, heterocyclylsulfonyl amino, alkylsulfonyloxy, cycloalkylsulfonyloxy, arylsulfonyloxy, heterocyclylsulfonyloxy, alkoxycarbonylamino, aryloxycarbonylamino, aralkyloxycarbonylamino, aminocarbonylamino, alkylaminocarbonylamino, alkoxyamino, hydroxyl amino, sulfinyl derivatives, sulfonyl derivatives, sulfonic acid and its derivatives.

When the substituents on any of ‘Cy’ or R₁, are further substituted, the substituents may be selected from one or more groups described above.

In a preferred embodiment, ‘Cy’ is saturated or partially unsaturated or unsaturated monocyclic or bicyclic, or spirocyclic groups containing 0-3 N, O, S atoms such as pyrrolidinyl, piperidinyl, piperazinyl, diazepinyl, oxazolyl, oxadiazolyl, indolinyl, pyridothienyl, hexahydrocyclopenta[c]pyrrol, hexahydropyrrolo[3,4-c]pyrrol, dihydropyrrolo[3,4-c]pyrazol, 5H-imidazo[4,5-c]pyridine, 5,6-dihydropyridin-, hexahydrocyclopenta[c]pyrrol, 3,9-diazaspiro[5.5]undecan, tetrahydropyrrolo[3,4-c]pyrrol, 3,4-dihydroisoquinolin-3,3a,6,6a-tetrahydrocyclopenta[c]pyrrol-, 3,6-diazabicyclo[3.1.1]heptan, dihydrothieno[3,2-c]pyridine, dihydrothiazolo[5,4-c]pyridine, diazaspiro[4.5]decan and the like.

In another preferred embodiment, ‘Y’ is selected form a bond, O, S(O)ₒ, CO, C(O)NR₅, wherein R₅ represents H.

The various groups, radicals and substituents used anywhere in the specification are described in the following paragraphs.

In a further preferred embodiment the groups, radicals described above may be selected from:
the “alkyl” group used either alone or in combination with other radicals, denotes a linear or branched radical containing one to six carbons, selected from methyl, ethyl, n-propyl, iso-propyl, n-butyl, sec-butyl, tert-butyl, amyl, t-amyl, n-pentyl, n-hexyl, and the like;
the “alkenyl” group used either alone or in combination with other radicals, is selected from a radical containing from two to six carbons, more preferably groups selected from vinyl, allyl, 2-butenyl, 3-butenyl, 2-pentenyl, 3-pentenyl, 4-pentenyl, 2-hexenyl, 3-hexenyl, 4-hexenyl and the like; the “alkenyl” group includes dienes and trienes of straight and branched chains;
the “cycloalkyl”, or “alicyclic” group used either alone or in combination with other radicals, is selected from a cyclic radical containing three to six carbons, more preferably cyclopropyl, cyclobutyl, cyclopentyl, cyclohexyl and the like;
the “cycloalkenyl” group used either alone or in combination with other radicals, are preferably selected from cyclopropenyl, 1-cyclobutenyl, 2-cyclobutenyl, 1-cyclopentenyl, 2-cyclopentenyl, 3-cyclopentenyl, 1-cyclohexenyl, 2-cyclohexenyl, 3-cyclohexenyl and the like; The terms “bicycloalkenyl” means more than one cycloalkenyl groups fused together;
the “alkoxy” group used either alone or in combination with other radicals, is selected from groups containing an alkyl radical, as defined above, attached directly to an oxygen atom, more preferably groups selected from methoxy, ethoxy, n-propoxy, iso-propoxy, n-butoxy, t-butoxy, iso-butoxy, pentyloxy, hexyloxy, and the like;
the “cycloalkoxy” group used either alone or in combination with other radicals, is selected from a cyclic radical containing three to seven carbons, more preferably cyclopropyloxy, cyclobutylxoy, cyclopentyloxy, cyclohexyloxy and the like; The terms “bicycloalkyloxy” means more than one cycloalkyl groups fused together;
the “alkenoxy” group used either alone or in combination with other radicals, is selected from groups containing an alkenyl radical, as defined above, attached to an oxygen atom, more preferably selected from vinyloxy, allyloxy, butenoxy, pentenoxy, hexenoxy, and the like;
the “haloalkyl” group is selected from an alkyl radical, as defined above, suitably substituted with one or more halogens; such as perhaloalkyl, more preferably, perfluoro(C₁-C₆)alkyl such as fluoromethyl, difluoromethyl, trifluoromethyl, fluoroethyl, difluoroethyl, trifluoroethyl, mono or polyhalo substituted methyl, ethyl, propyl, butyl, pentyl or hexyl groups;
the “haloalkoxy” group is selected from suitable haloalkyl, as defined above, directly attached to an oxygen atom, more preferably groups selected from fluoromethoxy, chloromethoxy, fluoroethoxy, chloroethoxy and the like;
the “aryl” or “aromatic” group used either alone or in combination with other radicals, is selected from a suitable aromatic system containing one, two or three rings wherein such rings may be attached together in a pendant manner or may be fused, more preferably the groups are selected from phenyl, naphthyl, tetrahydronaphthyl, indane, biphenyl, and the like;
the “aryloxy” group used either alone or in combination with other radicals, is selected from groups containing an aryl radical, as defined above, attached directly to an oxygen atom, more preferably groups selected from phenoxy, naphthyloxy, tetrahydronaphthyloxy, biphenyloxy, and the like;
the “heterocyclyl” or “heterocyclic” group used either alone or in combination with other radicals, is selected from suitable aromatic or non-aromatic radicals containing one or more hetero atoms selected from O, N or S. The non-aromatic radicals may be saturated, partially saturated or unsaturated mono, bi or tricyclic radicals, containing one or more heteroatoms selected from nitrogen, sulfur and oxygen, more preferably selected from aziridinyl, azetidinyl, pyrrolidinyl, imidazolidinyl, piperidinyl, piperazinyl, 2-oxopiperidinyl, 4-oxopiperidinyl, 2-oxopiperazinyl, 3-oxopiperazinyl, morpholinyl, thiomorpholinyl, 2-oxomorpholinyl, azepinyl, diazepinyl, oxapinyl, thiazepinyl, oxazolidinyl, thiazolidinyl, dihydrothiophene, dihydropyran, dihydrofuran, dihydrothiazole, benzopyranyl, benzopyranonyl, benzodihydrofuranyl, benzodihydrothienyl, pyrazolopyrimidonyl, azaquinazolinoyl, thienopyrimidonyl, quinazolonyl, pyrimidonyl, benzoxazinyl, benzoxazinonyl, benzothiazinyl, benzothiazinonyl, thieno piperidinyl, and the like; the aromatic radicals, may be selected from suitable single or fused mono, bi or tricyclic aromatic heterocyclic radicals containing one or more hetero atoms selected from O, N or S, more preferably the groups are selected from pyridyl, thienyl, furyl, pyrrolyl, oxazolyl, thiazolyl, isothiazolyl, imidazolyl, isoxazolyl, oxadiazolyl, thiadiazolyl, triazolyl, tetrazolyl, benzofuranyl, benzothienyl, indolinyl, indolyl, azaindolyl, azaindolinyl, pyrazolopyrimidinyl, azaquinazolinyl, pyridofuranyl, pyridothienyl, thienopyrimidyl, quinolinyl, pyrimidinyl, pyrazolyl, quinazolinyl, pyridazinyl, triazinyl, benzimidazolyl, benzotriazolyl, phthalazynil, naphthylidinyl, purinyl, carbazolyl, phenothiazinyl, phenoxazinyl, benzoxazolyl, benzothiazolyl hexahydrocyclopenta[c]pyrrol, hexahydropyrrolo[3,4-c]pyrrol, dihydropyrrolo[3,4-c]pyrazol, 5H-imidazo[4,5-c]pyridine, 5,6-dihydropyridin-, hexahydrocyclopenta[c]pyrrol, 3,9-diazaspiro[5.5]undecan, tetrahydropyrrolo[3,4-c]pyrrol, 3,4-dihydroisoquinolin-3,3a,6,6a-tetrahydrocyclopenta[c]pyrrol-, 3,6-diazabicyclo[3.1.1]heptan, dihydrothieno[3,2-c]pyridine, dihydrothiazolo[5,4-c]pyridine, diazaspiro[4.5]decan and the like;
the groups “heterocyclooxy”, “heterocyclylalkoxy” are selected from suitable heterocyclyl, heterocyclylalkyl groups respectively, as defined above, attached to an oxygen atom;
the “acyl” group used either alone or in combination with other radicals, is selected from a radical containing one to eight carbons, more preferably selected from formyl, acetyl, propanoyl, butanoyl, iso-butanoyl, pentanoyl, hexanoyl, heptanoyl, benzoyl and the like, which may be substituted;
the “acycloxy” group used either alone or in combination with other radicals, is selected from a suitable acyl group, as defined above, directly attached to an oxygen atom, more preferably such groups are selected from acetyloxy, propionyloxy, butanoyloxy, iso-butanoyloxy, benzoyloxy and the like;
the “acylamino” group used either alone or in combination with other radicals, is selected from a suitable acyl group as defined earlier, attached to an amino radical, more preferably such groups are selected from CH₃CONH, C₂H₅CONH, C₃H₇CONH, C₄H₉CONH, C₆H₅CONH and the like, which may be substituted;
the “mono-substituted amino” group used either alone or in combination with other radicals, represents an amino group substituted with one group selected from (C₁-C₆)alkyl, substituted alkyl, aryl, substituted aryl or arylalkyl groups as defined earlier, more preferably such groups are selected from methylamine, ethylamine, n-propylamine, n-butylamine, n-pentylamine and the like;
the ‘disubstituted amino” group used either alone or in combination with other radicals, represents an amino group, substituted with two radicals that may be same or different selected from (C₁-C₆)alkyl, substituted alkyl, aryl, substituted aryl, or arylalkyl groups, as defined above, more preferably the groups are selected from dimethylamino, methylethylamino, diethylamino, phenylmethyl amino and the like;
the “arylamino” used either alone or in combination with other radicals, represents an aryl group, as defined above, linked through amino having a free valence bond from the nitrogen atom, more preferably the groups are selected from phenylamino, naphthylamino, N-methyl anilino and the like;
the “oxo” or “carbonyl” group used either alone (—C═O—) or in combination with other radicals such as alkyl described above, for e.g. “alkylcarbonyl”, denotes a carbonyl radical (—C═O—) substituted with an alkyl radical described above such as acyl or alkanoyl;
the “carboxylic acid” group, used alone or in combination with other radicals, denotes a —COOH group, and includes derivatives of carboxylic acid such as esters and amides;
the “ester” group used alone or in combination with other radicals, denotes —COO— group, and includes carboxylic acid derivatives, more preferably the ester moieties are selected from alkoxycarbonyl, such as methoxycarbonyl, ethoxycarbonyl, and the like, which may optionally be substituted; aryloxycarbonyl group such as phenoxycarbonyl, napthyloxycarbonyl, and the like, which may optionally be substituted; aralkoxycarbonyl group such as benzyloxycarbonyl, phenethyloxycarbonyl, napthylmethoxycarbonyl, and the like, which may optionally be substituted; heteroaryloxycarbonyl, heteroaralkoxycarbonyl, wherein the heteroaryl group, is as defined above, which may optionally be substituted; heterocyclyloxycarbonyl, where the heterocyclic group, as defined earlier, which may optionally be substituted;
the “amide” group used alone or in combination with other radicals, represents an aminocarbonyl radical (H₂N—C═O), wherein the amino group is mono- or di-substituted or unsubstituted, more preferably the groups are selected from methyl amide, dimethyl amide, ethyl amide, diethyl amide, and the like;
the “aminocarbonyl” group used either alone or in combination with other radicals, may be selected from ‘aminocarbonyl’, ‘aminocarbonylalkyl”, “n-alkylaminocarbonyl”, “N-arylaminocarbonyl”, “N,N-dialkylaminocarbonyl”, “N-alkyl-N-arylaminocarbonyl”, “N-alkyl-N-hydroxyaminocarbonyl”, and “N-alkyl-N-hydroxyaminocarbonylalkyl”, each of them being optionally substituted. The terms “N-alkylaminocabonyl” and “N,N-dialkylaminocarbonyl” denotes aminocarbonyl radicals, as defined above, which have been substituted with one alkyl radical and with two alkyl radicals, respectively. Preferred are “lower alkylaminocarbonyl” having lower alkyl radicals as described above attached to aminocarbonyl radical. The terms “N-arylaminocarbonyl” and “N-alkyl-N-arylaminocarbonyl” denote aminocarbonyl radicals substituted, respectively, with one aryl radical, or one alkyl, and one aryl radical. The term “aminocarbonylalkyl” includes alkyl radicals substituted with aminocarbonyl radicals;
the “hydroxyalkyl” group used either alone or in combination with other radicals, is selected from an alkyl group, as defined above, substituted with one or more hydroxy radicals, more preferably the groups are selected from hydroxymethyl, hydroxyethyl, hydroxypropyl, hydroxybutyl, hydroxypentyl, hydroxyhexyl and the like;
the “aminoalkyl” group used alone or in combination with other radicals, denotes an amino (—NH₂) moiety attached to an alkyl radical, as defined above, which may be substituted, such as mono- and di-substituted aminoalkyl. The term “alkylamino” used herein, alone or in combination with other radicals, denotes an alkyl radical, as defined above, attached to an amino group, which may be substituted, such as mono- and di-substituted alkylamino;
the “alkoxyalkyl” group used alone or in combination with other radicals, denotes an alkoxy group, as defined above, attached to an alkyl group as defined above, more preferably the groups may be selected from methoxymethyl, ethoxymethyl, methoxyethyl, ethoxyethyl and the like;
the “alkylthio” group used either alone or in combination with other radicals, denotes a straight or branched or cyclic monovalent substituent comprising an alkyl group as defined above, linked through a divalent sulfur atom having a free valence bond from the sulfur atom, more preferably the groups may be selected from methylthio, ethylthio, propylthio,
the “thioalkyl” group used either alone or in combination with other radicals, denotes an alkyl group, as defined above, attached to a group of formula —SR′, where R′ represents hydrogen, alkyl or aryl group, e.g. thiomethyl, methylthiomethyl, phenylthiomethyl and the like, which may be optionally substituted.
the “alkoxycarbonylamino” group used alone or in combination with other radicals, is selected from a suitable alkoxycarbonyl group, as defined above, attached to an amino group, more preferably methoxycarbonylamino, ethoxycarbonylamino, and the like;
the “arylthio” group used either alone or in combination with other radicals, denotes a an aryl group as defined above, linked through a divalent sulfur atom having a free valence bond from the sulfur atom, more preferably the groups may be selected from phenylthio, naphthylthio, tetrahydronaphthylthio, indanethio, biphenylthio, and the like;
the “heterocyclylthio” group used either alone or in combination with other radicals, denotes an heterocyclyl group as defined above, linked through a divalent sulfur atom having a free valence bond from the sulfur atom, more preferably the groups may be selected from aziridinylthio, azetidinylthio, pyrrolidinylthio, imidazolidinylthio, piperidinylthio, piperazinylthio, 2-oxopiperidinylthio, 4-oxopiperidinylthio, 2-oxopiperazinylthio, 3-oxopiperazinylthio, morpholinylthio, thiomorpholinylthio, 2-oxomorpholinylthio, azepinylthio, diazepinylthio, oxapinylthio, thiazepinylthio, oxazolidinylthio, thiazolidinylthio, dihydrothiophenethio, dihydropyranthio, dihydrofuranthio, dihydrothiazolethio, benzopyranylthio, benzopyranonylthio, benzodihydrofuranylthio, benzodihydrothienylthio, pyrazolopyrimidonylthio, azaquinazolinoylthio, thienopyrimidonylthio, quinazolonylthio, pyrimidonylthio, benzoxazinylthio, benzoxazinonylthio, benzothiazinylthio, benzothiazinonylthio, thieno piperidinylthio, pyridylthio, thienylthio, furylthio, pyrrolylthio, oxazolylthio, thiazolylthio, isothiazolylthio, imidazolylthio, isoxazolylthio, oxadiazolylthio, thiadiazolylthio, triazolylthio, tetrazolylthio, benzofuranylthio, benzothienylthio, indolinylthio, indolylthio, azaindolylthio, azaindolinylthio, pyrazolopyrimidinylthio, azaquinazolinylthio, pyridofuranylthio, pyridothienylthio, thienopyrimidylthio, quinolinylthio, pyrimidinylthio, pyrazolylthio, quinazolinylthio, pyridazinylthio, triazinylthio, benzimidazolylthio, benzotriazolylthio, phthalazynilthio, naphthylidinylthio, purinylthio, carbazolylthio, phenothiazinylthio, phenoxazinylthio, benzoxazolylthio, benzothiazolylthio and the like;
the “alkoxycarbonylamino” group used alone or in combination with other radicals, is selected from a suitable alkoxycarbonyl group, as defined above, attached to an amino group, more preferably methoxycarbonylamino, ethoxycarbonylamino, and the like;
the “aminocarbonylamino”, “alkylaminocarbonylamino”, “dialkylaminocarbonylamino” groups used alone or in combination with other radicals, is a carbonylamino (—CONH₂) group, attached to amino(NH₂), alkylamino group or dialkylamino group respectively, where alkyl group is as defined above;
the “amidino” group used either alone or in combination with other radicals, represents a —C(═NH)—NH₂ radical; the “alkylamidino” group represents an alkyl radical, as described above, attached to an amidino group;
the “alkoxyamino” group used either alone or in combination with other radicals, represents a suitable alkoxy group as defined above, attached to an amino group;
the “hydroxyamino” group used either alone or in combination with other radicals, represents a —NHOH moiety, and may be optionally substituted with suitable groups selected from those described above;
the “sulfenyl” group or “sulfenyl derivatives” used alone or in combination with other radicals, represents a bivalent group, —SO— or RₓSO, where Rₓ is an optionally substituted alkyl, aryl, heteroaryl, heterocyclyl, group selected from those described above;
the “sulfonyl” group or “sulfones derivatives” used either alone or in combination with other radicals, with other terms such as alkylsulfonyl, represents a divalent radical —SO₂—, or RₓSO₂—, where Rₓ is as defined above. More preferably, the groups may be selected from “alkylsulfonyl” wherein suitable alkyl radicals, selected from those defined above, is attached to a sulfonyl radical, such as methylsulfonyl, ethylsulfonyl, propylsulfonyl and the like, “arylsulfonyl” wherein an aryl radical, as defined above, is attached to a sulfonyl radical, such as phenylsulfonyl and the like.
The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.
The phrase “therapeutically effective” is intended to qualify the amount of active ingredients used in the treatment of a disease or disorder. This amount will achieve the goal of reducing or eliminating the said disease or disorder.
The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.
As used herein, reference to “treatment” of a patient is intended to include prophylaxis. The term “patient” means all mammals including humans. Examples of patients include humans, cows, dogs, cats, goats, sheep, pigs, and rabbits. Preferably, the patient is a human.

Suitable groups and substituents on the groups may be selected from those described anywhere in the specification.

Particularly useful compounds may be selected from—
(4-(3-hydroxyoxetan-3-yl)phenyl)(4-(4-(trifluoromethyl)phenyl)piperidin-1-yl) methanone;
(4-(3-methoxyoxetan-3-yl)phenyl)(4-(4-(trifluoromethyl)phenyl)piperidin-1-yl) methanone;
N-(4-(3-hydroxyoxetan-3-yl)phenyl)-1-(4-(trifluoromethyl)phenyl)piperidine-4-carboxamide;
N-(4-(3-hydroxyoxetan-3-yl)phenyl)-4-(4-(trifluoromethyl)phenyl)piperidine-1-carboxamide;
N-(4-(3-methoxyoxetan-3-yl)phenyl)-4-(4-(trifluoromethyl)phenyl)piperidine-1-carboxamide;
(4-(3-hydroxyoxetan-3-yl)phenyl)(4-(4-(trifluoromethyl)phenyl)piperazin-1-yl) methanone;
3-(4-((4-(4-(trifluoromethyl)phenyl)piperazin-1-yl)sulfonyl)phenyl)oxetan-3-ol;
3-(4-(4-(4-(trifluoromethyl)phenyl)piperidin-1-yl)phenyl)oxetan-3-ol;
1-(4-(3-methoxyoxetan-3-yl)phenyl)-4-(4-(trifluoromethyl)phenyl)piperidine;
(4-(3-hydroxyoxetan-3-yl)phenyl)(5-(4-(trifluoromethyl)phenyl)hexahydrocyclopenta[c]pyrrol-2(1H)-yl)methanone;
(4-(3-hydroxyoxetan-3-yl)phenyl)(5-(4-(trifluoromethyl)phenyl)hexahydropyrrolo[3,4-c]pyrrol-2(1H)-yl)methanone;
(4-(3-(2-hydroxyethoxy)oxetan-3-yl)phenyl)(5-(4-(trifluoromethyl)phenyl)hexahydropyrrolo[3,4-c]pyrrol-2(1H)-yl)methanone;
2-((3-(4-(5-(4-(trifluoromethyl)phenyl)octahydropyrrolo[3,4-c]pyrrole-2-carbonyl) phenyl)oxetan-3-yl)oxy)acetic acid;
(4-(3-fluoro-5-(trifluoromethyl)phenyl)piperidin-1-yl)(4-(3-hydroxyoxetan-3-yl) phenyl)methanone;
(4-(3-hydroxyoxetan-3-yl)phenyl)(4-(3-(4-methyl-1H-imidazol-1-yl)-5-(trifluoromethyl)phenyl)piperidin-1-yl)methanone;
3-(4-(3-(3-fluoro-4-(trifluoromethyl)phenyl)-1,2,4-oxadiazol-5-yl)phenyl)oxetan-3-ol;
3-(4-((1-(4-(trifluoromethyl)phenyl)piperidin-4-yl)oxy)phenyl)oxetan-3-ol;
(4-(3-hydroxyoxetan-3-yl)phenyl)(2-(4-(trifluoromethyl)phenyl)-6,7-dihydrothieno [3,2-c]pyridin-5(4H)-yl)methanone;
(1-benzyl-3-(4-(trifluoromethyl)phenyl)-4,6-dihydropyrrolo[3,4-c]pyrazol-5(1H)-yl) (4-(3-hydroxyoxetan-3-yl)phenyl)methanone;
(4-(3-hydroxyoxetan-3-yl)phenyl)(1-methyl-3-(4-(trifluoromethyl)phenyl)-4,6-dihydropyrrolo[3,4-c]pyrazol-5(1H)-yl)methanone;
(4-(3-hydroxyoxetan-3-yl)phenyl)(2-methyl-3-(4-(trifluoromethyl)phenyl)-2,6-dihydropyrrolo[3,4-c]pyrazol-5(4H)-yl)methanone;
(4-(3-hydroxyoxetan-3-yl)phenyl)(4-(4-(trifluoromethoxy)phenyl)piperidin-1-yl) methanone;
(4-(3-hydroxyoxetan-3-yl)phenyl)(4-(p-tolyl)piperidin-1-yl)methanone;
(4-(3-hydroxyoxetan-3-yl)phenyl)(4-(3-(trifluoromethyl)phenyl)piperidin-1-yl) methanone;
(3-(3-hydroxyoxetan-3-yl)phenyl)(4-(p-tolyl)piperidin-1-yl)methanone;
(4-(3-chloro-4-methoxyphenyl)piperidin-1-yl)(4-(3-hydroxyoxetan-3-yl)phenyl) methanone;
(4-(3-hydroxyoxetan-3-yl)phenyl)(4-(4-methoxyphenyl)piperidin-1-yl)methanone;
(4-(3-fluoro-4-methoxy phenyl)piperidin-1-yl)(4-(3-hydroxyoxetan-3-yl)phenyl) methanone;
(4-(3-chloro-4-methylphenyl)piperidin-1-yl)(4-(3-hydroxyoxetan-3-yl)phenyl) methanone;
(4-(3-hydroxyoxetan-3-yl)phenyl)(4-(3-methoxy-4-(trifluoromethyl)phenyl) piperidin-1-yl)methanone;
(4-(3-fluoro-4-(trifluoromethyl)phenyl)piperidin-1-yl)(4-(3-hydroxyoxetan-3-yl) phenyl)methanone;
(4-(3-chloro-4-(trifluoromethyl)phenyl)piperidin-1-yl)(4-(3-hydroxyoxetan-3-yl) phenyl) methanone;
(4-(3-hydroxyoxetan-3-yl)phenyl)(4-(2-(trifluoromethyl)phenyl)piperidin-1-yl) methanone;
(4-(3-hydroxyoxetan-3-yl)phenyl)(3-(4-(trifluoromethyl)phenyl)piperidin-1-yl) methanone;
2-((3-(4-(4-(4-(trifluoromethyl)phenyl)piperidine-1-carbonyl)phenyl)oxetan-3-yl) oxy)acetic acid;
3-(4-(4-(4-(trifluoromethyl)phenyl)piperidine-1-carbonyl)phenyl) oxetan-3-yl acetate;
(4-(3-methoxyoxetan-3-yl)phenyl)(4-(3-(trifluoromethyl)phenyl)piperidin-1-yl) methanone;
(4-(3-fluoro-4-(trifluoromethyl)phenyl)piperidin-1-yl)(4-(3-methoxyoxetan-3-yl) phenyl) methanone;
(4-(3-methoxyoxetan-3-yl)phenyl)(3-(4-(trifluoromethyl)phenyl)piperidin-1-yl) methanone;
(4-(3-ethoxy oxetan-3-yl)phenyl)(4-(3-(trifluoromethyl)phenyl)piperidin-1-yl) methanone;
(4-(3-methoxyoxetan-3-yl)phenyl)(4-(2-(trifluoromethyl)phenyl)piperidin-1-yl) methanone;
(4-(3-ethoxy oxetan-3-yl)phenyl)(4-(3-fluoro-4-(trifluoromethyl)phenyl)piperidin-1-yl) methanone;
(4-(3-ethoxy oxetan-3-yl)phenyl)(4-(4-(trifluoromethyl)phenyl)piperidin-1-yl) methanone;
(4-(3-isopropoxyoxetan-3-yl)phenyl)(4-(4-(trifluoromethyl)phenyl)piperidin-1-yl) methanone;
(4-(3-isobutoxyoxetan-3-yl)phenyl)(4-(4-(trifluoromethyl)phenyl)piperidin-1-yl) methanone;
(4-(3-(cyclopropylmethoxy)oxetan-3-yl)phenyl)(4-(4-(trifluoromethyl)phenyl) piperidin-1-yl) methanone;
(3-(3-hydroxyoxetan-3-yl)phenyl)(4-(4-(trifluoromethyl)phenyl)piperidin-1-yl) methanone;
(3-(3-hydroxyoxetan-3-yl)phenyl)(3-(4-(trifluoromethyl)phenyl)piperidin-1-yl) methanone;
(5-(3-hydroxyoxetan-3-yl)pyridin-3-yl)(4-(4-(trifluoromethyl)phenyl)piperidin-1-yl) methanone;
(3-(3-methoxyoxetan-3-yl)phenyl)(4-(4-(trifluoromethyl)phenyl)piperidin-1-yl) methanone;
(3-(3-methoxyoxetan-3-yl)phenyl)(3-(4-(trifluoromethyl)phenyl)piperidin-1-yl) methanone;
N-(3-(3-hydroxyoxetan-3-yl)phenyl)-4-(4-(trifluoromethyl)phenyl)piperidine-1-carboxamide;
(4-(3-methoxyoxetan-3-yl)phenyl)(4-(4-(trifluoromethyl)phenyl)piperazin-1-yl) methanone;
3-(4-((4-(4-(trifluoromethyl)phenyl)piperidin-1-yl)sulfonyl)phenyl)oxetan-3-ol;
(3-(3-hydroxyoxetan-3-yl)phenyl)(5-(4-(trifluoromethyl)phenyl)hexahydropyrrolo[3,4-c]pyrrol-2(1H)-yl)methanone;
(3-(3-methoxyoxetan-3-yl)phenyl)(5-(4-(trifluoromethyl)phenyl)hexahydropyrrolo[3,4-c]pyrrol-2(1H)-yl)methanone;
(4-(3-methoxyoxetan-3-yl)phenyl)(5-(4-(trifluoromethyl)phenyl)hexahydropyrrolo[3,4-c]pyrrol-2(1H)-yl)methanone;
(4-(3-ethoxy oxetan-3-yl)phenyl)(5-(4-(trifluoromethyl)phenyl)hexahydropyrrolo[3,4-c]pyrrol-2(1H)-yl)methanone;
(4-(4-(di fluoromethyl) phenyl) piperidin-1-yl)(3-(3-(methoxy-d3) oxetan-3-yl) phenyl)methanone;
N-(4-(3-methoxyoxetan-3-yl) phenyl)-1-(4-(trifluoromethyl) phenyl) piperidine-4-carboxamide;
(4-(3-hydroxy-1,1-dioxidothietan-3-yl)phenyl)(5-(4-(trifluoromethyl)phenyl) hexahydropyrrolo[3,4-c]pyrrol-2(1H)-yl)methanone;
(4-(3-hydroxy-1,1-dioxidothietan-3-yl)phenyl)(4-(4-(trifluoromethyl)phenyl) piperidin-1-yl) methanone;
(4-(3-hydroxyoxetan-3-yl) phenyl)(4-(4-hydroxyphenyl) piperidin-1-yl)methanone;
(4-(3-hydroxyoxetan-3-yl)phenyl)(4-phenyl piperidin-1-yl)methanone;
(4-(4-(difluoromethyl)phenyl)piperidin-1-yl)(3-(3-methoxyoxetan-3-yl)phenyl) methanone;
(4-(4-(di fluoromethyl)phenyl)piperidin-1-yl)(3-(3-hydroxyoxetan-3-yl)phenyl) methanone;
(4-(3-hydroxyoxetan-3-yl)phenyl)(2-(2-methoxypyridin-4-yl)-4-(4-(trifluoromethyl) phenyl)piperazin-1-yl)methanone;
(4-(4-(difluoromethyl)phenyl)piperidin-1-yl)(4-(3-methoxyoxetan-3-yl)phenyl) methanone;
(4-(4-(di fluoromethyl)phenyl)piperidin-1-yl)(4-(3-hydroxyoxetan-3-yl)phenyl) methanone;
(4-(3-(but-2-yn-1-yloxy)oxetan-3-yl)phenyl)(4-(4-(trifluoromethyl)phenyl)piperidin-1-yl) methanone;
N-(4-(3-hydroxyoxetan-3-yl)phenyl)-5-phenyl-2-(4-(trifluoromethyl)phenyl)oxazole-4-carboxamide;
(4-(3-(prop-2-yn-1-yloxy)oxetan-3-yl)phenyl)(4-(4-(trifluoromethyl)phenyl) piperidin-1-yl) methanone;
4-(4-(3-methoxyoxetan-3-yl)phenyl)-1-(4-(trifluoromethyl)phenyl)piperidin-4-ol;
N-(4-(3-methoxyoxetan-3-yl)phenyl)-5-(4-(trifluoromethyl)phenyl) hexahydropyrrolo[3,4-c]pyrrole-2(1H)-carboxamide;
N-(4-(3-hydroxyoxetan-3-yl)phenyl)-5-(4-(trifluoromethyl)phenyl) hexahydropyrrolo[3,4-c]pyrrole-2(1H)-carboxamide;
(4-(3-(methoxy-d3)oxetan-3-yl)phenyl)(4-(4-(trifluoromethyl)phenyl)piperidin-1-yl) methanone;
5-(3-hydroxyoxetan-3-yl)-2-(4-(4-(trifluoromethyl) phenyl) piperidin-1-yl) benzoic acid;
3-(3-hydroxyoxetan-3-yl)-5-(4-(4-(trifluoromethyl)phenyl)piperidin-1-yl)benzoic acid;
(3-(3-hydroxyoxetan-3-yl)phenyl)(2-(4-(trifluoromethyl)phenyl)-1,4,6,7-tetrahydro-5H-imidazo[4,5-c]pyridin-5-yl)methanone;
(4-(3-hydroxyoxetan-3-yl)phenyl)(2-(4-(trifluoromethyl)phenyl)-1,4,6,7-tetrahydro-5H-imidazo[4,5-c]pyridin-5-yl)methanone;
(4-(3-hydroxyoxetan-3-yl)-2-morpholinophenyl)(4-(4-(trifluoromethyl)phenyl) piperidin-1-yl) methanone;
(4-(3-hydroxyoxetan-3-yl)-2-(piperazin-1-yl)phenyl)(4-(4-(trifluoromethyl)phenyl) piperidin-1-yl)methanone;
1-(4-(3-ethoxyoxetan-3-yl)phenyl)-4-(4-(trifluoromethyl)phenyl)piperidine;
3-(3-morpholino-5-(4-(4-(trifluoromethyl)phenyl)piperidin-1-yl)phenyl)oxetan-3-ol;
tert-butyl4-(5-(3-hydroxyoxetan-3-yl)-2-(4-(4-(trifluoromethyl)phenyl)piperidine-1-carbonyl) phenyl)piperazine-1-carboxylate;
(4-hydroxy-4-(4-(trifluoromethyl)phenyl)piperidin-1-yl)(4-(3-hydroxyoxetan-3-yl) phenyl) methanone;
(4-(3-hydroxyoxetan-3-yl)phenyl)(4-(4-(trifluoromethyl)phenyl)-5,6-dihydropyridin-1(2H)-yl)methanone;
(4-(3-hydroxyoxetan-3-yl)phenyl)(1-(4-(trifluoromethyl)phenyl)-4,6-dihydropyrrolo [3,4-c]pyrazol-5(1H)-yl)methanone;
(4-(3-hydroxyoxetan-3-yl)phenyl)(2-(4-(trifluoromethyl)phenyl)-2,6-dihydropyrrolo [3,4-c]pyrazol-5(4H)-yl)methanone;
(4-(3-methoxyoxetan-3-yl)phenyl)(2-(4-(trifluoromethyl)phenyl)-2,6-dihydropyrrolo [3,4-c]pyrazol-5(4H)-yl)methanone;
(4-(3-hydroxyoxetan-3-yl)phenyl)(5-methoxy-5-(4-(trifluoromethyl)phenyl) hexahydrocyclopenta[c]pyrrol-2(1H)-yl)methanone;
(5-methoxy-5-(4-(trifluoromethyl)phenyl)hexahydrocyclopenta[c]pyrrol-2(1H)-yl)(4-(3-methoxyoxetan-3-yl)phenyl)methanone;
(4-(3-hydroxyoxetan-3-yl)phenyl)(5-(4-(trifluoromethyl)phenyl)-3,5-dihydropyrrolo [3,4-c]pyrrol-2(1H)-yl)methanone;
(4-(3-methoxyoxetan-3-yl)phenyl)(5-(4-(trifluoromethyl)phenyl)-3,5-dihydropyrrolo[3,4-c]pyrrol-2(1H)-yl)methanone;
(4-(3-hydroxyoxetan-3-yl)phenyl)(5-(4-(trifluoromethyl)phenyl)-3,4,5,6-tetrahydropyrrolo[3,4-c]pyrrol-2(1H)-yl)methanone;
(3-(3-hydroxyoxetan-3-yl)phenyl)(5-(4-(trifluoromethyl)phenyl)-3,4,5,6-tetrahydropyrrolo[3,4-c]pyrrol-2(1H)-yl)methanone;
(3-(3-methoxyoxetan-3-yl)phenyl)(5-(4-(trifluoromethyl)phenyl)-3,4,5,6-tetrahydropyrrolo[3,4-c]pyrrol-2(1H)-yl)methanone;
3-(4-((5-(4-(trifluoromethyl)phenyl)hexahydropyrrolo[3,4-c]pyrrol-2(1H)-yl)sulfonyl) phenyl)oxetan-3-ol;
(4-(3-hydroxyoxetan-3-yl)phenyl)(9-(4-(trifluoromethyl)phenyl)-3,9-diazaspiro [5.5]undecan-3-yl)methanone;
(3-(3-hydroxyoxetan-3-yl)phenyl)(9-(4-(trifluoromethyl)phenyl)-3,9-diazaspiro [5.5]undecan-3-yl)methanone;
(4-(3-methoxyoxetan-3-yl)phenyl)(9-(4-(trifluoromethyl)phenyl)-3,9-diazaspiro [5.5]undecan-3-yl)methanone;
(4-(3-hydroxyoxetan-3-yl)phenyl)(3-(4-(trifluoromethyl)phenyl)pyrrolidin-1-yl) methanone;
45-(4-fluorophenyl)-3,3a,6,6a-tetrahydrocyclopenta[c]pyrrol-2(1H)-yl)(4-(3-hydroxyoxetan-3-yl)phenyl)methanone;
(4-(3-ethoxyoxetan-3-yl)phenyl)(5-(4-(trifluoromethyl)phenyl)-3,4,5,6-tetrahydropyrrolo[3,4-c]pyrrol-2(1H)-yl)methanone;
(5-hydroxy-5-(4-(trifluoromethyl)phenyl)hexahydrocyclopenta[c]pyrrol-2(1H)-yl)(4-(3-hydroxyoxetan-3-yl)phenyl)methanone;
(4-(3-hydroxyoxetan-3-yl)phenyl)(6-(4-(trifluoromethyl)phenyl)-3,4-dihydroisoquinolin-2(1H)-yl)methanone;
(4-(3-hydroxyoxetan-3-yl)phenyl)(5-(4-(trifluoromethyl)phenyl)indolin-1-yl) methanone;
(4-(3-methoxyoxetan-3-yl)phenyl)(4-(3-(4-methyl-1H-imidazol-1-yl)-4-(trifluoromethyl) phenyl)piperidin-1-yl)methanone;
2,2-dimethyl-7-((3-(4-(4-(4-(trifluoromethyl)phenyl)piperidine-1-carbonyl)phenyl) oxetan-3-yl)oxy)heptanoic acid;
(4-(methoxy methyl)-4-(4-(trifluoromethyl)phenyl)piperidin-1-yl)(4-(3-methoxyoxetan-3-yl) phenyl)methanone;
(4-(hydroxymethyl)-4-(4-(trifluoromethyl)phenyl)piperidin-1-yl)(4-(3-hydroxyoxetan-3-yl) phenyl)methanone;
1-(4-(3-methoxyoxetan-3-yl)benzoyl)-4-(4-(trifluoromethyl)phenyl)piperidine-4-carboxylic acid;
methyl 1-(4-(3-hydroxyoxetan-3-yl)benzoyl)-4-(4-(trifluoromethyl)phenyl) piperidine-4-carboxylate;
1-(4-(3-hydroxyoxetan-3-yl)benzoyl)-4-(4-(trifluoromethyl)phenyl)piperidine-4-carboxylic acid;
(4-(3-chloro-4-(trifluoromethyl)phenyl)piperidin-1-yl)(4-(3-methoxyoxetan-3-yl) phenyl)methanone;
1-(4-(3-hydroxyoxetan-3-yl)benzoyl)-4-(4-(trifluoromethyl)phenyl)piperidine-4-carbonitrile;
1-(4-(3-methoxyoxetan-3-yl)benzoyl)-4-(4-(trifluoromethyl)phenyl)piperidine-4-carbonitrile;
(4-(3-hydroxyoxetan-3-yl)phenyl)(4-methoxy-4-(4-(trifluoromethyl)phenyl) piperidin-1-yl)methanone;
(4-methoxy-4-(4-(trifluoromethyl)phenyl)piperidin-1-yl)(4-(3-methoxyoxetan-3-yl) phenyl)methanone;
(3-(3-hydroxyoxetan-3-yl)phenyl)(4-(3-(trifluoromethyl)phenyl)piperidin-1-yl) methanone;
(4-(3-fluoro-4-(trifluoromethyl)phenyl)piperidin-1-yl)(3-(3-hydroxyoxetan-3-yl) phenyl) methanone;
(3-(3-hydroxyoxetan-3-yl)phenyl)(4-(2-(trifluoromethyl)phenyl)piperidin-1-yl) methanone;
(4-(3-ethoxyoxetan-3-yl)phenyl)(3-(4-(trifluoromethyl)phenyl)piperidin-1-yl) methanone;
3-(3-(3-(4-(trifluoromethyl)phenyl)-1,2,4-oxadiazol-5-yl)phenyl)oxetan-3-ol;
5-(3-(3-methoxyoxetan-3-yl)phenyl)-3-(4-(trifluoromethyl)phenyl)-1,2,4-oxadiazole;
3-(4-(3-(4-(trifluoromethyl)phenyl)-1,2,4-oxadiazol-5-yl)phenyl)oxetan-3-ol;
5-(4-(3-methoxyoxetan-3-yl)phenyl)-3-(4-(trifluoromethyl)phenyl)-1,2,4-oxadiazole;
(4-(3-hydroxyoxetan-3-yl)phenyl)(4-(4-((trifluoromethyl)thio)phenyl)piperidin-1-yl) methanone;
(4-(3-methoxyoxetan-3-yl)phenyl)(4-(4-((trifluoromethyl)thio)phenyl)piperidin-1-yl) methanone;
(4-(3-hydroxyoxetan-3-yl)phenyl)(5-(p-tolyl)-3,3a,6,6a-tetrahydrocyclopenta[c]pyrrol-2(1H)-yl)methanone;
(4-(3-hydroxyoxetan-3-yl)phenyl)(5-(4-(methylthio)phenyl)-3,3a,4,6a-tetrahydrocyclopenta[c]pyrrol-2(1H)-yl)methanone;
(4-(3-hydroxyoxetan-3-yl)phenyl)(6-(4-(trifluoromethyl)phenyl)-3,6-diazabicyclo [3.1.1]heptan-3-yl)methanone;
(4-(3-(cyclopropylmethoxy)oxetan-3-yl)phenyl)(5-(4-(trifluoromethyl)phenyl) hexahydropyrrolo[3,4-c]pyrrol-2(1H)-yl)methanone;
(4-(3-(methoxy-d3)oxetan-3-yl)phenyl)(5-(4-(trifluoromethyl)phenyl) hexahydropyrrolo[3,4-c]pyrrol-2(1H)-yl)methanone;
(4-(3-isobutoxyoxetan-3-yl)phenyl)(5-(4-(trifluoromethyl)phenyl)hexahydropyrrolo [3,4-c]pyrrol-2(1H)-yl)methanone;
(4-(3-isopropoxyoxetan-3-yl)phenyl)(5-(4-(trifluoromethyl)phenyl) hexahydropyrrolo[3,4-c]pyrrol-2(1H)-yl)methanone;
(4-(3-(2-methoxyethoxy)oxetan-3-yl)phenyl)(5-(4-(trifluoromethyl)phenyl) hexahydropyrrolo[3,4-c]pyrrol-2(1H)-yl)methanone;
3-(4-(5-(4-(trifluoromethyl)phenyl)octahydropyrrolo[3,4-c]pyrrole-2-carbonyl) phenyl)oxetan-3-yl acetate;
3-(4-(5-(4-(trifluoromethyl)phenyl)octahydropyrrolo[3,4-c]pyrrole-2-carbonyl) phenyl)oxetan-3-yl pivalate;
tert-butyl 4-hydroxy-4-(4-(4-(4-(trifluoromethyl)phenyl)piperidine-1-carbonyl)phenyl) piperidine-1-carboxylate;
(4-(4-hydroxypiperidin-4-yl)phenyl)(4-(4-(trifluoromethyl)phenyl)piperidin-1-yl) methanone;
(4-(4-hydroxytetrahydro-2H-pyran-4-yl)phenyl)(4-(4-(trifluoromethyl)phenyl) piperidin-1-yl) methanone;
(4-(4-methoxytetrahydro-2H-pyran-4-yl)phenyl)(4-(4-(trifluoromethyl)phenyl) piperidin-1-yl) methanone;
(4-(4-methoxypiperidin-4-yl)phenyl)(4-(4-(trifluoromethyl)phenyl)piperidin-1-yl) methanone;
(4-(3-hydroxythietan-3-yl)phenyl)(4-(4-(trifluoromethyl)phenyl)piperidin-1-yl) methanone;
(4-(3-hydroxythietan-3-yl)phenyl)(5-(4-(trifluoromethyl)phenyl)hexahydropyrrolo [3,4-c]pyrrol-2(1H)-yl)methanone;
(4-(3-methoxythietan-3-yl)phenyl)(4-(4-(trifluoromethyl)phenyl)piperidin-1-yl) methanone;
(4-(3-ethoxythietan-3-yl)phenyl)(4-(4-(trifluoromethyl)phenyl)piperidin-1-yl) methanone;
(4-(3-methoxythietan-3-yl)phenyl)(5-(4-(trifluoromethyl)phenyl)hexahydropyrrolo [3,4-c]pyrrol-2(1H)-yl)methanone;
(4-(3-ethoxythietan-3-yl)phenyl)(5-(4-(trifluoromethyl)phenyl)hexahydropyrrolo [3,4-c]pyrrol-2(1H)-yl)methanone;
tert-butyl 3-hydroxy-3-(4-(4-(4-(trifluoromethyl)phenyl)piperidine-1-carbonyl) phenyl)pyrrolidine-1-carboxylate;
(4-(3-hydroxy pyrrol din-3-yl)phenyl)(4-(4-(trifluoromethyl)phenyl)piperidin-1-yl) methanone;
(4-(3-methoxypyrrolidin-3-yl)phenyl)(4-(4-(trifluoromethyl)phenyl)piperidin-1-yl) methanone;
tert-butyl 3-hydroxy-3-(4-(4-(4-(trifluoromethyl)phenyl)piperidine-1-carbonyl) phenyl)azetidine-1-carboxylate;
(4-(3-hydroxyazetidin-3-yl)phenyl)(4-(4-(trifluoromethyl)phenyl)piperidin-1-yl) methanone;
(4-(4-(methoxy-d3)piperidin-4-yl)phenyl)(4-(4-(trifluoromethyl)phenyl)piperidin-1-yl)methanone;
tert-butyl 4-methoxy-4-(4-(4-(4-(trifluoromethyl)phenyl)piperidine-1-carbonyl) phenyl)piperidine-1-carboxylate;
1-(4-methoxy-4-(4-(4-(4-(trifluoromethyl)phenyl)piperidine-1-carbonyl)phenyl) piperidin-1-yl)ethan-1-one;
ethyl 4-methoxy-4-(4-(4-(4-(trifluoromethyl)phenyl)piperidine-1-carbonyl)phenyl) piperidine-1-carboxylate;
(4-(4-methoxy-1-methylpiperidin-4-yl)phenyl)(4-(4-(trifluoromethyl)phenyl) piperidin-1-yl) methanone;
tert-butyl 4-ethoxy-4-(4-(4-(4-(trifluoromethyl)phenyl)piperidine-1-carbonyl)phenyl) piperidine-1-carboxylate;
(4-(4-ethoxypiperidin-4-yl)phenyl)(4-(4-(trifluoromethyl)phenyl)piperidin-1-yl) methanone;
1-(4-ethoxy-4-(4-(4-(4-(trifluoromethyl)phenyl)piperidine-1-carbonyl)phenyl) piperidin-1-yl) ethan-1-one;
ethyl 4-ethoxy-4-(4-(4-(4-(trifluoromethyl)phenyl)piperidine-1-carbonyl)phenyl) piperidine-1-carboxylate;
(4-(4-ethoxy-1-methylpiperidin-4-yl)phenyl)(4-(4-(trifluoromethyl)phenyl)piperidin-1-yl)methanone;
tert-butyl 4-hydroxy-4-(4-(5-(4-(trifluoromethyl)phenyl)octahydropyrrolo[3,4-c]pyrrole-2-carbonyl)phenyl)piperidine-1-carboxylate;
tert-butyl 4-methoxy-4-(4-(5-(4-(trifluoromethyl)phenyl)octahydropyrrolo[3,4-c]pyrrole-2-carbonyl)phenyl)piperidine-1-carboxylate;
(4-(4-methoxypiperidin-4-yl)phenyl)(5-(4-(trifluoromethyl)phenyl) hexahydropyrrolo[3,4-c]pyrrol-2(1H)-yl)methanone;
(4-(4-methoxy-1-(2,2,2-trifluoroethyl)piperidin-4-yl)phenyl)(4-(4-(trifluoromethyl) phenyl)piperidin-1-yl)methanone;
(4-(4-ethoxy-1-(2,2,2-trifluoroethyl)piperidin-4-yl)phenyl)(4-(4-(trifluoromethyl) phenyl)piperidin-1-yl)methanone;
(3-(3-hydroxyoxetan-3-yl)phenyl)(2-(4-(trifluoromethyl)phenyl)-6,7-dihydrothieno [3,2-c]pyridin-5(4H)-yl)methanone;
(4-(3-hydroxyoxetan-3-yl)phenyl)(3-(4-(trifluoromethyl)phenyl)-6,7-dihydrothieno [3,2-c]pyridin-5(4H)-yl)methanone;
(3-(3-hydroxyoxetan-3-yl)phenyl)(2-(4-(trifluoromethyl)phenyl)-6,7-dihydrothiazolo [5,4-c]pyridin-5(4H)-yl)methanone;
N-(4-(3-hydroxyoxetan-3-yl)phenyl)-2-(4-(trifluoromethyl)phenyl)-6,7-dihydrothiazolo[5,4-c]pyridine-5(4H)-carboxamide;
(4-(3-hydroxyoxetan-3-yl)phenyl)(2-(4-(trifluoromethyl)phenyl)-6,7-dihydrothiazolo [5,4-c]pyridin-5(4H)-yl)methanone;
(3-(3-hydroxyoxetan-3-yl)phenyl)(3-(4-(trifluoromethyl)phenyl)-6,7-dihydrothieno [3,2-c]pyridin-5(4H)-yl)methanone;
(1-benzyl-3-(4-(trifluoromethyl)phenyl)-4,6-dihydropyrrolo[3,4-c]pyrazol-5(1H)-yl) (3-(3-hydroxyoxetan-3-yl)phenyl)methanone;
(3-(3-ethoxyoxetan-3-yl)phenyl)(5-(4-(trifluoromethyl)phenyl)hexahydropyrrolo [3,4-c]pyrrol-2(1H)-yl)methanone;
ethyl 2-((3-(3-(5-(4-(trifluoromethyl)phenyl)octahydropyrrolo[3,4-c]pyrrole-2-carbonyl)phenyl)oxetan-3-yl)oxy)acetate;
(3-(3-isobutoxyoxetan-3-yl)phenyl)(5-(4-(trifluoromethyl)phenyl) hexahydropyrrolo [3,4-c]pyrrol-2(1H)-yl)methanone;
(3-(3-(cyclopropylmethoxy)oxetan-3-yl)phenyl)(5-(4-(trifluoromethyl)phenyl) hexahydropyrrolo[3,4-c]pyrrol-2(1H)-yl)methanone;
N-(3-(3-hydroxyoxetan-3-yl)phenyl)-5-(4-(trifluoromethyl)phenyl)hexahydropyrrolo [3,4-c]pyrrole-2(1H)-carboxamide;
N-(3-(3-methoxyoxetan-3-yl)phenyl)-5-(4-(trifluoromethyl)phenyl)hexahydropyrrolo [3,4-c]pyrrole-2(1H)-carboxamide;
(4-(3-hydroxyoxetan-3-yl)phenyl)(4-(4-(trifluoromethyl)phenyl)-1,4-diazepan-1-yl) methanone;
(4-(3-methoxyoxetan-3-yl)phenyl)(4-(4-(trifluoromethyl)phenyl)-1,4-diazepan-1-yl) methanone;
(4-(3-hydroxyoxetan-3-yl)phenyl)(8-(4-(trifluoromethyl)phenyl)-2,8-diazaspiro[4.5]decan-2-yl)methanone;
4-(3-hydroxyoxetan-3-yl)-N-(1-(4-(trifluoromethyl)phenyl)pyrrolidin-3-yl) benzamide;
(4-(3-(2-hydroxyethoxy)oxetan-3-yl)phenyl)(4-(4-(trifluoromethyl)phenyl)piperidin-1-yl) methanone;
(4-(3-(2-(ethylthio)ethoxy)oxetan-3-yl)phenyl)(4-(4-(trifluoromethyl)phenyl) piperidin-1-yl) methanone;
(4-(3-(2-(ethylsulfonyl)ethoxy)oxetan-3-yl)phenyl)(4-(4-(trifluoromethyl)phenyl) piperidin-1-yl)methanone;
(4-(3-hydroxyoxetan-3-yl)phenyl)(4-(4-(methylthio)phenyl)piperidin-1-yl) methanone;
(4-(3-methoxyoxetan-3-yl)phenyl)(4-(4-(methylthio)phenyl)piperidin-1-yl) methanone;
(4-(4-hydroxypiperidin-4-yl)phenyl)(4-(4-(methylthio)phenyl)piperidin-1-yl) methanone;
(4-(4-methoxypiperidin-4-yl)phenyl)(4-(4-(methylthio)phenyl)piperidin-1-yl) methanone;
(3-(3-(methoxy-d3)oxetan-3-yl)phenyl)(5-(4-(trifluoromethyl)phenyl) hexahydropyrrolo[3,4-c]pyrrol-2(1H)-yl)methanone;
(3-(3-isopropoxyoxetan-3-yl)phenyl)(5-(4-(trifluoromethyl)phenyl) hexahydropyrrolo[3,4-c]pyrrol-2(1H)-yl)methanone;
N-(3-(3-hydroxyoxetan-3-yl)-5-morpholinophenyl)-1-(4-(trifluoromethyl)phenyl) piperidine-4-carboxamide;
methyl 4-(5-hydroxy-2-(4-(3-hydroxyoxetan-3-yl)benzoyl)octahydrocyclopenta[c]pyrrol-5-yl)benzoate;
(4-(3-hydroxyoxetan-3-yl)phenyl)(5-(4-methoxyphenyl)-3,3a,4,6a-tetrahydrocyclopenta[c]pyrrol-2(1H)-yl)methanone;
(4-(3-hydroxyoxetan-3-yl)phenyl)(5-(4-(2-(methylthio)ethoxy)phenyl)-3,3a,4,6a-tetrahydrocyclopenta[c]pyrrol-2(1H)-yl)methanone;
N-(3-(3-hydroxyoxetan-3-yl)-5-(4-methylpiperazin-1-yl)phenyl)-1-(4-(trifluoromethyl)phenyl)piperidine-4-carboxamide;
(4-(3-methoxyoxetan-3-yl)phenyl)(5-(4-(trifluoromethyl)phenyl)-3,4,5,6-tetrahydropyrrolo[3,4-c]pyrrol-2(1H)-yl)methanone;
(5-hydroxy-5-(6-m ethoxypyridin-3-yl)hexahydrocyclopenta[c]pyrrol-2(1H)-yl)(4-(3-hydroxyoxetan-3-yl)phenyl)methanone;
3-(4-((1-(4-(trifluoromethyl)phenyl)piperidin-4-yl)amino)phenyl)oxetan-3-ol;

In another embodiment compounds are selected from following:
tert-butyl 4-methoxy-4-(4-(4-(5-(trifluoromethyl)pyridin-2-yl)piperidine-1-carbonyl) phenyl)piperidine-1-carboxylate;
(4-(4-methoxypiperidin-4-yl)phenyl)(4-(5-(trifluoromethyl)pyridin-2-yl)piperidin-1-yl)methanone;
(4-(3-methoxyoxetan-3-yl)phenyl)(5-(6-(trifluoromethyl)pyridin-3-yl) hexahydropyrrolo[3,4-c]pyrrol-2(1H)-yl)methanone;
(4-(3-hydroxyoxetan-3-yl)phenyl)(5-(5-(trifluoromethyl)pyridin-2-yl) hexahydropyrrolo[3,4-c]pyrrol-2(1H)-yl)methanone;
(3-(3-methoxyoxetan-3-yl)phenyl)(5-(5-(trifluoromethyl)pyridin-2-yl) hexahydropyrrolo[3,4-c]pyrrol-2(1H)-yl)methanone;
(4-(3-methoxyoxetan-3-yl)phenyl)(5-(5-(trifluoromethyl)pyridin-2-yl) hexahydropyrrolo[3,4-c]pyrrol-2(1H)-yl)methanone;
(3-(3-hydroxyoxetan-3-yl)phenyl)(5-(5-(trifluoromethyl)pyridin-2-yl) hexahydropyrrolo[3,4-c]pyrrol-2(1H)-yl)methanone;
(4-(3-methoxyoxetan-3-yl)phenyl)(4-(5-(trifluoromethyl)pyridin-2-yl)piperidin-1-yl) methanone;
(4-(3-hydroxyoxetan-3-yl)phenyl)(4-(5-(trifluoromethyl)pyridin-2-yl)piperidin-1-yl) methanone;
(4-(3-methoxyoxetan-3-yl)phenyl)(4-(6-(trifluoromethyl)pyridin-3-yl)piperidin-1-yl) methanone;
(4-(3-hydroxyoxetan-3-yl)phenyl)(4-(6-(trifluoromethyl)pyridin-3-yl)piperidin-1-yl) methanone;
(4-(3-hydroxyoxetan-3-yl)phenyl)(5-(6-(trifluoromethyl)pyridin-3-yl) hexahydropyrrolo[3,4-c]pyrrol-2(1H)-yl)methanone;
(4-(3-hydroxyoxetan-3-yl) phenyl)(4-(5-(trifluoromethyl)pyridin-2-yl) piperazin-1-yl)methanone;
(4-(3-hydroxyoxetan-3-yl) phenyl)(4-(6-(trifluoromethyl) pyridin-3-yl) piperazin-1-yl)methanone;
(4-(3-methoxyoxetan-3-yl)phenyl)(4-(5-(trifluoromethyl)pyridin-2-yl)piperazin-1-yl) methanone;
(4-(3-ethoxy oxetan-3-yl)phenyl)(4-(5-(trifluoromethyl)pyridin-2-yl)piperazin-1-yl) methanone;
(3-(3-hydroxyoxetan-3-yl)phenyl)(4-(5-(trifluoromethyl)pyridin-2-yl)piperazin-1-yl) methanone;
(3-(3-methoxyoxetan-3-yl)phenyl)(4-(5-(trifluoromethyl)pyridin-2-yl)piperazin-1-yl) methanone;
(4-(3-hydroxyoxetan-3-yl)phenyl)(4-(2-(trifluoromethyl)pyridin-4-yl)piperazin-1-yl) methanone;
(4-(3-hydroxyoxetan-3-yl)phenyl)(4-(2-(trifluoromethyl)pyridin-4-yl)piperidin-1-yl) methanone.

The novel compounds of this invention may be prepared using the reactions and techniques as shown in scheme below and described in this section. The reactions are performed in solvents appropriate to the reagents and materials employed and are suitable for the transformations being affected. It is understood by those skilled in the art that the nature and order of the synthetic steps presented may be varied for the purpose of optimizing the formation of the compounds of the present invention. It will also be well appreciated that one or more of the reactants may be protected and deprotected for facile synthesis by techniques known to persons skilled in the art. It will also be appreciated that one or more of the compounds of the present invention may exist in stereoisomeric and/or diastereomeric forms. Such stereoisomers and/or diastereoisomers as well as their optical antipodes are to be construed to be within the scope of the present invention. It will also be well appreciated that one or more of these compounds may be converted to their salts and other derivatives based on the specific groups present on the compounds, which can be well comprehended by persons skilled in the art. Such salts and/or other derivatives, as the case may be should also be construed to be within the scope of the present invention.

The compounds of general formula (Ia) & (Ib) wherein ‘Y’ represents C═O, and all other symbols are as defined earlier, may be prepared by reactions outlined in Scheme 1 below which comprises:

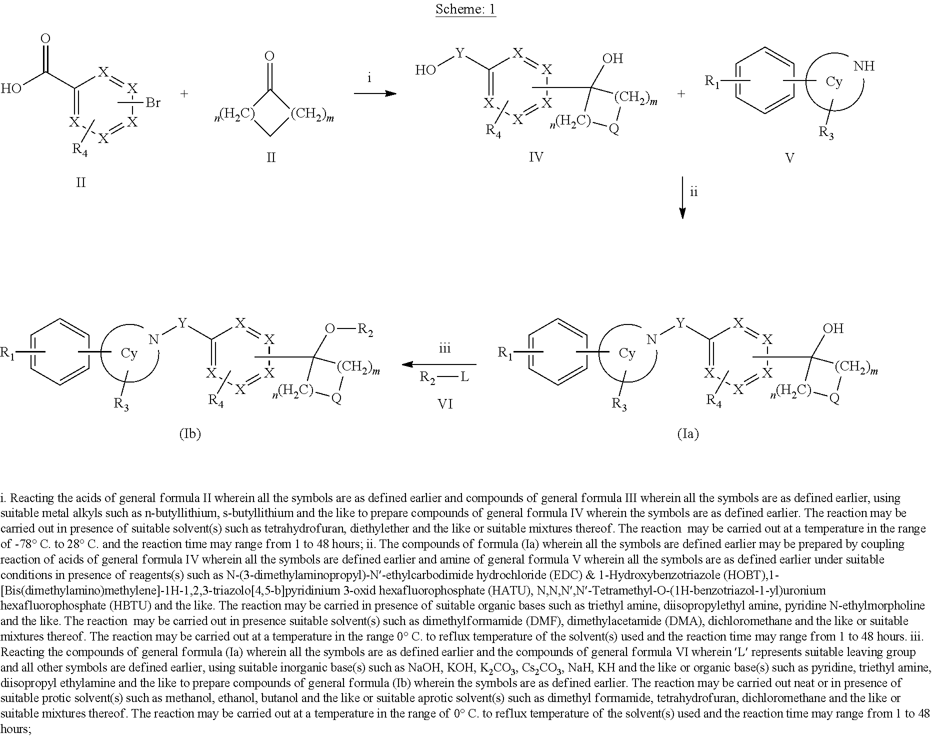

The compounds of general formula (Ic), (Id) & (Ie) wherein ‘Y’ represents —C(O)NH— and all other symbols are as defined earlier, may be prepared by reactions outlined in Scheme 2 below which comprises:

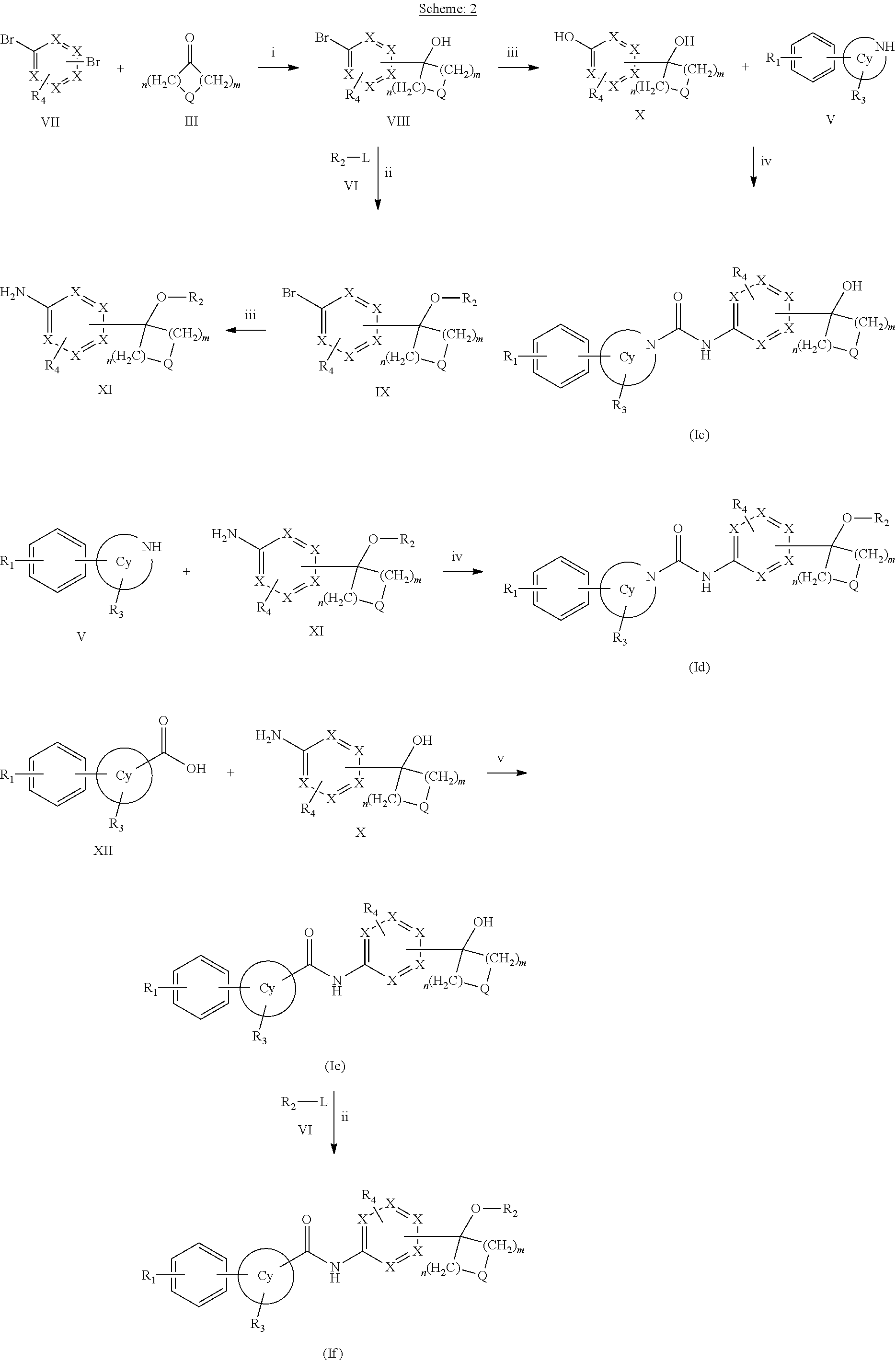

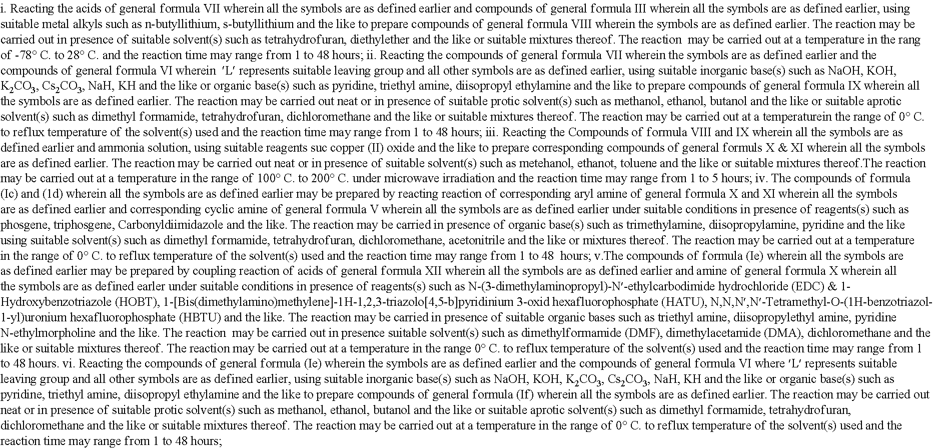

The compounds of general formula (If) & (Ig) wherein ‘Y’ represents a bond and all other symbols are as defined earlier, may be prepared by reactions outlined in Scheme 2 below which comprises:

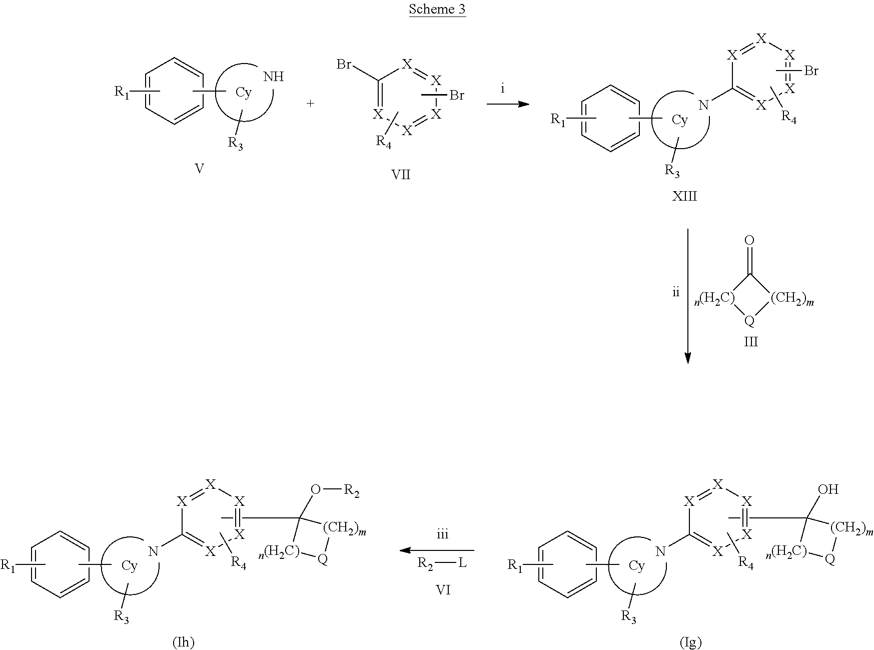

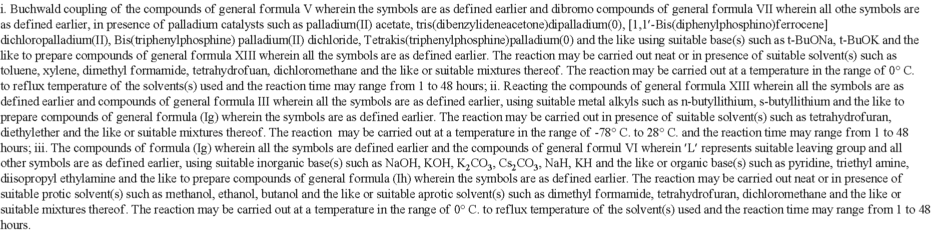

The invention is explained in greater detail by the examples given below, which are provided by way of illustration only and therefore should not be construed to limit the scope of the invention.

¹H NMR spectral data given in the examples (vide infra) are recorded using a 400 MHz spectrometer (Bruker AVANCE-400) and reported in 8 scale.

### Example 1: (4-(3-hydroxyoxetan-3-yl)phenyl)(4-(4-(trifluoromethyl)phenyl) piperidin-1-yl)methanone

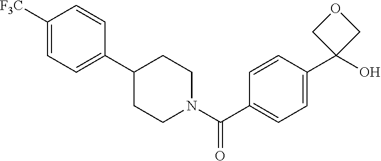 [SMILES: O=C(c1ccc(C2(O)COC2)cc1)N1CCC(c2ccc(C(F)(F)F)cc2)CC1]

### Step-a: Preparation of 4-(3-hydroxyoxetan-3-yl)benzoic acid

To a solution of 4-bromobenzoic acid (1.860 g, 9.25 mmol) in THF (25 ml), N-butyllithium (2.5 M in hexane) (8.14 ml, 20.35 mmol) was added dropwise under nitrogen atmosphere at −78° C. over a period of 30 minutes. The reaction mixture was stirred at the same temperature for 30 min. A solution of oxetan-3-one (1 g, 13.88 mmol) in THF (5 ml) was added dropwise at −78° C. The reaction mixture was allowed to warm to ambient temperature and stirred for 3 hrs under nitrogen atmosphere. The reaction mixture was poured into saturated aqueous NH₄Cl solution (20 mL), acidified with dil. HCl and extracted with ethyl acetate (3×25 ml). The combined organic layers was washed with water & brine, dried over Na₂SO₄ and evaporated under vacuum. The crude product was purified by triturating with diethyl ether to obtain pure 4-(3-hydroxyoxetan-3-yl)benzoic acid (410 mg, 23% Yield) as off white solid.

¹H NMR (DMSO-d₆) δ: 4.68 (d, J=6.8 Hz, 2H), 4.80 (d, J=6.8 Hz, 2H), 6.53 (s, 1H), 7.74 (d, J=6.8 Hz, 2H), 7.98 (d, J=6.8 Hz, 2H), 12.93 (br s, 1H); MS (ESI, pos. ion) m/z: 195.04 [M+H].

### Step b: Preparation of tert-butyl 4-hydroxy-4-(4-(trifluoromethyl) phenyl) piperidine-1-carboxylate

To, a solution of 1-bromo-4-(trifluoromethyl)benzene (5.0 g, 22.22 mmol) in THF (50 ml), N-butyllithium (2.5M in hexane) (18.67 ml, 46.7 mmol) was added dropwise maintaining the reaction temperature below −70° C. over a period of 20 min and stirred for 30 min at −78° C. under nitrogen atmosphere. A solution of tert-butyl 4-oxopiperidine-1-carboxylate (4.43 g, 22.22 mmol) in THF (20 ml) was added drop wise to the reaction mixture under nitrogen atmosphere at −78° C. over a period of 10 min. The reaction mixture was stirred under nitrogen atmosphere at 27° C. for 3 hrs. The reaction mixture was poured into saturated ammonium chloride solution (250 mL) and extracted with ethyl acetate (3×100 mL). The combined organic extract was washed with water (3×150 mL) & brine (150 mL), dried over Na₂SO₄ and evaporated under vacuum. The crude product was purified by column chromatography using 100-200 mesh silica gel column and 10% EtOAc in Hexane as an eluent to yield pure tert-butyl 4-hydroxy-4-(4-(trifluoromethyl)phenyl) piperidine-1-carboxylate (2.8 g, 8.11 mmol, 36.5% yield) as off white solid.

¹H NMR (DMSO-d₆) δ: 1.42 (s, 9H), 1.58 (d, J=12.4 Hz, 2H), 1.79-1.87 (m, 2H), 3.13 (br s, 2H), 3.87 (br s, 2H), 5.32 (s, 1H), 7.67-7.72 (m, 4H).

### Step c: Preparation of 4-(4-(trifluoromethyl)phenyl)piperidin-4-ol hydrochloride

HCl in 1,4-dioxane (10 ml) was added to a solution of tert-butyl 4-hydroxy-4-(4-(trifluoromethyl)phenyl)piperidine-1-carboxylate (1.1 g, 3.19 mmol) in 1,4-Dioxane (10 ml) under nitrogen atmosphere at 27° C. The reaction mixture was stirred under nitrogen atmosphere at 27° C. for 12 hrs. Solvent was removed under vacuum and the residue was diluted with diethylether (10 ml) and stirred for 30 min to get pure 4-(4-(trifluoromethyl)phenyl)piperidin-4-ol hydrochloride as off white solid.

¹H NMR (DMSO-d₆) δ: 1.48 (d, J=12.0 Hz, 2H), 1.76-1.83 (m, 2H), 2.66-2.74 (m, 2H), 2.88-2.95 (m, 2H), 5.01 (s, 1H), 7.67-7.70 (m, 4H).

### Step d: Preparation of 4-(4-(trifluoromethyl)phenyl)-1,2,3,6-tetrahydropyridine 4-methylbenzenesulfonate

Para-Toluenesulfonic acid (PTSA) (5.43 g, 28.5 mmol) was added to a solution of 4-(4-(trifluoromethyl)phenyl)piperidin-4-ol (1.4 g, 5.71 mmol) in Toluene (25 ml) under nitrogen atmosphere at 27° C. and the reaction mixture was refluxed for 12 hrs. The reaction mixture was cooled to ambient temperature and was poured into ice cold water (100 mL). Off white solid separated was filtered through Buchner funnel, washed with water and dried over P₂O₅ under vacuum to yield 4-(4-(trifluoromethyl)phenyl)-1,2,3,6-tetrahydropyridine 4-methylbenzenesulfonate (1.3 g, 5.05 mmol, 88% yield) as off white solid.

¹H NMR (DMSO-d6) δ: 2.27 (s, 3H), 2.67-2.71 (m, 2H), 3.35 (br s, 2H), 3.81 (br s, 2H), 6.36 (s, 1H), 7.11 (d, J=7.6 Hz, 2H), 7.48 (d, J=8.0 Hz, 2H), 7.70 (d, J=8.8 Hz, 2H), 7.75 (d, J=8.4 Hz, 2H), 8.81 (br s, 2H).

### Step e: Preparation of 4-(4-(trifluoromethyl)phenyl)piperidine 4-methylbenzenesulfonate

A solution of 4-(4-(trifluoromethyl)phenyl)-1,2,3,6-tetrahydropyridine (5.0 g, 22.00 mmol) in MeOH (100 ml) was added to a suspension of Pd/C (10%) (1.5 gm) in MeOH (10 ml) and the mixture was hydrogenated on a parr apparatus at 50 psi hydrogen pressure and 27° C. temperature for 3 hrs. The reaction mixture was filtered through celite bed and the filtrate was evaporated in rotavapor under reduced pressure to obtained 4-(4-(trifluoromethyl)phenyl)piperidine (5 μm, 99% yield) as white solid.

¹H NMR DMSO-d₆) δ: 1.74-1.85 (m, 2H), 1.94-1.98 (m, 2H), 2.29 (s, 3H), 2.94-3.07 (m, 3H), 3.34-3.41 (m, 2H), 7.12 (d, J=8.0 Hz, 2H), 7.45-7.50 (m, 4H), 7.71 (d, J=8.0 Hz, 2H), 8.30 (br s, 1H), 8.56 (br s, 2H).

### Step-f: Preparation of (4-(3-hydroxyoxetan-3-yl)phenyl)(4-(4-(trifluoromethyl) phenyl)piperidin-1-yl)methanone

To a solution of 4-(4-(trifluoromethyl)phenyl)piperidine 4-methylbenzenesulfonate (200 mg, 0.872 mmol) in DMF (3.0 ml), 4-(3-hydroxyoxetan-3-yl)benzoic acid (152 mg, 0.785 mmol) and HOBT (200 mg, 1.309 mmol) were added followed by addition of EDC (201 mg, 1.047 mmol) and N-ethylmorpholine (0.331 ml, 2.62 mmol) under nitrogen atmosphere at 27° C. The reaction mixture was stirred under nitrogen atmosphere at 27° C. for 2 hrs. The reaction mixture was poured into ice cold water (50 mL) and extracted with ethyl acetate (3×25 mL). The combined organic layers was washed with water (3×25 mL) & brine (25 mL), dried over Na₂SO₄ and evaporated in rotavapor under vacuum to yield crude product as thick liquid. The crude product was purified by column chromatography using 100-200 mesh silica gel column and 50% EtOAc in Hexane as an eluent to yield (4-(3-hydroxyoxetan-3-yl)phenyl)(4-(4-(trifluoromethyl)phenyl)piperidin-1-yl)methanone (130 mg, 0.316 mmol, 36% yield) as white solid.

¹H NMR (CDCl₃) δ: 1.64 (br s, 2H), 1.83 (br s, 2H), 2.01 (br s, 1H), 2.82-3.02 (m, 2H), 3.17 (br s, 1H), 3.58 (s, 1H), 3.92 (br s, 1H), 4.84 (d, J=7.2 Hz, 2H), 4.95 (d, J=7.2 Hz, 2H), 7.34-7.43 (m, 4H), 7.59-7.66 (m, 4H).

ESI-MS: m/z 406.15 (M+H)⁺, 100%.

### Example 2: (4-(3-methoxyoxetan-3-yl)phenyl)(4-(4-(trifluoromethyl)phenyl) piperidin-1-yl)methanone

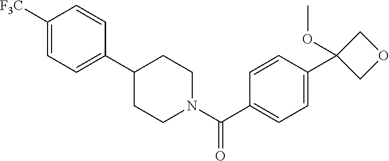 [SMILES: COC1(c2ccc(C(=O)N3CCC(c4ccc(C(F)(F)F)cc4)CC3)cc2)COC1]

To a suspension of sodium hydride (8.88 mg, 0.185 mmol) in THF (2 ml), a solution of (4-(3-hydroxyoxetan-3-yl)phenyl)(4-(4-(trifluoromethyl)phenyl)piperidin-1-yl)methanone (50 mg, 0.123 mmol) in THF (0.5 ml) was added dropwise at 0-10° C. over a period of 10 min and reaction mixture was stirred for 30 min. Iodomethane (9.25 μl, 0.148 mmol) was added to the reaction mixture and continued to stir at 28° C. for 3 hrs. The reaction mixture was poured into ice cold water (15 mL) and extracted with ethyl acetate (3×15 mL). The combined organic layers was washed with water (2×20 mL) & brine (20 mL), dried over Na₂SO₄ and evaporated under vacuum. The crude product was purified by column chromatography using 100-200 mesh silica gel column and 30% EtOAc in Hexane as an eluent to yield pure (4-(3-methoxyoxetan-3-yl)phenyl)(4-(4-(trifluoromethyl)phenyl)piperidin-1-yl)methanone (45 mg, 0.105 mmol, 85% yield) as pale yellow solid.

¹H NMR (DMSO-d₆) δ: 1.61-1.72 (m, 3H), 1.88 (br s, 1H), 2.87-2.98 (m, 2H), 3.05 (s, 3H), 3.19 (br s, 1H), 3.70 (br s, 1H), 4.65 (br s, 1H), 4.76-4.81 (m, 4H), 7.50-7.55 (m, 6H), 7.67 (d, J=8.0 Hz, 2H).

ESI-MS: m/z 420 (M+H)⁺, 100%.

### Example 3: N-(4-(3-hydroxyoxetan-3-yl)phenyl)-1-(4-(trifluoromethyl)phenyl) piperidine-4-carboxamide

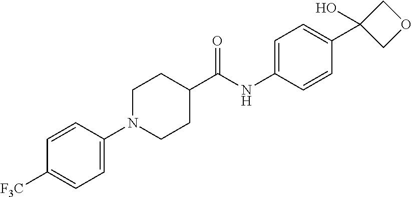

### Step a: Preparation of Ethyl 1-(4-(trifluoromethyl)phenyl)piperidine-4-carboxylate

To a solution of 1-bromo-4-(trifluoromethyl)benzene (3 g, 13.33 mmol) in Toluene (20 ml), ethyl piperidine-4-carboxylate (3.35 g, 21.33 mmol) was added followed by addition of sodium tert-butoxide (1.922 g, 20.00 mmol), BINAP (0.249 g, 0.400 mmol) and Pd₂(dba)₃ (0.122 g, 0.133 mmol) under nitrogen atmosphere at 28° C. The reaction mixture was stirred under nitrogen atmosphere at 120° C. for 16 hrs. Reaction mixture was cooled to room temperature and filtered through celite. The filtrate was diluted with ethyl acetate (40 mL) and water (40 mL). Organic layer was separated and aq. layer was extracted with ethyl acetate (2×20 mL). The combined organic layers was washed with water (2×60 mL) and brine (60 mL), dried over Na₂SO₄ and evaporated on rotavapor under vacuum to yield crude product as an oil. The crude product was purified by column chromatography using 100-200 mesh silica gel column and 10% EtOAc in Hexane as an eluent to obtained ethyl 1-(4-(trifluoromethyl)phenyl)piperidine-4-carboxylate (1.608 g, 40% yield) as light yellow oil.

¹H NMR (CDCl₃) δ: 1.29 (t, J=7.2 Hz, 3H), 1.81-1.91 (m, 2H), 2.02-2.07 (m, 2H), 2.47-2.55 (m, 1H), 2.89-2.95 (m, 2H), 3.74-3.79 (m, 2H), 4.18 (q, J=7.2 Hz, 2H), 6.94 (d, J=8.4 Hz, 2H), 7.48 (d, J=8.4 Hz, 2H).

ESI-MS: m/z 302.12 (M+H)⁺, 100%.

### Step b: Preparation of 1-(4-(trifluoromethyl)phenyl)piperidine-4-carboxylic acid

To a solution of ethyl 1-(4-(trifluoromethyl)phenyl)piperidine-4-carboxylate (1.2 g, 3.98 mmol) in THF (5 ml) and MeOH (5 ml), a solution of Lithium hydroxide monohydrate (0.334 g, 7.97 mmol) in water (5 mL) was added and the reaction mixture was stirred at ambient temperature for 3 hr. Reaction mixture was concentrated under vacuum, diluted with cold water (15 mL) and was acidified with dil HCl (pH 3-4) with vigorous stirring. The white solid obtained was filtered, washed with water and dried over P₂O₅ under vacuum to yield 1-(4-(trifluoromethyl)phenyl)piperidine-4-carboxylic acid (1 g, 92% yield) as off white solid.

¹H NMR (DMSO-d₆) δ: 1.61-1.66 (m, 2H), 1.86-1.88 (m, 2H), 2.44-2.49 (m, 1H), 2.88-2.99 (m, 2H), 3.78-3.82 (m, 2H), 7.05 (d, J=8.8 Hz, 2H), 7.55 (d, J=8.8 Hz, 2H), 12.30 (br s, 1H). ESI-MS: m/z 302.12 (M+H)⁺, 100%.

### Step c: Preparation of N-(4-(3-hydroxyoxetan-3-yl)phenyl)-1-(4-(trifluoromethyl) phenyl) piperidine-4-carboxamide

To a solution of 1-(4-(trifluoromethyl)phenyl)piperidine-4-carboxylic acid (0.220 g, 0.805 mmol) in DMF (4 ml), HATU (0.490 g, 1.288 mmol) was added followed by addition of 3-(4-aminophenyl)oxetan-3-ol (0.146 g, 0.886 mmol) and DIPEA (0.337 ml, 1.932 mmol) under nitrogen atmosphere at 28° C. The reaction mixture was stirred at 70° C. under nitrogen atmosphere for 16 h. Then it was cooled to room temperature, poured into ice cold water (25 ml) and extracted with ethyl acetate (3×25 ml). The combine organic extract was washed with water (3×30 mL) and brine (20 mL), dried over Na₂SO₄ and evaporated under vacuum to yield crude product as thick liquid. The crude product was purified by triturating with ethyl acetate to obtained pure N-(4-(3-hydroxyoxetan-3-yl)phenyl)-1-(4-(trifluoromethyl)phenyl) piperidine-4-carboxamide (0.2 g, 58.4% Yield) as off white solid.

¹H NMR (DMSO-d6) δ: 1.66-1.76 (m, 2H), 1.86-1.89 (m, 2H), 2.56-2.63 (m, 1H), 2.85-2.91 (m, 2H), 3.93-3.97 (m, 2H), 4.66 (d, J=6.4 Hz, 2H), 4.75 (d, J=6.4 Hz, 2H), 6.27 (s, 1H), 7.08 (d, J=8.8 Hz, 2H), 7.48-7.52 (m, 4H), 7.63 (d, J=8.8 Hz, 2H). 9.97 (s, 1H).

ESI-MS: m/z 421.15 (M+H)⁺, 100%.

### Example 4: N-(4-(3-hydroxyoxetan-3-yl)phenyl)-4-(4-(trifluoromethyl)phenyl) piperidine-1-carboxamide

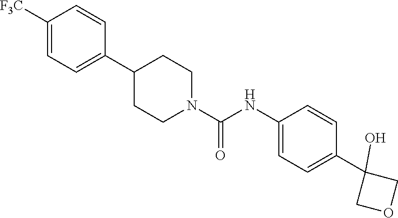

To a solution of 3-(4-aminophenyl)oxetan-3-ol (100 mg, 0.605 mmol) in THF (3.0 ml), an other solution of triphosgene (59.3 mg, 0.200 mmol) in THF (1.0 ml) was added followed by addition of a solution of 4-(4-(trifluoromethyl)phenyl)piperidine (180 mg, 0.787 mmol) and diisopropylethylamine (0.317 ml, 1.816 mmol) in acetonitrile (3.00 ml) under nitrogen atmosphere at 0° C. The reaction mixture was stirred at 27° C. for 12 hrs under nitrogen atmosphere. The reaction mixture was poured into water (50 mL) and extracted with ethyl acetate (2×25 mL). The combined organic layers was washed with water (2×25 mL) and brine (50 mL), dried over sodium sulfate and evaporated on rotavapor under vacuum to yield crude product as colorless liquid. The crude product was purified by column chromatography using 100-200 mesh silica gel column and 50% EtOAc in Hexane as an eluent to yield pure N-(4-(3-hydroxyoxetan-3-yl)phenyl)-4-(4-(trifluoromethyl)phenyl)piperidine-1-carboxamide (120 mg, 42% yield) as off white solid.

¹H NMR (DMSO-d6) δ: 1.55-1.65 (m, 2H), 1.82-1.84 (m, 2H), 2.84-2.92 (m, 3H), 4.28-4.31 (m, 2H), 4.67 (d, J=6.8 Hz, 2H), 4.72 (d, J=6.8 Hz, 2H), 6.20 (s, 1H), 7.40 (d, J=8.8 Hz, 2H), 7.43-7.53 (m, 4H), 7.67 (d, J=8.4 Hz, 2H), 8.51 (s, 1H).

ESI-MS: m/z 421 (M+H)⁺, 100%.

### Example 5: N-(4-(3-methoxyoxetan-3-yl)phenyl)-4-(4-(trifluoromethyl)phenyl) piperidine-1-carboxamide

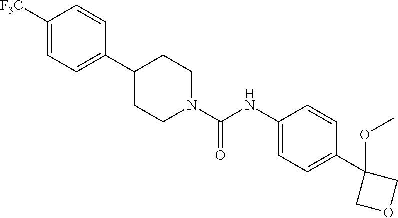

### Step a: Preparation of 3-(4-bromophenyl)-3-methoxyoxetane

To a suspension of NaH (27.2 mg, 0.568 mmol) in THF (1.0 ml) was added 3-(4-bromophenyl) oxetan-3-ol (100 mg, 0.437 mmol) in small portions under nitrogen atmosphere at 27° C. and stirred for 30 min. Iodomethane (0.033 ml, 0.524 mmol) was added drop wise and continued to stir for further 2 hr. The reaction mixture was poured into ice cold water (20 mL) and extracted with ethyl acetate (2×15 mL). The combined organic layer was washed with water (2×15 mL) & brine (15 mL), dried over Na₂SO₄ and evaporated on rotavapor under vacuum to yield 3-(4-bromophenyl)-3-methoxyoxetane (106 mg, 0.437 mmol, 100% yield) as yellow liquid.

¹H NMR (CDCl₃) δ: 3.16 (s, 3H), 4.79 (d, J=7.6 Hz, 2H), 4.93 (d, J=7.2 Hz, 2H), 7.30 (d, J=8.8 Hz, 2H), 7.54 (d, J=8.4 Hz, 2H).

### Step b: Preparation of 4-(3-methoxyoxetan-3-yl) aniline

A solution of 3-(4-bromophenyl)-3-methoxyoxetane (200 mg, 0.823 mmol) in saturated NH₄OH solution (1.0 ml) was placed into microwave vial. Copper (II) oxide (65.4 mg, 0.823 mmol) was added at 27° C. The reaction mixture was stirred under microwave radiation at 100° C. and 40 psi pressure for 1 hr. The reaction mixture was diluted with ethyl acetate (10 ml) and filtered through celite bed. The filtrate was dried over Na₂SO₄ and evaporated under reduced pressure to obtain crude product as thick liquid. The crude product was used for next step without purification.

### Step c: Preparation of N-(4-(3-methoxyoxetan-3-yl)phenyl)-4-(4-(trifluoromethyl)phenyl)piperidine-1-carboxamide

The title product was synthesized from 3-(4-bromophenyl)-3-methoxyoxetane and 4-(4-(trifluoromethyl)phenyl)piperidine following the procedure described in example 4.

¹H NMR (DMSO-d₆) δ: 1.56-1.65 (m, 2H), 1.82-1.85 (m, 2H), 2.85-2.93 (m, 3H), 2.99 (s, 3H), 4.30 (d, J=13.2 Hz, 2H), 4.72-4.76 (m, 4H), 7.28 (d, J=8.4 Hz, 2H), 7.51-7.55 (m, 4H), 7.67 (d, J=8.0 Hz, 2H), 8.64 (s, 1H).

ESI-MS: m/z, 435.4 (M+H)⁺, 100%.

### Example 6: (4-(3-hydroxyoxetan-3-yl)phenyl)(4-(4-(trifluoromethyl)phenyl) piperazin-1-yl)methanone

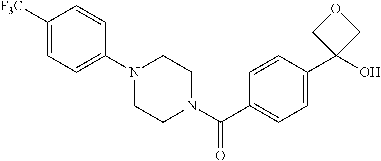 [SMILES: O=C(c1ccc(C2(O)COC2)cc1)N1CCN(c2ccc(C(F)(F)F)cc2)CC1]

### Step a: Preparation of tert-butyl 4-(4-(trifluoromethyl)phenyl)piperazine-1-carboxylate

To, a solution of 1-bromo-4-(trifluoromethyl)benzene (5.0 g, 22.22 mmol) in toluene (4.0 ml), tert-butyl piperazine-1-carboxylate (6.21 g, 33.3 mmol), Pd₂(dba)₃ (1.017 g, 1.11 mmol), BINAP (1.384 g, 2.22 mmol) and potassium tert-butoxide (7.48 g, 66.7 mmol) were added under nitrogen atmosphere at 27° C. The reaction mixture was stirred under nitrogen atmosphere at 80° C. for 3 hrs. The reaction mixture was cooled to room temperature and was filtered through celite bed. The filtrate was evaporated under reduced pressure. The residue was purified by column chromatography using 100-200 mesh silica gel column and 5% EtOAc in Hexane as an eluent to yield tert-butyl 4-(4-(trifluoromethyl)phenyl)piperazine-1-carboxylate (3.0 g, 41% yield) as white solid.

¹H NMR (DMSO-d₆) δ: 1.42 (s, 9H), 3.25-3.28 (m, 4H), 3.44-3.47 (m, 4H), 7.07 (d, J=8.8 Hz, 2H), 7.52 (d, J=8.8 Hz, 2H).

### Step b: Preparation of 1-(4-(trifluoromethyl)phenyl)piperazine hydrochloride

To a solution of tert-butyl 4-(4-(trifluoromethyl)phenyl)piperazine-1-carboxylate (100 mg, 0.303 mmol) in 1,4-Dioxane (1.0 ml), HCl in 1,4-dioxane (1.0 ml) was added and stirred at 27° C. for 12 hr. Solvent was removed from the reaction mixture under vacuum and the residue was triturated with diethyl ether (3 ml) to obtained 1-(4-(trifluoromethyl)phenyl) piperazine hydrochloride (80 mg, 99% yield) as off white solid.

¹H NMR (DMSO-d₆) δ: 3.33 (br s, 4H), 3.54 (br s, 4H), 3.98 (br s, 1H), 7.14 (d, J=8.8 Hz, 2H), 7.57 (d, J=8.4 Hz, 2H).

### Step c: Preparation of (4-(3-hydroxyoxetan-3-yl)phenyl)(4-(4-(trifluoromethyl) phenyl) piperazin-1-yl) methanone

The title compound was prepared by coupling reaction of 1-(4-(trifluoromethyl)phenyl)piperazine hydrochloride (250 mg, 0.825 mmol) and 4-(3-hydroxyoxetan-3-yl)benzoic acid (160 mg, 0.825 mmol) following the procedure described in step f of example 1 (60 mg, 0.139 mmol, 17% yield, white solid)

¹H NMR (DMSO-d₆) δ: 3.40 (br s, 2H), 3.60 (br s, 4H), 3.80 (br s, 2H), 4.70 (d, J=6.8 Hz, 2H), 4.80 (d, J=6.8 Hz, 2H), 6.47 (s, 1H), 7.08 (d, J=8.8 Hz, 2H), 7.48-7.54 (m, 4H), 7.70 (d, J=8.4 Hz, 2H).

ESI-MS: m/z, 407 (M+H)⁺, 100%.

### Example 7: 3-(4-((4-(4-(trifluoromethyl)phenyl)piperazin-1-yl)sulfonyl)phenyl) oxetan-3-ol

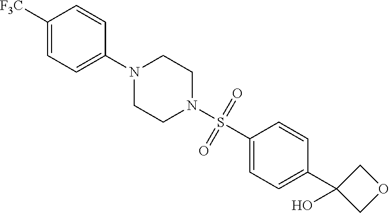 [SMILES: O=S(=O)(c1ccc(C2(O)COC2)cc1)N1CCN(c2ccc(C(F)(F)F)cc2)CC1]

### Step a: Preparation of 1-((4-bromophenyl)sulfonyl)-4-(4-(trifluoromethyl) phenyl) piperazine

To a solution of 4-(4-(trifluoromethyl)phenyl)piperidine (500 mg, 2.181 mmol) in dichloromethane (5.0 ml), triethylamine (0.912 ml, 6.54 mmol) was added followed by drop wise addition of 4-bromobenzene-1-sulfonyl chloride (446 mg, 1.745 mmol) under nitrogen atmosphere at 0-10° C. over a period of 10 min. The reaction mixture was stirred under nitrogen atmosphere at 27° C. for 1 hr. The reaction mixture was poured into ice cold water (50 mL) and extracted with ethyl acetate (3×50 mL). The combined organic layers was washed with water (2×50 mL) & brine (50 mL), dried over Na₂SO₄ and evaporated on rotavapor under vacuum to yield crude product as thick liquid. The crude product was purified by column chromatography using 100-200 mesh silica gel column and 6% EtOAc in Hexane as an eluent to yield 1-((4-bromophenyl) sulfonyl)-4-(4-(trifluoromethyl)phenyl)piperidine (400 mg, 41% yield) as white solid.

¹H NMR (CDCl₃) δ: 1.85-1.92 (m, 4H), 2.37-2.43 (m, 2H), 2.53 (br s, 1H), 3.96-3.99 (m, 2H), 4.28 (d, J=7.6 Hz, 2H), 7.58 (d, J=8.0 Hz, 2H), 7.67-7.74 (m, 4H).

### Step b: Preparation of 3-(4-((4-(4-(trifluoromethyl) phenyl) piperidin-1-yl) sulfonyl) phenyl)oxetan-3-ol

A solution of 1-((4-bromophenyl)sulfonyl)-4-(4-(trifluoromethyl)phenyl)piperidine (100 mg, 0.223 mmol) in THF (3.0 ml) was cooled to −78° C. and n-butyllithium (2.5M in hexane) (0.167 ml, 0.335 mmol) was added drop wise maintaining the temperature at −78° C. over a period of 15 min. The reaction mixture was stirred for further 15 min and 3-oxetanone (19.29 mg, 0.268 mmol) was added under nitrogen atmosphere at −78° C. and continued to stir for 1 hr. The reaction mixture was poured into saturated solution of NH₄Cl (25 mL) and extracted with ethyl acetate (2×15 mL). The combined organic layers was washed with water (2×15 mL) & brine (20 mL), dried over Na₂SO₄ and evaporated on rotavapor under vacuum. The residue was purified by column chromatography 230-400 mesh silica gel column and 15% EtOAc in Hexane as an eluent to yield pure 3-(4-((4-(4-(trifluoromethyl) phenyl) piperidin-1-yl)sulfonyl)phenyl)oxetan-3-ol (50 mg, 31% Yield) as off white solid.

¹H NMR (CDCl₃) δ: 3.25-3.27 (m, 4H), 3.36-3.38 (m, 4H), 4.72 (d, J=7.6 Hz, 2H), 5.06 (d, J=7.6 Hz, 2H), 6.26 (s, 1H), 7.06 (d, J=8.8 Hz, 2H), 7.49-7.53 (m, 3H), 7.58 (d, J=7.6 Hz, 1H), 7.67-7.71 (m, 1H), 7.86-7.89 (m, 1H).

ESI-MS: m/z 443 (M+H)⁺, 50%.

### Example 8: 3-(4-(4-(4-(trifluoromethyl)phenyl)piperidin-1-yl)phenyl)oxetan-3-ol

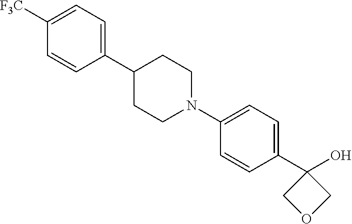 [SMILES: OC1(c2ccc(N3CCC(c4ccc(C(F)(F)F)cc4)CC3)cc2)COC1]

### Step a: Preparation of 1-(4-bromophenyl)-4-(4-(trifluoromethyl)phenyl)piperidine

To a solution of 4-(4-(trifluoromethyl)phenyl)piperidine 4-methylbenzenesulfonate (0.5 g, 1.246 mmol) in Toluene (10 ml), were added 1,4-dibromobenzene (1.469 g, 6.23 mmol) and potassium tert-butoxide (0.419 g, 3.74 mmol) followed by addition of Pd₂(dba)₃ (0.114 g, 0.125 mmol) and BINAP (0.155 g, 0.249 mmol) under nitrogen atmosphere at 27° C. The reaction mixture was stirred at 90° C. for 18 hr. The reaction mixture was diluted with ethyl acetate (20 ml) and filtered through celite bed. The filtrate was evaporated on rotavapor under reduced pressure to obtain crude product as an oil. The crude product was purified by column chromatography using 100-200 mesh silica gel and 10% EtOAc in Hexane as an eluent to yield pure 1-(4-bromophenyl)-4-(4-(trifluoromethyl)phenyl)piperidine (300 mg, 0.781 mmol, 62.7% yield) as off white solid.

¹H NMR (DMSO-d6) δ: 1.71-1.81 (m, 2H), 1.86-1.89 (m, 2H), 2.76-2.85 (m, 3H), 3.81-3.84 (m, 2H), 6.95 (d, J=9.2 Hz, 2H), 7.35 (d, J=9.2 Hz, 2H), 7.51 (d, J=8.4 Hz, 2H), 7.67 (d, J=8.4 Hz, 2H).

ESI-MS: m/z, 384.26 & 386.15 (M+H)⁺, 100%.

### Step b: Preparation of 3-(4-(4-(4-(trifluoromethyl)phenyl)piperidin-1-yl)phenyl)oxetan-3-ol

To a solution of 1-(4-bromophenyl)-4-(4-(trifluoromethyl)phenyl)piperidine (220 mg, 0.573 mmol) in THF (10 ml), n-BuLi (0.458 ml, 1.145 mmol) was added dropwise under nitrogen atmosphere at −78° C. over a period of 10 minute and the reaction mixture was stirred for further 15 min at the same temperature. A solution of oxetanone (83 mg, 1.145 mmol) in THF (0.5 ml) was added drop wise to reaction mixture at −78° C. and the reaction mixture was stirred for 1 hr. The reaction mixture was poured into saturated solution of aqueous NH₄Cl (25 mL) and extracted with ethyl acetate (2×20 mL). The combined organic phase was washed with water (2×20 mL) & brine (20 mL), dried over sodium sulphate and evaporated on rotavapor under vacuum to yield crude product as thick liquid. The crude product was purified by column chromatography using 100-200 mesh silica gel column and 20% EtOAc in Hexane as an eluent to yield pure 3-(4-(4-(4-(trifluoromethyl)phenyl)piperidin-1-yl)phenyl)oxetan-3-ol (100 mg, 0.253 mmol, 44.3% yield) as off white solid.

¹H NMR (DMSO-d₆) δ: 1.77-1.91 (m, 4H), 2.67-2.81 (m, 3H), 3.84 (d, J=12.4 Hz, 2H), 4.66 (d, J=6.4 Hz, 2H), 4.71 (d, J=6.8 Hz, 2H), 6.14 (s, 1H), 7.01 (d, J=8.8 Hz, 2H), 7.43 (d, J=6.8 Hz, 2H), 7.52 (d, J=8.0 Hz, 2H), 7.67 (d, J=8.0 Hz, 2H).

ESI-MS: m/z, 378.13 (M+H)⁺, 100%.

### Example 9: 1-(4-(3-methoxyoxetan-3-yl)phenyl)-4-(4-(trifluoromethyl)phenyl) piperidine

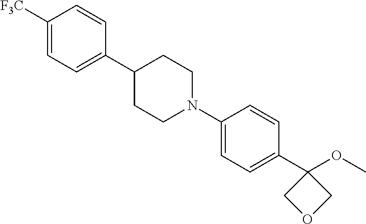 [SMILES: COC1(c2ccc(N3CCC(c4ccc(C(F)(F)F)cc4)CC3)cc2)COC1]

To a suspension of NaH (25.4 mg, 0.530 mmol) in THF (3.0 ml), 3-(4-(4-(4-(trifluoromethyl)phenyl)piperidin-1-yl)phenyl)oxetan-3-ol (100 mg, 0.265 mmol) was added in small portions under nitrogen atmosphere at 27° C. and the reaction mixture was stirred for 15 minutes. Iodomethane (0.020 ml, 0.318 mmol) was added to the reaction mixture under nitrogen atmosphere and was continued to stir for 12 hr. The reaction mixture was poured into ice cold water (10 mL) and extracted with ethyl acetate (2×10 mL). The combined organic layer was washed with water (2×15 mL) & brine (20 mL), dried over Na₂SO₄ and evaporated on rotavapor under vacuum to yield crude product as thick liquid. The crude product was purified by column chromatography using 100-200 mesh silica gel column and 15% EtOAc in Hexane as an eluent to yield pure 1-(4-(3-methoxyoxetan-3-yl) phenyl)-4-(4-(trifluoromethyl) phenyl) piperidine (102 mg, 0.254 mmol, 96% yield) as off white solid.

¹H NMR (CDCl₃) δ: 1.91-2.02 (m, 4H), 2.73-2.76 (m, 1H), 2.88 (t, J=12.0 Hz, 2H), 3.13 (s, 3H), 3.89 (d, J=12.4 Hz, 2H), 4.87 (d, J=7.2 Hz, 2H), 4.91 (d, J=7.2 Hz, 2H), 7.01 (d, J=8.8 Hz, 2H), 7.35 (d, J=8.8 Hz, 2H), 7.39 (d, J=8.4 Hz, 2H), 7.60 (d, J=8.0 Hz, 2H).

ESI-MS: m/z, 392.12 (M+H)⁺, 100%.

### Example 10: (4-(3-hydroxyoxetan-3-yl)phenyl)(5-(4-(trifluoromethyl)phenyl) hexahydrocyclopenta[c]pyrrol-2(1H)-yl)methanone

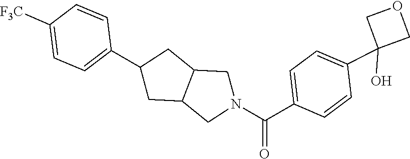 [SMILES: O=C(c1ccc(C2(O)COC2)cc1)N1CC2CC(c3ccc(C(F)(F)F)cc3)CC2C1]

### Step a: Preparation of 2-benzyl-1,2,3,3a,4,6a-hexahydrocyclopenta[c]pyrrol-5-yl trifluoromethane sulfonate

To a solution of 2-benzylhexahydrocyclopenta[c]pyrrol-5(1H)-one (100 mg, 0.464 mmol) in THF (5.0 mL), an other solution of lithium bis(trimethylsilyl)amide in THF (1.0 M, 0.6 mL) was added dropwise at −78° C. over a period of 15 min under nitrogen atmosphere and the mixture was continued to stir at −78° C. for 30 min. A solution of 1,1,1-trifluoro-N-phenyl-N((trifluoromethyl)sulfonyl) methanesulfonamide (166 mg, 0.464 mmol) in THF (1.5 mL) was added dropwise to the reaction mixture and continued to stir for an additional 1 hr at −78° C. and was then allowed to stir at 28° C. for 2 h. The mixture was concentrated under reduced pressure and the residue was purified by flash column chromatography using 0-50% Ethyl acetate in hexane gradient system as an eluent to give 2-benzyl-1,2,3,3a,4,6a-hexahydrocyclopenta[c]pyrrol-5-yl trifluoromethane sulfonate as a clear, viscous oil (100 mg, 62% yield).

¹H NMR (CDCl₃) δ 2.35-2.37 (m, 2H), 2.78-2.80 (m, 4H), 3.60-3.64 (m, 2H), 5.56 (s, 1H), 7.3-7.4 (m, 5H).

ESI-MS: m/z 347.99 (M+H)+, 100%.

### Step b: Preparation of 2-benzyl-5-(4-(trifluoromethyl)phenyl)-1,2,3,3a,4,6a-hexahydrocyclopenta[c]pyrrole

To a mixture of 2-benzyl-1,2,3,3a,4,6a-hexahydrocyclopenta[c]pyrrol-5-yl trifluoromethanesulfonate (100 mg, 0.263 mmol), 4-trifluoromethylphenylboronic acid (50 m g, 0.263 mmol) and 2M aqueous solution of Na₂CO₃ (2.5 mL) in DMF (5.0 mL) was added PdCl₂(PPh₃)₂ (2.15 mg, 2.63 μmol) under nitrogen atmosphere at 28° C. The mixture was stirred at 80° C. for 3 hrs under nitrogen atmosphere. The reaction mixture was cooled to ambient temperature, diluted with water (50 mL) and extracted with ethyl acetate (3×20 mL). The combined organic extracts were washed with water (50 mL) & brine (50 mL), dried over Na₂SO₄ and concentrated under reduced pressure. The residue was purified by flash column chromatography using 0-50% Ethyl acetate in hexanes as eluent to give 2-benzyl-5-(4-(trifluoromethyl)phenyl)-1,2,3,3a,4,6a-hexahydrocyclopenta[c]pyrrole, viscous oil (90 mg, 94% yield):

¹H NMR (CDCl₃) δ 2.35-2.37 (m, 2H), 2.57-2.60 (m, 1H), 2.82-2.85 (m, 2H), 2.95-2.98 (m, 2H), 3.6 (s, 2H), 6.18 (s, 1H), 7.2-7.25 (m, 1H), 7.27-7.30 (m, 4H), 7.33-7.57 (m, 4H).

ESI-MS: m/z 344.14 (M+H)+, 100%.

### Step c: Preparation of 5-(4-(trifluoromethyl)phenyl)octahydrocyclopenta[c]pyrrole

A solution of 2-benzyl-5-(4-(trifluoromethyl)phenyl)-1,2,3,3a,4,6a-hexahydrocyclopenta [c]pyrrole (90 mg, 0.262 mmol) in MeOH (20 ml) was added to a suspension of Pd/C (10%) (50 mg) in MeOH (2 mL). The reaction mixture was hydrogenated under 40 psi pressure of hydrogen gas using Parr Shaker apparatus at ambient temperature for 16 hrs. The mixture was filtered through Celite, washed with methanol and the filtrate was concentrated under reduced pressure to give 5-(4-(trifluoromethyl)phenyl)octahydrocyclopenta[c]pyrrole (65 mg, 97% yield) as a clear, viscous oil.

¹H NMR (CDCl₃) δ: 1.25-1.27 (m, 2H), 2.11-2.13 (m, 1H), 2.31-2.35 (m, 1H), 2.83-2.87 (m, 2H), 2.88-2.90 (m, 1H), 3.10-3.19 (m, 1H), 3.31-3.34 (m, 1H), 3.36-3.38 (m, 1H), 3.63-3.65 (m, 1H), 7.33 (d, J=8.0 Hz, 2H), 7.56 (d, J=8.0 Hz, 2H).

ESI-MS: m/z 256.13 (M+H)+, 100%.

### Step d: Preparation of (4-(3-hydroxyoxetan-3-yl)phenyl)(5-(4-(trifluoromethyl)phenyl) hexahydrocyclopenta[c]pyrrol-2(1H)-yl)methanone

The title compound was prepared by coupling reaction of 5-(4-(trifluoromethyl)phenyl)octahydrocyclopenta[c]pyrrole (0.065 g, 0.25 mmol) and 4-(3-hydroxyoxetan-3-yl)benzoic acid (0.08 g, 0.25 mmol) according to the procedure mentioned in step f of example 1 (60 mg, 55% yield( ) as white solid.

¹H NMR (CDCl₃) δ: 1.25-1.27 (m, 2H), 2.11-2.13 (m, 1H), 2.31-2.35 (m, 1H), 2.83-2.87 (m, 2H), 2.88-2.90 (m, 1H), 3.10-3.19 (m, 1H), 3.31-3.34 (m, 1H), 3.36-3.38 (m, 1H), 3.63-3.65 (m, 1H), 3.75-3.78 (m, 1H), 4.85 (d, J=7.6 Hz, 2H), 4.94 (d, J=7.6 Hz, 2H), 7.33 (d, J=8.0 Hz, 2H), 7.49-7.50 (m, 2H), 7.56 (d, J=8.0 Hz, 2H), 7.64-7.66 (m, 1H).

ESI-MS: m/z 432.18 (M+H)⁺, 100%.

### Example 11: (4-(3-hydroxyoxetan-3-yl)phenyl)(5-(4-(trifluoromethyl)phenyl) hexahydropyrrolo[3,4-c]pyrrol-2 (1H)-yl)methanone

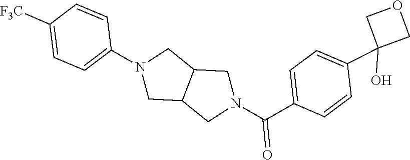 [SMILES: O=C(c1ccc(C2(O)COC2)cc1)N1CC2CN(c3ccc(C(F)(F)F)cc3)CC2C1]

### Step a: Preparation of 2-benzyl-5-(4-(trifluoromethyl)phenyl) octahydropyrrolo[3,4-c]pyrrole

To a mixture of 2-benzyloctahydropyrrolo[3,4-c]pyrrole. (0.475 g, 2.34 mmol), 1-bromo-4-(trifluoromethyl)benzene (0.634 g, 2.82 mmol), (2,2′-bis(diphenylphosphino)-1,1′-binaphthyl) (0.146 g, 0.235 mmol), and sodium tert-butoxide (0.451 g, 4.70 mmol) in toluene (10 mL) was added Tris(dibenzylideneacetone)dipalladium(O) (Pd₂(dba)₃) (0.251 g, 0.235 mmol) under nitrogen atmosphere at 28° C. The mixture was stirred at 110° C. for 16 hrs. The reaction mixture was cooled to ambient temperature, filtered through celite bed and was washed with ethyl acetate. The combined filtrate was concentrated under reduced pressure. The residue was purified by flash column chromatography using 0-100% Ethyl acetate in hexane as eluent to give 2-benzyl-5-(4-(trifluoromethyl)phenyl)octahydropyrrolo[3,4-c]pyrrole (0.320 gm, 40% yield) as white solid.

¹H NMR (DMSO-d₆) δ: 1.74-1.78 (m, 2H), 1.94-1.98 (m, 2H), 2.33 (s, 2H), 2.94-3.01 (m, 4H), 3.41-3.60 (m, 2H), 7.11 (d, J=8.0 Hz, 2H), 7.45-7.47 (m, 4H), 7.70 (d, J=8.0 Hz, 2H).

ESI-MS: m/z 347.17 [M+H]⁺, 100%.

### Step b: Preparation of 2-(4-(trifluoromethyl)phenyl)octahydropyrrolo[3,4-c]pyrrole

A solution of 2-benzyl-5-(4-(trifluoromethyl)phenyl)octahydropyrrolo[3,4-c]pyrrol e (0.320 g, 0.92 mmol) in methanol (15 ml) was added to a suspension of Pd/C (10%) (50 mg) in methanol (5 ml). A solution of ammonium formate (0.582 g, 9.2 mmol) in 0.6 ml of water was added dropwise to the reaction mixture with stirring at 28° C. The mixture was refluxed for 3 hours. Reaction mixture was cooled ambient temperature, filtered through celite bed and washed with methanol. The combined filterate was concentrated in vacuum and the residue was taken up into water, adjusted to pH 9-10 with saturated sodium bicarbonate solution. The resulting solid was filtered, washed with water and dried over P₂O₅ under vacuum to yield 2-(4-(trifluoromethyl)phenyl)octahydropyrrolo[3,4-c]pyrrole (220 mg, 84% yield) as off white solid.

¹H NMR (DMSO-d6) δ 2.68 (br s, 1H), 3.04-3.08 (m, 4H), 3.28-3.32 (m, 1H), 3.65-3.69 (m, 4H), 6.64 (d, J=6.8 Hz, 2H), 7.44 (d, J=8.8 Hz, 2H).

ESI-MS: m/z 257.12 [M+H]⁺, 100%.

### Step c: Preparation of (4-(3-hydroxyoxetan-3-yl)phenyl)(5-(4-(trifluoromethyl) phenyl)hexahydro pyrrolo[3,4-c]pyrrol-2(1H)-yl)methanone

The title compound was prepared from 2-(4-(trifluoromethyl)phenyl) octahydropyrrolo[3,4-c]pyrrole (200 mg, 0.78 mmol) and 4-(3-hydroxyoxetan-3-yl)benzoic acid (151 mg, 0.78 mmol) following the procedure described in step-f, Example 1 to yield the product (215 mg, 64% yield) as off white solid.

¹H NMR (DMSO-d₆) δ: 2.50-2.51 (m, 2H), 3.04-3.09 (m, 1H), 3.34-3.39 (m, 2H), 3.45-3.47 (m, 2H), 3.50-3.51 (m, 1H), 3.75-3.76 (m, 1H), 3.80-3.85 (m, 1H), 4.67 (d, J=8.0 Hz, 2H), 4.78 (d, J=8.0 Hz, 2H), 6.45 (s, 1H), 6.62 (d, J=8.4 Hz, 2H), 7.45 (d, J=8.0 Hz, 2H), 7.55 (d, J=8.0 Hz, 2H), 7.64 (d, J=8.4 Hz, 2H).

ESI-MS: m/z 433.10 (M+H)⁺, 100%.

### Example 12: (4-(3-(2-hydroxyethoxy)oxetan-3-yl)phenyl)(5-(4-(trifluoromethyl) phenyl)hexahydropyrrolo[3,4-c]pyrrol-2(1H)-yl)methanone

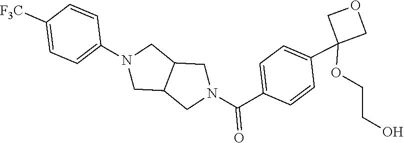 [SMILES: O=C(c1ccc(C2(OCCO)COC2)cc1)N1CC2CN(c3ccc(C(F)(F)F)cc3)CC2C1]

### Step a: Preparation of Ethyl 2-((3-(4-(5-(4-(trifluoromethyl)phenyl) octahydropyrrolo[3,4-c]pyrrole-2-carbonyl)phenyl) oxetan-3-yl) oxy)acetate

The title compound was prepared from (4-(3-hydroxyoxetan-3-yl)phenyl)(5-(4-(trifluoromethyl)phenyl)hexahydropyrrolo[3,4-c]pyrrol-2(1H)-yl)methanone (1 gm, 2.31 mmol) and ethyl bromoacetate (0.309 ml, 2.77 mmol) using the procedure described in example 2 to yield the product (1 gm, 83% yield) as off white solid. The product was directly used for the next step without further purification.

### Step b: Preparation of (4-(3-(2-hydroxyethoxy)oxetan-3-yl)phenyl)(5-(4-(trifluoromethyl)phenyl)hexahydro pyrrolo[3,4-c]pyrrol-2(1H)-yl)methanone

To an ice cold solution of ethyl 2-((3-(4-(5-(4-(trifluoromethyl)phenyl) octahydropyrrolo[3,4-c]pyrrole-2-carbonyl)phenyl) oxetan-3-yl) oxy)acetate (900 mg, 1.78 mmol) in THF (20 ml), lithiumaluminumhydride (81 mg, 2.14 mmol) wad added in small portions over a period of 10 min under nitrogen atmosphere at 0° C. and stirred for further 10 min. Excess LiAlH₄ was quenched by addition of saturated sodium sulfate soliton until white solid separated out. The solid was filtered and washed with ethyl acetate (50 ml). The combined filtrate was dried over sodium sulfate and evaporated on rotavapor under vacuum to yield the product (770 mg, 91% yield) as off white solid.

¹H NMR (CDCl₃) δ: 3.10-3.20 (m, 3H), 3.32-3.80 (m, 4H), 3.40-3.42 (m, 1H), 3.71-3.85 (m, 5H), 4.00-4.02 (m, 1H), 4.84 (d, J=7.2 Hz, 2H), 4.97 (d, J=7.2 Hz, 2H), 6.56 (d, J=8.8 Hz, 2H), 7.47 (d, J=8.4 Hz, 2H), 7.51 (d, J=8.4 Hz, 2H), 7.58 (d, J=8.4 Hz, 2H).

ESI-MS: m/z 477.23 (M+H)⁺, 100%.

### Example 13: 2-((3-(4-(5-(4-(trifluoromethyl)phenyl)octahydropyrrolo[3,4-c]pyrrole-2-carbonyl)phenyl)oxetan-3-yl)oxy)acetic acid

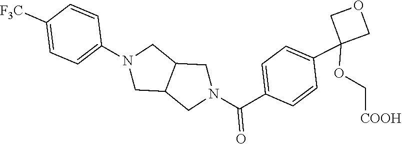 [SMILES: O=C(O)COC1(c2ccc(C(=O)N3CC4CN(c5ccc(C(F)(F)F)cc5)CC4C3)cc2)COC1]

To a solution of 1-((4-bromophenyl)sulfonyl)-4-(4-(trifluoromethyl)phenyl) piperazine (product of step a, example 6) (100 mg, 0.193 mmol) in THF (2.0 ml) and MeOH (0.6 ml), a solution of lithium hydroxide (23 mg, 0.96 mmol) in water (0.6 ml) was added and the reaction mixture was stirred for 12 hrs at 28° C. Solvent was evaporated under vacuum, the residue was diluted with water (20 ml) and neutralized by adding dilute HCl. The solid separated was filtered, washed with water and dried over P205 under vacuum to yield title product (85 mg. 89% Yield) as off white solid.

¹H NMR (DMSO-d₆) δ: 3.03-3.16 (m, 2H), 3.33-3.36 (m, 5H), 3.43-3.45 (m, 2H), 3.48-3.50 (m, 2H), 3.80-3.81 (m, 1H), 3.83-4.0 (m, 1H), 4.66 (d, J=7.2 Hz, 2H), 4.88 (d, J=7.2 Hz, 2H), 6.61 (d, J=8.8 Hz, 2H), 7.45 (d, J=8.8 Hz, 2H), 7.57 (d, J=8.4 Hz, 4H).

ESI-MS: m/z 491.16 (M+H)⁺, 100%.

### Example 14: (4-(3-fluoro-5-(trifluoromethyl)phenyl)piperidin-1-yl)(4-(3-hydroxyoxetan-3-yl)phenyl)methanone

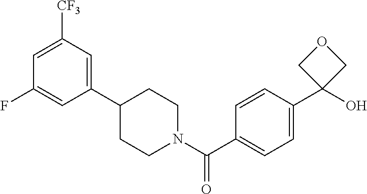 [SMILES: O=C(c1ccc(C2(O)COC2)cc1)N1CCC(c2cc(F)cc(C(F)(F)F)c2)CC1]

The title compound was synthesized according to the procedure given in Example 1 by using 3-Bromo-5-fluorobenzotrifluoride as a starting material.

¹H NMR (DMSO-d₆) δ: 1.57-1.85 (m, 4H), 2.89-3.10 (m, 1H), 3.21-3.23 (s, 2H), 3.71 (s, 1H), 4.52-4.71 (m, 3H), 4.79 (d, J=6.8 Hz, 2H), 6.45 (s, OH), 7.47 (d, J=7.2 Hz, 2H), 7.52-7.69 (m, 5H).

ESI-MS: m/z, 424.15 (M+H)⁺, 100%.

### Example 15: (4-(3-hydroxyoxetan-3-yl)phenyl)(4-(3-(4-methyl-1H-imidazol-1-yl)-5-(trifluoromethyl)phenyl)piperidin-1-yl)methanone

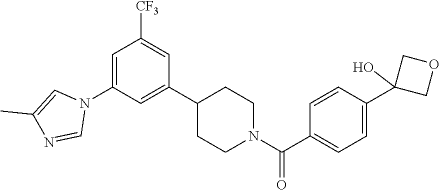 [SMILES: Cc1cn(-c2cc(C3CCN(C(=O)c4ccc(C5(O)COC5)cc4)CC3)cc(C(F)(F)F)c2)cn1]

To a solution of 4-Methylimidazole (65.9 mg, 0.803 mmol) in N-Methyl-2-pyrrolidinone (1.0 ml), sodium hydride (57.8 mg, 1.205 mmol) was added and the reaction mixture was stirred at 30° C. for 30 min. To this (4-(3-fluoro-5-(trifluoromethyl)phenyl)piperidin-1-yl) (4-(3-hydroxyoxetan-3-yl)phenyl)methanone (170 mg, 0.402 mmol) (Example 14) was added in small portions under nitrogen atmosphere. The reaction mixture was stirred at 30° C. for 20 hours and then was further stirred at 80° C. for additional 5 hours. The reaction mixture was poured into water (25 mL) and extracted with ethyl acetate (2×25 mL). The combined organic layer was washed with water (3×15 mL) & brine (15 mL), dried over sodium sulfate and evaporated on rotavapor under vacuum to yield crude product as thick liquid. The crude product was purified by column chromatography using 230-400 mesh silica gel column and 1-5% methanol in chloroform as eluent to yield pure (4-(3-hydroxyoxetan-3-yl)phenyl)(4-(3-(4-methyl-1H-imidazol-1-yl)-5-(trifluoromethyl)phenyl)piperidin-1-yl)methanone (60 mg, 28.6% yield) as white solid

¹H NMR (DMSO-d₆) δ: 1.47-1.95 (m, 4H), 2.19 (s, 3H), 2.67 (s, 2H), 2.81-3.20 (m, 1H), 3.47-3.51 (m, 1H), 4.52-4.60 (m, 1H), 4.70 (d, J=5.6 Hz, 2H), 4.78 (d, J=6.0 Hz, 2H), 6.43 (s, OH), 7.16 (s, 1H), 7.47 (d, J=7.2 Hz, 2H), 7.66-7.75 (m, 4H), 7.84-7.90 (m, 2H).

ESI-MS: m/z, 486.26 (M+H)⁺, 100%.

### Example 16: 3-(4-(3-(3-fluoro-4-(trifluoromethyl)phenyl)-1,2,4-oxadiazol-5-yl)phenyl) oxetan-3-ol

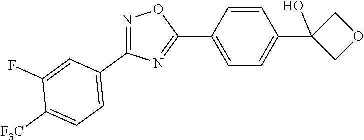 [SMILES: OC1(c2ccc(-c3nc(-c4ccc(C(F)(F)F)c(F)c4)no3)cc2)COC1]

### Step a: Preparation of (Z)-3-fluoro-N′-((4-(3-hydroxyoxetan-3-yl)benzoyl)oxy)-4-(trifluoromethyl)benzimidamide

To a solution of 4-(3-hydroxyoxetan-3-yl)benzoic acid (219 mg, 1.125 mmol) and HBTU (469 mg, 1.238 mmol) in N,N-dimethylformamide (2.0 ml), Diisopropylethylamine (0.590 ml, 3.38 mmol) was added and the reaction mixture was stirred at 30° C. for 5 min. 3-Fluoro-N-hydroxy-4-(trifluoromethyl)benzimidamide (250 mg, 1.125 mmol) was added and the reaction mixture was stirred overnight at 30° C. The reaction mixture was poured into water (50 mL). Solid separated was filtered through Buchner funnel, washed with water and dried over P₂O₅ under vacuum to yield (Z)-3-fluoro-N′-((4-(3-hydroxyoxetan-3-yl)benzoyl)oxy)-4-(trifluoromethyl)benzimidamide (400 mg, 89% yield) as Pale brown solid which was directly used in the next step without purification.

### Step b: Preparation of 3-(4-(3-(3-fluoro-4-(trifluoromethyl)phenyl)-1,2,4-oxadiazol-5-yl)phenyl)oxetan-3-ol

A solution of (Z)-3-fluoro-N′-((4-(3-hydroxyoxetan-3-yl)benzoyl)oxy)-4-(trifluoromethyl) benzimidamide (400 mg, 1.004 mmol) in Dimethylformamide (5.0 ml) was heated under nitrogen atmosphere at 120° C. for 5 hours. Solvent was evaporated from the reaction mixture under reduced pressure, residue was dissolved in ethyl acetate (25 ml) and washed with 1N HCl (10 ml), NaHCO₃ (10 ml) & brine (20 ml). The organic phase was dried over sodium sulfate and evaporated under reduced pressure to yield crude product as tick liquid. The crude product was purified by column chromatography using 230-400 mesh silica gel column and 25% Ethyl acetate in hexane as eluent to yield pure 3-(4-(3-(3-fluoro-4-(trifluoromethyl)phenyl)-1,2,4-oxadiazol-5-yl)phenyl)oxetan-3-ol (200 mg, 51.8% yield) as white solid.

¹H NMR (CDCl₃) δ: 4.71 (d, J=6.8 Hz, 2H), 4.85 (d, J=6.8 Hz, 2H), 6.67 (s, —OH), 7.92 (d, J=8.4 Hz, 2H), 8.03-8.07 (m, 1H), 8.13 (d, J=9.6 Hz, 2H), 8.25 (d, J=8.4 Hz, 2H).

ESI-MS: m/z, 381.12 (M+H)⁺, 100%.

### Example 17: 3-(4-((1-(4-(trifluoromethyl)phenyl)piperidin-4-yl)oxy)phenyl) oxetan-3-ol

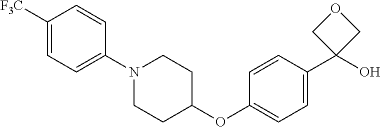 [SMILES: OC1(c2ccc(OC3CCN(c4ccc(C(F)(F)F)cc4)CC3)cc2)COC1]

### Step a: Preparation of 1-(4-(trifluoromethyl)phenyl)piperidin-4-ol

To a solution of 1-bromo-4-(trifluoromethyl)benzene (5.56 g, 24.72 mmol) in Toluene (20 ml), piperidin-4-ol (3 g, 29.7 mmol) was added followed by addition of sodium tert-butoxide (3.56 g, 37.10 mmol), BINAP (0.462 g, 0.741 mmol) & Pd₂(dba)₃ (0.226 g, 0.247 mmol) under nitrogen atmosphere at rt. The reaction mixture was stirred under nitrogen atmosphere at 120° C. for 16 hrs. After completion of reaction, reaction mixture was cooled to ambient temperature and filtered through celite bed. The filtrate was diluted with ethyl acetate (40 mL) and water (40 mL). Organic layer was separated and aqeuous layer was extracted with ethyl acetate (2×20 mL). The combined organic layer was washed with water (2×60 mL) & brine (60 mL), dried over Na₂SO₄ and evaporated on rotavapor under vacuum to yield crude product as an oil. The crude product was purified by column chromatography using 100-200 mesh silica gel column and 20% Ethyl acetate in n-hexane as eluent to obtained 1-(4-(trifluoromethyl)phenyl)piperidin-4-ol (3.9 g, 64% yield) as thick liquid.

¹H NMR (CDCl₃) δ: 1.54 (s, 1H), 1.64-1.72 (m, 2H), 1.99-2.05 (m, 2H), 3.03-3.09 (m, 2H), 3.65-3.71 (m, 2H), 3.90-3.96 (m, 1H), 6.95 (d, J=8.4 Hz, 2H), 7.48 (d, J=8.4 Hz, 2H).

ESI-MS: m/z 246.1 (M+H)⁺, 100%.

### Step b: Preparation of 4-(4-bromophenoxy)-1-(4-(trifluoromethyl)phenyl)piperidine

To a suspension of sodium hydride (0.245 g, 5.10 mmol) in DMF (4 ml), a solution of 1-(4-(trifluoromethyl)phenyl)piperidin-4-ol (0.5 g, 2.039 mmol) in DMF (0.5 ml) was added dropwise at 0-10° C. over a period of 10 min and reaction mixture was stirred for 30 min. 1-bromo-4-fluorobenzene (0.428 g, 2.447 mmol) was added to the reaction mixture and stirred at 120° C. for 8 hrs. The reaction mixture was poured into ice cold water (25 mL) and precipitated solid was collected by filtration. Then it was washed with hexane and dried over P₂O₅ overnight to obtain 4-(4-bromophenoxy)-1-(4-(trifluoromethyl)phenyl)piperidine (0.648 g, 79% yield) as off white solid.

¹H NMR (CDCl₃) δ: 1.60-1.97 (m, 2H), 2.06-2.12 (m, 2H), 3.23-3.29 (m, 2H), 3.56-3.62 (m, 2H), 4.47-4.52 (m, 1H), 6.83 (d, J=8.8 Hz, 2H), 6.97 (d, J=8.8 Hz, 2H), 7.40 (d, J=8.4 Hz, 2H), 7.51 (d, J=8.4 Hz, 2H).

ESI-MS: m/z 400.2 (M)⁺, 100%.

### Step c: Preparation of 3-(4-((1-(4-(trifluoromethyl)phenyl)piperidin-4-yl)oxy) phenyl)oxetan-3-ol

A solution of 4-(4-bromophenoxy)-1-(4-(trifluoromethyl)phenyl)piperidine (0.640 g, 1.599 mmol) in THF (8 ml) was cooled to −78° C. and n-butyllithium (2.5M in hexane) (1.599 ml, 4.00 mmol) was added drop wise maintaining the temperature at −78° C. over a period of 15 min. The reaction mixture was stirred further for 15 min and a solution of oxetan-3-one (0.173 g, 2.399 mmol) in THF (1 ml) was added under nitrogen atmosphere at −78° C. The reaction mixture was stirred at −78° C. for 3 hr. The reaction mixture was poured into saturated NH₄Cl solution (25 mL) and extracted with ethyl acetate (2×20 mL). The combined organic layers was washed with water (2×15 mL) & brine (20 mL), dried over Na₂SO₄ and evaporated on rotavapor under vacuum. The residue was purified by column chromatography using 100-200 mesh silica gel column and 20% Ethyl acetate in Hexane as eluent to yield 3-(4-((1-(4-(trifluoromethyl)phenyl)piperidin-4-yl)oxy)phenyl)oxetan-3-ol (127 mg, 20% Yield) as off white solid.

¹H NMR (CDCl₃) δ: 1.92-2.00 (m, 2H), 2.06-2.15 (m, 2H), 2.75 (brs, 1H), 3.25-3.31 (m, 2H), 3.58-3.64 (m, 2H), 4.54-4.59 (m, 1H), 4.91-4.94 (m, 4H), 6.96-7.01 (m, 4H), 7.49-7.54 (m, 4H).

ESI-MS: m/z, 394.30 (M+H)⁺, 100%.

### Example 18: (4-(3-hydroxyoxetan-3-yl)phenyl)(2-(4-(trifluoromethyl)phenyl)-6,7-dihydrothieno[3,2-c]pyridin-5(4H)-yl)methanone

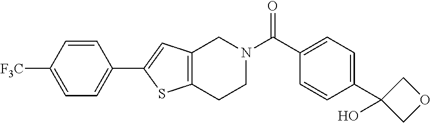 [SMILES: O=C(c1ccc(C2(O)COC2)cc1)N1CCc2sc(-c3ccc(C(F)(F)F)cc3)cc2C1]

### Step a: Preparation of tert-butyl 2-(4-(trifluoromethyl)phenyl)-6,7-dihydrothieno[3,2-c]pyridine-5(4H)-carboxylate

To a stirred solution of tert-butyl 2-bromo-6,7-dihydrothieno[3,2-c]pyridine-5(4H)-carboxylate (0.5 g, 1.571 mmol) in DMF (10 mL), Dichlorobis(triphenylphosphine)-palladium(II) (0.110 g, 0.157 mmol) was added and the mixture was heated at 90° C. for 1 hour. (4-(Trifluoromethyl)phenyl)boronic acid (0.298 g, 1.571 mmol) was added and the reaction mixture was further stirred for 30 min at the same temperature. Then a solution of potassium bicarbonate (0.944 g, 9.43 mmol) in water (4.00 mL) was added and stirring was continued for 3 h at 90° C. The reaction mixture was poured into water (20 mL) and extracted with ethyl acetate (2×15 mL). The combined organic extract was washed with water (30 mL) & brine (30 mL), dried over Na₂SO₄ and evaporated on rotavapor under vacuum. The residue was purified by column chromatography using 100-200 mesh silica gel column and 10% Ethyl acetate in Hexane as eluent to yield tert-butyl 2-(4-(trifluoromethyl)phenyl)-6,7-dihydrothieno[3,2-c]pyridine-5(4H)-carboxylate (390 mg, 64.7% Yield) as off white solid.

¹H NMR (CDCl₃) δ: 1.52 (s, 9H), 2.89 (brs, 2H), 3.77 (brs, 2H), 4.53 (brs, 2H), 7.10 (s, 1H), 7.61-7.66 (m, 4H).

### Step b: Preparation of 2-(4-(trifluoromethyl)phenyl)-4,5,6,7-tetrahydrothieno[3,2-c]pyridine hydrochloride

The title product was synthesized form tert-butyl 2-(4-(trifluoromethyl)phenyl)-6,7-dihydrothieno[3,2-c]pyridine-5(4H)-carboxylate following the procedure described in step b of example 6, (311 mg, 91.0% Yield).

¹H NMR (DMSO-d₆) δ: 3.09 (t, J=6.0 Hz, 2H), 3.44 (t, J=6.0 Hz, 2H), 4.28 (s, 2H), 7.51 (s, 1H), 7.78 (d, J=8.4 Hz, 2H), 7.82 (d, J=8.4 Hz, 2H), 9.36 (brs, 2H).

### Step c: Preparation of (4-(3-hydroxyoxetan-3-yl)phenyl)(2-(4-(trifluoromethyl)phenyl)-6,7-dihydrothieno [3,2-c]pyridin-5 (4H)-yl)methanone

The title product was synthesized form 2-(4-(trifluoromethyl)phenyl)-4,5,6,7-tetrahydrothieno[3,2-c]pyridine hydrochloride and 4-(3-hydroxyoxetan-3-yl)benzoic acid using the procedure described in step f of Example 1, (110 mg, 60.4% Yield)

¹H NMR (DMSO-d₆) δ: 2.93 (brs, 2H), 3.65 (brs, 1H), 3.94 (brs, 1H), 4.55 (brs, 1H), 4.71 (d, J=6.8 Hz, 2H), 4.81 (d, J=6.8 Hz, 3H), 6.41 (s, 1H), 7.50-7.56 (m, 3H), 7.70-7.81 (m, 6H).

ESI-MS: m/z, 460.08 (M+H)⁺, 30%.

### Example 19: (1-benzyl-3-(4-(trifluoromethyl) phenyl)-4,6-dihydropyrrolo[3,4-c]pyrazol-5(1H)-yl)(4-(3-hydroxyoxetan-3-yl)phenyl)methanone

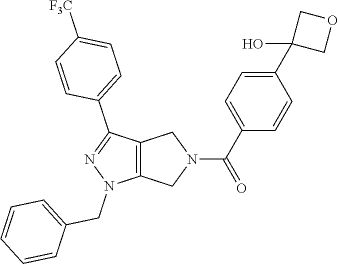 [SMILES: O=C(c1ccc(C2(O)COC2)cc1)N1Cc2c(-c3ccc(C(F)(F)F)cc3)nn(Cc3ccccc3)c2C1]

### Step a: Preparation of tert-butyl 1-benzyl-3-(4-(trifluoromethyl)phenyl)-4,6-dihydropyrrolo[3,4-c]pyrazole-5 (1H)-carboxylate

To a stirred solution of tert-butyl 1-benzyl-3-bromo-4,6-dihydropyrrolo[3,4-c]pyrazole-5(1H)-carboxylate (1.5 g, 3.97 mmol) in DMF (30 mL), dichlorobis(triphenylphosphine)-palladium(II) (0.278 g, 0.397 mmol) was added and the mixture was heated to 90° C. for an hour. (4-(trifluoromethyl)phenyl)boronic acid (0.828 g, 4.36 mmol) was added and the reaction mixture was further stirred for 30 min. Then a solution of potassium bicarbonate (2.382 g, 23.79 mmol) in Water (12 mL) was added and stirring was continued for 3 h at 90° C. The reaction mixture was diluted with water (40 mL) and extracted with ethyl acetate (2×30 mL). The combined organic extracts were washed with water (50 mL) & brine (50 mL), dried over Na₂SO₄ and evaporated on rotavapor under vacuum. The residue was purified by column chromatography using 100-200 silica gel column and 8% ethyl acetate in hexane as eluent to yield tert-butyl 1-benzyl-3-(4-(trifluoromethyl)phenyl)-4,6-dihydropyrrolo[3,4-c]pyrazole-5(1H)-carboxylate (1.56 g, 89% Yield) as thick gum.

¹H NMR (CDCl₃) δ: 1.53 (d, J=8.0 Hz, 9H), 4.48-4.60 (m, 4H), 5.32-5.37 (m, 2H), 7.08-7.09 (m, 2H), 7.27-7.35 (m, 3H), 7.43 (d, J=7.6 Hz, 2H) 7.67-7.71 (m, 2H).

ESI-MS: m/z, 444.14 (M+H)⁺, 100%.

### Step b: Preparation of 1-benzyl-3-(4-(trifluoromethyl)phenyl)-1,4,5,6-tetrahydropyrrolo[3,4-c]pyrazole 2,2,2-trifluoroacetate

Trifluoroacetic acid (0.174 mL, 2.255 mmol) was added to a solution of tert-butyl 1-benzyl-3-(4-(trifluoromethyl)phenyl)-4,6-dihydropyrrolo[3,4-c]pyrazole-5(1H)-carboxylate (0.1 g, 0.225 mmol) in dichloromethane (30 ml) under nitrogen atmosphere at ambient temperature and stirred for 2 hr. Solvent was evaporated on rotavapor under vacuum and the residue was diluted with diisopropylether (5 ml) and stirred for 30 min to obtain pure 1-benzyl-3-(4-(trifluoromethyl)phenyl)-1,4,5,6-tetrahydropyrrolo[3,4-c]pyrazole 2,2,2-trifluoroacetate (102 mg, 99% Yield) as thick liquid.

ESI-MS: m/z, 344.10 (M+H)⁺, 100%.

### Step c: Preparation of (1-benzyl-3-(4-(trifluoromethyl)phenyl)-4,6-dihydropyrrolo[3,4-c]pyrazol-5(1H)-yl)(4-(3-hydroxyoxetan-3-yl)phenyl)methanone

The title product was synthesized form 1-benzyl-3-(4-(trifluoromethyl)phenyl)-1,4,5,6-tetrahydropyrrolo[3,4-c]pyrazole 2,2,2-trifluoroacetate and 4-(3-hydroxyoxetan-3-yl)benzoic acid using the procedure described in step f of Example 1, (55 mg, 46.3% Yield)

¹H NMR (DMSO-d₆) δ: 4.66-4.72 (m, 5H), 4.78-4.81 (m, 3H), 5.45 (d, J=7.6 Hz, 2H), 6.47 (d, J=10.4 Hz, 1H), 7.01-7.04 (m, 2H), 7.24-7.29 (m, 3H), 7.62-7.79 (m 7H), 7.85 (d, J=8.0 Hz, 1H).

ESI-MS: m/z, 520.13 (M+H)⁺, 100%.

### Example 20: (4-(3-hydroxyoxetan-3-yl)phenyl)(1-methyl-3-(4-(trifluoromethyl) phenyl)-4,6-dihydropyrrolo[3,4-c]pyrazol-5(1H)-yl)methanone

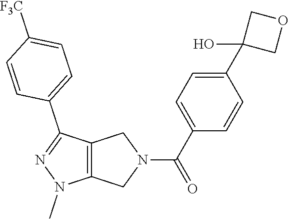 [SMILES: Cn1nc(-c2ccc(C(F)(F)F)cc2)c2c1CN(C(=O)c1ccc(C3(O)COC3)cc1)C2]

### Step a: Preparation of tert-butyl 3-(4-(trifluoromethyl)phenyl)-4,6-dihydropyrrolo[3,4-c]pyrazole-5(1H)-carboxylate

A solution of tert-butyl 1-benzyl-3-(4-(trifluoromethyl)phenyl)-4,6-dihydropyrrolo[3,4-c]pyrazole-5(1H)-carboxylate (0.8 g, 1.804 mmol) in EtOH (15 ml) was added to a suspension of Pd(OH)₂ (20%) (0.6 g, 0.854 mmol) in EtOH (10 ml) and the reaction mixture was hydrogenated in Paar hydrogenation apparatus under 50 psi hydrogen pressure at ambient temperature for 24 hrs. The reaction mixture was filtered through celite bed and the filtrate was evaporated on rotavapor under reduced pressure to obtained tert-butyl 3-(4-(trifluoromethyl)phenyl)-4,6-dihydropyrrolo[3,4-c]pyrazole-5(1H)-carboxylate (100 mg, 15.69% yield) as sticky solid which was directly used for the next step without purification.

### Step b: Preparation of tert-butyl 1-methyl-3-(4-(trifluoromethyl)phenyl)-4,6-dihydropyrrolo[3,4-c]pyrazole-5(1H)-carboxylate and tert-butyl 2-methyl-3-(4-(trifluoromethyl)phenyl)-2,6-dihydropyrrolo[3,4-c]pyrazole-5(4H)-carboxylate

To a suspension of NaH (0.019 g, 0.425 mmol) in THF (2 ml), a solution of tert-butyl 3-(4-(trifluoromethyl)phenyl)-4,6-dihydropyrrolo[3,4-c]pyrazole-5 (1H)-carboxylate (0.1 g, 0.283 mmol) in THF (2 ml) was added dropwise at 0-10° C. followed by addition of iodomethane (0.027 ml, 0.425 mmol) and the reaction mixture was stirred at 55° C. for 16 hrs. The reaction mixture was poured into ice cold water (10 mL) and extracted with ethyl acetate (2×10 mL). The combined organic layer was washed with water (2×15 mL) & brine (15 mL), dried over Na₂SO₄ and evaporated under vacuum to obtain mixture of tert-butyl 1-methyl-3-(4-(trifluoromethyl)phenyl)-4,6-dihydropyrrolo[3,4-c]pyrazole-5 (1H)-carboxylate and tert-butyl 2-methyl-3-(4-(trifluoromethyl)phenyl)-2,6-dihydropyrrolo[3,4-c]pyrazole-5(4H)-carboxylate (100 mg, 98% yield) as thick liquid which was directly used for the next step without purification.

### Step c: Preparation of 1-methyl-3-(4-(trifluoromethyl)phenyl)-1,4,5,6-tetrahydropyrrolo[3,4-c]pyrazole hydrochloride and 2-methyl-3-(4-(trifluoromethyl) phenyl)-2,4,5,6-tetrahydropyrrolo[3,4-c]pyrazole hydrochloride

HCl in 1,4-dioxane (3 ml) was added to a solution of mixture of products of step b (0.230 g, 0.626 mmol) in dichloromethane (3 ml) under nitrogen atmosphere at 27° C. The reaction mixture was stirred at ambient temperature for 3 hrs. Solvent was removed under vacuum and the residue was triturated in diethylether (4 ml) to obtained mixture of 1-methyl-3-(4-(trifluoromethyl)phenyl)-1,4,5,6-tetrahydropyrrolo[3,4-c]pyrazole hydrochloride and 2-methyl-3-(4-(trifluoromethyl)phenyl)-2,4,5,6-tetrahydropyrrolo[3,4-c]pyrazole hydrochloride (190 mg, 100% Yield) as off white solid.

ESI-MS: m/z, 268.10 (M+H)⁺, 100%.

### Step d: Preparation of (4-(3-hydroxyoxetan-3-yl)phenyl)(1-methyl-3-(4-(trifluoromethyl)phenyl)-4,6-dihydropyrrolo[3,4-c]pyrazol-5 (1H)-yl)methanone

To a solution of product of step c (0.180 g, 0.593 mmol) in DMF (3 ml), 4-(3-hydroxyoxetan-3-yl)benzoic acid (0.127 g, 0.652 mmol) and HBTU (0.462 g, 0.889 mmol) was added followed by addition of DIPEA (0.311 ml, 1.778 mmol) under nitrogen atmosphere at 27° C. The reaction mixture was stirred under nitrogen atmosphere for 16 hrs. The reaction mixture was poured into ice cold water (15 mL) and extracted by ethyl acetate (3×10 mL). The combined organic layer was washed with water (3×25 mL) & brine (25 mL), dried over Na₂SO₄ and evaporated on rotavapor under vacuum to yield crude product as thick liquid. The crude product was purified by preparative HPLC to yield (4-(3-hydroxyoxetan-3-yl)phenyl)(1-methyl-3-(4-(trifluoromethyl)phenyl)-4,6-dihydropyrrolo[3,4-c]pyrazol-5 (1H)-yl)methanone (30 mg, 10.88%) as off white solid.

¹H NMR (DMSO-d₆) δ: 3.93 (s, 3H), 4.68-4.72 (m, 6H), 4.77-4.82 (m, 2H), 6.65 (brs, 1H), 7.62-7.75 (m, 6H), 7.81-7.91 (m, 2H).

ESI-MS: m/z, 444.17 (M+H)⁺, 100%.

### Example 21: (4-(3-hydroxyoxetan-3-yl)phenyl)(2-methyl-3-(4-(trifluoromethyl) phenyl)-2,6-dihydropyrrolo[3,4-c]pyrazol-5(4H)-yl)methanone

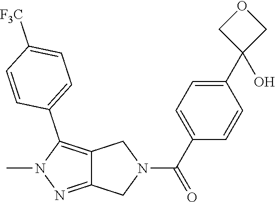 [SMILES: Cn1nc2c(c1-c1ccc(C(F)(F)F)cc1)CN(C(=O)c1ccc(C3(O)COC3)cc1)C2]

The title compound is isolated during preparative HPLC of mixture obtained in step d of Example 20 as off white solid (32 mg, 12% yield).

¹H NMR (DMSO-d₆) δ: 3.78-3.90 (m, 3H), 4.71-4.74 (m, 3H), 4.81-4.83 (m, 4H), 4.89 (s, 1H), 6.50-6.51 (m, 1H), 7.65-7.74 (m, 6H), 7.78 (d, J=8.4 Hz, 1H), 7.88 (d, J=8.0 Hz, 1H).

ESI-MS: m/z, 444.17 (M+H)⁺, 100%.

The following examples were prepared following the general procedures given in the Examples 1-21 with suitable modifications, alterations and other process variations which are within the scope of a person skilled in the art

### Example 22: (4-(3-hydroxyoxetan-3-yl)phenyl)(4-(4-(trifluoromethoxy)phenyl) piperidin-1-yl)methanone

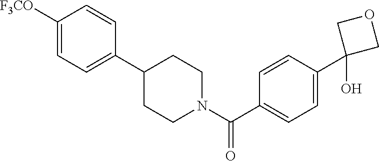 [SMILES: O=C(c1ccc(C2(O)COC2)cc1)N1CCC(c2ccc(OC(F)(F)F)cc2)CC1]

¹H NMR (DMSO-d₆) δ: 1.58-1.85 (m, 4H), 2.85-2.91 (m, 2H), 3.10-3.16 (m, 1H), 3.66-3.74 (m, 1H), 4.64-4.67 (m, 1H), 4.70 (d, J=6.8 Hz, 2H), 4.79 (d, J=6.8 Hz, 2H), 6.43 (s, 1H), 7.30 (d, J=8.0 Hz, 2H), 7.43 (d, J=8.8 Hz, 2H), 7.47 (d, J=8.0 Hz, 2H), 7.68 (d, J=8.8 Hz, 2H).

ESI-MS: m/z 422.14 (M+H)⁺, 40%.

### Example 23: (4-(3-hydroxyoxetan-3-yl)phenyl)(4-(p-tolyl)piperidin-1-yl) methanone

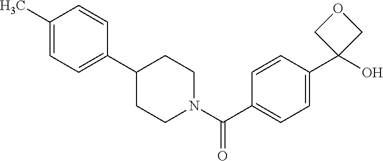 [SMILES: Cc1ccc(C2CCN(C(=O)c3ccc(C4(O)COC4)cc3)CC2)cc1]

¹H NMR (DMSO-d₆) δ: 1.54-1.82 (m, 4H), 2.26 (s, 3H), 2.73-2.89 (m, 2H), 3.14 (brs, 1H), 3.69 (bs, 1H), 4.63 (brs, 1H), 4.70 (d, J=6.8 Hz, 2H), 4.79 (d, J=6.4 Hz, 2H), 6.43 (s, 1H), 7.10-7.17 (m, 4H), 7.46 (d, J=8.0 Hz, 2H), 7.68 (d, J=8.4 Hz, 2H).

ESI-MS: m/z, 452.17 (M+H)⁺, 100%.

### Example 24: (4-(3-hydroxyoxetan-3-yl)phenyl)(4-(3-(trifluoromethyl)phenyl) piperidin-1-yl)methanone

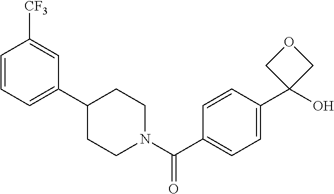 [SMILES: O=C(c1ccc(C2(O)COC2)cc1)N1CCC(c2cccc(C(F)(F)F)c2)CC1]

¹H NMR (DMSO-d6) δ: 1.64-1.87 (m, 4H), 2.87-2.99 (m, 2H), 3.16 (brs, 1H), 3.71 (brs, 1H), 4.69-4.71 (m, 3H), 4.79 (d, J=6.4 Hz, 2H), 6.43 (s, 1H), 7.49 (d, J=8.4 Hz, 2H), 7.53-7.69 (m, 6H).

ESI-MS: m/z, 406.13 (M+H)⁺, 100%.

### Example 25: (3-(3-hydroxyoxetan-3-yl)phenyl)(4-(p-tolyl)piperidin-1-yl) methanone

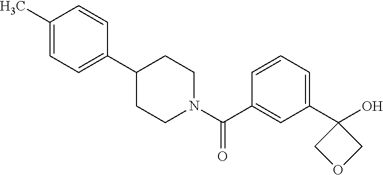 [SMILES: Cc1ccc(C2CCN(C(=O)c3cccc(C4(O)COC4)c3)CC2)cc1]

¹H NMR (DMSO-d₆) δ: 1.58-1.83 (m, 4H), 2.26 (s, 3H), 2.74-2.89 (m, 2H), 3.15 (brs, 1H), 3.68 (bs, 1H), 4.64-4.68 (m, 3H), 4.79 (d, J=6.4 Hz, 2H), 6.44 (s, 1H), 7.09-7.17 (m, 4H), 7.35-7.38 (m, 1H), 7.48 (d, J=7.6 Hz, 1H), 7.70 (s, 1H), 7.95 (s, 1H).

ESI-MS: m/z, 352.17 (M+H)⁺, 100%.

### Example 26: (4-(3-chloro-4-methoxyphenyl)piperidin-1-yl)(4-(3-hydroxyoxetan-3-yl)phenyl)methanone

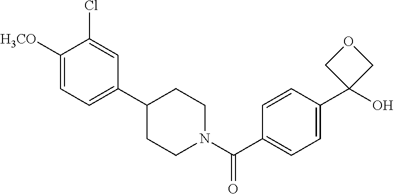 [SMILES: COc1ccc(C2CCN(C(=O)c3ccc(C4(O)COC4)cc3)CC2)cc1Cl]

¹H NMR (DMSO-d₆) δ: 1.54-1.81 (m, 4H), 2.74-2.80 (m, 2H), 3.15 (brs, 1H), 3.64 (brs, 1H), 3.82 (s, 3H), 4.59-4.62 (m, 1H), 4.69 (d, J=6.8 Hz, 2H), 4.79 (d, J=6.8 Hz, 2H), 6.44 (s, 1H), 7.07 (d, J=8.4 Hz, 1H), 7.22 (dd, J=8.4 & 2.0 Hz, 1H), 7.35 (d, J=2.0 Hz, 1H), 7.47 (dd, J=6.8 & 2.0 Hz, 2H), 7.68 (dd, J=6.4 & 1.6 Hz, 2H).

ESI-MS: m/z, 402.13 (M+H)⁺, 100%.

### Example 27: (4-(3-hydroxyoxetan-3-yl)phenyl)(4-(4-methoxyphenyl)piperidin-1-yl)methanone

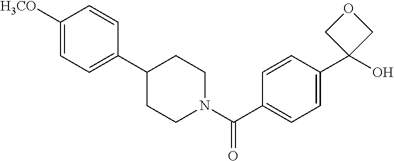 [SMILES: COc1ccc(C2CCN(C(=O)c3ccc(C4(O)COC4)cc3)CC2)cc1]

¹H NMR (DMSO-d₆) δ: 1.56-1.82 (m, 4H), 2.67-2.89 (m, 2H), 3.14 (brs, 1H), 3.72 (s, 4H), 4.62-4.64 (m, 1H), 4.69 (d, J=6.4 Hz, 2H), 4.79 (d, J=6.4 Hz, 2H), 6.43 (s, 1H), 7.85 (d, J=8.4 Hz, 2H), 7.19 (d, J=8.4, 2H), 7.46 (d, J=8.0 Hz, 2H), 7.67 (d, J=8.0 Hz, 2H).

ESI-MS: m/z, 338.17 (M+H)⁺, 100%.

### Example 28: (4-(3-fluoro-4-methoxyphenyl)piperidin-1-yl)(4-(3-hydroxyoxetan-3-yl)phenyl)methanone

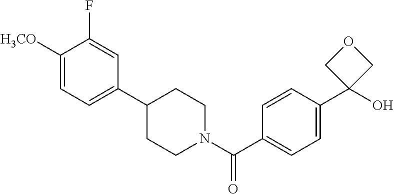 [SMILES: COc1ccc(C2CCN(C(=O)c3ccc(C4(O)COC4)cc3)CC2)cc1F]

¹H NMR (DMSO-d₆) δ: 1.60-1.83 (m, 4H), 2.77 bs, 2H), 3.11-3.14 (brs, 1H), 3.73 (s, 1H), 3.80 (s, 3H), 4.70 (bs, 3H), 4.79 (s, 2H), 6.43 (s, 1H), 7.15-7.35 (m, 3H), 7.47 (s, 2H), 7.67 (s, 2H).

ESI-MS: m/z, 386.17 (M+H)⁺, 100%.

### Example 29: (4-(3-chloro-4-methylphenyl)piperidin-1-yl)(4-(3-hydroxyoxetan-3-yl)phenyl)methanone

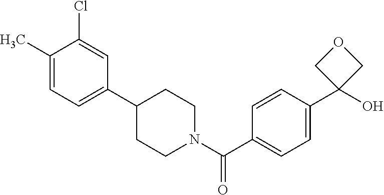 [SMILES: Cc1ccc(C2CCN(C(=O)c3ccc(C4(O)COC4)cc3)CC2)cc1Cl]

¹H NMR (DMSO-d₆) δ: 1.65-2.06 (m, 4H), 2.44 (s, 3H), 2.65-2.90 (m, 2H), 3.14 (brs, 1H), 3.87 (s, 1H), 3.82-3.95 (m, 5H), 7.02 (d, J=8.0 Hz, 1H), 7.12-7.28 (m, 2H), 7.44 (d, J=9.6 Hz, 2H), 7.64 (d, J=8.0 Hz, 2H).

ESI-MS: m/z, 386.14 (M+H)⁺, 100%.

### Example 30: (4-(3-hydroxyoxetan-3-yl)phenyl)(4-(3-methoxy-4-(trifluoromethyl) phenyl)piperidin-1-yl)methanone

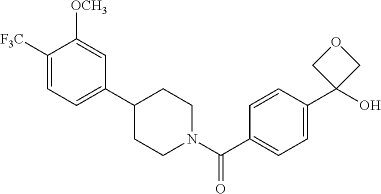 [SMILES: COc1cc(C2CCN(C(=O)c3ccc(C4(O)COC4)cc3)CC2)ccc1C(F)(F)F]

¹H NMR (DMSO-d₆) δ: 1.68-1.95 (m, 4H), 2.80-3.00 (brs, 2H), 3.25 (brs, 1H), 3.81 (s, 1H), 3.92 (s, 3H), 4.69-4.71 (m, 3H), 4.79 (d, J=6.8 Hz, 2H), 6.44 (s, 1H), 7.32 (t, J=7.60 Hz, 1H), 7.48-7.54 (m, 3H), 7.68 (d, J=8.4 Hz, 2H), 7.76 (d, J=7.2 Hz, 1H).

ESI-MS: m/z, 336.12 (M+H)⁺, 100%.

### Example 31: (4-(3-fluoro-4-(trifluoromethyl)phenyl)piperidin-1-yl)(4-(3-hydroxyoxetan-3-yl)phenyl)methanone

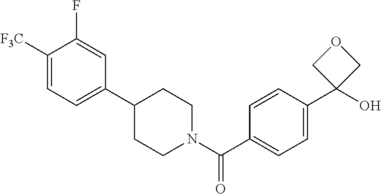 [SMILES: O=C(c1ccc(C2(O)COC2)cc1)N1CCC(c2ccc(C(F)(F)F)c(F)c2)CC1]

¹H NMR (CDCl₃) δ: 1.76-2.00 (m, 4H), 2.90 (brs, 1H), 3.21-3.32 (m, 1H), 3.90 (brs, 1H), 4.14 (brs, 1H), 4.79-4.94 (m, 5H), 7.13-7.50 (m, 5H), 7.62 (d, J=6.8 Hz, 2H).

ESI-MS: m/z, 424.10 (M+H)⁺, 100%.

### Example 32: (4-(3-chloro-4-(trifluoromethyl)phenyl)piperidin-1-yl)(4-(3-hydroxyoxetan-3-yl)phenyl)methanone

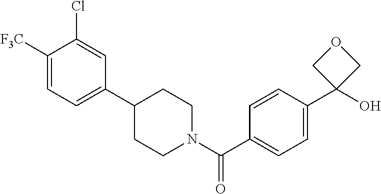 [SMILES: O=C(c1ccc(C2(O)COC2)cc1)N1CCC(c2ccc(C(F)(F)F)c(Cl)c2)CC1]

¹H NMR (DMSO-d₆) δ: 1.53-1.86 (m, 4H), 2.85 (brs, 1H), 2.96 (brs, 1H), 3.15-3.25 (m, 1H), 3.71 (brs, 1H), 4.61-4.80 (m, 3H), 4.90 (d, J=13.2 Hz, 2H), 6.43 (s, 1H), 7.48-7.56 (m, 3H), 7.67-7.80 (m, 3H), 7.88 (d, J=9.6 Hz, 1H).

ESI-MS: m/z, 440.07 (M+H)⁺, 100%.

### Example 33: (4-(3-hydroxyoxetan-3-yl)phenyl)(4-(2-(trifluoromethyl)phenyl) piperidin-1-yl)methanone

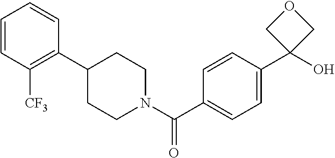 [SMILES: O=C(c1ccc(C2(O)COC2)cc1)N1CCC(c2ccccc2C(F)(F)F)CC1]

¹H NMR (CDCl₃) δ: 1.64-2.06 (m, 4H), 2.90 (brs, 1H), 3.21-3.27 (m, 2H), 3.91 (brs, 1H), 4.84 (d, J=7.2 Hz, 2H), 4.87-4.96 (m, 3H), 7.29-7.38 (m, 1H), 7.43-7.67 (m, 7H).

ESI-MS: m/z, 406.20 (M+H)⁺, 100%.

### Example 34: (4-(3-hydroxyoxetan-3-yl)phenyl)(3-(4-(trifluoromethyl)phenyl) piperidin-1-yl)methanone

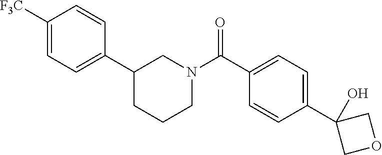 [SMILES: O=C(c1ccc(C2(O)COC2)cc1)N1CCCC(c2ccc(C(F)(F)F)cc2)C1]

¹H NMR (DMSO-d6) δ: 1.61-1.91 (m, 4H), 2.92-2.97 (m, 2H), 3.17 (brs, 1H), 3.71 (brs, 1H), 4.67-4.71 (m, 3H), 4.80 (d, J=6.4 Hz, 2H), 6.44 (s, 1H), 7.48 (d, J=8.4 Hz, 2H), 7.52 (d, J=8.4 Hz, 2H), 7.66-7.69 (m, 4H).

ESI-MS: m/z, 406.20 (M+H)⁺, 100%.

### Example 35: 2-((3-(4-(4-(4-(trifluoromethyl)phenyl)piperidine-1-carbonyl) phenyl)oxetan-3-yl)oxy)acetic acid

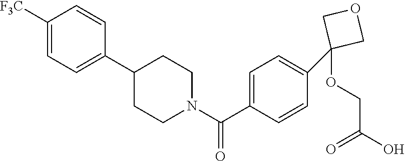 [SMILES: O=C(O)COC1(c2ccc(C(=O)N3CCC(c4ccc(C(F)(F)F)cc4)CC3)cc2)COC1]

¹H NMR (DMSO-d₆) δ: 1.23-1.79 (m, 4H), 2.88-2.97 (m, 2H), 3.14 (brs, 1H), 3.45 (s, 2H), 3.83 (brs, 1H), 4.70 (d, J=6.8 Hz, 3H), 4.89 (d, J=7.2 Hz, 2H), 7.49 (d, J=8.0 Hz, 2H), 7.54 (d, J=8.0 Hz, 2H), 7.60 (d, J=8.4 Hz, 2H) 7.67 (d, J=8.4 Hz, 2H).

ESI-MS: m/z 464.18 (M+H)⁺, 100%.

### Example 36: 3-(4-(4-(4-(trifluoromethyl)phenyl)piperidine-1-carbonyl)phenyl) oxetan-3-yl acetate

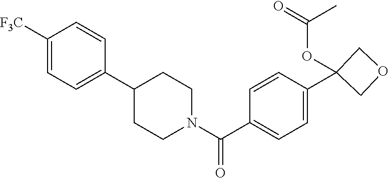 [SMILES: CC(=O)OC1(c2ccc(C(=O)N3CCC(c4ccc(C(F)(F)F)cc4)CC3)cc2)COC1]

¹H NMR (DMSO-d6) δ: 1.62-1.76 (m, 3H), 1.88 (br s, 1H), 2.16 (s, 3H), 2.92-2.98 (m, 2H), 3.19 (br s, 1H), 3.68 (br s, 1H), 4.64 (br s, 1H), 4.83 (d, J=8.0 Hz, 2H), 4.95 (d, J=8.0 Hz, 2H), 7.49-7.59 (m, 6H), 7.67 (d, J=8.4 Hz, 2H).

ESI-MS: m/z 448 (M+H)⁺, 100%.

### Example 37: (4-(3-methoxyoxetan-3-yl)phenyl)(4-(3-(trifluoromethyl)phenyl) piperidin-1-yl)methanone

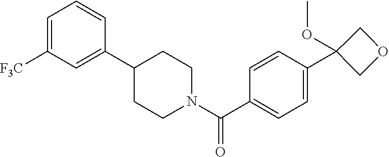 [SMILES: COC1(c2ccc(C(=O)N3CCC(c4cccc(C(F)(F)F)c4)CC3)cc2)COC1]

¹H NMR (CDCl₃) δ: 1.61-2.06 (m, 4H), 2.86-2.93 (m, 2H), 3.18 (brs, 4H), 3.95 (brs, 1H), 4.83 (d, J=7.2 Hz, 2H), 4.90-4.97 (m, 3H), 7.42-7.57 (m, 8H).

ESI-MS: m/z, 420.21 (M+H)⁺, 100%.

### Example 38: (4-(3-fluoro-4-(trifluoromethyl)phenyl)piperidin-1-yl)(4-(3-methoxyoxetan-3-yl)phenyl)methanone

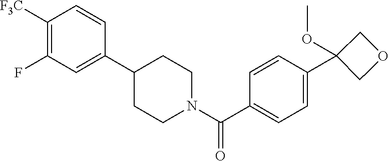 [SMILES: COC1(c2ccc(C(=O)N3CCC(c4ccc(C(F)(F)F)c(F)c4)CC3)cc2)COC1]

¹H NMR (CDCl₃) δ: 1.64-2.06 (m, 4H), 2.94-3.00 (brs, 1H), 3.17 (s, 3H), 3.21-3.28 (m, 2H), 3.95 (brs, 1H), 4.83 (d, J=7.2 Hz, 2H), 4.90-4.97 (m, 3H), 7.22-7.28 (m, 1H), 7.44-7.56 (m, 6H).

ESI-MS: m/z, 438.19 (M+H)⁺, 100%.

### Example 39: (4-(3-methoxyoxetan-3-yl)phenyl)(3-(4-(trifluoromethyl)phenyl) piperidin-1-yl)methanone

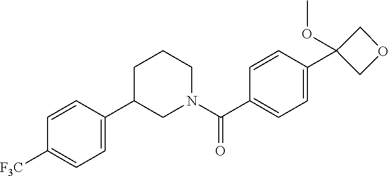 [SMILES: COC1(c2ccc(C(=O)N3CCCC(c4ccc(C(F)(F)F)cc4)C3)cc2)COC1]

¹H NMR (CDCl₃) δ: 1.64-2.06 (m, 4H), 2.86-2.92 (m, 2H), 3.18 (s, 4H), 3.96 (brs, 1H), 4.83 (d, J=7.2 Hz, 2H), 4.87-4.97 (m, 3H), 7.36 (d, J=8.0 Hz, 2H), 7.51-7.57 (m, 4H), 7.60 (d, J=8.4 Hz, 2H).

ESI-MS: m/z, 420.19 (M+H)⁺, 100%.

### Example 40: (4-(3-ethoxyoxetan-3-yl)phenyl)(4-(3-(trifluoromethyl)phenyl) piperidin-1-yl)methanone

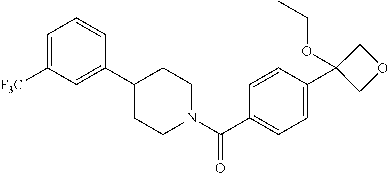 [SMILES: CCOC1(c2ccc(C(=O)N3CCC(c4cccc(C(F)(F)F)c4)CC3)cc2)COC1]

¹H NMR (CDCl₃) δ: 1.25 (t, J=6.8 Hz, 3H), 1.61-2.02 (m, 4H), 2.87-2.93 (m, 2H), 2.99-3.05 (m, 1H), 3.26 (q, J=7.2 Hz, 2H), 3.96-4.01 (m, 1H), 4.83 (d, J=6.8 Hz, 2H), 4.90-4.99 (m, 3H), 7.37-7.62 (m, 8H).

ESI-MS: m/z, 434.22 (M+H)⁺, 100%.

### Example 41: (4-(3-methoxyoxetan-3-yl)phenyl)(4-(2-(trifluoromethyl)phenyl) piperidin-1-yl)methanone

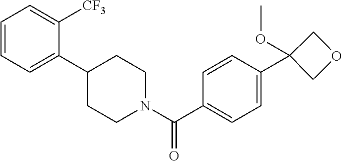 [SMILES: COC1(c2ccc(C(=O)N3CCC(c4ccccc4C(F)(F)F)CC3)cc2)COC1]

¹H NMR (DMSO-d₆) δ: 1.65-1.80 (m, 4H), 2.87 (brs, 1H), 3.05 (s, 3H), 3.19-3.23 (m, 2H), 3.70 (brs, 1H), 4.68 (brs, 1H), 4.76-4.81 (m, 4H), 7.41-7.44 (m, 1H), 7.64-7.69 (m, 2H), 7.54 (s, 4H), 7.75 (d, J=7.6 Hz, 1H);

ESI-MS: m/z, 420.20 (M+H)⁺, 100%

### Example 42: (4-(3-ethoxyoxetan-3-yl)phenyl)(4-(3-fluoro-4-(trifluoromethyl) phenyl) piperidin-1-yl)methanone

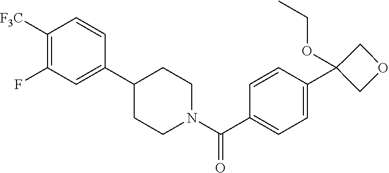 [SMILES: CCOC1(c2ccc(C(=O)N3CCC(c4ccc(C(F)(F)F)c(F)c4)CC3)cc2)COC1]

¹H NMR (CDCl₃) δ: 1.13 (t, J=6.8 Hz, 3H), 1.68-1.85 (m, 4H), 2.91 (brs, 1H), 3.17-3.33 (m, 4H), 3.72 (brs, 1H), 4.65 (brs, 1H), 4.75-4.81 (m, 4H), 7.38-7.42 (m, 1H), 7.51-7.55 (m, 4H), 7.63-7.67 (m, 1H), 7.78-7.81 (m, 1H).

ESI-MS: m/z, 452.18 (M+H)⁺, 100%.

### Example 43: (4-(3-ethoxyoxetan-3-yl)phenyl)(4-(4-(trifluoromethyl)phenyl) piperidin-1-yl)methanone

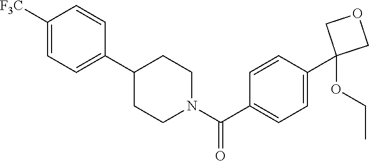 [SMILES: CCOC1(c2ccc(C(=O)N3CCC(c4ccc(C(F)(F)F)cc4)CC3)cc2)COC1]

¹H NMR (DMSO-d₆) δ: 1.18 (t, J=6.8 Hz, 3H), 1.61-1.71 (m, 3H), 1.88 (br s, 1H), 2.88-2.98 (m, 2H), 3.17-3.22 (m, 3H), 3.70 (br s, 1H), 4.74 (br s, 1H), 4.75-4.81 (m, 4H), 7.49-7.55 (m, 6H), 7.67 (d, J=8.0 Hz, 2H).

ESI-MS: m/z 434 (M+H)⁺, 100%.

### Example 44: (4-(3-isopropoxyoxetan-3-yl)phenyl)(4-(4-(trifluoromethyl)phenyl) piperidin-1-yl)methanone

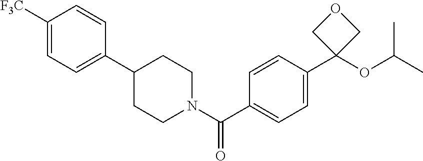 [SMILES: CC(C)OC1(c2ccc(C(=O)N3CCC(c4ccc(C(F)(F)F)cc4)CC3)cc2)COC1]

¹H NMR (DMSO-d6) δ: 0.95 (d, J=6.0 Hz, 6H), 1.62-1.72 (m, 2H), 1.88-1.91 (m, 2H), 2.87-2.98 (m, 2H), 3.03 (br s, 1H), 3.40-3.51 (m, 1H), 3.70 (br s, 1H), 4.65 (br s, 1H), 4.81 (s, 4H), 7.50-7.57 (m, 6H), 7.67 (d, J=8.4 Hz, 2H).

ESI-MS: m/z 448 (M+H)⁺, 100%.

### Example 45: (4-(3-isobutoxyoxetan-3-yl)phenyl)(4-(4-(trifluoromethyl)phenyl) piperidin-1-yl)methanone

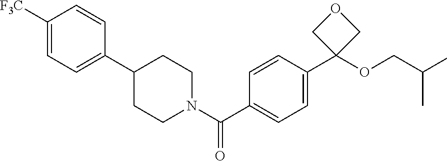 [SMILES: CC(C)COC1(c2ccc(C(=O)N3CCC(c4ccc(C(F)(F)F)cc4)CC3)cc2)COC1]

¹H NMR (CDCl₃) δ: 0.95 (d, J=6.4 Hz, 6H), 1.70 (br s, 1H), 1.84-1.94 (m, 3H), 2.01 (br s, 1H), 2.87-2.93 (m, 2H), 2.97 (d, J=6.4 Hz, 2H), 3.19 (br s, 1H), 3.98 (br s, 1H), 4.81 (d, J=7.2 Hz, 2H), 4.96 (br s, 1H), 4.99 (d, J=11.2 Hz, 2H), 7.36 (d, J=8.0 Hz, 2H), 7.50-7.57 (m, 4H), 7.60 (d, J=8.0 Hz, 2H).

ESI-MS: m/z 462 (M+H)⁺, 100%.

### Example 46: (4-(3-(cyclopropylmethoxy)oxetan-3-yl)phenyl)(4-(4-(trifluoromethyl)phenyl)piperidin-1-yl)methanone

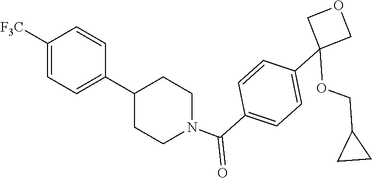 [SMILES: O=C(c1ccc(C2(OCC3CC3)COC2)cc1)N1CCC(c2ccc(C(F)(F)F)cc2)CC1]

¹H NMR (CDCl₃) δ: 0.16-0.20 (m, 2H), 0.55-0.59 (m, 2H), 0.85-0.90 (m, 1H), 1.03-1.10 (m, 2H), 1.12 (br s, 2H), 1.61-1.70 (br s, 3H), 2.01 (br s, 1H), 2.86-2.96 (m, 2H), 3.04 (d, J=6.8 Hz, 2H), 3.19 (br s, 1H), 3.95 (br s, 1H), 4.81 (d, J=6.8 Hz, 2H), 4.93 (br s, 1H), 4.99 (d, J=7.2 Hz, 2H), 7.36 (d, J=8.4 Hz, 2H), 7.50-7.65 (m, 2H).

ESI-MS: m/z 460 (M+H)⁺, 100%.

### Example 47: (3-(3-hydroxyoxetan-3-yl)phenyl)(4-(4-(trifluoromethyl)phenyl) piperidin-1-yl)methanone

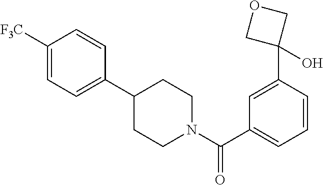 [SMILES: O=C(c1cccc(C2(O)COC2)c1)N1CCC(c2ccc(C(F)(F)F)cc2)CC1]

¹H NMR (DMSO-d6) δ: 1.65-1.89 (m, 4H), 2.89-2.98 (m, 2H), 3.16-3.20 (m, 1H), 3.70-3.76 (m, 1H), 4.62-4.70 (m, 3H), 4.79 (d, J=6.8 Hz, 2H), 6.45 (s, 1H), 7.38 (d, J=7.6 Hz, 1H), 7.54 (d, J=7.6 Hz, 1H), 7.53 (d, J=8.4 Hz, 2H), 7.63-7.71 (m, 4H).

ESI-MS: m/z 406.15 (M+H)⁺, 100%.

### Example 48: (3-(3-hydroxyoxetan-3-yl)phenyl)(3-(4-(trifluoromethyl)phenyl) piperidin-1-yl)methanone

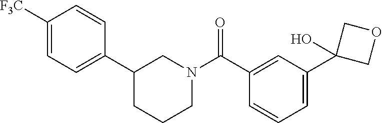 [SMILES: O=C(c1cccc(C2(O)COC2)c1)N1CCCC(c2ccc(C(F)(F)F)cc2)C1]

¹H NMR (DMSO-d₆) δ: 1.66-2.03 (m, 4H), 2.86-2.92 (m, 2H), 3.19 (brs, 1H), 3.70 (brs, 1H), 4.64-4.70 (m, 3H), 4.80 (d, J=6.8 Hz, 2H), 6.45 (s, 1H), 7.32-7.39 (m, 1H), 7.47-7.54 (m, 4H), 7.63-7.71 (m, 3H).

ESI-MS: m/z, 406.22 (M+H)⁺, 100%.

### Example 49: (5-(3-hydroxyoxetan-3-yl)pyridin-3-yl)(4-(4-(trifluoromethyl) phenyl)piperidin-1-yl)methanone

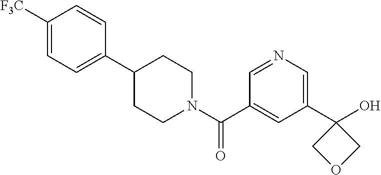 [SMILES: O=C(c1cncc(C2(O)COC2)c1)N1CCC(c2ccc(C(F)(F)F)cc2)CC1]

¹H NMR (DMSO-d₆) δ: 1.82-1.87 (m, 3H), 2.04 (bs, 1H), 2.88-2.94 (m, 2H), 3.15-3.28 (m, 1H), 3.88-3.90 (m, 1H), 4.85 (d, J=7.2 Hz, 2H), 4.88-4.96 (m, 1H), 4.97 (d, J=7.2 Hz, 2H), 7.35 (d, J=8.0 Hz, 2H), 7.60 (d, J=8.0 Hz, 2H), 8.06 (s, 1H), 8.64 (d, J=1.6 Hz, 1H), 8.98 (d, J=2.0 Hz, 1H).

ESI-MS: m/z 407.1 (M+H)⁺, 100%.

### Example 50: (3-(3-methoxyoxetan-3-yl)phenyl)(4-(4-(trifluoromethyl)phenyl) piperidin-1-yl)methanone

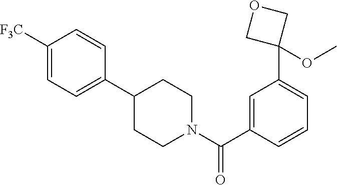 [SMILES: COC1(c2cccc(C(=O)N3CCC(c4ccc(C(F)(F)F)cc4)CC3)c2)COC1]

¹H NMR (DMSO-d₆) δ: 1.67-1.91 (m, 4H), 2.90-2.97 (m, 2H), 3.04 (s, 3H), 3.18-3.22 (m, 1H), 3.63-3.69 (m, 1H), 4.65-4.69 (m, 1H), 4.77 (d, J=7.2 Hz, 2H), 4.80 (d, J=7.2 Hz, 2H), 7.43-7.48 (m, 2H), 7.52-7.55 (m, 4H), 7.67 (d, J=8 Hz, 2H).

ESI-MS: m/z 420.15 (M+H)⁺, 100%.

### Example 51: (3-(3-methoxyoxetan-3-yl)phenyl)(3-(4-(trifluoromethyl)phenyl) piperidin-1-yl)methanone

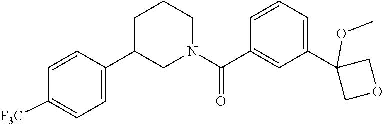 [SMILES: COC1(c2cccc(C(=O)N3CCCC(c4ccc(C(F)(F)F)cc4)C3)c2)COC1]

¹H NMR (CDCl₃) δ: 1.65-2.06 (m, 4H), 2.86-2.92 (m, 2H), 3.11-3.18 (m, 4H), 3.91 (brs, 1H), 4.83 (d, J=6.8 Hz, 2H), 4.90-4.97 (m, 3H), 7.36 (d, J=8.0 Hz, 2H), 7.43-7.49 (m, 1H), 7.51-7.61 (m, 5H).

ESI-MS: m/z, 420.19 (M+H)⁺, 100%.

### Example 52: N-(3-(3-hydroxyoxetan-3-yl)phenyl)-4-(4-(trifluoromethyl)phenyl) piperidine-1-carboxamide

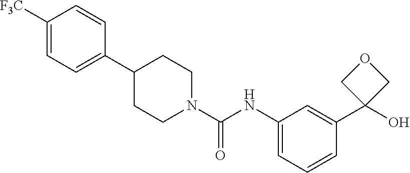 [SMILES: O=C(Nc1cccc(C2(O)COC2)c1)N1CCC(c2ccc(C(F)(F)F)cc2)CC1]

¹H NMR (DMSO-d₆) δ: 1.56-1.65 (m, 2H), 1.83 (d, J=12.0 Hz, 2H), 2.86-2.92 (m, 3H), 4.31 (d, J=13.2 Hz, 2H), 4.66 (d, J=6.8 Hz, 2H), 4.76 (d, J=6.4 Hz, 2H), 6.28 (s, 1H), 7.16 (d, J=7.6 Hz, 1H), 7.48-7.53 (m, 3H), 7.67 (d, J=8.0 Hz, 2H), 7.72 (d, J=2.0 Hz, 1H), 8.59 (s, 1H).

ESI-MS: m/z 421 (M+H)⁺, 100%.

### Example 53: (4-(3-methoxyoxetan-3-yl)phenyl)(4-(4-(trifluoromethyl)phenyl) piperazin-1-yl)methanone

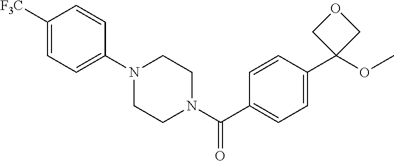 [SMILES: COC1(c2ccc(C(=O)N3CCN(c4ccc(C(F)(F)F)cc4)CC3)cc2)COC1]

¹H NMR (DMSO-d₆) δ: 3.05 (s, 3H), 3.36 (br s, 4H), 3.61-3.66 (m, 2H), 3.76 (br s, 2H), 4.76-4.81 (m, 4H), 7.08 (d, J=8.8 Hz, 2H), 7.45-7.56 (m, 6H).

ESI-MS: m/z 421 (M+H)⁺, 100%.

### Example 54: 3-(4-((4-(4-(trifluoromethyl)phenyl)piperidin-1-yl)sulfonyl)phenyl) oxetan-3-ol

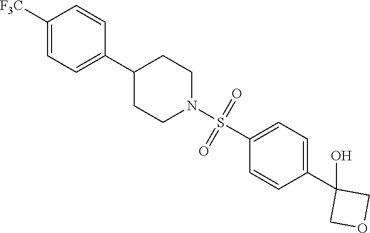 [SMILES: O=S(=O)(c1ccc(C2(O)COC2)cc1)N1CCC(c2ccc(C(F)(F)F)cc2)CC1]

¹H NMR (CDCl₃) δ: 1.86-1.94 (m, 4H), 2.37-2.45 (m, 2H), 2.50-2.53 (m, 1H), 3.99-4.02 (m, 2H), 4.91 (d, J=7.6 Hz, 2H), 4.99 (d, J=7.6 Hz, 2H), 7.28 (d, J=7.6 Hz, 2H), 7.57 (d, J=7.6 Hz, 2H), 7.85-7.90 (m, 4H).

ESI-MS: m/z 442 (M+H)⁺, 100%.

### Example 55: (3-(3-hydroxyoxetan-3-yl)phenyl)(5-(4-(trifluoromethyl)phenyl) hexahydropyrrolo[3,4-c]pyrrol-2(1H)-yl)methanone

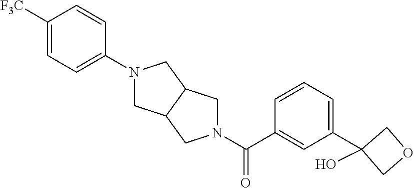 [SMILES: O=C(c1cccc(C2(O)COC2)c1)N1CC2CN(c3ccc(C(F)(F)F)cc3)CC2C1]

¹H NMR (DMSO-d6) δ: 2.67-3.18 (m, 3H), 3.34-3.36 (m, 2H), 3.45-3.50 (m, 2H), 3.55-3.60 (m, 1H), 3.71-3.75 (m, 1H), 3.81-3.86 (m, 1H), 4.68 (d, J=6.8 Hz, 2H), 4.78 (d, J=6.8 Hz, 2H), 6.44 (s, 1H), 6.64 (d, J=8.8 Hz, 2H), 7.45-7.47 (m, 4H), 7.68-7.71 (m, 2H).

ESI-MS: m/z 433.23 (M+H)⁺, 100%.

### Example 56: (3-(3-methoxyoxetan-3-yl)phenyl)(5-(4-(trifluoromethyl)phenyl) hexahydropyrrolo[3,4-c]pyrrol-2(1H)-yl)methanone

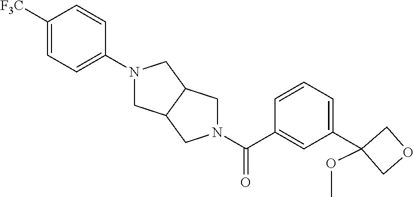 [SMILES: COC1(c2cccc(C(=O)N3CC4CN(c5ccc(C(F)(F)F)cc5)CC4C3)c2)COC1]

¹H NMR (DMSO-d6) δ: 3.02 (s, 3H), 3.04-3.09 (m, 2H), 3.15-3.20 (m, 1H), 3.34-3.36 (m, 2H), 3.47-3.49 (m, 2H), 3.55-3.60 (m, 1H), 3.71-3.75 (m, 1H), 3.81-3.86 (m, 1H), 4.75-4.79 (m, 4H), 6.64 (d, J=8.8 Hz, 2H), 7.46 (d, J=8.4 Hz, 2H), 7.49-7.55 (m, 4H).

ESI-MS: m/z 447.16 (M+H)⁺, 100%.

### Example 57: (4-(3-methoxyoxetan-3-yl)phenyl)(5-(4-(trifluoromethyl)phenyl) hexahydropyrrolo[3,4-c]pyrrol-2(1H)-yl)methanone

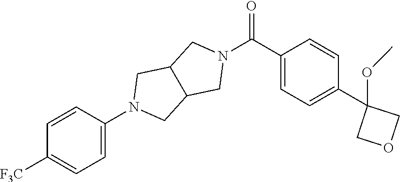 [SMILES: COC1(c2ccc(C(=O)N3CC4CN(c5ccc(C(F)(F)F)cc5)CC4C3)cc2)COC1]

¹H NMR (DMSO-d₆) δ: 3.04 (s, 3H), 3.05-3.10 (m, 2H), 3.14-3.19 (m, 1H), 3.33-3.39 (m, 2H), 3.45-3.51 (m, 2H), 3.56-3.60 (m, 1H), 3.72-3.76 (m, 1H), 3.82-3.85 (m, 1H), 4.74 (d, J=7.2 Hz, 2H), 4.78 (d, J=7.2 Hz, 2H), 6.63 (d, J=8.4 Hz, 2H), 7.45-7.51 (m, 4H), 7.59 (d, J=8.4 Hz, 2H).

ESI-MS: m/z 447.2 (M+H)⁺, 100%.

### Example 58: (4-(3-ethoxyoxetan-3-yl)phenyl)(5-(4-(trifluoromethyl)phenyl) hexahydropyrrolo[3,4-c]pyrrol-2 (1H)-yl)methanone

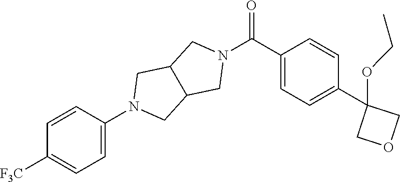 [SMILES: CCOC1(c2ccc(C(=O)N3CC4CN(c5ccc(C(F)(F)F)cc5)CC4C3)cc2)COC1]

¹H NMR (DMSO-d₆) δ: 1.10 (t, J=7.0 Hz, 3H), 3.05-3.09 (m, 2H), 3.15-3.21 (m, 3H), 3.36-3.39 (m, 2H), 3.45-3.50 (m, 2H), 3.56-3.60 (m, 1H), 3.72-3.76 (m, 1H), 3.82-3.85 (m, 1H), 4.74 (d, J=6.8 Hz, 2H), 4.78 (d, J=7.2 Hz, 2H), 6.63 (d, J=8.4 Hz, 2H), 7.45-7.52 (m, 4H), 7.58 (d, J=8.4 Hz, 2H).

ESI-MS: m/z 461.2 (M+H)⁺, 100%.

### Example 59: (4-(4-(difluoromethyl) phenyl) piperidin-1-yl)(3-(3-(methoxy-d3) oxetan-3-yl) phenyl) methanone

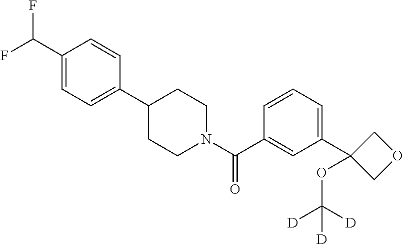 [SMILES: [2H]C([2H])([2H])OC1(c2cccc(C(=O)N3CCC(c4ccc(C(F)F)cc4)CC3)c2)COC1]

¹H NMR (DMSO-d6) δ: 1.66-1.82 (m, 3H), 1.87-1.91 (m, 1H), 2.86-2.92 (m, 2H), 3.18-3.28 (m, 1H), 3.67 (brs, 1H), 4.67 (brs, 1H), 4.76-4.80 (m, 4H), 6.99 (t, J=16.0 Hz, 1H), 7.42-7.54 (m, 8H).

ESI-MS: m/z, 405.25 (M+H)⁺, 100%.

### Example 60: N-(4-(3-methoxyoxetan-3-yl) phenyl)-1-(4-(trifluoromethyl) phenyl) piperidine-4-carboxamide

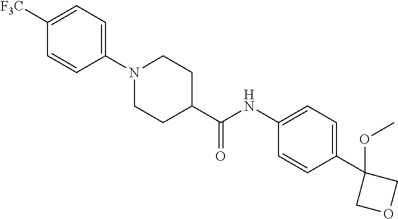 [SMILES: COC1(c2ccc(NC(=O)C3CCN(c4ccc(C(F)(F)F)cc4)CC3)cc2)COC1]

¹H NMR (DMSO-d₆) δ: 1.69-1.72 (m, 2H), 1.86-1.89 (m, 2H), 2.57-2.61 (m, 1H), 2.85-2.88 (m, 2H), 2.97 (s, 3H), 3.96 (brs, 2H), 4.73 (s, 4H), 7.09 (d, J=8.8 Hz, 2H), 7.35 (d, J=8.8 Hz, 2H), 7.50 (d, J=8.8 Hz, 2H), 7.67 (d, J=8.8 Hz, 2H), 10.04 (s, 1H);

ESI-MS: m/z, 435.24 (M+H)⁺, 100%

### Example 61: (4-(3-hydroxy-1,1-dioxidothietan-3-yl) phenyl)(5-(4-(trifluoromethyl)phenyl)hexahydropyrrolo[3,4-c]pyrrol-2(1H)-yl)methanone

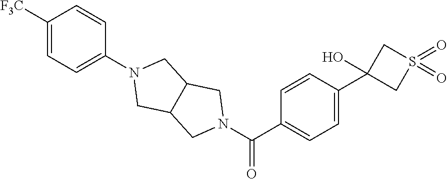 [SMILES: O=C(c1ccc(C2(O)CS(=O)(=O)C2)cc1)N1CC2CN(c3ccc(C(F)(F)F)cc3)CC2C1]

¹H NMR (DMSO-d₆) δ: 3.04-3.16 (m, 2H), 3.48 (d, J=11.2 Hz, 2H), 3.57 (d, J=7.6 Hz, 2H), 3.71 (d, J=7.2 Hz, 2H), 3.81 (d, J=6.4 Hz, 2H), 4.39 (d, J=15.2 Hz, 2H), 4.69 (d, J=14.8 Hz, 2H), 6.64 (d, J=8.4 Hz, 2H), 6.82 (s, 1H), 7.47 (d, J=8.0 Hz, 2H), 7.56-7.59 (m, 4H);

ESI-MS: m/z, 481.13 (M+H)⁺, 100%

### Example 62: (4-(3-hydroxy-1,1-dioxidothietan-3-yl)phenyl)(4-(4-(trifluoromethyl)phenyl)piperidin-1-yl)methanone

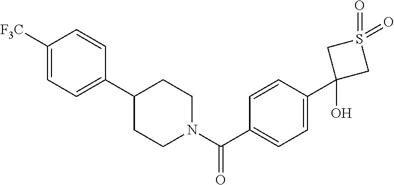 [SMILES: O=C(c1ccc(C2(O)CS(=O)(=O)C2)cc1)N1CCC(c2ccc(C(F)(F)F)cc2)CC1]

¹H NMR (DMSO-d6) δ: 1.65-1.70 (m. 3H), 1.88-1.91 (m, 1H), 2.89-2.95 (m, 2H), 3.16 (brs, 1H), 3.71 (brs, 1H), 4.39 (d, J=15.2 Hz, 2H), 4.67 (brs, 1H), 4.72 (d, J=15.2 Hz, 2H), 6.82 (s, 1H), 7.49 (d, J=8.4 Hz, 2H), 7.54 (d, J=8.4 Hz, 2H), 7.62 (d, J=8.4 Hz, 2H), 7.67 (d, J=8.4 Hz, 2H).

ESI-MS: m/z, 454.12 (M+H)⁺, 100%.

### Example 63: (4-(3-hydroxyoxetan-3-yl) phenyl)(4-(4-hydroxyphenyl) piperidin-1-yl)methanone

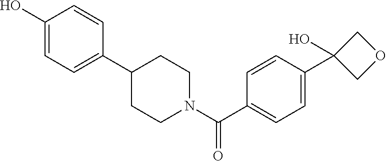 [SMILES: O=C(c1ccc(C2(O)COC2)cc1)N1CCC(c2ccc(O)cc2)CC1]

¹H NMR (DMSO-d₆) δ: 1.52-1.57 (m, 2H), 1.67 (brs. 1H), 1.80 (brs, 1H), 2.66-2.69 (m, 1H), 2.72 (brs, 1H), 3.13 (brs, 1H), 3.68 (brs, 1H), 4.62 (brs, 1H), 4.70 (d, J=6.8 Hz, 2H), 4.78 (d, J=7.2 Hz, 2H), 6.44 (s, 1H), 6.68 (d, J=8.4 Hz, 2H), 7.06 (d, J=8.4 Hz, 2H), 7.45 (d, J=8.4 Hz, 2H), 7.65 (d, J=8.4 Hz, 2H), 9.17 (s, 1H);

ESI-MS: m/z, 354.16 (M+H)⁺, 100%

### Example 64: (4-(3-hydroxyoxetan-3-yl)phenyl)(4-phenylpiperidin-1-yl) methanone

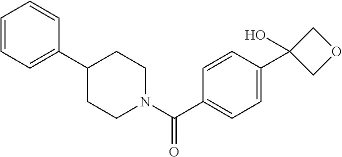 [SMILES: O=C(c1ccc(C2(O)COC2)cc1)N1CCC(c2ccccc2)CC1]

¹H NMR (DMSO-d₆) δ: 1.52-1.57 (m, 2H), 1.67 (brs. 1H), 1.80 (brs, 1H), 2.66-2.69 (m, 1H), 2.72 (brs, 1H), 3.13 (brs, 1H), 3.68 (brs, 1H), 4.62 (brs, 1H), 4.70 (d, J=6.8 Hz, 2H), 4.79 (d, J=6.8 Hz, 2H), 6.44 (s, 1H), 7.18-7.22 (m, 1H), 7.27-7.33 (m, 4H), 7.47 (d, J=8.4 Hz, 2H), 7.68 (d, J=8.4 Hz, 2H).

ESI-MS: m/z, 338.17 (M+H)⁺, 100%.

### Example 65: (4-(4-(difluoromethyl)phenyl)piperidin-1-yl)(3-(3-methoxyoxetan-3-yl) phenyl)methanone

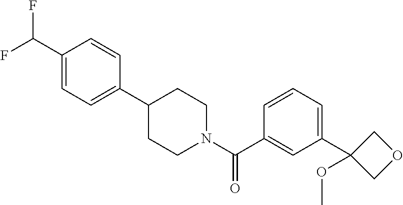 [SMILES: COC1(c2cccc(C(=O)N3CCC(c4ccc(C(F)F)cc4)CC3)c2)COC1]

¹H NMR (CDCl₃) δ: 1.66-1.82 (m, 3H), 1.87-1.91 (m, 1H), 2.86-2.92 (m, 2H), 3.18 (s, 3H), 3.19-3.28 (m, 1H), 3.67 (brs, 1H), 4.67 (brs, 1H), 4.83 (d, J=7.2 Hz, 2H), 4.97 (d, J=7.2 Hz, 2H), 6.65 (t, J=16.4 Hz, 1H), 7.33 (d, J=8.0 Hz, 2H), 7.42-7.58 (m, 6H);

ESI-MS: m/z, 402.18 (M+H)⁺, 100%.

### Example 66: (4-(4-(difluoromethyl)phenyl)piperidin-1-yl)(3-(3-hydroxyoxetan-3-yl) phenyl)methanone

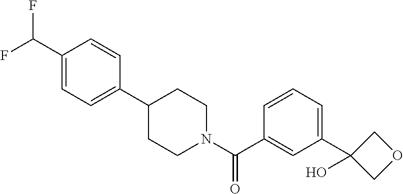 [SMILES: O=C(c1cccc(C2(O)COC2)c1)N1CCC(c2ccc(C(F)F)cc2)CC1]

¹H NMR (DMSO-d6) δ: 1.63-1.71 (m, 3H), 1.87-1.91 (m, 1H), 2.87-2.93 (m, 2H), 3.18 (brs, 1H), 3.70 (brs, 1H), 4.67 (brs, 1H), 4.69 (d, J=6.8 Hz, 2H), 4.79 (d, J=6.4 Hz, 2H), 6.46 (s, 1H), 6.99 (t, J=16.2 Hz, 1H), 7.38 (d, J=7.8 Hz, 1H), 7.43-7.52 (m, 5H), 7.63 (s, 1H), 7.70 (d, J=8.0 Hz, 1H).

ESI-MS: m/z, 388.17 (M+H)⁺, 100%.

### Example 67: (4-(3-hydroxyoxetan-3-yl)phenyl)(2-(2-methoxypyridin-4-yl)-4-(4-(trifluoromethyl)phenyl)piperazin-1-yl)methanone

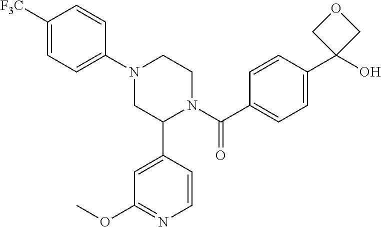 [SMILES: COc1cc(C2CN(c3ccc(C(F)(F)F)cc3)CCN2C(=O)c2ccc(C3(O)COC3)cc2)ccn1]

¹H NMR (DMSO-d₆) δ: 3.07 (t, J=10.4 Hz, 2H), 3.40-3.49 (m, 2H), 3.70 (brs, 2H), 3.82 (s, 3H), 4.33 (d, J=9.8 Hz, 1H), 4.70 (d, J=6.4 Hz, 2H), 4.80 (d, J=6.8 Hz, 2H), 6.48 (s, 1H), 6.83 (d, J=8.8 Hz, 1H), 7.08 (d, J=8.8 Hz, 2H), 7.53 (d, J=8.8 Hz, 4H), 7.71 (d, J=7.6 Hz, 2H), 7.76 (d, J=7.2 Hz, 1H), 8.20-8.25 (m, 1H).

ESI-MS: m/z, 514.19 (M+H)⁺, 100%.

### Example 68: (4-(4-(difluoromethyl)phenyl)piperidin-1-yl)(4-(3-methoxyoxetan-3-yl) phenyl)methanone

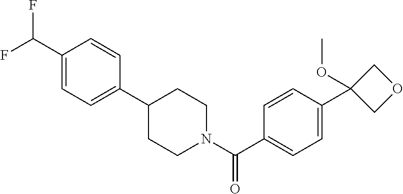 [SMILES: COC1(c2ccc(C(=O)N3CCC(c4ccc(C(F)F)cc4)CC3)cc2)COC1]

¹H NMR (DMSO-d₆) δ: 1.61-1.70 (m, 3H), 1.86 (brs. 1H), 2.87-2.93 (m, 2H), 3.05 (s, 3H), 3.17 (brs, 1H), 3.70 (brs, 1H), 4.64 (brs, 1H), 4.76-4.80 (m, 4H), 7.00 (t, J=56.0 Hz, 1H), 7.43-7.54 (m, 8H).

ESI-MS: m/z, 402.18 (M+H)⁺, 100%.

### Example 69: (4-(4-(difluoromethyl)phenyl)piperidin-1-yl)(4-(3-hydroxyoxetan-3-yl) phenyl)methanone

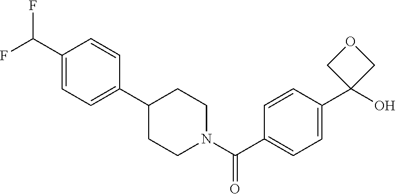 [SMILES: O=C(c1ccc(C2(O)COC2)cc1)N1CCC(c2ccc(C(F)F)cc2)CC1]

¹H NMR (DMSO-d₆) δ: 1.60-1.76 (m, 3H), 1.87 (brs. 1H), 2.86-2.92 (m, 2H), 3.17 (s, 1H), 3.70 (brs, 1H), 4.64 (brs, 1H), 4.70 (d, J=6.4 Hz, 2H), 4.79 (d, J=7.6 Hz, 2H), 6.44 (s, 1H), 7.00 (t, J=56.0 Hz, 1H), 7.43-7.52 (m, 6H), 7.68 (d, J=8.0 Hz, 2H).

ESI-MS: m/z, 388.17 (M+H)⁺, 100%.

### Example 70: (4-(3-(but-2-yn-1-yloxy)oxetan-3-yl)phenyl)(4-(4-(trifluoromethyl) phenyl)piperidin-1-yl)methanone

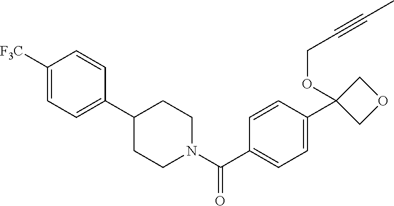 [SMILES: CC#CCOC1(c2ccc(C(=O)N3CCC(c4ccc(C(F)(F)F)cc4)CC3)cc2)COC1]

¹H NMR (DMSO-d₆) δ: 1.62-1.72 (m, 3H), 1.80 (s, 3H), 1.90 (brs. 1H), 2.88-2.98 (m, 2H), 3.28 (brs, 1H), 3.70 (brs, 1H), 3.86 (s, 2H), 4.66 (brs, 1H), 4.79 (d, J=7.2 Hz, 2H), 4.87 (d, J=7.2 Hz, 2H), 7.51-7.55 (m, 6H), 7.67 (d, J=8.0 Hz, 2H).

ESI-MS: m/z, 458.19 (M+H)⁺, 100%.

### Example 71: N-(4-(3-hydroxyoxetan-3-yl)phenyl)-5-phenyl-2-(4-(trifluoromethyl)phenyl)oxazole-4-carboxamide

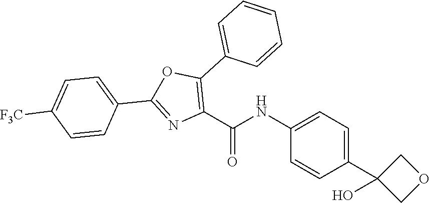 [SMILES: O=C(Nc1ccc(C2(O)COC2)cc1)c1nc(-c2ccc(C(F)(F)F)cc2)oc1-c1ccccc1]

¹H NMR (DMSO-d₆) δ: 4.71 (d, J=6.4 Hz, 2H), 4.78 (d, J=6.4 Hz, 2H), 6.34 (s, 1H), 7.54-7.59 (m, 5H), 7.88 (d, J=8.4 Hz, 2H), 8.02 (d, J=8.4 Hz, 2H), 8.35 (d, J=8.4 Hz, 2H), 8.45 (d, J=8.4 Hz, 2H), 10.36 (s, 1H).

ESI-MS: m/z, 483.13 (M+H)⁺, 100%.

### Example 72: (4-(3-(prop-2-yn-1-yloxy)oxetan-3-yl)phenyl)(4-(4-(trifluoromethyl)phenyl) piperidin-1-yl)methanone

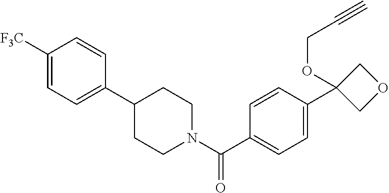 [SMILES: C#CCOC1(c2ccc(C(=O)N3CCC(c4ccc(C(F)(F)F)cc4)CC3)cc2)COC1]

¹H NMR (DMSO-d₆) δ: 1.63-1.71 (m, 3H), 1.88 (brs. 1H), 2.92-2.95 (m, 2H), 3.18 (brs, 1H), 3.38 (s, 1H), 3.70 (brs, 1H), 3.93 (s, 2H), 4.65 (brs, 1H), 4.81 (d, J=7.2 Hz, 2H), 4.89 (d, J=7.2 Hz, 2H), 7.51-7.55 (m, 6H), 7.67 (d, J=8.4 Hz, 2H).

ESI-MS: m/z, 443.17 (M+H)⁺, 100%.

### Example 73: 4-(4-(3-methoxyoxetan-3-yl)phenyl)-1-(4-(trifluoromethyl)phenyl) piperidin-4-ol

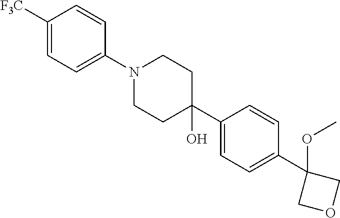 [SMILES: COC1(c2ccc(C3(O)CCN(c4ccc(C(F)(F)F)cc4)CC3)cc2)COC1]

¹H NMR (DMSO-d₆) δ: 1.71-1.74 (m, 2H), 1.98-2.06 (m, 2H), 3.00 (s, 3H), 3.23-3.29 (m, 2H), 3.78 (d, J=13.2 Hz, 2H), 4.73-4.77 (m, 4H), 5.16 (s, 1H), 7.12 (d, J=8.8 Hz, 2H), 7.39 (d, J=8.4 Hz, 2H), 7.50 (d, J=8.4 Hz, 2H), 7.55 (d, J=8.8 Hz, 2H).

ESI-MS: m/z, 408.17 (M+H)⁺, 100%.

### Example 74: N-(4-(3-methoxyoxetan-3-yl)phenyl)-5-(4-(trifluoromethyl)phenyl) hexahydropyrrolo[3,4-c]pyrrole-2(1H)-carboxamide

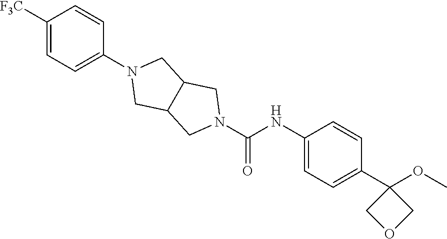 [SMILES: COC1(c2ccc(NC(=O)N3CC4CN(c5ccc(C(F)(F)F)cc5)CC4C3)cc2)COC1]

¹H NMR (DMSO-d₆) δ: 2.96 (s, 3H), 3.09 (brs, 2H), 3.22-3.26 (m, 2H), 3.35-3.38 (m, 2H), 3.55-3.59 (m, 2H), 3.69-3.73 (m, 2H), 4.71-4.75 (m, 4H), 6.67 (d, J=8.4 Hz, 2H), 7.27 (d, J=8.8 Hz, 2H), 7.47 (d, J=8.4 Hz, 2H), 7.55 (d, J=8.8 Hz, 2H), 8.26 (s, 1H).

ESI-MS: m/z, 462.4 (M+H)⁺, 100%.

### Example 75: N-(4-(3-hydroxyoxetan-3-yl)phenyl)-5-(4-(trifluoromethyl)phenyl) hexahydropyrrolo[3,4-c]pyrrole-2(1H)-carboxamide

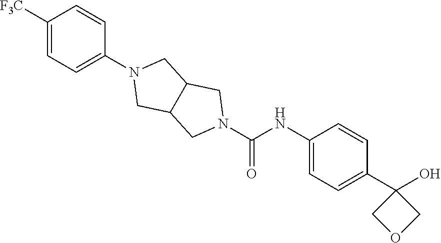 [SMILES: O=C(Nc1ccc(C2(O)COC2)cc1)N1CC2CN(c3ccc(C(F)(F)F)cc3)CC2C1]

¹H NMR (DMSO-d₆) δ: 3.09 (brs, 2H), 3.22-3.26 (m, 2H), 3.35-3.38 (m, 2H), 3.55-3.59 (m, 2H), 3.69-3.73 (m, 2H), 4.71-4.75 (m, 4H), 6.19 (s, 1H), 6.67 (d, J=8.4 Hz, 2H), 7.27 (d, J=8.8 Hz, 2H), 7.47 (d, J=8.4 Hz, 2H), 7.55 (d, J=8.8 Hz, 2H), 8.19 (s, 1H).

ESI-MS: m/z, 448.3 (M+H)⁺, 100%.

### Example 76: (4-(3-(methoxy-d3)oxetan-3-yl)phenyl)(4-(4-(trifluoromethyl) phenyl)piperidin-1-yl)methanone

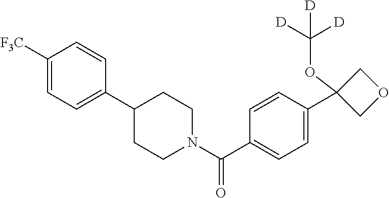 [SMILES: [2H]C([2H])([2H])OC1(c2ccc(C(=O)N3CCC(c4ccc(C(F)(F)F)cc4)CC3)cc2)COC1]

¹H NMR (DMSO-d6) δ: 1.62-1.72 (m, 3H), 1.77-1.82 (m, 1H), 2.88-2.98 (m, 2H), 3.16 (brs, 1H), 3.69 (brs, 1H), 4.65 (brs, 1H), 4.76-4.81 (m, 4H), 7.50-7.55 (m, 6H), 7.63 (d, J=8.4 Hz, 2H).

ESI-MS: m/z, 423.23 (M+H)⁺, 100%.

### Example 77: 5-(3-hydroxyoxetan-3-yl)-2-(4-(4-(trifluoromethyl) phenyl) piperidin-1-yl)benzoic acid

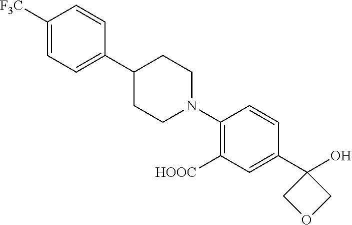 [SMILES: O=C(O)c1cc(C2(O)COC2)ccc1N1CCC(c2ccc(C(F)(F)F)cc2)CC1]

¹H NMR (DMSO-d₆) δ: 1.86-1.88 (m, 2H), 2.06-2.09 (m, 2H), 3.04 (brs, 1H), 3.20-3.30 (m, 4H), 4.72-4.82 (m, 4H), 6.65 (s, 1H), 7.57 (d, J=8.0 Hz, 2H), 7.68-7.73 (m, 3H), 7.90 (s, 1H), 8.09 (d, J=8.0 Hz, 1H), 17.59 (s, 1H).

ESI-MS: m/z, 422.12 (M+H)⁺, 100%.

### Example 78: 3-(3-hydroxyoxetan-3-yl)-5-(4-(4-(trifluoromethyl)phenyl) piperidin-1-yl) benzoic acid

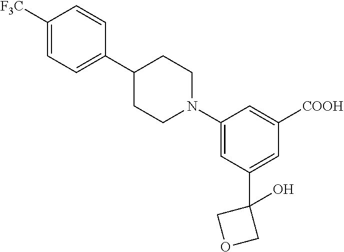 [SMILES: O=C(O)c1cc(N2CCC(c3ccc(C(F)(F)F)cc3)CC2)cc(C2(O)COC2)c1]

¹H NMR (DMSO-d6) δ: 1.81-1.92 (m, 2H), 1.95-2.07 (m, 2H), 2.83-2.89 (m, 3H), 3.86-3.96 (m, 2H), 4.68-4.78 (m, 4H), 6.41 (s, 1H), 7.17 (d, J=7.6 Hz, 2H), 7.53 (d, J=8.0 Hz, 2H), 7.64-7.68 (m, 3H).

ESI-MS: m/z, 422.15 (M+H)⁺, 100%.

### Example 79: (3-(3-hydroxyoxetan-3-yl)phenyl)(2-(4-(trifluoromethyl)phenyl)-1,4,6,7-tetrahydro-5H-imidazo[4,5-c]pyridin-5-yl)methanone

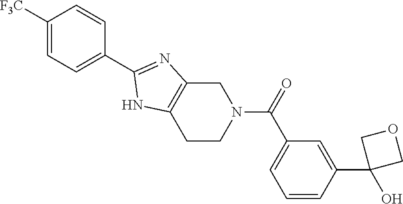 [SMILES: O=C(c1cccc(C2(O)COC2)c1)N1CCc2[nH]c(-c3ccc(C(F)(F)F)cc3)nc2C1]

¹H NMR (DMSO-d6) δ: 2.73-2.78 (m, 2H), 3.63 (brs, 1H), 3.98 (brs, 1H), 4.41 (brs, 1H), 4.64 (brs, 1H), 4.69-4.79 (m, 4H), 6.48 (s, 1H), 7.41 (d, J=6.4 Hz, 1H), 7.52 (t, J=7.6 Hz, 1H), 7.65 (s, 1H), 7.73-7.79 (m, 3H), 8.01-8.10 (m, 2H), 12.74 (s, 1H).

ESI-MS: m/z, 444.25 (M+H)⁺, 100%.

### Example 80: (4-(3-hydroxyoxetan-3-yl)phenyl)(2-(4-(trifluoromethyl)phenyl)-1,4,6,7-tetrahydro-5H-imidazo[4,5-c]pyridin-5-yl)methanone

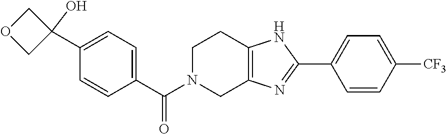 [SMILES: O=C(c1ccc(C2(O)COC2)cc1)N1CCc2[nH]c(-c3ccc(C(F)(F)F)cc3)nc2C1]

¹H NMR (DMSO-d₆) δ: 2.73-2.78 (m, 2H), 3.63 (brs, 1H), 3.98 (brs, 1H), 4.41 (brs, 1H), 4.64 (brs, 1H), 4.70-4.81 (m, 4H), 6.47 (s, 1H), 7.51 (d, J=8.0 Hz, 2H), 7.65 (d, J=8.4 Hz, 2H), 7.71 (d, J=8.0 Hz, 2H), 7.80-8.08 (m, 2H), 12.74 (s, 1H).

ESI-MS: m/z, 444.22 (M+H)⁺, 100%.

### Example 81: (4-(3-hydroxyoxetan-3-yl)-2-morpholinophenyl)(4-(4-(trifluoromethyl)phenyl)piperidin-1-yl)methanone

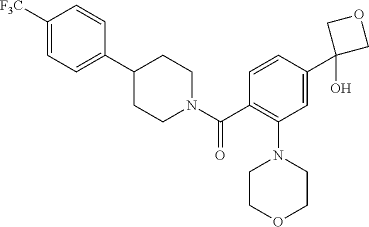 [SMILES: O=C(c1ccc(C2(O)COC2)cc1N1CCOCC1)N1CCC(c2ccc(C(F)(F)F)cc2)CC1]

¹H NMR (DMSO-d₆) δ: 1.63-1.89 (m, 4H), 2.08-2.84 (m, 3H), 2.94-3.00 (m, 2H), 3.08-3.12 (m, 1H), 3.23-3.27 (m, 2H), 3.70-3.77 (m, 4H), 4.68-4.77 (m, 5H), 6.40 (s, 1H), 7.15-7.24 (m, 1H), 7.31-7.35 (m, 2H), 7.50 (d, J=8.0 Hz, 2H), 7.65 (d, J=8.4 Hz, 1H), 7.72 (d, J=8.0 Hz, 1H).

ESI-MS: m/z, 491.21 (M+H)⁺, 100%.

### Example 82: (4-(3-hydroxyoxetan-3-yl)-2-(piperazin-1-yl)phenyl)(4-(4-(trifluoromethyl)phenyl)piperidin-1-yl)methanone

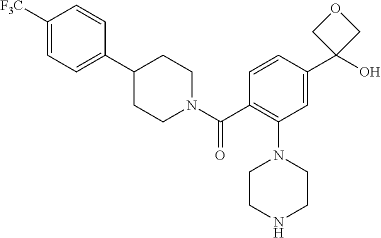 [SMILES: O=C(c1ccc(C2(O)COC2)cc1N1CCNCC1)N1CCC(c2ccc(C(F)(F)F)cc2)CC1]

¹H NMR (DMSO-d₆) δ: 1.61-1.69 (m, 2H), 1.89-1.99 (m, 2H), 2.80-2.99 (m, 9H), 3.04-3.13 (m, 4H), 4.68-4.77 (m, 5H), 6.41 (s, 1H), 7.15-7.24 (m, 1H), 7.31-7.35 (m, 2H), 7.50 (d, J=8.0 Hz, 2H), 7.65 (d, J=8.4 Hz, 1H), 7.72 (d, J=8.0 Hz, 1H).

ESI-MS: m/z, 490.21 (M+H)⁺, 100%.

### Example 83: 1-(4-(3-ethoxyoxetan-3-yl)phenyl)-4-(4-(trifluoromethyl)phenyl) piperidine

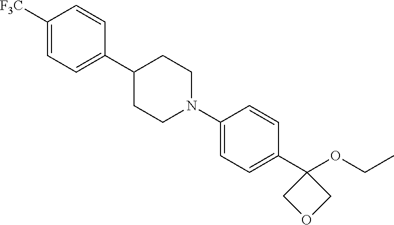 [SMILES: CCOC1(c2ccc(N3CCC(c4ccc(C(F)(F)F)cc4)CC3)cc2)COC1]

¹H NMR (CDCl₃) δ: 1.23 (t, J=7.0 Hz, 3H), 1.87-2.02 (m, 4H), 2.72-2.79 (m, 1H), 2.88 (t, J=12.0 Hz, 2H), 3.24 (q, J=7.2 & 14.0 Hz, 2H), 3.88 (d, J=12.4 Hz, 2H), 4.86 (d, J=6.4 Hz, 2H), 4.93 (d, J=6.8 Hz, 2H), 7.03 (d, J=8.8 Hz, 2H), 7.35 (d, J=8.8 Hz, 2H), 7.39 (d, J=8.4 Hz, 2H), 7.60 (d, J=8.0 Hz, 2H).

ESI-MS: m/z, 406.17 (M+H)⁺, 100%.

### Example 84: 3-(3-morpholino-5-(4-(4-(trifluoromethyl)phenyl)piperidin-1-yl)phenyl) oxetan-3-ol

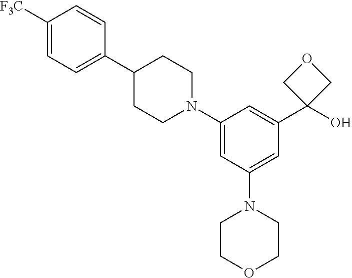 [SMILES: OC1(c2cc(N3CCOCC3)cc(N3CCC(c4ccc(C(F)(F)F)cc4)CC3)c2)COC1]

¹H NMR (DMSO-d6) δ: 1.76-1.83 (m, 2H), 1.88-1.91 (m, 2H), 2.75-2.81 (m, 3H), 3.10-3.12 (m, 4H), 3.72-3.74 (m, 4H), 3.81-3.84 (m, 2H), 4.70 (s, 4H), 6.15 (s, 1H), 6.46 (s, 1H), 6.62 (s, 1H), 6.69 (s, 1H), 7.53 (d, J=8.4 Hz, 2H), 7.67 (d, J=8.4 Hz, 2H).

ESI-MS: m/z, 463.19 (M+H)⁺, 100%.

### Example 85: tert-butyl 4-(5-(3-hydroxyoxetan-3-yl)-2-(4-(4-(trifluoromethyl) phenyl) piperidine-1-carbonyl)phenyl)piperazine-1-carboxylate

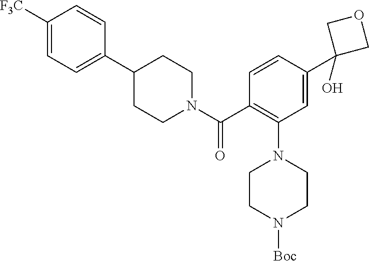 [SMILES: CC(C)(C)OC(=O)N1CCN(c2cc(C3(O)COC3)ccc2C(=O)N2CCC(c3ccc(C(F)(F)F)cc3)CC2)CC1]

¹H NMR (DMSO-d₆) δ: 1.43 (s, 9H), 1.61-1.69 (m, 2H), 1.89-1.99 (m, 2H), 2.80-2.99 (m, 9H), 3.04-3.13 (m, 4H), 4.68-4.76 (m, 5H), 6.40 (s, 1H), 7.15-7.24 (m, 1H), 7.31-7.35 (m, 2H), 7.50 (d, J=8.0 Hz, 2H), 7.65 (d, J=8.4 Hz, 1H), 7.72 (d, J=8.0 Hz, 1H).

ESI-MS: m/z, 590.26 (M+H)⁺, 100%.

### Example 86: (4-hydroxy-4-(4-(trifluoromethyl)phenyl)piperidin-1-yl)(4-(3-hydroxyoxetan-3-yl)phenyl)methanone

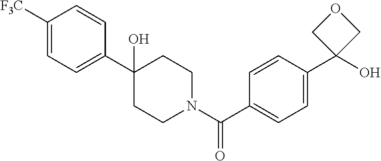 [SMILES: O=C(c1ccc(C2(O)COC2)cc1)N1CCC(O)(c2ccc(C(F)(F)F)cc2)CC1]

¹H NMR (DMSO-d6) δ: 1.77-1.91 (m, 4H), 2.67-2.81 (m, 2H), 3.84 (d, J=12.4 Hz, 2H), 4.70 (d, J=6.8 Hz, 2H), 4.79 (d, J=6.8 Hz, 2H), 5.44 (brs, 1H), 6.44 (brs, 1H), 7.01 (d, J=8.8 Hz, 2H), 7.43 (d, J=6.8 Hz, 2H), 7.52 (d, J=8.0 Hz, 2H), 7.67 (d, J=8.0 Hz, 2H).

ESI-MS: m/z, 422.15 (M+H)⁺, 100%.

### Example 87: (4-(3-hydroxyoxetan-3-yl)phenyl)(4-(4-(trifluoromethyl)phenyl)-5,6-dihydropyridin-1(2H)-yl)methanone

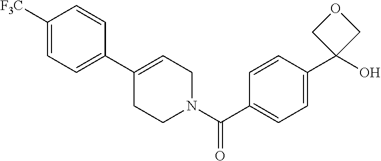 [SMILES: O=C(c1ccc(C2(O)COC2)cc1)N1CC=C(c2ccc(C(F)(F)F)cc2)CC1]

¹H NMR (DMSO-d₆) δ: 2.67-2.81 (m, 2H), 3.50-3.90 (m, 2H), 4.01-4.30 (m, 2H), 4.70 (d, J=6.4 Hz, 2H), 4.80 (d, J=6.4 Hz, 2H), 6.47 (brs, 1H), 6.50 (s, 1H), 7.51 (d, J=8.4 Hz, 2H), 7.70 (d, J=7.6 Hz, 6H).

ESI-MS: m/z, 404.13 (M+H)⁺, 100%.

### Example 88: (4-(3-hydroxyoxetan-3-yl) phenyl)(1-(4-(trifluoromethyl)phenyl)-4,6-dihydropyrrolo[3,4-c]pyrazol-5(1H)-yl)methanone

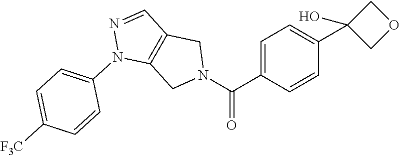 [SMILES: O=C(c1ccc(C2(O)COC2)cc1)N1Cc2cnn(-c3ccc(C(F)(F)F)cc3)c2C1]

¹H NMR (DMSO-d₆) δ: 4.59 (s, 1H), 4.67-4.73 (m, 3H), 4.82 (d, J=6.4 Hz, 2H), 5.14 (d, J=12.8 Hz, 2H), 6.51 (s, 1H), 7.64-7.75 (m, 6H), 7.85 (d, J=8.4 Hz, 1H), 7.09 (s, 2H).

ESI-MS: m/z, 430.13 (M+H)⁺, 100%.

### Example 89: (4-(3-hydroxyoxetan-3-yl)phenyl)(2-(4-(trifluoromethyl)phenyl)-2,6-dihydropyrrolo[3,4-c]pyrazol-5(4H)-yl)methanone

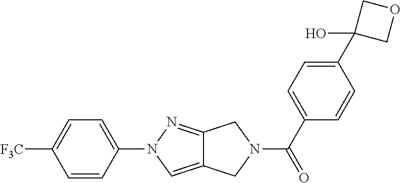 [SMILES: O=C(c1ccc(C2(O)COC2)cc1)N1Cc2cn(-c3ccc(C(F)(F)F)cc3)nc2C1]

¹H NMR (DMSO-d₆) δ: 4.63 (s, 1H), 4.72 (d, J=9.2 Hz, 4H), 4.75 (s, 1H), 4.81 (d, J=6.8 Hz, 2H), 6.49 (s, 1H), 7.65-7.73 (m, 4H), 7.86 (d, J=8.8 Hz, 2H), 8.06 (d, J=8.4 Hz, 2H), 8.43-8.54 (m, 1H).

ESI-MS: m/z, 430.13 (M+H)⁺, 100%.

### Example 90: (4-(3-methoxyoxetan-3-yl)phenyl)(2-(4-(trifluoromethyl)phenyl)-2,6-dihydropyrrolo[3,4-c]pyrazol-5(4H)-yl)methanone

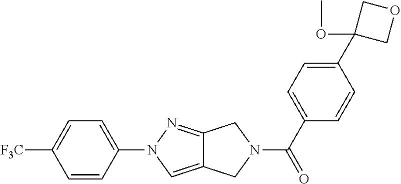 [SMILES: COC1(c2ccc(C(=O)N3Cc4cn(-c5ccc(C(F)(F)F)cc5)nc4C3)cc2)COC1]

¹H NMR (DMSO-d₆) δ: 3.07 (s, 3H), 4.63 (s, 1H), 4.70-4.75 (m, 3H), 4.79 (d, J=7.6 Hz, 2H), 4.82 (d, J=6.8 Hz, 2H), 7.56 (d, J=8.4 Hz, 2H), 7.70 (d, J=8.0 Hz, 2H), 7.86 (d, J=8.8 Hz, 2H), 8.05 (t, J=9.2 Hz, 2H), 8.42 (s, 0.5H), 8.54 (s, 0.5H).

ESI-MS: m/z, 444.11 (M+H)⁺, 100%.

### Example 91: (4-(3-hydroxyoxetan-3-yl)phenyl)(5-methoxy-5-(4-(trifluoromethyl)phenyl) hexahydrocyclopenta[c]pyrrol-2(1H)-yl)methanone

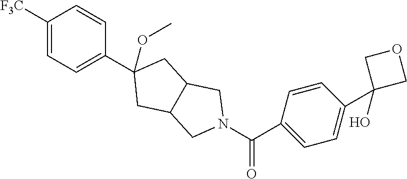 [SMILES: COC1(c2ccc(C(F)(F)F)cc2)CC2CN(C(=O)c3ccc(C4(O)COC4)cc3)CC2C1]

¹H NMR (DMSO-d₆) δ: 1.98-2.01 (m, 1H), 2.12-2.15 (m, 2H), 2.25-2.27 (m, 1H), 2.68-2.70 (m, 1H), 2.82-2.84 (m, 1H), 2.85 (s, 3H), 3.34-3.37 (m, 1H), 3.40-3.67 (m, 3H), 4.69 (d, J=6.4 Hz, 2H), 4.78 (d, J=6.4 Hz, 2H), 6.45 (s, 1H), 7.52-7.54 (m, 2H), 7.56-7.58 (m, 2H), 7.61-7.63 (m, 2H), 7.71-7.73 (m, 2H).

ESI-MS: m/z 462.2 (M+H)⁺, 100%.

### Example 92: (5-methoxy-5-(4-(trifluoromethyl)phenyl) hexahydrocyclopenta

[c]pyrrol-2(1H)-yl)(4-(3-methoxyoxetan-3-yl)phenyl)methanone

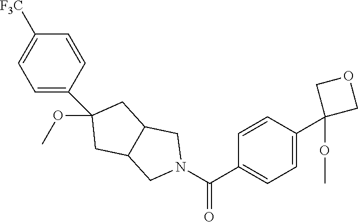 [SMILES: COC1(c2ccc(C(=O)N3CC4CC(OC)(c5ccc(C(F)(F)F)cc5)CC4C3)cc2)COC1]

¹H NMR (DMSO-d₆) δ: 1.98-2.01 (m, 1H), 2.12-2.15 (m, 2H), 2.25-2.27 (m, 1H), 2.68-2.70 (m, 1H), 2.82-2.84 (m, 1H), 2.85 (s, 3H), 3.01 (s, 3H), 3.34-3.37 (m, 1H), 3.40-3.67 (m, 3H), 4.69 (d, J=6.4 Hz, 2H), 4.78 (d, J=6.4 Hz, 2H), 7.52-7.54 (m, 2H), 7.56-7.58 (m, 2H), 7.61-7.63 (m, 2H), 7.71-7.73 (m, 2H).

ESI-MS: m/z 476.2 (M+H)⁺, 75%.

### Example 93: (4-(3-hydroxyoxetan-3-yl)phenyl)(5-(4-(trifluoromethyl)phenyl)-3,5-dihydropyrrolo[3,4-c]pyrrol-2(1H)-yl)methanone

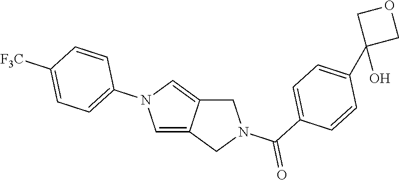 [SMILES: O=C(c1ccc(C2(O)COC2)cc1)N1Cc2cn(-c3ccc(C(F)(F)F)cc3)cc2C1]

¹H NMR (DMSO-d₆) δ: 4.56 (s, 2H), 4.67 (s, 2H), 4.70 (d, J=8.0 Hz, 2H), 4.80 (d, J=8.0 Hz, 2H), 6.51 (s, 1H), 7.28 (s, 1H), 7.40 (s, 1H), 7.63 (d, J=8.4 Hz, 2H), 7.69 (d, J=8.0 Hz, 2H), 7.77-7.82 (m, 4H).

ESI-MS: m/z 429.20 (M+H)⁺, 100%.

### Example 94: (4-(3-methoxyoxetan-3-yl)phenyl)(5-(4-(trifluoromethyl)phenyl)-3,5-dihydropyrrolo[3,4-c]pyrrol-2(1H)-yl)methanone

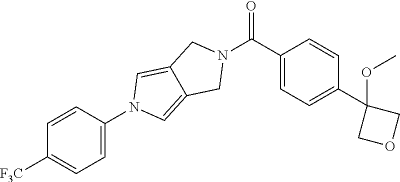 [SMILES: COC1(c2ccc(C(=O)N3Cc4cn(-c5ccc(C(F)(F)F)cc5)cc4C3)cc2)COC1]

¹H NMR (DMSO-d₆) δ: 3.07 (s, 3H), 4.56 (s, 2H), 4.68 (s, 2H), 4.78-4.82 (m, 4H), 7.28 (s, 1H), 7.40 (s, 1H), 7.54 (d, J=8.0 Hz, 2H), 7.67 (d, J=8.0 Hz, 2H), 7.77-7.82 (m, 4H).

ESI-MS: m/z 445.2 (M+H)⁺, 100%.

### Example 95: (4-(3-hydroxyoxetan-3-yl)phenyl)(5-(4-(trifluoromethyl)phenyl)-3,4,5,6-tetrahydropyrrolo[3,4-c]pyrrol-2(1H)-yl)methanone

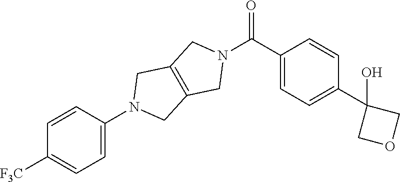 [SMILES: O=C(c1ccc(C2(O)COC2)cc1)N1CC2=C(C1)CN(c1ccc(C(F)(F)F)cc1)C2]

¹H NMR (DMSO-d6) δ: 4.00 (s, 2H), 4.18 (s, 2H), 4.25 (s, 2H), 4.41 (s, 2H), 4.70 (d, J=8.0 Hz, 2H), 4.80 (d, J=8.0 Hz, 2H), 6.48 (s, 1H), 6.62 (d, J=8.0 Hz, 2H), 7.48 (d, J=8.8 Hz, 2H), 7.61 (d, J=8.8 Hz, 2H), 7.69 (d, J=8.0 Hz, 2H).

ESI-MS: m/z 431.15 (M+H)⁺, 100%.

### Example 96: (3-(3-hydroxyoxetan-3-yl)phenyl)(5-(4-(trifluoromethyl)phenyl)-3,4,5,6-tetrahydropyrrolo[3,4-c]pyrrol-2(1H)-yl)methanone

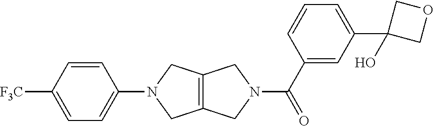 [SMILES: O=C(c1cccc(C2(O)COC2)c1)N1CC2=C(C1)CN(c1ccc(C(F)(F)F)cc1)C2]

¹H NMR (DMSO-d₆) δ: 3.90 (s, 2H), 4.016 (s, 2H), 4.29 (s, 2H), 4.56 (s, 2H), 4.70 (d, J=8.0 Hz, 2H), 4.79 (d, J=8.0 Hz, 2H), 6.50 (s, 1H), 6.62 (d, J=8.8 Hz, 2H), 7.50-7.53 (m, 4H), 7.70-7.76 (m, 2H).

ESI-MS: m/z 431.2 (M+H)⁺, 100%.

### Example 97: (3-(3-methoxyoxetan-3-yl)phenyl)(5-(4-(trifluoromethyl)phenyl)-3,4,5,6-tetrahydropyrrolo[3,4-c]pyrrol-2(1H)-yl)methanone

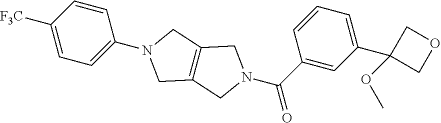 [SMILES: COC1(c2cccc(C(=O)N3CC4=C(C3)CN(c3ccc(C(F)(F)F)cc3)C4)c2)COC1]

¹H NMR (DMSO-d₆) δ: 3.05 (s, 3H), 4.08 (s, 2H), 4.19 (s, 2H), 4.32 (s, 2H), 4.42 (s, 2H), 4.76 (m, 4H), 6.62 (d, J=8.0 Hz, 2H), 7.32 (d, J=8.4 Hz, 2H), 7.49 (d, J=8.8 Hz, 2H), 7.65 (d, J=8.4 Hz, 2H).

ESI-MS: m/z 445.1 (M+H)⁺, 100%.

### Example 98: 3-(4-((5-(4-(trifluoromethyl)phenyl)hexahydropyrrolo[3,4-c]pyrrol-2(1H)-yl)sulfonyl)phenyl)oxetan-3-ol

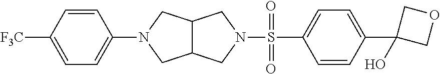 [SMILES: O=S(=O)(c1ccc(C2(O)COC2)cc1)N1CC2CN(c3ccc(C(F)(F)F)cc3)CC2C1]

¹H NMR (DMSO-d₆) δ: 2.94-2.96 (m, 4H), 3.07-3.10 (m, 2H), 3.36-3.38 (m, 4H), 4.63 (d, J=6.8 Hz, 2H), 4.80 (d, J=8.0 Hz, 2H), 6.51 (d, J=8.8 Hz, 2H), 7.41 (d, J=8.8 Hz, 2H), 7.82-7.87 (m, 4H).

ESI-MS: m/z 469.2 (M+H)⁺, 100%.

### Example 99: (4-(3-hydroxyoxetan-3-yl)phenyl)(9-(4-(trifluoromethyl)phenyl)-3,9-diazaspiro[5.5]undecan-3-yl)methanone

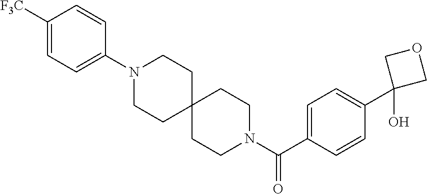 [SMILES: O=C(c1ccc(C2(O)COC2)cc1)N1CCC2(CC1)CCN(c1ccc(C(F)(F)F)cc1)CC2]

¹H NMR (CDCl₃) δ: 1.52-1.60 (m, 2H), 1.66-1.71 (m, 6H), 3.29-3.31 (m, 4H), 3.46-3.50 (m, 2H), 3.78-3.80 (m, 2H), 4.87 (d, J=7.2 Hz, 2H), 4.91 (d, J=7.2 Hz, 2H), 7.01 (d, J=8.0 Hz, 2H), 7.43-7.51 (m, 4H), 7.74-7.76 (m, 2H).

ESI-MS: m/z 475.2 (M+H)⁺, 100%.

### Example 100: (3-(3-hydroxyoxetan-3-yl)phenyl)(9-(4-(trifluoromethyl)phenyl)-3,9-diazaspiro[5.5]undecan-3-yl)methanone

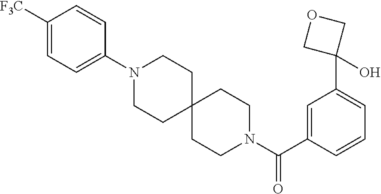 [SMILES: O=C(c1cccc(C2(O)COC2)c1)N1CCC2(CC1)CCN(c1ccc(C(F)(F)F)cc1)CC2]

¹H NMR (CDCl₃) δ: 1.14-1.16 (m, 2H), 1.56-1.59 (m, 2H), 1.80-2.00 (m, 4H), 3.13-3.18 (m, 4H), 3.31-3.36 (m, 2H), 3.71-3.80 (m, 2H), 4.85 (d, J=7.2 Hz, 2H), 4.92 (d, J=7.2 Hz, 2H), 6.62 (d, J=8.0 Hz, 2H), 7.33-7.35 (m, 1H), 7.46-7.48 (m, 3H), 7.53-7.56 (m, 1H), 7.70-7.72 (m, 1H).

ESI-MS: m/z 475.2 (M+H)⁺, 100%.

### Example 101: (4-(3-methoxyoxetan-3-yl)phenyl)(9-(4-(trifluoromethyl)phenyl)-3,9-diazaspiro[5.5]undecan-3-yl)methanone

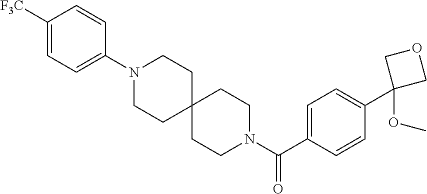 [SMILES: COC1(c2ccc(C(=O)N3CCC4(CC3)CCN(c3ccc(C(F)(F)F)cc3)CC4)cc2)COC1]

¹H NMR (CDCl₃) δ: 1.70-1.73 (m, 4H), 1.98-2.02 (m, 4H), 3.12 (s, 3H), 3.22-3.23 (m, 4H), 3.45-3.51 (m, 2H), 3.68-3.80 (m, 2H), 4.81 (d, J=7.2 Hz, 2H), 4.95 (d, J=7.2 Hz, 2H), 6.95 (d, J=8.0 Hz, 2H), 7.49-7.53 (m, 4H), 7.54-7.60 (m, 2H).

ESI-MS: m/z 489.2 (M+H)⁺, 100%.

### Example 102: (4-(3-hydroxyoxetan-3-yl)phenyl)(3-(4-(trifluoromethyl)phenyl) pyrrolidin-1-yl)methanone

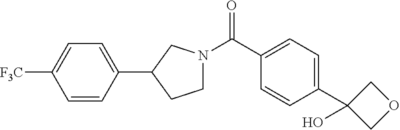 [SMILES: O=C(c1ccc(C2(O)COC2)cc1)N1CCC(c2ccc(C(F)(F)F)cc2)C1]

¹H NMR (CDCl₃) δ: 1.98-2.04 (m, 1H), 3.45-3.55 (m, 4H), 3.57-3.65 (m, 1H), 4.00-4.02 (m, 1H), 4.67-4.69 (m, 2H), 4.76-4.80 (m, 2H), 6.44 (d, J=6.0 Hz, 1H), 7.50-7.52 (m, 1H), 7.58-7.60 (m, 3H), 7.65-7.72 (m, 4H).

ESI-MS: m/z 392.14 (M+H)⁺, 100%.

### Example 103: ((5-(4-fluorophenyl)-3,3a,6,6a-tetrahydrocyclopenta[c]pyrrol-2(1H)-yl)(4-(3-hydroxyoxetan-3-yl)phenyl)methanone

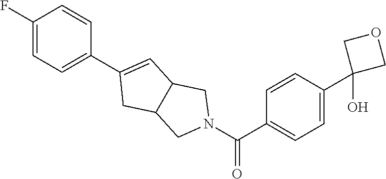 [SMILES: O=C(c1ccc(C2(O)COC2)cc1)N1CC2C=C(c3ccc(F)cc3)CC2C1]

¹H NMR (DMSO-d₆) δ: 2.51-2.54 (m, 1H), 2.63-2.67 (m, 1H), 3.02-3.05 (m, 1H), 3.18-3.21 (m, 1H), 3.55-3.65 (m, 2H), 3.75-3.80 (m, 2H), 4.68 (d, J=7.6 Hz, 2H), 4.78 (d, J=7.6 Hz, 2H), 6.45 (m, 1H), 7.18 (t, J=8.0 Hz, 2H), 7.49-7.50 (m, 4H), 7.63-7.66 (m, 2H).

ESI-MS: m/z 380.17 (M+H)⁺, 100%.

### Example 104: (4-(3-ethoxyoxetan-3-yl)phenyl)(5-(4-(trifluoromethyl)phenyl)-3,4,5,6-tetrahydropyrrolo[3,4-c]pyrrol-2(1H)-yl)methanone

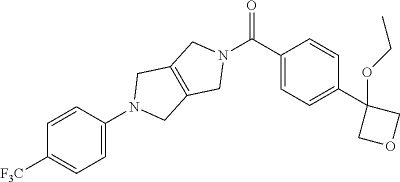 [SMILES: CCOC1(c2ccc(C(=O)N3CC4=C(C3)CN(c3ccc(C(F)(F)F)cc3)C4)cc2)COC1]

¹H NMR (DMSO-d6) δ: 1.2 (t, J=8.0 Hz, 3H), 3.05 (q, J=8.0 Hz, 2H), 4.00 (s, 2H), 4.18 (s, 2H), 4.25 (s, 2H), 4.41 (s, 2H), 4.70 (d, J=8.0 Hz, 2H), 4.80 (d, J=8.0 Hz, 2H), 6.62 (d, J=8.0 Hz, 2H), 7.48 (d, J=8.8 Hz, 2H), 7.61 (d, J=8.8 Hz, 2H), 7.69 (d, J=8.0 Hz, 2H).

ESI-MS: m/z 459.18 (M+H)⁺, 75%.

### Example 105: (5-hydroxy-5-(4-(trifluoromethyl)phenyl) hexahydrocyclopenta

[c]pyrrol-2(1H)-yl)(4-(3-hydroxyoxetan-3-yl)phenyl)methanone

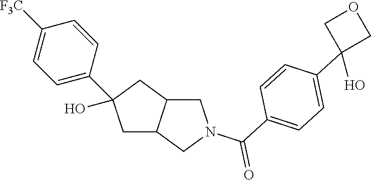 [SMILES: O=C(c1ccc(C2(O)COC2)cc1)N1CC2CC(O)(c3ccc(C(F)(F)F)cc3)CC2C1]

¹H NMR (CDCl₃) δ: 2.03-2.07 (m, 2H), 2.14-2.17 (m, 2H), 2.36-2.40 (m, 2H), 3.03 (s, 1H), 3.84-3.87 (m, 3H), 4.85 (d, J=7.2 Hz, 2H), 4.93 (d, J=7.2 Hz, 2H), 7.50-7.55 (m, 2H), 7.55-7.57 (m, 4H), 7.61-7.65 (m, 3H).

ESI-MS: m/z 448.16 (M+H)⁺, 60%.

### Example 106: (4-(3-hydroxyoxetan-3-yl)phenyl)(6-(4-(trifluoromethyl)phenyl)-3,4-dihydroisoquinolin-2(1H)-yl)methanone

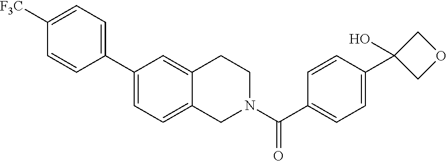 [SMILES: O=C(c1ccc(C2(O)COC2)cc1)N1CCc2cc(-c3ccc(C(F)(F)F)cc3)ccc2C1]

¹H NMR (DMSO-d6) δ: 2.86-2.96 (m, 2H), 3.75-3.79 (m, 1H), 3.89 (s, 1H), 4.71-4.98 (m, 6H), 6.49 (s, 1H), 7.44-7.58 (m, 5H), 7.71 (d, J=7.6 Hz, 2H), 7.80-7.89 (m, 4H).

ESI-MS: m/z, 454.16 (M+H)⁺, 100%.

### Example 107: (4-(3-hydroxyoxetan-3-yl)phenyl)(5-(4-(trifluoromethyl)phenyl) indolin-1-yl)methanone

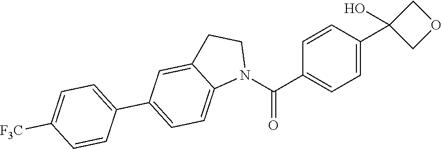 [SMILES: O=C(c1ccc(C2(O)COC2)cc1)N1CCc2cc(-c3ccc(C(F)(F)F)cc3)ccc21]

¹H NMR (DMSO-d₆) δ: 3.18 (t, J=8.0 Hz, 2H), 4.10 (t, J=8.0 Hz, 2H), 4.72 (d, J=6.8 Hz, 2H), 4.82 (d, J=6.4 Hz, 2H), 6.51 (s, 1H), 7.33-7.46 (m, 4H), 7.60-7.80 (m, 4H), 7.89 (d, J=8.0 Hz, 2H).

ESI-MS: m/z, 440.14 (M+H)⁺, 100%.

### Example 108: (4-(3-methoxyoxetan-3-yl)phenyl)(4-(3-(4-methyl-1H-imidazol-1-yl)-4-(trifluoromethyl)phenyl)piperidin-1-yl)methanone

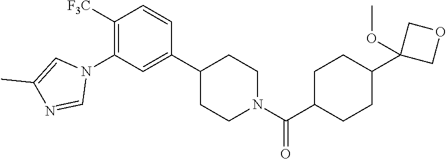 [SMILES: COC1(C2CCC(C(=O)N3CCC(c4ccc(C(F)(F)F)c(-n5cnc(C)c5)c4)CC3)CC2)COC1]

¹H NMR (DMSO-d₆) δ: 1.38-1.71 (m, 4H), 1.87-1.91 (m, 1H), 1.99.2.09 (m, 1H), 2.19 (s, 3H), 2.67-2.86 (m, 1H), 3.04 (s, 3H), 3.61-3.74 (m, 1H), 4.58-4.68 (m, 1H), 4.75-4.80 (m, 4H), 7.04 (s, 1H), 7.50-7.57 (s, 4H), 7.65-7.85 (m, 3H), 7.96-8.04 (m, 1H).

ESI-MS: m/z, 500.21 (M+H)⁺, 100%.

### Example 109: 2,2-dimethyl-7-((3-(4-(4-(4-(trifluoromethyl)phenyl)piperidine-1-carbonyl)phenyl)oxetan-3-yl)oxy)heptanoic acid

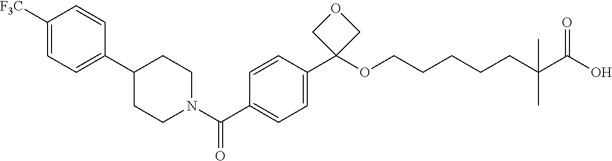 [SMILES: CC(C)(CCCCCOC1(c2ccc(C(=O)N3CCC(c4ccc(C(F)(F)F)cc4)CC3)cc2)COC1)C(=O)O]

¹H NMR (DMSO-d₆) δ: 0.83-0.89 (m, 2H), 1.12 (s, 6H), 1.23-1.39 (m, 3H), 1.45-1.59 (m, 3H), 1.60-2.06 (m, 3H), 2.88-2.98 (m, 2H), 3.06-3.09 (m, 2H), 3.21-3.23 (m, 1H), 3.98-4.01 (m, 1H), 4.90-4.99 (m, 5H), 7.36 (d, J=8.0 Hz, 2H), 7.45 (d, J=8.4 Hz, 2H), 7.54 (d, J=8.4 Hz, 2H), 7.61 (d, J=8.0 Hz, 2H).

ESI-MS: m/z, 562.27 (M+H)⁺, 100%.

### Example 110: (4-(methoxymethyl)-4-(4-(trifluoromethyl)phenyl)piperidin-1-yl)(4-(3-methoxyoxetan-3-yl)phenyl)methanone

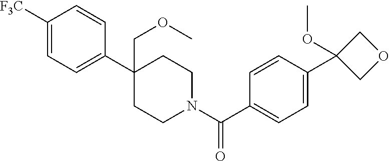 [SMILES: COCC1(c2ccc(C(F)(F)F)cc2)CCN(C(=O)c2ccc(C3(OC)COC3)cc2)CC1]

¹H NMR (DMSO-d₆) δ: 1.82-1.91 (m, 2H), 2.11-2.20 (m, 2H), 3.04 (s, 3H), 3.13-3.33 (m, 5H), 3.33-3.43 (m, 3H), 3.99-4.02 (m, 1H), 4.75-4.80 (m, 4H), 7.43-7.51 (m, 4H), 7.63-7.71 (m, 4H).

ESI-MS: m/z, 464.20 (M+H)⁺, 100%.

### Example 111: (4-(hydroxymethyl)-4-(4-(trifluoromethyl)phenyl)piperid in-1-yl)(4-(3-hydroxyoxetan-3-yl)phenyl)methanone

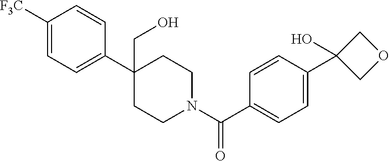 [SMILES: O=C(c1ccc(C2(O)COC2)cc1)N1CCC(CO)(c2ccc(C(F)(F)F)cc2)CC1]

¹H NMR (DMSO-d6) δ: 1.66-1.86 (m, 2H), 1.99-2.40 (m, 2H), 3.21-3.45 (m, 1H), 3.59-3.79 (m, 3H), 3.33-3.43 (m, 3H), 4.15-4.30 (m, 1H), 4.83 (d, J=7.2 Hz, 2H), 4.93 (d, J=7.2 Hz, 2H), 7.36 (d, J=8.4 Hz, 2H), 7.50 (d, J=8.4 Hz, 2H), 7.63 (d, J=8.4 Hz, 2H), 7.68 (d, J=8.4 Hz, 2H).

ESI-MS: m/z, 436.17 (M+H)⁺, 100%.

### Example 112: 1-(4-(3-methoxyoxetan-3-yl)benzoyl)-4-(4-(trifluoromethyl) phenyl)piperidine-4-carboxylic acid

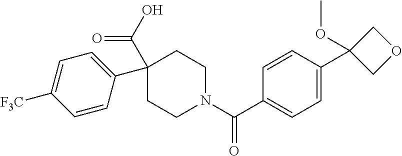 [SMILES: COC1(c2ccc(C(=O)N3CCC(C(=O)O)(c4ccc(C(F)(F)F)cc4)CC3)cc2)COC1]

¹H NMR (DMSO-d₆) δ: 1.91-1.99 (m, 2H), 2.40-2.50 (m, 2H), 3.04 (s, 3H), 3.12-3.13 (m, 2H), 3.43-3.49 (m, 1H), 4.22-4.36 (m, 1H), 4.75-4.80 (m, 4H), 7.50 (s, 4H), 7.75 (d, J=7.6 Hz, 2H), 8.01 (d, J=8.4 Hz, 2H).

ESI-MS: m/z, 464.16 (M+H)⁺, 100%

### Example 113: Methyl 1-(4-(3-hydroxyoxetan-3-yl)benzoyl)-4-(4-(trifluoromethyl)phenyl)piperidine-4-carboxylate

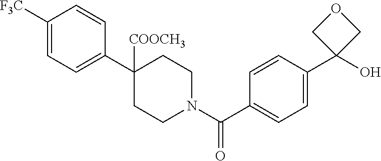 [SMILES: COC(=O)C1(c2ccc(C(F)(F)F)cc2)CCN(C(=O)c2ccc(C3(O)COC3)cc2)CC1]

¹H NMR (DMSO-d₆) δ: 1.97-1.99 (m, 2H), 2.40-2.51 (m, 2H), 2.85-3.30 (m, 2H), 3.57-3.60 (m, 1H), 3.65 (s, 3H), 4.28 (s, 1H), 4.69 (d, J=6.8 Hz, 2H), 4.79 (d, J=6.8 Hz, 2H), 6.45 (s, —OH), 7.45 (d, J=8.4 Hz, 2H), 7.62-7.67 (m, 4H), 7.75 (d, J=8.4 Hz, 2H).

ESI-MS: m/z, 464.15 (M+H)⁺, 100%

### Example 114: 1-(4-(3-hydroxyoxetan-3-yl)benzoyl)-4-(4-(trifluoromethyl) phenyl)piperidine-4-carboxylic acid

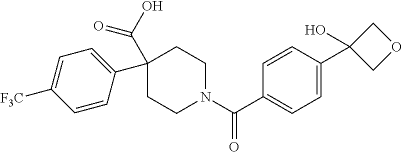 [SMILES: O=C(c1ccc(C2(O)COC2)cc1)N1CCC(C(=O)O)(c2ccc(C(F)(F)F)cc2)CC1]

¹H NMR (DMSO-d₆) δ: 1.53-1.57 (m, 2H), 2.23-2.50 (m, 2H), 3.08-3.17 ((m, 1H), 3.34-3.51 (m, 2H), 4.35 (s, 1H), 4.69 (d, J=6.8 Hz, 2H), 4.79 (d, J=6.8 Hz, 2H), 6.49 (s, —OH), 7.40 (d, J=8.0, 2H), 7.62-7.67 (m, 6H).

ESI-MS: m/z, 450.20 (M+H)⁺, 70%.

### Example 115: (4-(3-chloro-4-(trifluoromethyl)phenyl)piperidin-1-yl)(4-(3-methoxyoxetan-3-yl)phenyl)methanone

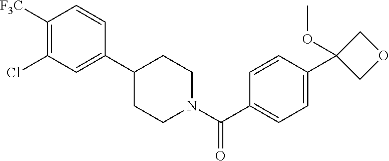 [SMILES: COC1(c2ccc(C(=O)N3CCC(c4ccc(C(F)(F)F)c(Cl)c4)CC3)cc2)COC1]

¹H NMR (DMSO-d₆) δ: 1.68-1.73 (m, 4H), 2.84-3.00 (m, 2H), 3.05 (s, 3H), 3.10-3.17 (m, 1H), 3.69 (s, 1H), 4.65 (s, 1H), 4.76 (d, J=6.8 Hz, 2H), 4.80 (d, J=6.8 Hz, 2H), 7.49-7.53 (m, 5H), 7.71 (s, 1H), 7.79 (d, J=8.0 Hz, 1H).

ESI-MS: m/z, 454.11 (M+H)⁺, 100%.

### Example 116: 1-(4-(3-hydroxyoxetan-3-yl)benzoyl)-4-(4-(trifluoromethyl) phenyl)piperidine-4-carbonitrile

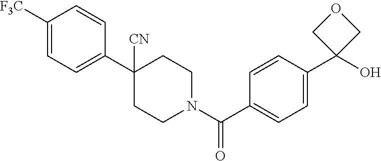 [SMILES: N#CC1(c2ccc(C(F)(F)F)cc2)CCN(C(=O)c2ccc(C3(O)COC3)cc2)CC1]

¹H NMR (DMSO-d6) δ: 1.92-2.27 (m, 4H), 3.15-3.70 (m, 3H), 3.98-4.16 (m, 1H), 4.87 (d, J=6.8 Hz, 2H), 4.95-5.15 (m, 3H), 7.48 (d, J=8.00 Hz, 2H), 7.61-7.74 (m, 6H).

ESI-MS: m/z, 431.14 (M+H)⁺, 100%.

### Example 117: 1-(4-(3-methoxyoxetan-3-yl)benzoyl)-4-(4-(trifluoromethyl) phenyl) piperidine-4-carbonitrile

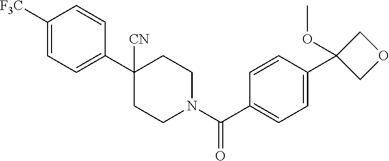 [SMILES: COC1(c2ccc(C(=O)N3CCC(C#N)(c4ccc(C(F)(F)F)cc4)CC3)cc2)COC1]

¹H NMR (DMSO-d₆) δ: 2.17-2.33 (m, 4H), 3.05-3.20 (m, 5H), 3.43.-3.60 (m, 1H), 3.80-3.82 (m, 1H), 4.76 (d, J=9.2 Hz, 2H), 4.80 (d, J=6.8 Hz, 2H), 7.53-7.59 (m, 4H), 7.86 (s, 4H).

ESI-MS: m/z, 445.15 (M+H)⁺, 100%

### Example 118: (4-(3-hydroxyoxetan-3-yl)phenyl)(4-methoxy-4-(4-(trifluoromethyl)phenyl)piperidin-1-yl)methanone

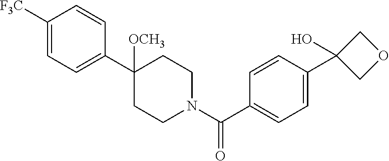 [SMILES: COC1(c2ccc(C(F)(F)F)cc2)CCN(C(=O)c2ccc(C3(O)COC3)cc2)CC1]

¹H NMR (DMSO-d₆) δ: 1.93-2.08 (m, 4H), 2.95 (s, 3H), 3.07-3.10 (m, 1H), 3.33-3.52 (m, 2H), 4.45 (s, 1H), 4.69 (d, J=6.4 Hz, 2H), 4.79 (d, J=6.4 Hz, 2H), 6.45 (s, —OH), 7.49 (d, J=8.0 Hz, 2H), 7.64-7.68 (m, 4H), 7.76 (d, J=8.4 Hz, 2H).

ESI-MS: m/z, 436.17 (M+H)⁺, 100%.

### Example 119: (4-methoxy-4-(4-(trifluoromethyl)phenyl)piperidin-1-yl)(4-(3-methoxyoxetan-3-yl)phenyl)methanone

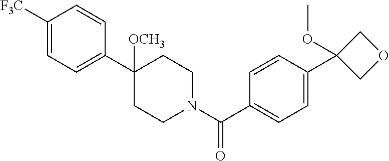 [SMILES: COC1(c2ccc(C(F)(F)F)cc2)CCN(C(=O)c2ccc(C3(OC)COC3)cc2)CC1]

¹H NMR (DMSO-d6) δ: 1.95-2.08 (m, 4H), 2.95 (s, 3H), 3.05 (s, 3H), 3.12-3.20 (m, 1H), 3.41-3.50 (m, 2H), 4.45 (s, 1H), 4.76 (d, J=7.2 Hz, 2H), 4.79 (d, J=6.8 Hz, 2H), 7.52 (s, 4H), 7.65 (d, J=8.0 Hz, 2H), 7.76 (d, J=8.0 Hz, 2H).

ESI-MS: m/z, 450.3 (M+H)⁺, 100%,

### Example 120: (3-(3-hydroxyoxetan-3-yl)phenyl)(4-(3-(trifluoromethyl) phenyl) piperidin-1-yl)methanone

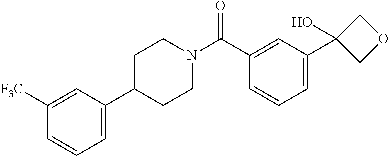 [SMILES: O=C(c1cccc(C2(O)COC2)c1)N1CCC(c2cccc(C(F)(F)F)c2)CC1]

¹H NMR (DMSO-d₆) δ: 1.17-1.88 (m, 4H), 2.86-2.99 (m, 2H), 3.17 (s, 1H), 3.70 (s, 1H), 4.66-4.71 (m, 3H), 4.79 (d, J=6.4 Hz, 2H), 6.46 (s, —OH), 7.26-7.79 (m, 8H).

ESI-MS: m/z, 406.19 (M+H)⁺, 100%.

### Example 121: (4-(3-fluoro-4-(trifluoromethyl)phenyl)piperidin-1-yl)(3-(3-hydroxyoxetan-3-yl)phenyl)methanone

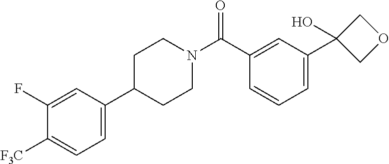 [SMILES: O=C(c1cccc(C2(O)COC2)c1)N1CCC(c2ccc(C(F)(F)F)c(F)c2)CC1]

¹H NMR (DMSO-d₆) δ: 1.71-1.85 (m, 4H), 2.85-2.91 (m, 1H), 3.20-3.26 (m, 2H), 3.70 (s, 1H), 4.66-4.70 (m, 3H), 4.79 (d, J=6.4 Hz, 2H), 6.46 (s, —OH), 7.37-7.45 (m, 2H), 7.47-7.51 (m, 1H), 7.63-7.71 (m, 3H), 7.77-7.80 (m, 1H).

ESI-MS: m/z, 424.15 (M+H)⁺, 100%.

### Example 122: (3-(3-hydroxyoxetan-3-yl)phenyl)(4-(2-(trifluoromethyl)phenyl) piperidin-1-yl)methanone

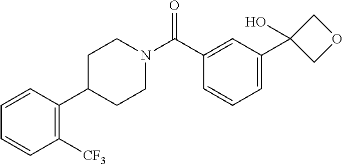 [SMILES: O=C(c1cccc(C2(O)COC2)c1)N1CCC(c2ccccc2C(F)(F)F)CC1]

¹H NMR (DMSO-d₆) δ: 1.65-1.78 (m, 4H), 2.87 (s, 1H), 3.11-3.18 (m, 2H), 3.71-3.72 (m, 1H), 4.66-4.71 (m, 3H), 4.79 (d, J=6.8 Hz, 2H), 6.46 (s, —OH), 7.40-7.44 (m, 2H), 7.48-7.51 (m, 1H), 7.64-7.76 (m, 5H).

ESI-MS: m/z, 406.18 (M+H)⁺, 100%.

### Example 123: (4-(3-ethoxyoxetan-3-yl)phenyl)(3-(4-(trifluoromethyl)phenyl) piperidin-1-yl)methanone

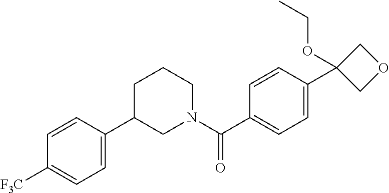 [SMILES: CCOC1(c2ccc(C(=O)N3CCCC(c4ccc(C(F)(F)F)cc4)C3)cc2)COC1]

¹H NMR (DMSO-d6) δ: 1.13 (t, J=7.2 Hz, 3H), 1.62-1.74 (m, 4H), 2.92-2.98 (m, 2H), 3.17-3.32 (m, 3H), 3.70-3.71 (m, 1H), 4.68-4.71 (m, 1H), 4.76 (d, J=6.8 Hz, 2H), 4.79 (d, J=6.8 Hz, 2H), 7.50-7.55 (m, 6H), 7.67 (d, J=8.0 Hz, 2H).

ESI-MS: m/z, 434.22 (M+H)⁺, 100%.

### Example 124: 3-(3-(3-(4-(trifluoromethyl)phenyl)-1,2,4-oxadiazol-5-yl)phenyl) oxetan-3-ol

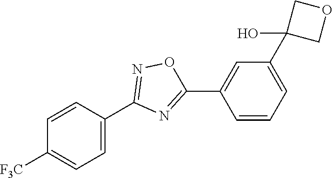 [SMILES: OC1(c2cccc(-c3nc(-c4ccc(C(F)(F)F)cc4)no3)c2)COC1]

¹H NMR (DMSO-d₆) δ: 4.74 (d, J=6.8 Hz, 2H), 4.86 (d, J=6.8 Hz, 2H), 6.70 (s, —OH), 7.73 (t, J=7.6 Hz, 1H), 7.97-8.03 (m, 3H), 8.16 (d, J=8.0 Hz, 1H), 8.33 (d, J=8.0 Hz, 2H), 8.43 (d, J=3.2 Hz, 1H).

ESI-MS: m/z, 363.09 (M+H)⁺, 100%.

### Example 125: 5-(3-(3-methoxyoxetan-3-yl)phenyl)-3-(4-(trifluoromethyl) phenyl)-1,2,4-oxadiazole

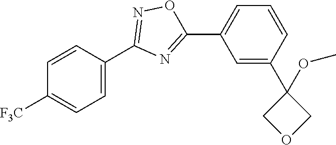 [SMILES: COC1(c2cccc(-c3nc(-c4ccc(C(F)(F)F)cc4)no3)c2)COC1]

¹H NMR (DMSO-d₆) δ: 3.12 (s, 3H), 4.82-4.88 (m, 4H), 7.76-7.79 (m, 1H), 7.86-7.89 (m 1H), 8.00 (d, J=4.4 Hz, 2H), 7.21-7.24 (m, 2H), 8.34 (d, J=8.0 Hz, 2H).

ESI-MS: m/z, 377.17 (M+H)⁺, 100%.

### Example 126: 3-(4-(3-(4-(trifluoromethyl)phenyl)-1,2,4-oxadiazol-5-yl)phenyl) oxetan-3-ol

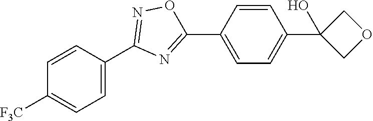 [SMILES: OC1(c2ccc(-c3nc(-c4ccc(C(F)(F)F)cc4)no3)cc2)COC1]

¹H NMR (DMSO-d₆) δ: 4.72 (d, J=7.2 Hz, 2H), 4.85 (d, J=6.8 Hz, 2H), 6.66 (s, —OH), 7.92 (dd, J=6.8 & 1.6 Hz, 2H), 7.99 (d, J=8.4 Hz, 2H), 8.25 (d, J=6.8 Hz, 2H), 8.32 (d, J=8.4 Hz, 2H).

ESI-MS: m/z, 363.09 (M+H)⁺, 100%.

### Example 127: 5-(4-(3-methoxyoxetan-3-yl)phenyl)-3-(4-(trifluoromethyl) phenyl)-1,2,4-oxadiazole

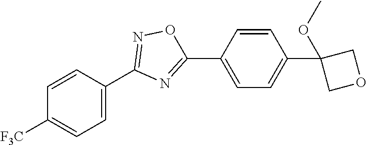 [SMILES: COC1(c2ccc(-c3nc(-c4ccc(C(F)(F)F)cc4)no3)cc2)COC1]

¹H NMR (DMSO-d₆) δ: 3.11 (s, 3H), 4.79 (d, J=7.6 Hz, 2H), 4.86 (d, J=7.2 Hz, 2H), 7.79 (d, J=8.4 Hz, 2H), 8.00 (d, J=4.4 Hz, 2H), 8.28 (d, J=8.4 Hz, 2H), 8.32 (d, J=8.0 Hz, 2H).

ESI-MS: m/z, 377.10 (M+H)⁺, 100%.

### Example 128: (4-(3-hydroxyoxetan-3-yl)phenyl)(4-(4-((trifluoromethyl)thio) phenyl) piperidin-1-yl)methanone

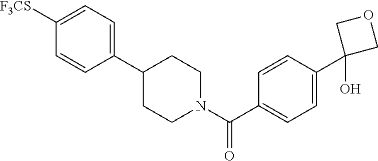

¹H NMR (CDCl₃) δ: 1.62-2.01 (m, 4H), 2.82-2.90 (m, 2H), 3.17 (brs, 1H), 3.90 (brs, 1H), 4.86-4.94 (m, 5H), 7.23-7.35 (m, 2H), 7.45 (d, J=8.4 Hz, 2H), 7.63 (d, J=8.0 Hz, 2H), 7.66 (d, J=8.0 Hz, 2H).

ESI-MS: m/z, 438.13 (M+H)⁺, 100%.

### Example 129: (4-(3-methoxyoxetan-3-yl)phenyl)(4-(4-((trifluoromethyl)thio) phenyl) piperidin-1-yl)methanone

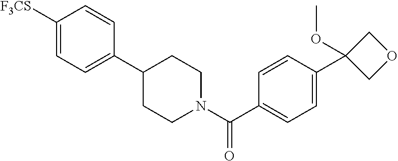

¹H NMR (CDCl₃) δ: 1.60-1.88 (m, 4H), 2.89-2.95 (m, 2H), 3.05 (s, 3H), 3.18-3.23 (m, 1H), 3.70 (brs, 1H), 4.64-4.70 (m, 1H), 4.76-4.81 (m, 4H), 7.44-7.54 (m, 6H), 7.63 (d, J=8.4 Hz, 2H).

ESI-MS: m/z, 452.2 (M+H)⁺, 100%.

### Example 130: (4-(3-hydroxyoxetan-3-yl)phenyl)(5-(p-tolyl)-3,3a,6,6a-tetrahydrocyclopenta[c]pyrrol-2(1H)-yl)methanone

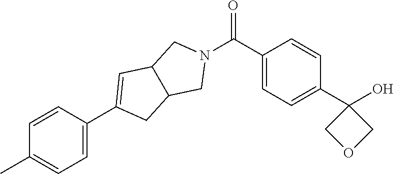 [SMILES: Cc1ccc(C2=CC3CN(C(=O)c4ccc(C5(O)COC5)cc4)CC3C2)cc1]

¹H NMR (CDCl₃) δ: 2.29 (s, 3H), 2.71-2.80 (m, 1H), 2.98-3.02 (m, 1H), 2.18-2.22 (m, 1H), 3.41-3.53 (m, 2H), 3.68-3.77 (m, 3H), 4.69 (s, 2H), 4.77 (d, J=6.0 Hz, 2H), 6.43 (s, 1H), 7.14 (d, J=8.0 Hz, 2H), 7.55 (d, J=8.0 Hz, 2H), 7.48 (d, J=7.2 Hz, 2H), 7.64 (s, 2H).

ESI-MS: m/z, 376.18, 100%.

### Example 131: ((4-(3-hydroxyoxetan-3-yl)phenyl)(5-(4-(methylthio)phenyl)-3,3a,4,6a-tetrahydrocyclopenta[c]pyrrol-2(1H)-yl)methanone

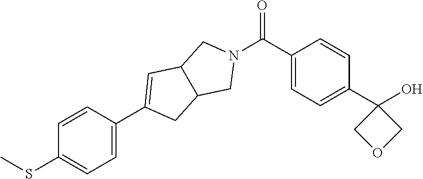 [SMILES: CSc1ccc(C2=CC3CN(C(=O)c4ccc(C5(O)COC5)cc4)CC3C2)cc1]

¹H NMR (CDCl₃) δ: 2.47 (s, 3H), 2.81-2.90 (m, 1H), 2.98-3.02 (m, 1H), 2.18-2.22 (m, 1H), 3.45-3.58 (m, 2H), 3.70-3.82 (m, 3H), 4.69 (s, 2H), 4.77 (d, J=6.0 Hz, 2H), 6.43 (s, 1H), 7.22 (dd, J=8.4 & 1.8 Hz, 2H), 7.40 (d, J=8.4 Hz, 2H), 7.48 (d, J=8.0 Hz, 2H), 7.64 (s, 2H).

ESI-MS: m/z, 408.16, 100%.

### Example 132: (4-(3-hydroxyoxetan-3-yl)phenyl)(6-(4-(trifluoromethyl)phenyl)-3,6-diazabicyclo[3.1.1]heptan-3-yl)methanone

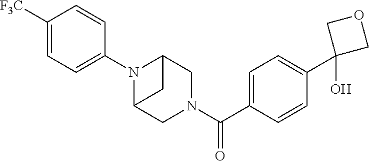 [SMILES: O=C(c1ccc(C2(O)COC2)cc1)N1CC2CC(C1)N2c1ccc(C(F)(F)F)cc1]

¹H NMR (CDCl₃) δ: 1.72 (d, J=8.8 Hz, 1H), 2.85-2.90 (m, 1H), 3.39 (d, J=11.2 Hz, 1H), 3.78 (d, J=14.0 Hz, 1H), 3.94 (d, J=13.2 Hz, 1H), 4.26-4.30 (m, 2H), 4.45-4.48 (m, 1H), 4.87 (d, J=7.2 Hz, 2H), 4.93 (d, J=7.2 Hz, 2H), 6.56 (d, J=8.4 Hz, 2H), 7.19 (dd, J=6.8 & 2.0 Hz, 2H), 7.50-7.54 (m, 2H), 7.62 (dd, J=6.6 & 1.8 Hz, 2H).

ESI-MS: m/z, 419.15 (M+H)⁺, 100%.

### Example 133: (4-(3-(cyclopropylmethoxy)oxetan-3-yl)phenyl)(5-(4-(trifluoromethyl)phenyl)hexahydropyrrolo[3,4-c]pyrrol-2(1H)-yl)methanone

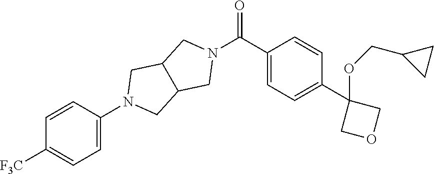 [SMILES: O=C(c1ccc(C2(OCC3CC3)COC2)cc1)N1CC2CN(c3ccc(C(F)(F)F)cc3)CC2C1]

¹H NMR (DMSO-d₆): 0.10-0.12 (m, 2H), 0.43-0.47 (m, 2H), 0.96-1.00 (m, 1H), 2.94-2.98 (m, 2H), 3.05-3.09 (m, 2H), 3.15-3.18 (m, 1H), 3.36-3.39 (m, 2H), 3.45-3.50 (m, 2H), 3.56-3.60 (m, 1H), 3.74-3.80 (m, 1H), 3.82-3.85 (m, 1H), 4.74 (d, J=7.2 Hz, 2H), 4.78 (d, J=8.0 Hz, 2H), 6.63 (d, J=7.6 Hz, 2H), 7.45-7.51 (m, 4H), 7.58 (d, J=8.0 Hz, 2H).

ESI-MS: m/z 487.2 (M+H)⁺, 100%.

### Example 134: (4-(3-(methoxy-d3)oxetan-3-yl)phenyl)(5-(4-(trifluoromethyl) phenyl)hexahydropyrrolo[3,4-c]pyrrol-2(1H)-yl)methanone

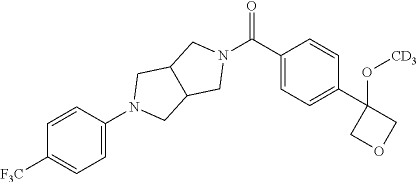 [SMILES: [2H]C([2H])([2H])OC1(c2ccc(C(=O)N3CC4CN(c5ccc(C(F)(F)F)cc5)CC4C3)cc2)COC1]

¹H NMR (DMSO-d₆): 3.04-3.09 (m, 2H), 3.15-3.18 (m, 1H), 3.38-3.39 (m, 2H), 3.45-3.50 (m, 2H), 3.56-3.60 (m, 1H), 3.74-3.80 (m, 1H), 3.82-3.85 (m, 1H), 4.74 (d, J=7.2 Hz, 2H), 4.78 (d, J=6.8 Hz, 2H), 6.63 (d, J=8.8 Hz, 2H), 7.45-7.51 (m, 4H), 7.59 (d, J=8.4 Hz, 2H).

ESI-MS: m/z 450.2 (M+H)⁺, 100%.

### Example 135: (4-(3-isobutoxyoxetan-3-yl)phenyl)(5-(4-(trifluoromethyl)phenyl) hexahydropyrrolo[3,4-c]pyrrol-2(1H)-yl)methanone

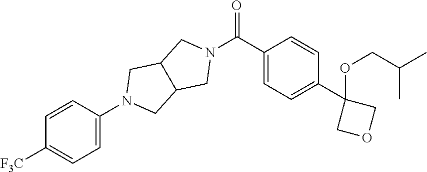 [SMILES: CC(C)COC1(c2ccc(C(=O)N3CC4CN(c5ccc(C(F)(F)F)cc5)CC4C3)cc2)COC1]

¹H NMR (CDCl₃): 0.95 (d, J=6.8 Hz, 6H), 1.81-1.91 (m, 1H), 2.91-2.96 (m, 2H), 3.11-3.26 (m, 3H), 3.38-3.39 (m, 1H), 3.46-3.47 (m, 1H), 3.55-3.59 (m, 1H), 3.55-3.71 (m, 2H), 3.74-3.86 (m, 1H), 4.03-4.06 (m, 1H), 4.79 (d, J=6.4 Hz, 2H), 4.95 (d, J=6.8 Hz, 2H), 6.57 (d, J=8.8 Hz, 2H), 7.28-7.68 (m, 6H).

ESI-MS: m/z 489.2 (M+H)⁺, 100%.

### Example 136: (4-(3-isopropoxyoxetan-3-yl)phenyl)(5-(4-(trifluoromethyl) phenyl)hexahydropyrrolo[3,4-c]pyrrol-2(1H)-yl)methanone

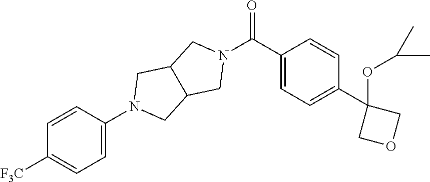 [SMILES: CC(C)OC1(c2ccc(C(=O)N3CC4CN(c5ccc(C(F)(F)F)cc5)CC4C3)cc2)COC1]

¹H NMR (DMSO-d6): 0.95 (d, J=6.0 Hz, 6H), 3.05-3.09 (m, 2H), 3.16-3.18 (m, 1H), 3.37-3.41 (m, 2H), 3.43-3.59 (m, 3H), 3.56-3.60 (m, 1H), 3.73-3.86 (m, 2H), 4.80 (s, 4H), 6.63 (d, J=8.4 Hz, 2H), 7.45-7.60 (m, 6H).

ESI-MS: m/z 475.2 (M+H)⁺, 100%.

### Example 137: (4-(3-(2-methoxyethoxy)oxetan-3-yl)phenyl)(5-(4-(trifluoromethyl)phenyl)hexahydropyrrolo[3,4-c]pyrrol-2(1H)-yl)methanone

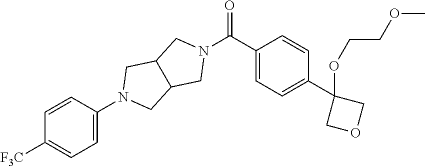 [SMILES: COCCOC1(c2ccc(C(=O)N3CC4CN(c5ccc(C(F)(F)F)cc5)CC4C3)cc2)COC1]

¹H NMR (DMSO-d6): 3.10-3.25 (m, 2H), 3.36-3.38 (m, 4H), 3.49 (s, 3H), 3.50-3.61 (m, 3H), 3.68-3.81 (m, 4H), 3.96-4.01 (m, 1H), 4.79 (d, J=7.2 Hz, 2H), 5.00 (d, J=7.2 Hz, 2H), 6.56 (d, J=8.8 Hz, 2H), 7.47-7.49 (m, 2H), 7.55-7.60 (m, 4H).

ESI-MS: m/z 491.2 (M+H)⁺, 100%.

### Example 138: 3-(4-(5-(4-(trifluoromethyl)phenyl)octahydropyrrolo[3,4-c]pyrrole-2-carbonyl)phenyl)oxetan-3-yl acetate

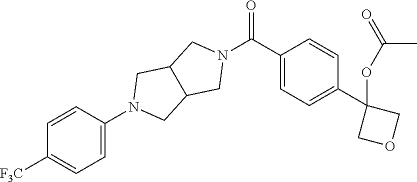 [SMILES: CC(=O)OC1(c2ccc(C(=O)N3CC4CN(c5ccc(C(F)(F)F)cc5)CC4C3)cc2)COC1]

¹H NMR (DMSO-d₆): 2.15 (s, 3H), 3.05-3.18 (m, 3H), 3.33-3.35 (m, 2H), 3.37-3.38 (m, 2H), 3.45-3.49 (m, 1H), 3.71-3.82 (m, 2H), 4.80 (d, J=7.6 Hz, 2H), 4.94 (d, J=8.0 Hz, 2H), 6.62 (d, J=8.4 Hz, 2H), 7.45 (d, J=8.8 Hz, 2H), 7.53-7.59 (m, 4H).

ESI-MS: m/z 475.3 (M+H)⁺, 100%.

### Example 139: 3-(4-(5-(4-(trifluoromethyl)phenyl)octahydropyrrolo[3,4-c]pyrrole-2-carbonyl)phenyl)oxetan-3-yl pivalate

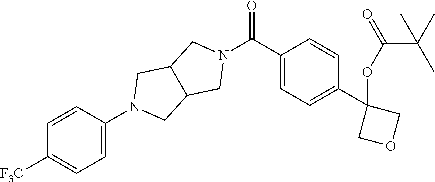 [SMILES: CC(C)(C)C(=O)OC1(c2ccc(C(=O)N3CC4CN(c5ccc(C(F)(F)F)cc5)CC4C3)cc2)COC1]

¹H NMR (CDCl₃): 1.28 (s, 9H), 3.09-3.24 (m, 3H), 3.38-3.47 (m, 2H), 3.51-3.54 (m, 1H), 3.66-3.84 (m, 3H), 4.00-4.05 (m, 1H), 4.90 (d, J=6.8 Hz, 2H), 5.01 (d, J=7.6 Hz, 2H), 6.56 (d, J=8.4 Hz, 2H), 7.47-7.58 (m, 6H).

ESI-MS: m/z 517.4 (M+H)⁺, 100%.

### Example 140: tert-butyl 4-hydroxy-4-(4-(4-(4-(trifluoromethyl)phenyl) piperidine-1-carbonyl)phenyl)piperidine-1-carboxylate

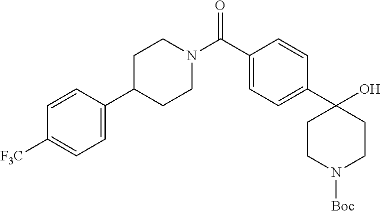 [SMILES: CC(C)(C)OC(=O)N1CCC(O)(c2ccc(C(=O)N3CCC(c4ccc(C(F)(F)F)cc4)CC3)cc2)CC1]

¹H NMR (CDCl₃): 1.47 (s, 9H), 1.60-1.88 (m, 5H), 1.91-2.01 (m, 4H), 2.85-88 (m, 2H), 3.02-3.24 (m, 3H), 4.04-4.11 (m, 3H), 7.34 (d, J=8.0 Hz, 2H), 7.42 (d, J=8.4 Hz, 2H), 7.52 (d, J=8.0 Hz, 2H), 7.59 (d, J=8.0 Hz, 2H).

ESI-MS: m/z 533.2 (M+H)⁺, 30%.

### Example 141: (4-(4-hydroxypiperidin-4-yl)phenyl)(4-(4-(trifluoromethyl) phenyl)piperidin-1-yl)methanone

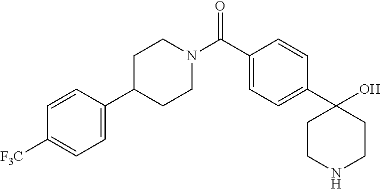 [SMILES: O=C(c1ccc(C2(O)CCNCC2)cc1)N1CCC(c2ccc(C(F)(F)F)cc2)CC1]

¹H NMR (DMSO-d₆): 1.59-1.69 (m, 2H), 1.77-1.81 (m, 3H), 1.83-1.93 (m, 3H), 2.73-2.75 (m, 2H), 2.80-2.86 (m, 3H), 3.04-3.31 (m, 3H), 3.71-3.73 (m, 1H), 7.30 (d, J=8.4 Hz, 2H), 7.45 (d, J=7.2 Hz, 2H), 7.52 (d, J=8.0 Hz, 2H), 7.66 (d, J=8.0 Hz, 2H).

ESI-MS: m/z 433.2 (M+H)⁺, 15%.

### Example 142: (4-(4-hydroxytetrahydro-2H-pyran-4-yl)phenyl)(4-(4-(trifluoromethyl)phenyl)piperidin-1-yl)methanone

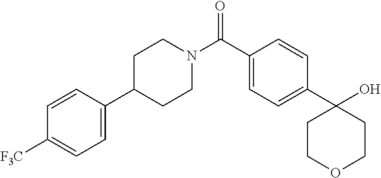 [SMILES: O=C(c1ccc(C2(O)CCOCC2)cc1)N1CCC(c2ccc(C(F)(F)F)cc2)CC1]

¹H NMR (DMSO-d₆): 1.52-2.00 (m, 9H), 2.90-2.94 (m, 2H), 3.01-3.09 (m, 1H), 3.70-3.82 (m, 4H), 4.61-4.63 (m, 1H), 7.40 (d, J=8.4 Hz, 2H), 7.53 (d, J=8.4 Hz, 2H), 7.55 (d, J=8.4 Hz, 2H), 7.66 (d, J=8.0 Hz, 2H).

ESI-MS: m/z 434.2 (M+H)⁺, 100%.

### Example 143: (4-(4-methoxytetrahydro-2H-pyran-4-yl)phenyl)(4-(4-(trifluoromethyl)phenyl)piperidin-1-yl)methanone

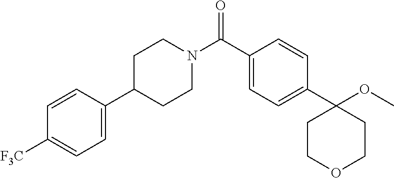 [SMILES: COC1(c2ccc(C(=O)N3CCC(c4ccc(C(F)(F)F)cc4)CC3)cc2)CCOCC1]

¹H NMR (DMSO-d₆): 1.60-1.99 (m, 9H), 2.84-2.97 (m, 5H), 3.10-3.19 (m, 1H), 3.66-3.72 (m, 4H), 4.61-4.63 (m, 1H), 7.47-7.50 (m, 4H), 7.53 (d, J=8.0 Hz, 2H), 7.66 (d, J=8.4 Hz, 2H).

ESI-MS: m/z 448.2 (M+H)⁺, 100%.

### Example 144: (4-(4-methoxypiperidin-4-yl)phenyl)(4-(4-(trifluoromethyl)phenyl) piperidin-1-yl)methanone

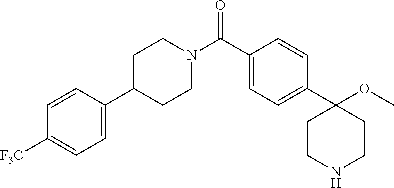 [SMILES: COC1(c2ccc(C(=O)N3CCC(c4ccc(C(F)(F)F)cc4)CC3)cc2)CCNCC1]

¹H NMR (DMSO-d6): 1.61-1.68 (m, 2H), 1.70-1.94 (m, 3H), 1.99-2.08 (m, 3H), 2.17-2.20 (m, 2H), 2.89 (s, 3H), 2.95-3.01 (m, 1H), 3.10-3.61 (m, 5H), 3.61-3.69 (m, 1H), 7.44-7.54 (m, 6H), 7.66 (d, J=8.4 Hz, 2H).

ESI-MS: m/z 447.2 (M+H)⁺, 90%.

### Example 145: (4-(3-hydroxythietan-3-yl)phenyl)(4-(4-(trifluoromethyl)phenyl) piperidin-1-yl)methanone

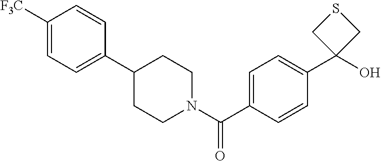 [SMILES: O=C(c1ccc(C2(O)CSC2)cc1)N1CCC(c2ccc(C(F)(F)F)cc2)CC1]

¹H NMR (DMSO-d₆): 1.61-1.87 (m, 4H), 1.80-1.87 (m, 1H), 2.89-2.97 (m, 2H), 3.16-3.90 (m, 1H), 3.38 (d, J=10.4 Hz, 2H), 3.61 (d, J=10.0 Hz, 2H), 3.71-3.72 (m, 1H), 7.46 (d, J=8.4 Hz, 2H), 7.53 (d, J=8.0 Hz, 2H), 7.66 (d, J=8.4 Hz, 2H), 7.78 (d, J=8.4 Hz, 2H).

ESI-MS: m/z 422.1 (M+H)⁺, 100%.

### Example 146: (4-(3-hydroxythietan-3-yl)phenyl)(5-(4-(trifluoromethyl)phenyl) hexahydropyrrolo[3,4-c]pyrrol-2(1H)-yl)methanone

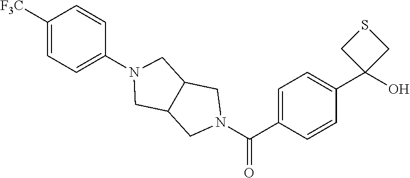 [SMILES: O=C(c1ccc(C2(O)CSC2)cc1)N1CC2CN(c3ccc(C(F)(F)F)cc3)CC2C1]

¹H NMR (DMSO-d₆): 3.04-3.09 (m, 2H), 3.15-3.19 (m, 1H), 3.31-3.38 (m, 4H), 3.45-3.51 (m, 2H), 3.56-3.63 (m, 3H), 3.72-3.76 (m, 1H), 3.80-3.85 (m, 1H), 6.63 (d, J=8.4 Hz, 2H), 7.45 (d, J=8.4 Hz, 2H), 7.54 (d, J=8.4 Hz, 2H), 7.75 (d, J=8.4 Hz, 2H).

ESI-MS: m/z 449.2 (M+H)⁺, 100%.

### Example 147: (4-(3-methoxythietan-3-yl)phenyl)(4-(4-(trifluoromethyl)phenyl) piperidin-1-yl)methanone

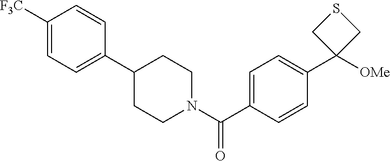 [SMILES: COC1(c2ccc(C(=O)N3CCC(c4ccc(C(F)(F)F)cc4)CC3)cc2)CSC1]

¹H NMR (DMSO-d₆): 1.68-1.99 (m, 4H), 2.89 (s, 3H), 2.92-2.98 (m, 2H), 3.13-3.20 (m, 1H), 3.37 (d, J=10.4 Hz, 2H), 3.64 (d, J=10.4 Hz, 2H), 3.70-3.73 (m, 1H), 4.65-4.66 (m, 1H), 7.46-7.55 (m, 4H), 7.64-7.68 (m, 4H).

ESI-MS: m/z: 436.2 (M+H)⁺, 100%.

### Example 148: (4-(3-ethoxythietan-3-yl)phenyl)(4-(4-(trifluoromethyl)phenyl) piperidin-1-yl)methanone

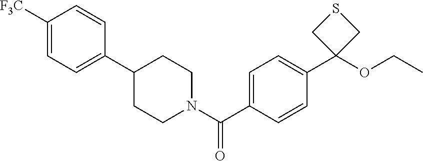 [SMILES: CCOC1(c2ccc(C(=O)N3CCC(c4ccc(C(F)(F)F)cc4)CC3)cc2)CSC1]

¹H NMR (DMSO-d₆): 1.20 (t, J=12.0 Hz, 3H), 1.71-2.03 (m, 4H), 2.87-2.93 (m, 2H), 3.10-3.23 (m, 3H), 3.31 (dd, J=8.8 & 1.6 Hz, 2H), 3.78 (dd, J=8.4 & 1.6 Hz, 2H), 3.78-3.81 (m, 1H), 4.88-4.93 (m, 1H), 7.35 (d, J=8.0 Hz, 2H), 7.51 (dd, J=6.6 & 1.8 Hz, 2H), 7.6 (d, J=8.0 Hz, 2H), 7.69 (dd, J=6.6 & 1.8 Hz, 2H).

ESI-MS: m/z: 450.2 (M+H)⁺, 100%.

### Example 149: (4-(3-methoxythietan-3-yl)phenyl)(5-(4-(trifluoromethyl)phenyl) hexahydropyrrolo[3,4-c]pyrrol-2(1H)-yl)methanone

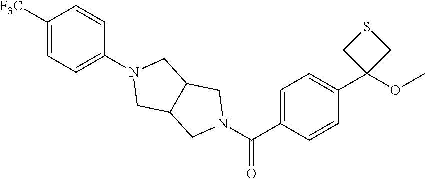 [SMILES: COC1(c2ccc(C(=O)N3CC4CN(c5ccc(C(F)(F)F)cc5)CC4C3)cc2)CSC1]

¹H NMR (DMSO-d₆): 2.88 (s, 3H), 2.91-3.08 (m, 2H), 3.10-3.19 (m, 1H), 3.33-3.45 (m, 4H), 3.45-3.50 (m, 2H), 3.56-3.60 (m, 1H), 3.64 (d, J=10.4 Hz, 2H), 3.78-3.86 (m, 2H), 6.63 (d, J=8.4 Hz, 2H), 7.46-7.50 (m, 2H), 7.59-7.64 (m, 4H).

ESI-MS: m/z: 463.2 (M+H)⁺, 100%.

### Example 150: (4-(3-ethoxythietan-3-yl)phenyl)(5-(4-(trifluoromethyl)phenyl) hexahydropyrrolo[3,4-c]pyrrol-2(1H)-yl)methanone

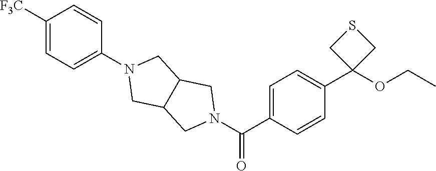 [SMILES: CCOC1(c2ccc(C(=O)N3CC4CN(c5ccc(C(F)(F)F)cc5)CC4C3)cc2)CSC1]

¹H NMR (DMSO-d₆): 1.16 (t, J=7.2 Hz, 3H), 3.00-3.05 (m, 4H), 3.08-3.10 (m, 1H), 3.33-3.43 (m, 4H), 3.45-3.50 (m, 2H), 3.56-3.58 (m, 1H), 3.65 (d, J=10.4 Hz, 2H), 3.78-3.86 (m, 2H), 6.63 (d, J=8.8 Hz, 2H), 7.42-7.48 (m, 2H), 7.56-7.65 (m, 4H).

ESI-MS: m/z: 477.2 (M+H)⁺, 100%.

### Example 151: tert-butyl 3-hydroxy-3-(4-(4-(4-(trifluoromethyl)phenyl) piperidine-1-carbonyl)phenyl)pyrrolidine-1-carboxylate

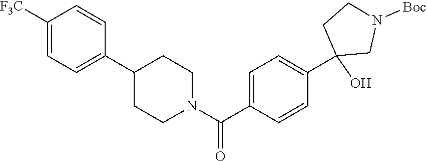 [SMILES: CC(C)(C)OC(=O)N1CCC(O)(c2ccc(C(=O)N3CCC(c4ccc(C(F)(F)F)cc4)CC3)cc2)C1]

¹H NMR (DMSO-d6): 1.49 (s, 9H), 1.78-1.83 (m, 2H), 1.92-1.98 (m, 1H), 2.06-2.20 (m, 2H), 2.51-2.60 (m, 1H), 2.85-2.91 (m, 2H), 3.02-3.17 (m, 1H), 3.59-3.80 (m, 4H), 3.92-4.02 (m, 1H), 4.83-4.93 (m, 1H), 7.34-7.81 (m, 8H).

ESI-MS: m/z: 463.2, 100%.

### Example 152: (4-(3-hydroxypyrrolidin-3-yl)phenyl)(4-(4-(trifluoromethyl) phenyl)piperidin-1-yl)methanone

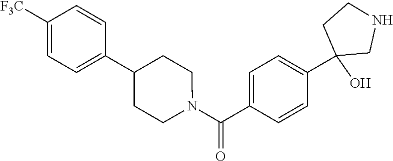 [SMILES: O=C(c1ccc(C2(O)CCNC2)cc1)N1CCC(c2ccc(C(F)(F)F)cc2)CC1]

¹H NMR (DMSO-d₆): 1.66-1.72 (m, 1H), 1.92-1.98 (m, 2H), 2.01-2.08 (m, 1H), 2.22-2.29 (m, 2H), 2.85-2.87 (m, 2H), 3.02-3.40 (m, 5H), 3.81-4.02 (m, 1H), 4.83-4.93 (m, 1H), 7.22-7.60 (m, 8H).

ESI-MS: m/z: 419.2, (M+H)⁺100%.

### Example 153: (4-(3-methoxypyrrolidin-3-yl)phenyl)(4-(4-(trifluoromethyl) phenyl)piperidin-1-yl)methanone

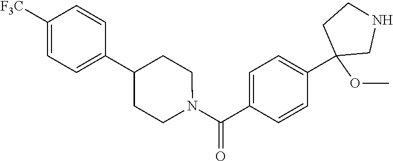 [SMILES: COC1(c2ccc(C(=O)N3CCC(c4ccc(C(F)(F)F)cc4)CC3)cc2)CCNC1]

¹H NMR (DMSO-d₆): 1.61-1.86 (m, 3H), 1.96-2.03 (m, 1H), 2.01-2.18 (m, 1H), 2.50 (s, 3H), 2.85-2.87 (m, 2H), 2.95-3.02 (m, 4H), 3.02-3.07 (m, 1H), 3.18-3.17 (m, 2H), 4.68-4.73 (m, 1H), 7.22-7.60 (m, 8H).

ESI-MS: m/z: 433.2 (M+H)⁺, 100%.

### Example 154: tert-butyl 3-hydroxy-3-(4-(4-(4-(trifluoromethyl) phenyl)piperidine-1-carbonyl)phenyl)azetidine-1-carboxylate

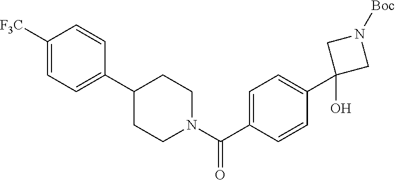 [SMILES: CC(C)(C)OC(=O)N1CC(O)(c2ccc(C(=O)N3CCC(c4ccc(C(F)(F)F)cc4)CC3)cc2)C1]

¹H NMR (DMSO-d6): 1.49 (s, 9H), 1.78-1.83 (m, 2H), 2.01-2.08 (m, 1H), 2.85-2.91 (m, 2H), 3.11-3.17 (m, 1H), 3.72 (s, 1H), 3.88-3.90 (m, 1H), 4.14-4.22 (m, 4H), 4.92-4.94 (m, 1H), 7.34 (d, J=8.0 Hz, 2H), 7.39 (d, J=8.4 Hz, 2H), 7.52 (d, J=8.4 Hz, 2H), 7.59 (d, J=8.0 Hz, 2H).

ESI-MS: m/z: 505.2 (M+H)⁺, 30%.

### Example 155: (4-(3-hydroxyazetidin-3-yl)phenyl)(4-(4-(trifluoromethyl)phenyl) piperidin-1-yl)methanone

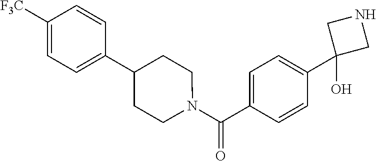 [SMILES: O=C(c1ccc(C2(O)CNC2)cc1)N1CCC(c2ccc(C(F)(F)F)cc2)CC1]

¹H NMR (DMSO-d6): 1.75-1.89 (m, 3H), 1.89-1.93 (m, 1H), 2.87-2.95 (m, 2H), 3.17-3.19 (m, 1H), 3.65-3.73 (m, 1H), 4.09 (s, 2H), 4.37 (d, J=10.4 Hz, 2H), 4.63-4.65 (m, 1H), 7.50-7.55 (m, 4H), 7.62-7.69 (m, 4H).

ESI-MS: m/z: 405.2 (M+H)⁺, 100%.

### Example 156: (4-(4-(methoxy-d3)piperidin-4-yl)phenyl)(4-(4-(trifluoromethyl) phenyl) piperidin-1-yl)methanone

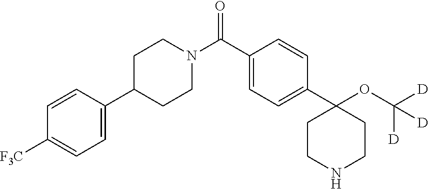 [SMILES: [2H]C([2H])([2H])OC1(c2ccc(C(=O)N3CCC(c4ccc(C(F)(F)F)cc4)CC3)cc2)CCNCC1]

¹H NMR (DMSO-d₆): 1.61-1.68 (m, 2H), 1.73-1.99 (m, 2H), 2.07-2.18 (m, 2H), 2.27-2.35 (m, 2H), 2.86-2.92 (m, 2H), 3.18-3.25 (m, 1H), 3.36-3.39 (m, 4H), 3.89-3.92 (m, 1H), 7.35 (d, J=8.4 Hz, 2H), 7.44 (d, J=8.4 Hz, 2H), 7.49 (d, J=8.8 Hz, 2H), 7.60 (d, J=8.4 Hz, 2H).

ESI-MS: m/z 450.2 (M+H)⁺, 90%.

### Example 157: tert-butyl 4-methoxy-4-(4-(4-(4-(trifluoromethyl) phenyl)piperidine-1-carbonyl)phenyl)piperidine-1-carboxylate

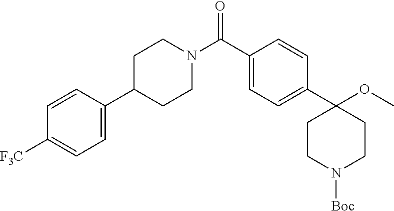 [SMILES: COC1(c2ccc(C(=O)N3CCC(c4ccc(C(F)(F)F)cc4)CC3)cc2)CCN(C(=O)OC(C)(C)C)CC1]

¹H NMR (DMSO-d₆): 1.41 (s, 9H), 1.61-1.81 (m, 6H), 1.96-1.99 (m, 2H), 2.91 (s, 3H), 2.85-2.92 (m, 2H), 3.06-3.18 (m, 3H), 3.71-3.88 (m, 3H), 4.58-4.63 (m, 1H), 7.43-7.48 (m, 4H), 7.52 (d, J=8.4 Hz, 2H), 7.66 (d, J=8.0 Hz, 2H).

ESI-MS: m/z: 547.2 (M+H)⁺, 10%.

### Example 158: 1-(4-methoxy-4-(4-(4-(4-(trifluoromethyl)phenyl)piperidine-1-carbonyl) phenyl)piperidin-1-yl)ethan-1-one

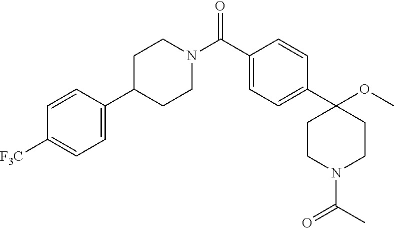 [SMILES: COC1(c2ccc(C(=O)N3CCC(c4ccc(C(F)(F)F)cc4)CC3)cc2)CCN(C(C)=O)CC1]

¹H NMR (DMSO-d₆): 1.63-1.75 (m, 4H), 1.70-1.76 (m, 2H), 1.98-2.02 (m, 5H), 2.82-2.96 (m, 6H), 3.06-3.18 (m, 1H), 3.37-3.38 (m, 1H), 3.68-3.72 (m, 2H), 4.28-4.31 (m, 1H), 4.61-4.68 (m, 1H), 7.43-7.48 (m, 4H), 7.52 (d, J=8.4 Hz, 2H), 7.66 (d, J=8.4 Hz, 2H).

ESI-MS: m/z: 489.2 (M+H)⁺, 100%.

### Example 159: Ethyl 4-methoxy-4-(4-(4-(4-(trifluoromethyl)phenyl)piperidine-1-carbonyl)phenyl)piperidine-1-carboxylate

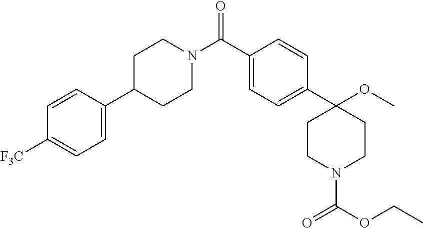 [SMILES: CCOC(=O)N1CCC(OC)(c2ccc(C(=O)N3CCC(c4ccc(C(F)(F)F)cc4)CC3)cc2)CC1]

¹H NMR (DMSO-d₆): 1.17 (t, J=7.0 Hz, 3H), 1.63-1.71 (m, 2H), 1.76-1.84 (m, 4H), 1.98-2.01 (m, 3H), 2.92-2.96 (m, 4H), 3.10-3.12 (m, 3H), 3.68-3.72 (m, 1H), 3.87-3.90 (m, 2H), 4.02 (q, J=7.2 Hz, 2H), 4.61-4.68 (m, 1H), 7.43-7.48 (m, 4H), 7.52 (d, J=8.4 Hz, 2H), 7.66 (d, J=8.4 Hz, 2H).

ESI-MS: m/z: 519.2 (M+H)⁺, 100%.

### Example 160: (4-(4-methoxy-1-methylpiperidin-4-yl)phenyl)(4-(4-(trifluoromethyl)phenyl)piperidin-1-yl)methanone

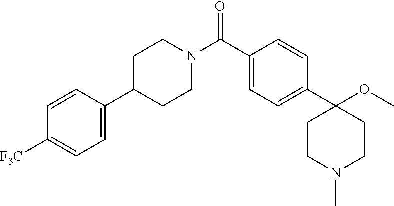 [SMILES: COC1(c2ccc(C(=O)N3CCC(c4ccc(C(F)(F)F)cc4)CC3)cc2)CCN(C)CC1]

¹H NMR (DMSO-d₆): 1.63-1.68 (m, 2H), 1.88-1.99 (m, 5H), 2.27 (s, 3H), 2.33-2.34 (m, 3H), 2.92-2.96 (m, 5H), 3.10-3.15 (m, 3H), 3.68-3.72 (m, 1H), 4.61-4.68 (m, 1H), 7.43-7.48 (m, 4H), 7.52 (d, J=8.0 Hz, 2H), 7.66 (d, J=8.0 Hz, 2H).

ESI-MS: m/z: 461.2 (M+H)⁺, 100%.

### Example 161: tert-butyl 4-ethoxy-4-(4-(4-(4-(trifluoromethyl)phenyl)piperidine-1-carbonyl) phenyl) piperidine-1-carboxylate

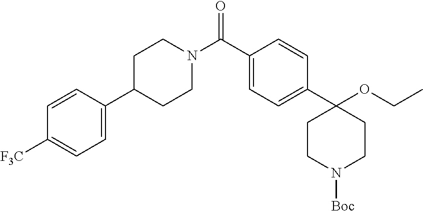 [SMILES: CCOC1(c2ccc(C(=O)N3CCC(c4ccc(C(F)(F)F)cc4)CC3)cc2)CCN(C(=O)OC(C)(C)C)CC1]

¹H NMR (DMSO-d₆): 1.15-1.18 (m, 3H), 1.48 (s, 9H), 1.68-1.88 (m, 5H), 1.95-2.02 (m, 3H), 2.85-2.91 (m, 2H), 3.10-3.21 (m, 5H), 3.90-3.97 (m, 3H), 4.63-4.69 (m, 1H), 7.34 (d, J=8.0 Hz, 2H), 7.43-7.48 (m, 4H), 7.59 (d, J=8.0 Hz, 2H).

ESI-MS: m/z: 505.2, 100%.

### Example 162: (4-(4-ethoxypiperidin-4-yl)phenyl)(4-(4-(trifluoromethyl)phenyl) piperidin-1-yl)methanone

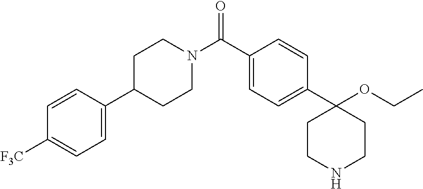 [SMILES: CCOC1(c2ccc(C(=O)N3CCC(c4ccc(C(F)(F)F)cc4)CC3)cc2)CCNCC1]

¹H NMR (DMSO-d₆): 1.14-1.19 (m, 3H), 1.68-2.02 (m, 8H), 2.44-2.61 (m, 3H), 2.82-2.89 (m, 2H), 3.02-3.21 (m, 5H), 3.90-3.97 (m, 1H), 7.35 (d, J=8.0 Hz, 2H), 7.44-7.49 (m, 4H), 7.59 (d, J=8.0 Hz, 2H).

ESI-MS: m/z: 461.2 (M+H)⁺, 10%.

### Example 163: 1-(4-ethoxy-4-(4-(4-(4-(trifluoromethyl)phenyl)piperidine-1-carbonyl) phenyl)piperidin-1-yl)ethan-1-one

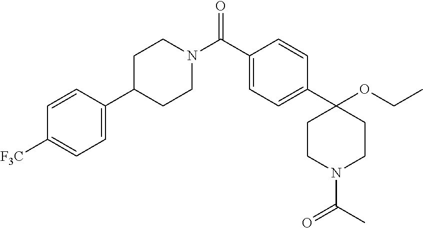 [SMILES: CCOC1(c2ccc(C(=O)N3CCC(c4ccc(C(F)(F)F)cc4)CC3)cc2)CCN(C(C)=O)CC1]

¹H NMR (DMSO-d₆): 1.16-1.20 (m, 3H), 1.76-1.92 (m, 5H), 1.98-2.09 (m, 3H), 2.14 (s, 3H), 2.86-2.92 (m, 2H), 2.99-3.22 (m, 4H), 3.56-3.71 (m, 2H), 3.90-3.97 (m, 1H), 4.55-4.59 (m, 1H), 4.90-4.93 (m, 1H), 7.34 (d, J=8.4 Hz, 2H), 7.43-7.47 (m, 4H), 7.59 (d, J=8.4 Hz, 2H).

ESI-MS: m/z: 503.5 (M+H)⁺, 25%.

### Example 164: Ethyl 4-ethoxy-4-(4-(4-(4-(trifluoromethyl)phenyl)piperidine-1-carbonyl) phenyl)piperidine-1-carboxylate

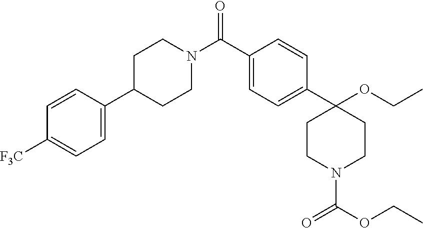 [SMILES: CCOC(=O)N1CCC(OCC)(c2ccc(C(=O)N3CCC(c4ccc(C(F)(F)F)cc4)CC3)cc2)CC1]

¹H NMR (DMSO-d6): 1.11-1.19 (m, 3H), 1.25-1.30 (m, 3H), 1.63-1.64 (m, 2H), 1.69-1.92 (m, 4H), 1.98-2.09 (m, 3H), 2.85-2.91 (m, 2H), 3.05-3.11 (m, 3H), 3.22-3.27 (m, 2H), 4.03-4.17 (m, 4H), 4.90-4.93 (m, 1H), 7.34 (d, J=8.4 Hz, 2H), 7.43-7.47 (m, 4H), 7.59 (d, J=8.0 Hz, 2H).

ESI-MS: m/z: 533.2 (M+H)⁺, 40%.

### Example 165: (4-(4-ethoxy-1-methylpiperidin-4-yl)phenyl)(4-(4-(trifluoromethyl)phenyl)piperidin-1-yl)methanone

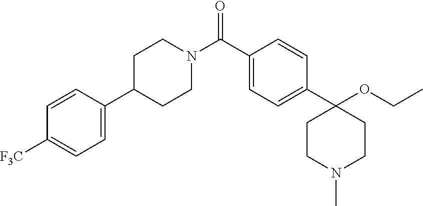 [SMILES: CCOC1(c2ccc(C(=O)N3CCC(c4ccc(C(F)(F)F)cc4)CC3)cc2)CCN(C)CC1]

¹H NMR (DMSO-d₆): 1.11-1.19 (m, 3H), 1.74-2.06 (m, 7H), 2.26-2.55 (m, 6H), 2.72-2.75 (m, 2H), 2.85-2.91 (m, 2H), 3.02-3.14 (m, 3H), 4.03-4.17 (m, 1H), 4.85-4.91 (m, 1H), 7.34 (d, J=8.0 Hz, 2H), 7.43-7.49 (m, 4H), 7.59 (d, J=8.0 Hz, 2H).

ESI-MS: m/z: 475.2 (M+H)⁺, 100%.

### Example 166: tert-butyl 4-hydroxy-4-(4-(5-(4-(trifluoromethyl)phenyl) octahydropyrrolo[3,4-c]pyrrole-2-carbonyl)phenyl)piperidine-1-carboxylate

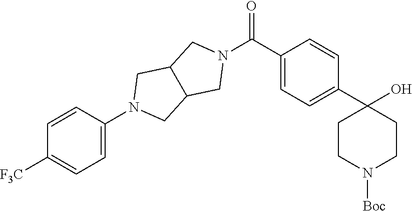 [SMILES: CC(C)(C)OC(=O)N1CCC(O)(c2ccc(C(=O)N3CC4CN(c5ccc(C(F)(F)F)cc5)CC4C3)cc2)CC1]

¹H NMR (DMSO-d₆): 1.50 (s, 9H), 1.66-1.73 (m, 4H), 2.00-2.08 (m, 2H), 3.08-3.24 (m, 6H), 3.35-3.44 (m, 2H), 3.65-3.84 (m, 2H), 3.99-4.04 (m, 2H), 6.56 (d, J=8.8 Hz, 2H), 7.47-7.54 (m, 6H).

ESI-MS: m/z: 560.2 (M+H)⁺, 15%.

### Example 167: tert-butyl 4-methoxy-4-(4-(5-(4-(trifluoromethyl)phenyl) octahydropyrrolo[3,4-c]pyrrole-2-carbonyl)phenyl)piperidine-1-carboxylate

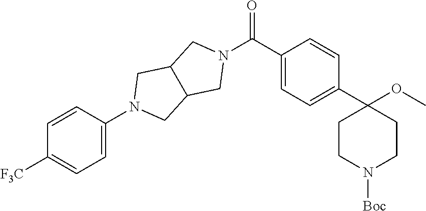 [SMILES: COC1(c2ccc(C(=O)N3CC4CN(c5ccc(C(F)(F)F)cc5)CC4C3)cc2)CCN(C(=O)OC(C)(C)C)CC1]

¹H NMR (DMSO-d₆): 1.41 (s, 9H), 1.71-1.79 (m, 2H), 1.95-1.98 (m, 2H), 2.90 (s, 3H), 3.04-3.18 (m, 5H), 3.21-3.30 (m, 2H), 3.44-3.59 (m, 3H), 3.71-3.84 (m, 4H), 6.56 (d, J=8.8 Hz, 2H), 7.47-7.54 (m, 6H).

ESI-MS: m/z: 518.3, 100%.

### Example 168: (4-(4-methoxypiperidin-4-yl)phenyl)(5-(4-(trifluoromethyl) phenyl) hexahydropyrrolo[3,4-c]pyrrol-2(1H)-yl)methanone

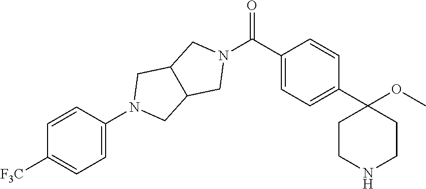 [SMILES: COC1(c2ccc(C(=O)N3CC4CN(c5ccc(C(F)(F)F)cc5)CC4C3)cc2)CCNCC1]

¹H NMR (DMSO-d₆): 1.72-1.79 (m, 2H), 1.91-1.96 (m, 2H), 2.91 (s, 3H), 3.06-3.17 (m, 5H), 3.21-3.31 (m, 2H), 3.46-3.63 (m, 3H), 3.73-3.80 (m, 4H), 6.56 (d, J=8.8 Hz, 2H), 7.46-7.53 (m, 6H).

ESI-MS: m/z: 474.3 (M+H)⁺, 100%.

### Example 169: (4-(4-methoxy-1-(2,2,2-trifluoroethyl)piperidin-4-yl)phenyl)(4-(4-(trifluoromethyl)phenyl)piperidin-1-yl)methanone

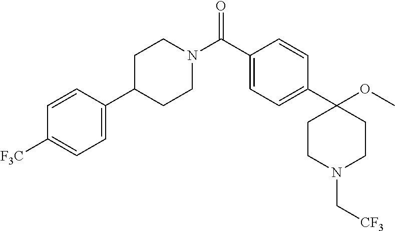 [SMILES: COC1(c2ccc(C(=O)N3CCC(c4ccc(C(F)(F)F)cc4)CC3)cc2)CCN(CC(F)(F)F)CC1]

¹H NMR (DMSO-d6): 1.60-1.71 (m, 2H), 1.77-1.97 (m, 6H), 2.68-2.78 (m, 4H), 2.89 (s, 3H), 2.93 (s, 2H), 3.16-3.24 (m, 3H), 3.61-3.66 (m, 1H), 3.69-3.71 (m, 1H), 7.43-7.48 (m, 4H), 7.52 (d, J=8.4 Hz, 2H), 7.66 (d, J=8.4 Hz, 2H).

ESI-MS: m/z: 529.3 (M+H)⁺, 100%.

### Example 170: (4-(4-ethoxy-1-(2,2,2-trifluoroethyl)piperidin-4-yl)phenyl)(4-(4-(trifluoromethyl)phenyl)piperidin-1-yl)methanone

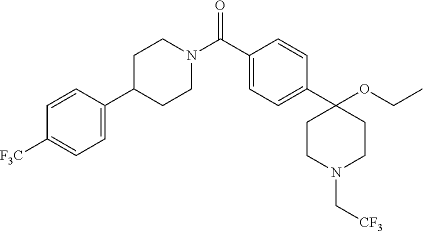 [SMILES: CCOC1(c2ccc(C(=O)N3CCC(c4ccc(C(F)(F)F)cc4)CC3)cc2)CCN(CC(F)(F)F)CC1]

¹H NMR (DMSO-d₆): 1.09-1.12 (m, 3H), 1.61-1.71 (m, 2H), 1.80-2.00 (m, 6H), 2.71-2.75 (m, 3H), 2.91-2.94 (m, 2H), 3.01-3.08 (m, 3H), 3.15-3.23 (m, 3H), 3.61-3.68 (m, 1H), 3.89-3.91 (m, 1H), 7.42-7.49 (m, 4H), 7.52 (d, J=8.0 Hz, 2H), 7.66 (d, J=8.0 Hz, 2H).

ESI-MS: m/z: 543.3 (M+H)⁺, 100%.

### Example 171: (3-(3-hydroxyoxetan-3-yl)phenyl)(2-(4-(trifluoromethyl)phenyl)-6,7-dihydrothieno[3,2-c]pyridin-5(4H)-yl)methanone

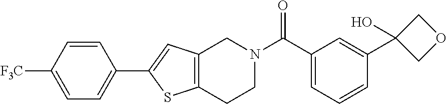 [SMILES: O=C(c1cccc(C2(O)COC2)c1)N1CCc2sc(-c3ccc(C(F)(F)F)cc3)cc2C1]

¹H NMR (DMSO-d₆) δ: 2.92 (brs, 2H), 3.49 (brs, 1H), 3.96 (brs, 1H), 4.52 (brs, 1H), 4.71 (d, J=6.8 Hz, 2H), 4.81 (d, J=6.8 Hz, 3H), 6.47 (s, 1H), 7.41 (d, J=7.2 Hz, 2H), 7.50-7.54 (m, 2H), 7.66 (s 1H), 7.73-7.75 (m, 4H).

ESI-MS: m/z, 460.01 (M+H)⁺, 100%.

### Example 172: (4-(3-hydroxyoxetan-3-yl)phenyl)(3-(4-(trifluoromethyl)phenyl)-6,7-dihydrothieno[3,2-c]pyridin-5(4H)-yl)methanone

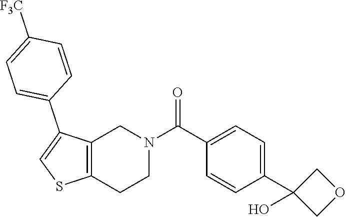 [SMILES: O=C(c1ccc(C2(O)COC2)cc1)N1CCc2scc(-c3ccc(C(F)(F)F)cc3)c2C1]

¹H NMR (DMSO-d6) δ: 2.96 (brs, 2H), 3.68 (brs, 2H), 4.70-4.78 (m, 6H), 6.45 (s, 1H), 7.48-7.56 (m, 3H), 7.63-7.69 (m, 5H), 7.83 (brs 1H).

ESI-MS: m/z, 460.09 (M+H)⁺, 100%.

### Example 173: (3-(3-hydroxyoxetan-3-yl)phenyl)(2-(4-(trifluoromethyl)phenyl)-6,7-dihydrothiazolo[5,4-c]pyridin-5(4H)-yl)methanone

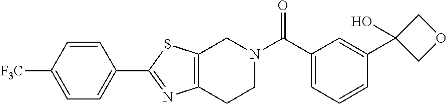 [SMILES: O=C(c1cccc(C2(O)COC2)c1)N1CCc2nc(-c3ccc(C(F)(F)F)cc3)sc2C1]

¹H NMR (DMSO-d₆) δ: 2.95 (brs, 2H), 3.71-3.74 (m, 1H), 3.93-4.01 (m, 1H), 4.71 (d, J=6.8 Hz, 2H), 4.79-4.85 (m, 3H), 4.96 (brs, 1H), 6.50 (s, 1H), 7.44 (s 1H), 7.53 (t, J=7.6 Hz, 1H), 7.68 (s 1H), 7.75 (d, J=7.6 Hz, 1H), 7.86 (d, J=8.0 Hz, 2H), 8.11 (brs 2H).

ESI-MS: m/z, 461.06 (M+H)⁺, 100%.

### Example 174: N-(4-(3-hydroxyoxetan-3-yl)phenyl)-2-(4-(trifluoromethyl) phenyl)-6,7-dihydrothiazolo[5,4-c]pyridine-5(4H)-carboxamide

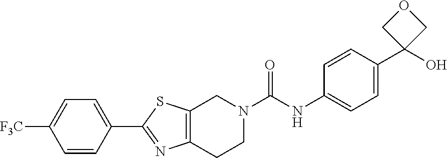 [SMILES: O=C(Nc1ccc(C2(O)COC2)cc1)N1CCc2nc(-c3ccc(C(F)(F)F)cc3)sc2C1]

¹H NMR (DMSO-d₆) δ: 2.95 (brs, 2H), 3.88 (t, J=5.6 Hz, 2H), 4.67 (d, J=6.8 Hz, 2H), 4.74 (d, J=5.6 Hz, 2H), 4.82 (s 2H), 6.22 (s 1H), 7.45-7.51 (m 4H), 7.86 (d, J=8.4 Hz, 2H), 8.12 (d, J=8.0 Hz, 2H), 8.81 (s 1H).

ESI-MS: m/z, 476.07 (M+H)⁺, 100%.

### Example 175: (4-(3-hydroxyoxetan-3-yl)phenyl)(2-(4-(trifluoromethyl)phenyl)-6,7-dihydrothiazolo[5,4-c]pyridin-5(4H)-yl)methanone

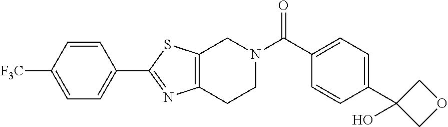 [SMILES: O=C(c1ccc(C2(O)COC2)cc1)N1CCc2nc(-c3ccc(C(F)(F)F)cc3)sc2C1]

¹H NMR (DMSO-d₆) δ: 2.96 (brs, 2H), 3.71 (brs, 1H), 4.00 (brs, 1H), 4.71 (d, J=6.8 Hz, 2H), 4.81 (d, J=6.8 Hz, 2H), 4.94 (brs 2H), 6.49 (s 1H), 7.53 (d, J=8.0 Hz, 2H), 7.72 (d, J=8.0 Hz, 2H), 7.86 (d, J=8.0 Hz, 2H), 8.48 (d, J=8.0 Hz, 2H).

ESI-MS: m/z, 461.09 (M+H)⁺, 100%.

### Example 176: (3-(3-hydroxyoxetan-3-yl)phenyl)(3-(4-(trifluoromethyl)phenyl)-6,7-dihydrothieno[3,2-c]pyridin-5(4H)-yl)methanone

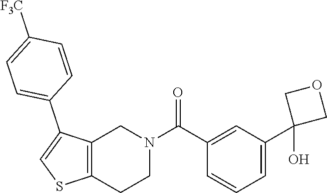 [SMILES: O=C(c1cccc(C2(O)COC2)c1)N1CCc2scc(-c3ccc(C(F)(F)F)cc3)c2C1]

¹H NMR (DMSO-d6) δ: 2.95 (brs, 2H), 3.54-3.67 (m, 5H), 3.99 (brs, 1H), 4.61-4.73 (m, 2H), 4.29-7.39 (m, 2H), 7.54-7.71 (m 6H), 7.84 (brs 1H).

ESI-MS: m/z, 460.07 (M+H)⁺, 100%.

### Example 177: (1-benzyl-3-(4-(trifluoromethyl)phenyl)-4,6-dihydropyrrolo[3,4-c]pyrazol-5(1H)-yl)(3-(3-hydroxyoxetan-3-yl)phenyl)methanone

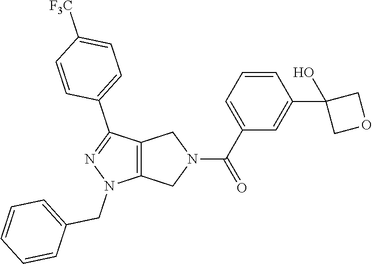 [SMILES: O=C(c1cccc(C2(O)COC2)c1)N1Cc2c(-c3ccc(C(F)(F)F)cc3)nn(Cc3ccccc3)c2C1]

¹H NMR (DMSO-d₆) δ: 4.63 (s, 2H), 4.68-4.73 (m, 3H), 4.77-4.80 (m, 3H), 5.44 (d, J=8.4 Hz, 2H), 6.45 (d, J=9.2 Hz, 1H), 7.03 (d, J=6.8 Hz, 2H), 7.24-7.31 (m, 3H), 7.46-7.58 (m, 2H), 7.63 (d, J=8.4 Hz, 1H), 7.70-7.79 (m, 4H), 7.85 (d, J=7.6 Hz, 1H).

ESI-MS: m/z, 520.20 (M+H)⁺, 100%.

### Example 178: (3-(3-ethoxyoxetan-3-yl)phenyl)(5-(4-(trifluoromethyl)phenyl) hexahydropyrrolo[3,4-c]pyrrol-2(1H)-yl)methanone

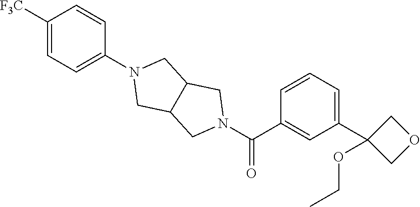 [SMILES: CCOC1(c2cccc(C(=O)N3CC4CN(c5ccc(C(F)(F)F)cc5)CC4C3)c2)COC1]

¹H NMR (DMSO-d₆) δ: 1.10 (t, J=7.0 Hz, 3H), 3.05-3.14 (m, 2H), 3.16-3.20 (m, 3H), 3.34-3.35 (m, 2H), 3.45-3.49 (m, 2H), 3.55-3.60 (m, 1H), 3.70-3.75 (m, 1H), 3.81-3.86 (m, 1H), 3.74-4.79 (m, 4H), 6.64 (t, J=8.4 Hz, 2H), 7.48 (t, J=8.8 Hz, 2H), 7.49-7.56 (m, 4H).

ESI-MS: m/z, 461.17 (M+H)⁺, 100%.

### Example 179: Ethyl 2-((3-(3-(5-(4-(trifluoromethyl)phenyl) octahydropyrrolo[3,4-c]pyrrole-2-carbonyl)phenyl)oxetan-3-yl)oxy)acetate

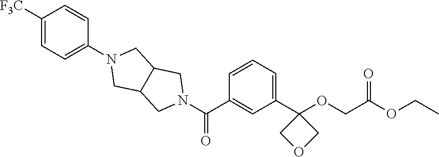 [SMILES: CCOC(=O)COC1(c2cccc(C(=O)N3CC4CN(c5ccc(C(F)(F)F)cc5)CC4C3)c2)COC1]

¹H NMR (CDCl₃) δ: 1.26 (t, J=7.2 Hz, 3H), 3.17-3.26 (m, 3H), 3.37-3.45 (m, 2H), 3.55-3.59 (m, 1H), 3.66-3.75 (m, 2H), 3.78-3.83 (m, 1H), 3.90 (s, 2H), 4.01-4.06 (m, 1H), 4.18 (q, J=7.2 Hz, 2H), 4.83-4.86 (m, 2H), 5.04 (t, J=7.2 Hz, 2H), 6.58 (t, J=8.8 Hz, 2H), 7.47-7.54 (m, 4H), 7.58-7.61 (m, 1H), 7.66-7.67 (m, 1H).

ESI-MS: m/z, 519.21 (M+H)⁺, 100%.

### Example 180: (3-(3-iso-butoxyoxetan-3-yl)phenyl)(5-(4-(trifluoromethyl)phenyl) hexahydropyrrolo[3,4-c]pyrrol-2(1H)-yl)methanone

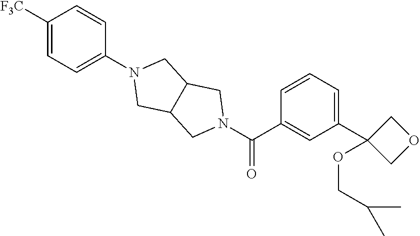 [SMILES: CC(C)COC1(c2cccc(C(=O)N3CC4CN(c5ccc(C(F)(F)F)cc5)CC4C3)c2)COC1]

¹H NMR (DMSO-d6) δ: 0.83 (d, J=6.4 Hz, 6H), 1.71-1.76 (m, 1H), 2.88-2.91 (m, 2H), 3.02-3.16 (m, 3H), 3.33-3.35 (m, 2H), 3.47-3.50 (m, 2H), 3.56-3.59 (m, 1H), 3.65-3.72 (m, 1H), 3.77-3.81 (m, 1H), 4.74-4.77 (m, 4H), 6.63 (d, J=8.4 Hz, 2H), 7.45-7.55 (m, 6H).

ESI-MS: m/z, 489.30 (M+H)⁺, 100%.

### Example 181: (3-(3-(cyclopropylmethoxy)oxetan-3-yl)phenyl)(5-(4-(trifluoromethyl)phenyl)hexahydropyrrolo[3,4-c]pyrrol-2(1H)-yl)methanone

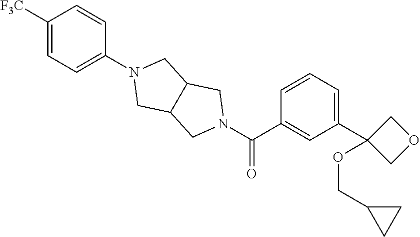 [SMILES: O=C(c1cccc(C2(OCC3CC3)COC2)c1)N1CC2CN(c3ccc(C(F)(F)F)cc3)CC2C1]

¹H NMR (DMSO-d₆) δ: 0.06-0.10 (m, 2H), 0.40-0.44 (m, 2H), 0.92-0.97 (m, 1H), 2.96 (t, J=6.8 Hz, 2H), 3.05-3.09 (m, 2H), 3.14-3.18 (m, 1H), 3.33-3.38 (m, 2H), 3.45-3.48 (m, 2H), 3.56-3.60 (m, 1H), 3.69-3.74 (m, 1H), 3.80-3.85 (m, 1H), 4.72-4.79 (m, 4H), 6.64 (d, J=8.8 Hz, 2H), 7.45-7.55 (m, 6H).

ESI-MS: m/z, 487.24 (M+H)⁺, 100%.

### Example 182: N-(3-(3-hydroxyoxetan-3-yl)phenyl)-5-(4-(trifluoromethyl)phenyl) hexahydropyrrolo[3,4-c]pyrrole-2(1H)-carboxamide

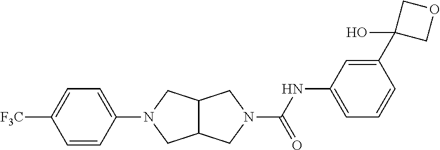 [SMILES: O=C(Nc1cccc(C2(O)COC2)c1)N1CC2CN(c3ccc(C(F)(F)F)cc3)CC2C1]

¹H NMR (DMSO-d₆) δ: 3.09 (brs, 2H), 3.22-3.25 (m, 2H), 3.34-3.37 (m, 2H), 3.55-3.59 (m, 2H), 3.68-3.73 (m, 2H), 4.64 (d, J=6.4 Hz, 2H), 4.75 (d, J=6.4 Hz, 2H), 6.28 (s, 1H), 6.67 (d, J=8.4 Hz, 2H), 7.15 (d, J=7.2 Hz, 1H), 7.21-7.25 (m, 1H), 7.46-7.51 (m, 3H), 7.72 (s, 1H), 8.24 (s, 1H).

ESI-MS: m/z, 448.18 (M+H)⁺, 100%.

### Example 183: N-(3-(3-methoxyoxetan-3-yl)phenyl)-5-(4-(trifluoromethyl) phenyl) hexahydropyrrolo[3,4-c]pyrrole-2(1H)-carboxamide

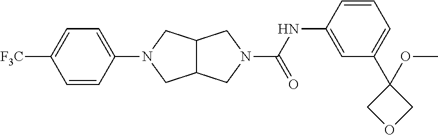 [SMILES: COC1(c2cccc(NC(=O)N3CC4CN(c5ccc(C(F)(F)F)cc5)CC4C3)c2)COC1]

¹H NMR (DMSO-d6) δ: 3.00 (s, 3H), 3.09 (brs, 2H), 3.22-3.25 (m, 2H), 3.35-3.38 (m, 2H), 3.55-3.59 (m, 2H), 3.68-3.73 (m, 2H), 4.70 (d, J=6.8 Hz, 2H), 4.74 (d, J=6.8 Hz, 2H), 6.66 (d, J=8.4 Hz, 2H), 6.98-7.00 (m, 1H), 7.26-7.30 (m, 1H), 7.47 (d, J=8.4 Hz, 2H), 7.54-7.55 (m, 2H), 8.25 (s, 1H).

ESI-MS: m/z, 462.30 (M+H)⁺, 100%.

### Example 184: (4-(3-hydroxyoxetan-3-yl)phenyl)(4-(4-(trifluoromethyl)phenyl)-1,4-diazepan-1-yl)methanone

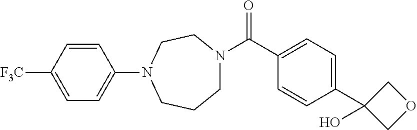 [SMILES: O=C(c1ccc(C2(O)COC2)cc1)N1CCCN(c2ccc(C(F)(F)F)cc2)CC1]

¹H NMR (DMSO-d₆) δ: 1.64-1.94 (m, 2H), 3.26-3.45 (m, 2H), 3.56-3.81 (m, 6H), 4.63-4.68 (m, 2H), 4.77-4.78 (m, 2H), 6.43 (s, 1H), 6.75 (d, J=8.0 Hz, 1H), 6.84-6.92 (m, 2H), 7.28-7.38 (m, 2H), 7.48-7.50 (m, 2H), 7.62-7.64 (m, 1H).

ESI-MS: m/z, 421.17 (M+H)⁺, 100%.

### Example 185: (4-(3-methoxyoxetan-3-yl)phenyl)(4-(4-(trifluoromethyl)phenyl)-1,4-diazepan-1-yl)methanone

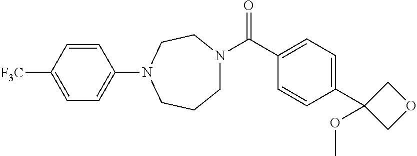 [SMILES: COC1(c2ccc(C(=O)N3CCCN(c4ccc(C(F)(F)F)cc4)CC3)cc2)COC1]

¹H NMR (DMSO-d₆) δ: 1.65-1.94 (m, 2H), 3.03 (s, 3H), 3.27-3.48 (m, 2H), 3.58-3.67 (m, 3H), 3.72-3.82 (m, 3H), 4.71-4.79 (m, 4H), 6.74 (d, J=8.4 Hz, 1H), 6.87-6.92 (m, 2H), 7.29-7.35 (m, 4H), 7.46-7.49 (m, 1H).

ESI-MS: m/z, 435.18 (M+H)⁺, 100%.

### Example 186: (4-(3-hydroxyoxetan-3-yl)phenyl)(8-(4-(trifluoromethyl)phenyl)-2,8-diazaspiro[4.5]decan-2-yl)methanone

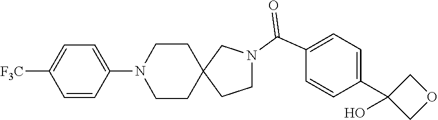 [SMILES: O=C(c1ccc(C2(O)COC2)cc1)N1CCC2(CCN(c3ccc(C(F)(F)F)cc3)CC2)C1]

¹H NMR (DMSO-d6) δ: 1.55 (brs, 2H), 1.64-1.68 (m, 2H), 1.76-1.90 (m, 2H), 3.27 (brs, 2H), 3.34-3.442 (m, 4H), 3.50-3.60 (m, 2H), 4.66-4.68 (m, 2H), 4.77-4.80 (m, 2H), 6.45 (d, J=6.8 Hz, 1H), 7.01-7.09 (m, 2H), 7.44-7.50 (m, 2H), 7.54-7.59 (m, 2H), 7.66 (d, J=7.6 Hz, 2H).

ESI-MS: m/z, 461.20 (M+H)⁺, 100%.

### Example 187: 4-(3-hydroxyoxetan-3-yl)-N-(1-(4-(trifluoromethyl)phenyl) pyrrolidin-3-yl)benzamide

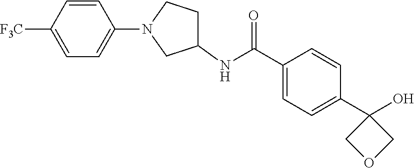 [SMILES: O=C(NC1CCN(c2ccc(C(F)(F)F)cc2)C1)c1ccc(C2(O)COC2)cc1]

¹H NMR (DMSO-d₆) δ: 2.07-2.25 (m, 1H), 2.26-2.33 (m, 1H), 3.28-3.32 (m, 1H), 3.34-3.38 (m, 1H), 3.40-3.53 (m, 1H), 3.64-3.68 (m, 1H), 4.63-4.68 (m, 3H), 4.79 (d, J=6.8 Hz, 2H), 6.49 (s, 1H), 6.66 (d, J=8.8 Hz, 2H), 7.47 (d, J=8.8 Hz, 2H), 7.69 (d, J=8.4 Hz, 2H), 7.91 (d, J=8.4 Hz, 2H), 8.65 (d, J=6.8 Hz, 1H).

ESI-MS: m/z, 407.15 (M+H)⁺, 100%.

### Example 188: (4-(3-(2-hydroxyethoxy)oxetan-3-yl)phenyl)(4-(4-(trifluoromethyl)phenyl)piperidin-1-yl)methanone

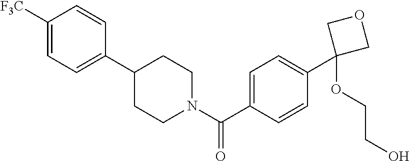 [SMILES: O=C(c1ccc(C2(OCCO)COC2)cc1)N1CCC(c2ccc(C(F)(F)F)cc2)CC1]

¹H NMR (DMSO-d6) δ: 1.62-1.76 (m, 3H), 1.88 (brs, 1H), 2.74-2.98 (m, 2H), 3.19 (t, J=5.2 Hz, 3H), 3.49-3.53 (m, 2H), 3.70-3.75 (m, 1H), 4.66 (brs, 1H), 4.70 (t, J=5.6 Hz, 1H), 4.76 (d, J=6.8 Hz, 2H), 4.83 (d, J=6.8 Hz, 2H), 7.50-7.59 (m, 6H), 7.67 (d, J=8.4 Hz, 2H).

ESI-MS: m/z, 450.18.15 (M+H)⁺, 100%.

### Example 189: (4-(3-(2-(ethylthio)ethoxy)oxetan-3-yl)phenyl)(4-(4-(trifluoromethyl)phenyl)piperidin-1-yl)methanone

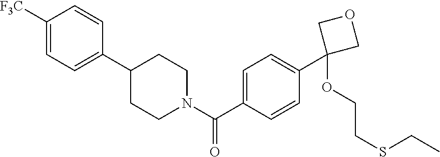 [SMILES: CCSCCOC1(c2ccc(C(=O)N3CCC(c4ccc(C(F)(F)F)cc4)CC3)cc2)COC1]

¹H NMR (CDCl₃) δ: 1.26 (t, J=7.2 Hz, 3H), 1.95-2.09 (m, 4H), 2.59 (q, J=7.2 Hz, 2H), 2.75 (t, J=6.8 Hz, 2H), 2.85-2.91 (m, 2H), 3.13-3.20 (m, 1H), 3.40 (t, J=6.8 Hz, 2H), 3.94 (brs, 1H), 4.83 (d, J=7.2 Hz, 2H), 4.95 (brs, 1H), 4.98 (d, J=7.2 Hz, 2H), 7.36 (d, J=8.0 Hz, 2H), 7.51-7.61 (m, 6H).

ESI-MS: m/z, 494.26 (M+H)⁺, 100%.

### Example 190: (4-(3-(2-(ethylsulfonyl)ethoxy)oxetan-3-yl)phenyl)(4-(4-(trifluoromethyl)phenyl)piperidin-1-yl)methanone

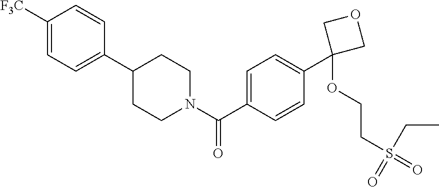 [SMILES: CCS(=O)(=O)CCOC1(c2ccc(C(=O)N3CCC(c4ccc(C(F)(F)F)cc4)CC3)cc2)COC1]

¹H NMR (CDCl₃) δ: 1.48 (t, J=7.6 Hz, 3H), 1.70-2.04 (m, 6H), 2.83-2.90 (m, 2H), 3.18-3.25 (m, 4H), 3.64 (t, J=6.8 Hz, 2H), 3.89-3.91 (m, 1H), 4.87 (d, J=7.2 Hz, 2H), 4.90 (d, J=7.2 Hz, 2H), 7.36 (d, J=8.0 Hz, 2H), 7.47 (d, J=8.4 Hz, 2H), 7.54 (d, J=8.4 Hz, 2H), 7.60 (d, J=8.0 Hz, 2H).

ESI-MS: m/z, 526.18 (M+H)⁺, 100%.

### Example 191: (4-(3-hydroxyoxetan-3-yl)phenyl)(4-(4-(methylthio) phenyl) piperidin-1-yl)methanone

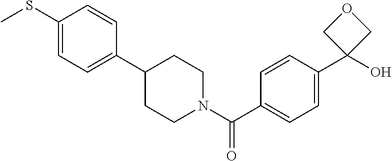 [SMILES: CSc1ccc(C2CCN(C(=O)c3ccc(C4(O)COC4)cc3)CC2)cc1]

¹H NMR (DMSO-d6) δ: 1.54-1.70 (m, 2H), 1.82-1.84 (m, 1H), 1.96-1.99 (m, 1H), 2.45 (s, 3H), 2.73-2.81 (m, 2H), 3.09-3.15 (m, 1H), 3.69 (brs, 1H), 4.62 (brs, 1H), 4.70 (d, J=6.8 Hz, 2H), 4.79 (d, J=6.8 Hz, 2H), 6.44 (s, 1H), 7.19-7.25 (m, 4H), 7.50 (d, J=7.6 Hz, 2H), 7.70 (d, J=7.6 Hz, 2H).

ESI-MS: m/z, 384.16 (M+H)⁺, 100%.

### Example 192: (4-(3-methoxyoxetan-3-yl)phenyl)(4-(4-(methylthio)phenyl) piperidin-1-yl)methanone

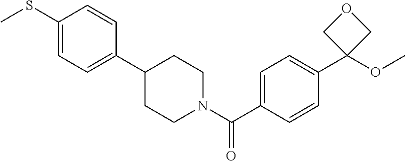

¹H NMR (CDCl₃) δ: 1.73-2.01 (m, 4H), 2.51 (s, 3H), 2.75-2.81 (m, 1H), 2.90 (brs, 1H), 3.17 (s, 4H), 3.93 (brs, 1H), 4.83 (d, J=6.8 Hz, 2H), 4.91 (brs, 1H), 4.96 (d, J=6.8 Hz, 2H), 7.17 (d, J=8.0 Hz, 2H), 7.25 (d, J=8.0 Hz, 2H), 7.51-7.55 (m, 4H).

ESI-MS: m/z, 398.17 (M+H)⁺, 100%.

### Example 193: (4-(4-hydroxypiperidin-4-yl)phenyl)(4-(4-(methylthio)phenyl) piperidin-1-yl)methanone

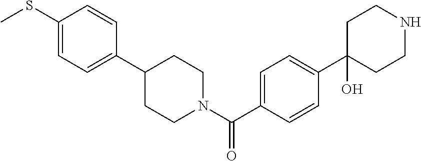 [SMILES: CSc1ccc(C2CCN(C(=O)c3ccc(C4(O)CCNCC4)cc3)CC2)cc1]

¹H NMR (DMSO-d₆) δ: 1.52-1.62 (m, 4H), 1.66-1.87 (m, 4H), 2.45 (s, 3H), 2.74-2.80 (m, 4H), 2.87-2.97 (m, 2H), 3.15-3.22 (m, 1H), 3.68-3.75 (m, 2H), 4.61 (brs, 1H), 4.90 (s, 1H), 7.19-7.25 (m, 4H), 7.38 (d, J=8.4 Hz, 2H), 7.53 (d, J=8.0 Hz, 2H).

ESI-MS: m/z, 411.21 (M+H)⁺, 100%.

### Example 194: (4-(4-methoxypiperidin-4-yl)phenyl)(4-(4-(methylthio) phenyl) piperidin-1-yl)methanone

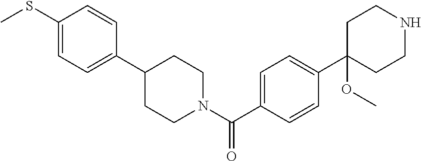 [SMILES: COC1(c2ccc(C(=O)N3CCC(c4ccc(SC)cc4)CC3)cc2)CCNCC1]

¹H NMR (CDCl₃) δ: 1.63-1.87 (m, 4H), 2.14-2.19 (m, 2H), 2.25-2.27 (m, 2H), 2.49 (s, 3H), 2.88 (brs, 1H), 3.01 (s, 3H), 3.16-3.18 (m, 3H), 3.30-3.37 (m, 4H), 3.90 (brs, 1H), 4.87-4.90 (m, 1H), 7.16 (d, J=8.4 Hz, 2H), 7.25 (d, J=8.4 Hz, 2H), 7.43-7.49 (m, 4H).

ESI-MS: m/z, 425.22 (M+H)⁺, 5%.

### Example 195: (3-(3-(methoxy-d3)oxetan-3-yl)phenyl)(5-(4-(trifluoromethyl) phenyl)hexahydropyrrolo[3,4-c]pyrrol-2(1H)-yl)methanone

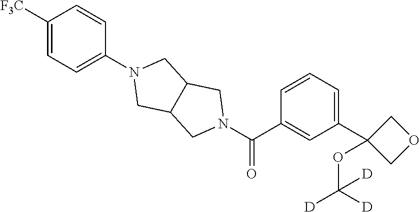 [SMILES: [2H]C([2H])([2H])OC1(c2cccc(C(=O)N3CC4CN(c5ccc(C(F)(F)F)cc5)CC4C3)c2)COC1]

¹H NMR (DMSO-d₆) δ: 3.05-3.09 (m, 2H), 3.16-3.18 (m, 1H), 3.34-3.36 (m, 2H), 3.45-3.49 (m, 2H), 3.55-3.57 (m, 1H), 3.71-3.74 (m, 1H), 3.79-3.83 (m, 1H), 4.75-4.79 (m, 4H), 6.64 (d, J=8.8 Hz, 2H), 7.47 (d, J=8.8 Hz, 2H), 7.49-7.55 (m, 4H).

ESI-MS: m/z, 450.23 (M+H)⁺, 100%.

### Example 196: (3-(3-isopropoxyoxetan-3-yl)phenyl)(5-(4-(trifluoromethyl) phenyl)hexahydropyrrolo[3,4-c]pyrrol-2(1H)-yl)methanone

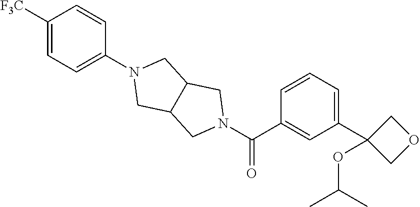 [SMILES: CC(C)OC1(c2cccc(C(=O)N3CC4CN(c5ccc(C(F)(F)F)cc5)CC4C3)c2)COC1]

¹H NMR (DMSO-d₆) δ: 0.92 (t, J=6.0 Hz, 6H), 3.05-3.17 (m, 3H), 3.34-3.35 (m, 2H), 3.42-3.49 (m, 3H), 3.56-3.60 (m, 1H), 3.69-3.73 (m, 1H), 3.81-3.86 (m, 1H), 4.77-5.76 (m, 4H), 6.64 (d, J=8.8 Hz, 2H), 7.45-7.58 (m, 6H).

ESI-MS: m/z, 475.25 (M+H)⁺, 100%.

### Example 197: N-(3-(3-hydroxyoxetan-3-yl)-5-morpholinophenyl)-1-(4-(trifluoromethyl)phenyl)piperidine-4-carboxamide

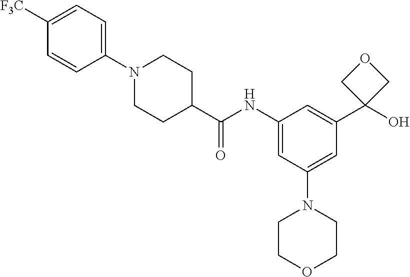 [SMILES: O=C(Nc1cc(N2CCOCC2)cc(C2(O)COC2)c1)C1CCN(c2ccc(C(F)(F)F)cc2)CC1]

¹H NMR (DMSO-d₆): 1.66-1.74 (m, 2H), 1.85-1.91 (m, 2H), 2.57-2.60 (m, 1H), 2.84-2.90 (m, 2H), 3.06-3.08 (m, 4H), 3.73-3.75 (m, 4H), 3.93-3.96 (m, 2H), 4.63 (d, J=6.4 Hz, 2H), 4.72 (d, J=6.4 Hz, 2H), 6.26 (s, 1H), 6.85 (s, 1H), 7.07 (d, J=9.2 Hz, 2H), 7.31 (s, 1H), 7.49 (d, J=8.8 Hz, 2H).

ESI-MS: m/z: 506.3 (M+H)⁺, 100%.

### Example 198: Methyl 4-(5-hydroxy-2-(4-(3-hydroxyoxetan-3-yl)benzoyl) octahydrocyclopenta[c]pyrrol-5-yl)benzoate

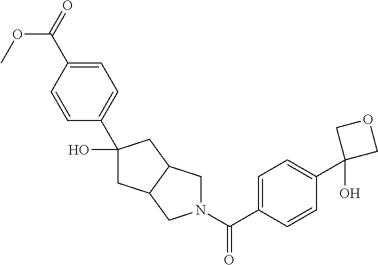 [SMILES: COC(=O)c1ccc(C2(O)CC3CN(C(=O)c4ccc(C5(O)COC5)cc4)CC3C2)cc1]

¹H NMR (DMSO-d₆) δ: 1.86-1.98 (m, 2H), 2.09-2.33 (m, 2H), 2.91 (brs, 2H), 3.56-3.67 (m, 4H), 3.84 (s, 3H), 4.62 (d, J=6.8 Hz, 2H), 4.80 (d, J=6.8 Hz, 2H), 5.28 (s, 1H), 6.47 (s, 1H), 7.52 (d, J=7.2 Hz, 2H), 7.61 (d, J=7.6 Hz, 2H), 7.67 (d, J=7.6 Hz, 2H), 7.91 (d, J=7.2 Hz, 2H).

ESI-MS: m/z, 438.18 (M+H)⁺, 50%.

### Example 199: (4-(3-hydroxyoxetan-3-yl)phenyl)(5-(4-methoxyphenyl)-3,3a,4,6a-tetrahydrocyclopenta[c]pyrrol-2(1H)-yl)methanone

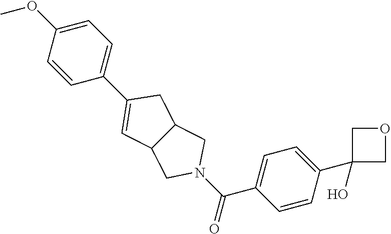 [SMILES: COc1ccc(C2=CC3CN(C(=O)c4ccc(C5(O)COC5)cc4)CC3C2)cc1]

¹H NMR (DMSO-d6) δ: 2.55-2.65 (m, 1H), 2.79-2.91 (m, 1H), 3.12-3.16 (m, 1H), 3.37-3.47 (m, 2H), 3.67-3.75 (m, 6H), 4.69 (d, J=6.4 Hz, 2H), 4.78 (d, J=6.4 Hz, 2H), 5.92 (brs, 0.5H), 6.15 (brs, 0.5H), 6.43 (s, 1H), 6.90 (d, J=8.4 Hz, 2H), 7.40 (d, J=8.4 Hz, 2H), 7.49 (d, J=7.6 Hz, 2H), 7.64 (d, J=7.6 Hz, 2H).

ESI-MS: m/z, 392.17 (M+H)⁺, 100%.

### Example 200: (4-(3-hydroxyoxetan-3-yl)phenyl)(5-(4-(2-(methylthio)ethoxy) phenyl)-3,3a,4,6a-tetrahydrocyclopenta[c]pyrrol-2(1H)-yl)methanone

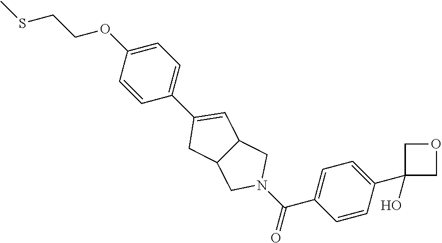 [SMILES: CSCCOc1ccc(C2=CC3CN(C(=O)c4ccc(C5(O)COC5)cc4)CC3C2)cc1]

¹H NMR (DMSO-d6) δ: 2.15 (s, 3H), 2.61-2.67 (m, 1H), 2.84 (d, J=6.4 Hz, 3H), 2.99-3.01 (m, 1H), 3.15-3.19 (m, 1H), 3.41-3.53 (m, 2H), 3.67-3.76 (m, 2H), 4.15 (t, J=6.4 Hz, 2H), 4.68 (brs, 2H), 4.79 (brs, 2H), 5.94 (brs, 0.5H), 6.08 (brs, 0.5H), 6.45 (s, 1H), 6.92 (d, J=8.8 Hz, 2H), 7.39 (d, J=8.8 Hz, 2H), 7.49 (d, J=7.6 Hz, 2H), 7.64 (d, J=8.4 Hz, 2H).

ESI-MS: m/z, 452.14 (M+H)⁺, 100%.

### Example 201: N-(3-(3-hydroxyoxetan-3-yl)-5-(4-methylpiperazin-1-yl) phenyl)-1-(4-(trifluoromethyl) phenyl)piperidine-4-carboxamide

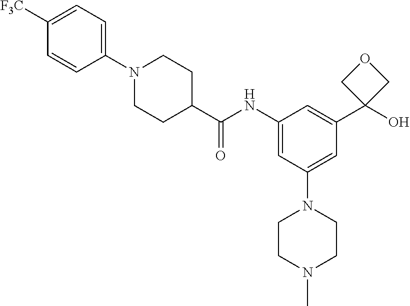 [SMILES: CN1CCN(c2cc(NC(=O)C3CCN(c4ccc(C(F)(F)F)cc4)CC3)cc(C3(O)COC3)c2)CC1]

¹H NMR (DMSO-d6) δ: 1.65-1.74 (m, 2H), 1.84-1.91 (m, 2H), 2.75 (brs, 2H), 2.78-2.95 (m, 3H), 3.09 (s, 3H), 3.19-3.25 (m, 4H), 3.45-3.54 (m, 2H), 3.91-3.4.12 (m, 2H), 4.64 (d, J=6.4 Hz, 2H), 4.74 (d, J=6.4 Hz, 2H), 6.34 (s, 1H), 6.89 (s, 1H), 7.09 (d, J=8.4 Hz, 2H), 7.27 (s, 1H), 7.31 (s, 1H), 7.50 (d, J=8.4 Hz, 2H), 9.98 (s, 1H).

ESI-MS: m/z, 452.14 (M+H)⁺, 100%.

### Example 202: (4-(3-methoxyoxetan-3-yl)phenyl)(5-(4-(trifluoromethyl)phenyl)-3,4,5,6-tetrahydropyrrolo[3,4-c]pyrrol-2(1H)-yl)methanone

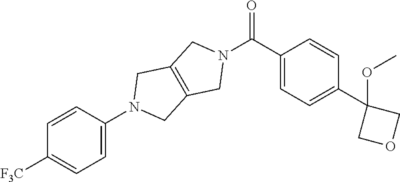 [SMILES: COC1(c2ccc(C(=O)N3CC4=C(C3)CN(c3ccc(C(F)(F)F)cc3)C4)cc2)COC1]

¹H NMR (DMSO-d₆) δ: 3.06 (s, 3H), 4.08 (s, 2H), 4.19 (s, 2H), 4.32 (s, 2H), 4.42 (s, 2H), 4.76-4.82 (m, 4H), 6.62 (d, J=8.8 Hz, 2H), 7.49 (d, J=8.8 Hz, 2H), 7.53 (d, J=8.4 Hz, 2H), 7.65 (d, J=8.4 Hz, 2H).

### Example 203: (5-hydroxy-5-(6-methoxypyridin-3-yl) hexahydrocyclopenta[c]pyrrol-2(1H)-yl)(4-(3-hydroxyoxetan-3-yl)phenyl)methanone

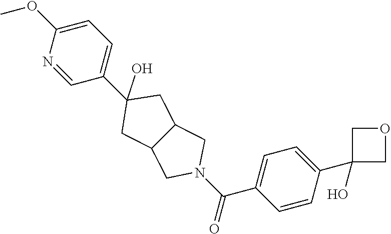 [SMILES: COc1ccc(C2(O)CC3CN(C(=O)c4ccc(C5(O)COC5)cc4)CC3C2)cn1]

¹H NMR (DMSO-d₆) δ: 1.79-1.95 (m, 2H), 2.09-2.11 (m, 2H), 2.85-2.88 (m, 2H), 3.51-3.53 (m, 1H), 3.66-3.68 (m, 2H), 3.82-3.87 (m, 4H), 4.70 (d, J=6.4 Hz, 2H), 4.79 (d, J=6.4 Hz, 2H), 5.14 (s, 1H), 6.45 (s, 1H), 6.76 (d, J=8.4 Hz, 1H), 7.51 (d, J=8.4 Hz, 2H), 7.66 (d, J=8.4 Hz, 2H), 7.75 (dd, J=8.4 & 2.4 Hz, 1H), 8.21 (d, J=2.4 Hz, 1H).

ESI-MS: m/z, 411.19 (M+H)⁺, 100%.

### Example 204: tert-butyl 4-methoxy-4-(4-(4-(5-(trifluoromethyl)pyridin-2-yl)piperidine-1-carbonyl)phenyl)piperidine-1-carboxylate

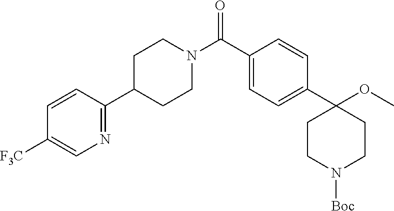 [SMILES: COC1(c2ccc(C(=O)N3CCC(c4ccc(C(F)(F)F)cn4)CC3)cc2)CCN(C(=O)OC(C)(C)C)CC1]

¹H NMR (DMSO-d₆): 1.49 (s, 9H), 1.70-1.75 (m, 2H), 1.89-2.06 (m, 8H), 3.00 (s, 3H), 3.10-3.19 (m, 4H), 3.92-4.01 (m, 3H), 7.32 (d, J=8.0 Hz, 1H), 7.42-7.48 (m, 4H), 7.89 (dd, J=8.0 & 2.0 Hz, 2H), 7.84 (s, 2H).

ESI-MS: m/z: 492.2, 100%.

### Example 205: (4-(4-methoxypiperidin-4-yl)phenyl)(4-(5-(trifluoromethyl) pyridin-2-yl)piperidin-1-yl)methanone

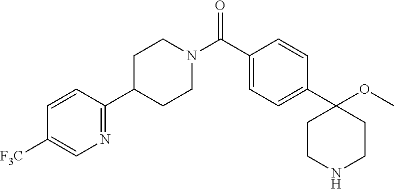 [SMILES: COC1(c2ccc(C(=O)N3CCC(c4ccc(C(F)(F)F)cn4)CC3)cc2)CCNCC1]

¹H NMR (DMSO-d₆): 1.89-2.13 (m, 9H), 3.01-3.24 (m, 10H), 3.96 (brs, 1H), 7.32 (d, J=8.0 Hz, 1H), 7.42-7.49 (m, 4H), 7.89 (dd, J=8.2 & 1.8 Hz, 2H), 7.84 (s, 2H).

ESI-MS: m/z: 417.2, 100%.

### Example 206: (4-(3-methoxyoxetan-3-yl)phenyl)(5-(6-(trifluoromethyl)pyridin-3-yl) hexahydropyrrolo[3,4-c]pyrrol-2(1H)-yl)methanone

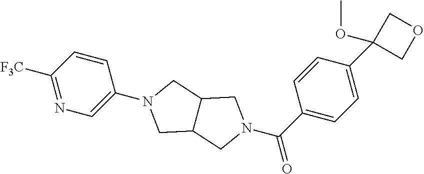 [SMILES: COC1(c2ccc(C(=O)N3CC4CN(c5ccc(C(F)(F)F)nc5)CC4C3)cc2)COC1]

¹H NMR (DMSO-d₆) δ: 2.95-3.06 (m, 5H), 3.26-3.27 (m, 1H), 3.35-3.41 (m, 2H), 3.54 (brs, 2H), 3.64-3.66 (m, 1H), 3.75-3.87 (m, 2H), 4.76-4.82 (m, 4H), 7.02 (d, J=6.8 Hz, 1H), 7.54 (d, J=8.0 Hz, 2H), 7.62 (d, J=7.6 Hz, 3H), 8.05 (s, 1H).

ESI-MS: m/z, 448.22 (M+H)⁺, 100%.

### Example 207: (4-(3-hydroxyoxetan-3-yl)phenyl)(5-(5-(trifluoromethyl)pyridin-2-yl)hexahydropyrrolo[3,4-c]pyrrol-2(1H)-yl)methanone

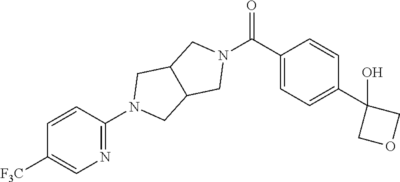 [SMILES: O=C(c1ccc(C2(O)COC2)cc1)N1CC2CN(c3ccc(C(F)(F)F)cn3)CC2C1]

¹H NMR (DMSO-d₆) δ: 3.03-3.08 (m, 2H), 3.33-3.40 (m, 1H), 3.47-3.49 (m, 2H), 3.57-3.84 (m, 4H), 4.67 (d, J=6.8 Hz, 2H), 4.78 (d, J=6.4 Hz, 2H), 6.45 (s, 1H), 6.58 (d, J=9.2 Hz, 1H), 7.56 (d, J=8.0 Hz, 2H), 7.65 (d, J=8.4 Hz, 2H), 7.77 (dd, J=8.8 & 2.0 Hz, 1H), 8.39 (s, 1H).

ESI-MS: m/z, 434.16 (M+H)⁺, 100%.

### Example 208: (3-(3-methoxyoxetan-3-yl)phenyl)(5-(5-(trifluoromethyl)pyridin-2-yl)hexahydropyrrolo[3,4-c]pyrrol-2 (1H)-yl)methanone

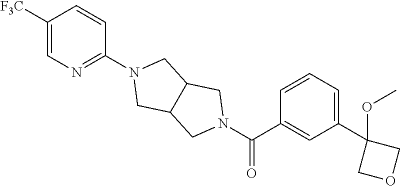 [SMILES: COC1(c2cccc(C(=O)N3CC4CN(c5ccc(C(F)(F)F)cn5)CC4C3)c2)COC1]

¹H NMR (DMSO-d₆) δ: 3.00-3.06 (m, 6H), 3.25-3.38 (m, 1H), 3.42-3.46 (m, 2H), 3.58-3.83 (m, 4H), 4.72-4.76 (m, 4H), 6.55 (d, J=8.8 Hz, 1H), 7.45-7.52 (m, 4H), 7.75 (dd, J=8.8 & 2.4 Hz, 1H), 8.36 (s, 1H).

ESI-MS: m/z, 448.18 (M+H)⁺, 100%.

### Example 209: (4-(3-methoxyoxetan-3-yl)phenyl)(5-(5-(trifluoromethyl)pyridin-2-yl) hexahydropyrrolo[3,4-c]pyrrol-2(1H)-yl)methanone

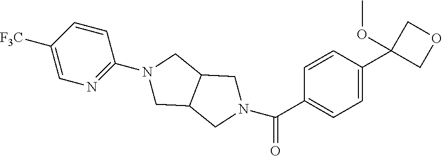 [SMILES: COC1(c2ccc(C(=O)N3CC4CN(c5ccc(C(F)(F)F)cn5)CC4C3)cc2)COC1]

¹H NMR (DMSO-d₆) δ: 3.01-3.06 (m, 5H), 3.31-3.38 (m, 2H), 3.44-3.47 (m, 2H), 3.58-3.63 (m, 1H), 3.68-3.82 (m, 3H), 4.72 (d, J=6.8 Hz, 2H), 4.75 (d, J=6.8 Hz, 2H), 6.55 (d, J=8.8 Hz, 1H), 7.47 (d, J=8.0 Hz, 2H), 7.57 (d, J=8.8 Hz, 2H), 7.75 (dd, J=9.2 & 2.4 Hz, 1H), 8.37 (s, 1H).

ESI-MS: m/z, 448.19 (M+H)⁺, 100%.

### Example 210: (3-(3-hydroxyoxetan-3-yl)phenyl)(5-(5-(trifluoromethyl)pyridin-2-yl) hexahydropyrrolo[3,4-c]pyrrol-2(1H)-yl)methanone

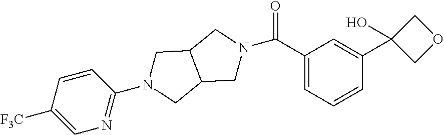 [SMILES: O=C(c1cccc(C2(O)COC2)c1)N1CC2CN(c3ccc(C(F)(F)F)cn3)CC2C1]

¹H NMR (DMSO-d₆) δ: 3.03-3.09 (m, 2H), 3.34 (s, 2H), 3.42-3.49 (m, 2H), 3.61-3.85 (m, 4H), 4.68 (d, J=6.8 Hz, 2H), 4.75 (d, J=6.8 Hz, 2H), 6.44 (s, 1H), 6.58 (d, J=9.2 Hz, 1H), 7.46 (d, J=4.8 Hz, 2H), 7.70 (d, J=8.4 Hz, 2H), 7.77 (dd, J=8.8 & 2.4 Hz, 1H), 8.39 (s, 1H).

ESI-MS: m/z, 434.22 (M+H)⁺, 100%.

### Example 211: (4-(3-methoxyoxetan-3-yl)phenyl)(4-(5-(trifluoromethyl)pyridin-2-yl) piperidin-1-yl)methanone

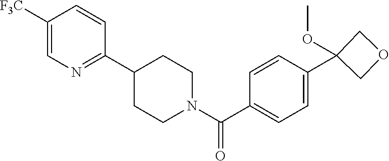 [SMILES: COC1(c2ccc(C(=O)N3CCC(c4ccc(C(F)(F)F)cn4)CC3)cc2)COC1]

¹H NMR (DMSO-d₆) δ: 1.72-1.98 (m, 4H), 2.93 (brs, 1H), 3.05 (s, 3H), 3.11-3.28 (m, 2H), 3.72 (brs, 1H), 4.63 (brs, 1H), 4.77 (d, J=6.8 Hz, 2H), 4.80 (d, J=7.2 Hz, 2H), 7.42-7.54 (m, 4H), 7.60 (d, J=8.4 Hz, 1H), 8.16 (d, J=6.8 Hz, 1H), 8.91 (s, 1H).

ESI-MS: m/z, 421.17 (M+H)⁺, 100%.

### Example 212: (4-(3-hydroxyoxetan-3-yl)phenyl)(4-(5-(trifluoromethyl)pyridin-2-yl) piperidin-1-yl)methanone

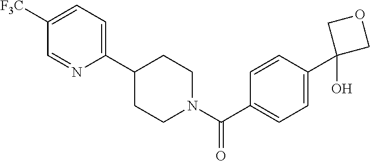 [SMILES: O=C(c1ccc(C2(O)COC2)cc1)N1CCC(c2ccc(C(F)(F)F)cn2)CC1]

¹H NMR (DMSO-d6) δ: 1.71-1.95 (m, 4H), 2.92 (brs, 1H), 3.11-3.17 (m, 2H), 3.73 (brs, 1H), 4.62 (brs, 1H), 4.70 (d, J=6.4 Hz, 2H), 4.79 (d, J=6.4 Hz, 2H), 6.45 (s, 1H), 7.46 (d, J=8.0 Hz, 2H), 7.60 (d, J=8.4 Hz, 1H), 7.69 (d, J=8.0 Hz, 2H), 8.16 (d, J=6.8 Hz, 1H), 8.91 (s, 1H).

ESI-MS: m/z, 407.15 (M+H)⁺, 100%.

### Example 213: (4-(3-methoxyoxetan-3-yl)phenyl)(4-(6-(trifluoromethyl)pyridin-3-yl) piperidin-1-yl)methanone

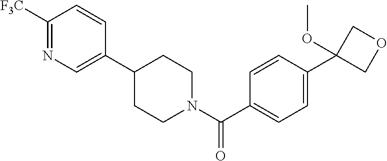 [SMILES: COC1(c2ccc(C(=O)N3CCC(c4ccc(C(F)(F)F)nc4)CC3)cc2)COC1]

¹H NMR (DMSO-d6) δ: 1.70-1.99 (m, 4H), 2.87-2.99 (m, 1H), 3.05 (s, 4H), 3.20-3.23 (m, 1H), 3.72 (brs, 1H), 4.60-4.67 (m, 1H), 4.77 (d, J=7.2 Hz, 2H), 4.80 (d, J=7.2 Hz, 2H), 7.42-7.53 (m, 4H), 7.86 (d, J=8.0 Hz, 1H), 8.04 (d, J=6.8 Hz, 1H), 8.74 (s, 1H).

ESI-MS: m/z, 421.17 (M+H)⁺, 100%.

### Example 214: (4-(3-hydroxyoxetan-3-yl)phenyl)(4-(6-(trifluoromethyl)pyridin-3-yl) piperidin-1-yl)methanone

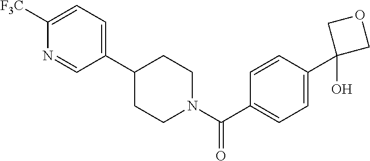 [SMILES: O=C(c1ccc(C2(O)COC2)cc1)N1CCC(c2ccc(C(F)(F)F)nc2)CC1]

¹H NMR (DMSO-d6) δ: 1.70-1.99 (m, 4H), 2.87-2.99 (m, 1H), 3.05 (s, 4H), 3.20-3.23 (m, 1H), 3.72 (brs, 1H), 4.60-4.67 (m, 1H), 4.77 (d, J=7.2 Hz, 2H), 4.80 (d, J=7.2 Hz, 2H), 7.42-7.53 (m, 4H), 7.86 (d, J=8.0 Hz, 1H), 8.04 (d, J=6.8 Hz, 1H), 8.74 (s, 1H).

ESI-MS: m/z, 407.21 (M+H)⁺, 100%.

### Example 215: (4-(3-hydroxyoxetan-3-yl)phenyl)(5-(6-(trifluoromethyl)pyridin-3-yl) hexahydropyrrolo[3,4-c]pyrrol-2(1H)-yl)methanone

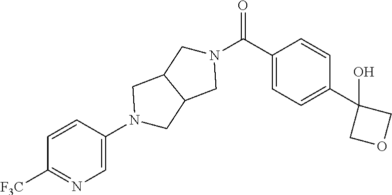 [SMILES: O=C(c1ccc(C2(O)COC2)cc1)N1CC2CN(c3ccc(C(F)(F)F)nc3)CC2C1]

¹H NMR (DMSO-d₆) δ: 3.06-3.11 (m, 2H), 3.21-3.25 (m, 1H), 3.37-3.40 (m, 2H), 3.48-3.54 (m, 2H), 3.61-3.66 (m, 1H), 3.71-3.85 (m, 2H), 4.69 (d, J=6.8 Hz, 2H), 4.79 (d, J=6.8 Hz, 2H), 6.45 (s, 1H), 6.99 (d, J=8.4 & 2.4 Hz, 1H), 7.54-7.64 (m, 5H), 8.03 (d, J=2.8 Hz, 1H).

ESI-MS: m/z, 434.5 (M+H)⁺, 100%.

### Example 216: (4-(3-hydroxyoxetan-3-yl) phenyl)(4-(5-(trifluoromethyl) pyridin-2-yl) piperazin-1-yl)methanone

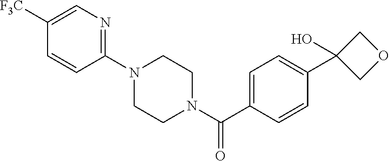 [SMILES: O=C(c1ccc(C2(O)COC2)cc1)N1CCN(c2ccc(C(F)(F)F)cn2)CC1]

¹H NMR (DMSO-d₆) δ: 3.48 (brs, 2H), 3.60 (brs, 6H), 4.70 (d, J=6.8 Hz, 2H), 4.80 (d, J=6.8 Hz, 2H), 6.48 (s, 1H), 6.97 (d, J=9.2 Hz, 1H), 7.49 (d, J=8.4 Hz, 2H), 7.70 (d, J=8.0 Hz, 2H), 7.84 (d, J=9.2 Hz, 1H), 8.44 (s, 1H);

ESI-MS: m/z, 408.14 (M+H)⁺, 100%.

### Example 217: (4-(3-hydroxyoxetan-3-yl) phenyl)(4-(6-(trifluoromethyl) pyridin-3-yl) piperazin-1-yl) methanone

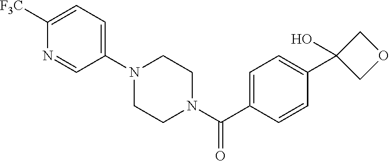 [SMILES: O=C(c1ccc(C2(O)COC2)cc1)N1CCN(c2ccc(C(F)(F)F)nc2)CC1]

¹H NMR (DMSO-d₆) δ: 3.43 (brs, 6H), 3.77 (brs, 2H), 4.70 (d, J=6.8 Hz, 2H), 4.80 (d, J=6.8 Hz, 2H), 6.48 (s, 1H), 7.44-7.50 (m, 3H), 7.67-7.71 (m, 3H), 8.45 (s, 1H).

ESI-MS: m/z, 408.14 (M+H)⁺, 100%.

### Example 218: (4-(3-methoxyoxetan-3-yl)phenyl)(4-(5-(trifluoromethyl)pyridin-2-yl) piperazin-1-yl)methanone

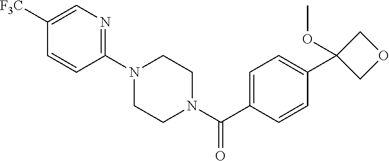 [SMILES: COC1(c2ccc(C(=O)N3CCN(c4ccc(C(F)(F)F)cn4)CC3)cc2)COC1]

¹H NMR (DMSO-d₆) δ: 3.05 (s, 3H), 3.48 (brs, 2H), 3.73 (brs, 6H), 4.77 (d, J=7.2 Hz, 2H), 4.80 (d, J=7.2 Hz, 2H), 6.97 (d, J=9.2 Hz, 1H), 7.51-7.56 (m, 4H), 7.84 (d, J=9.2 Hz, 1H), 8.43 (s, 1H).

ESI-MS: m/z, 422.14 (M+H)⁺, 100%.

### Example 219: (4-(3-ethoxyoxetan-3-yl)phenyl)(4-(5-(trifluoromethyl)pyridin-2-yl) piperazin-1-yl)methanone

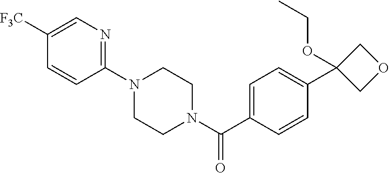 [SMILES: CCOC1(c2ccc(C(=O)N3CCN(c4ccc(C(F)(F)F)cn4)CC3)cc2)COC1]

¹H NMR (DMSO-d₆) δ: 1.14 (t, J=7.0 Hz, 3H), 3.19 (q, J=9.8 Hz, 2H), 3.48 (brs, 2H), 3.73 (brs, 6H), 4.77 (d, J=7.2 Hz, 2H), 4.81 (d, J=7.2 Hz, 2H), 6.97 (d, J=9.2 Hz, 1H), 7.51-7.57 (m, 4H), 7.84 (d, J=8.8 Hz, 1H), 8.44 (s, 1H).

ESI-MS: m/z, 436.18 (M+H)⁺, 100%.

### Example 220: (3-(3-hydroxyoxetan-3-yl)phenyl)(4-(5-(trifluoromethyl)pyridin-2-yl) piperazin-1-yl)methanone

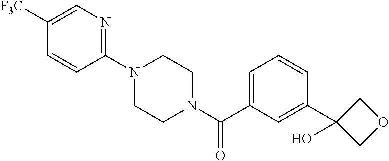 [SMILES: O=C(c1cccc(C2(O)COC2)c1)N1CCN(c2ccc(C(F)(F)F)cn2)CC1]

¹H NMR (DMSO-d₆) δ: 3.40 (brs, 2H), 3.67-3.75 (m, 6H), 4.70 (d, J=6.8 Hz, 2H), 4.80 (d, J=6.8 Hz, 2H), 6.48 (s, 1H), 6.97 (d, J=9.2 Hz, 1H), 7.39 (d, J=7.6 Hz, 1H), 7.52 (d, J=7.6 Hz, 1H), 7.65 (s, 1H), 7.72 (d, J=8.0 Hz, 1H), 7.84 (d, J=9.2 Hz, 1H), 8.44 (s, 1H).

ESI-MS: m/z, 408.15 (M+H)⁺, 100%.

### Example 221: (3-(3-methoxyoxetan-3-yl)phenyl)(4-(5-(trifluoromethyl)pyridin-2-yl) piperazin-1-yl)methanone

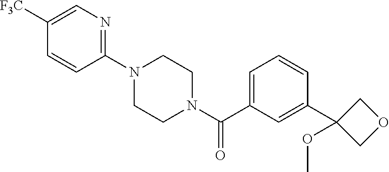 [SMILES: COC1(c2cccc(C(=O)N3CCN(c4ccc(C(F)(F)F)cn4)CC3)c2)COC1]

¹H NMR (CDCl₃) δ: 3.19 (s, 3H), 3.60 (brs, 2H), 3.73 (brs, 4H), 3.85 (brs, 2H), 4.82 (d, J=7.2 Hz, 2H), 4.97 (d, J=7.2 Hz, 2H), 6.69 (d, J=9.2 Hz, 1H), 7.44 (d, J=7.2 Hz, 1H), 7.54 (t, J=4.0 Hz, 1H), 7.58-7.61 (m, 2H), 7.69 (d, J=9.2 Hz, 1H), 8.43 (s, 1H).

ESI-MS: m/z, 422.16 (M+H)⁺, 100%.

### Example 222: (4-(3-hydroxyoxetan-3-yl)phenyl)(4-(2-(trifluoromethyl)pyridin-4-yl) piperazin-1-yl)methanone

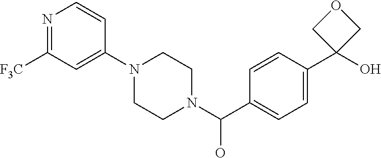 [SMILES: OC(c1ccc(C2(O)COC2)cc1)N1CCN(c2ccnc(C(F)(F)F)c2)CC1]

¹H NMR (DMSO-d6) δ: 3.44-3.55 (m, 6H), 3.74 (brs, 2H), 4.70 (d, J=6.8 Hz, 2H), 4.80 (d, J=6.8 Hz, 2H), 6.48 (s, 1H), 7.04 (d, J=6.4 Hz, 1H), 7.23 (s, 1H), 7.49 (d, J=8.4 Hz, 2H), 7.70 (d, J=8.4 Hz, 2H), 8.30 (d, J=6.0 Hz, 1H).

ESI-MS: m/z, 408.15 (M+H)⁺, 100%.

### Example 223: (4-(3-hydroxyoxetan-3-yl)phenyl)(4-(2-(trifluoromethyl)pyridin-4-yl) piperidin-1-yl)methanone

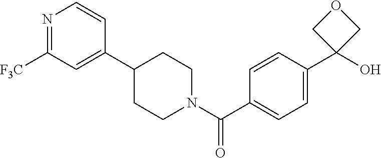 [SMILES: O=C(c1ccc(C2(O)COC2)cc1)N1CCC(c2ccnc(C(F)(F)F)c2)CC1]

¹H NMR (DMSO-d₆) δ: 1.73-1.82 (m, 3H), 1.88-1.92 (m, 1H), 2.67 (brs, 1H), 2.81 (brs, 1H), 3.03 (s, 1H), 3.74 (brs, 1H), 4.62 (brs, 1H), 4.70 (d, J=6.8 Hz, 2H), 4.79 (d, J=6.8 Hz, 2H), 5.76 (s, 1H), 7.49 (d, J=8.4 Hz, 2H), 7.68 (d, J=8.4 Hz, 3H), 7.88 (s, 1H), 8.69 (d, J=4.8 Hz, 1H).

ESI-MS: m/z, 407.21 (M+H)⁺, 100%.

BODIPY-LDL Uptake Assay in HepG2 Cells.

An established assay for PCSK9 inhibition features uptake of fluorescently labeled LDL nanoparticles (BODIPY-LDL) by hepatocytes and uptake of the BODIPY-containing particles is maximized in the absence of PCSK9.

In the LDL uptake assay, HepG2 cells were seeded at a density of 6×104 cells/well in a 96-well plate. After 24 h, the test compounds at various concentrations were pre-incubated with 5 μg/ml PCSK9 in 0.2% DMSO for 60 min prior to the addition on to the cells. After 16 h the medium was removed and 1 μg/ml BODIPY-LDL (Invitrogen) in serum free media was added and incubated for 5 h. Then the cells were washed twice with 0.25% BSA in PBS. Then the fluorescence of the cells in PBS was measured using TECAN multimode reader (Excitation: 485 nm and Emission: 520 nm). The readings were taken in triplicate and the final values were normalized with the protein concentration from respective wells. The wells without test compound containing external PCSK9 is considered as zero uptake and the one without PCSK9 was considered as 100% uptake. The percentage increase in LDL uptake for the test compounds were calculated using these values. The results are presented in the table 1 below.

TABLE 1

| | % increase in LDL Uptake | | |
|---|---|---|---|
| Example | 1 μM | 10 μM | 30 μM |
| 1 | 25 | 135 | 130 |
| 2 | 13 | 86 | 172 |
| 3 | −2 | 39 | 34 |
| 4 | 38 | 58 | 77 |
| 5 | 41 | 76 | 72 |
| 6 | 0 | 106 | 179 |
| 7 | 28 | 63 | 128 |
| 8 | 0 | 73 | 114 |
| 10 | 0 | 273 | 347 |
| 11 | 6 | 59 | 116 |
| 12 | 21 | 32 | 34 |
| 13 | 29 | 43 | 33 |
| 14 | 0 | 20 | 122 |
| 16 | 38 | 22 | 19 |
| 20 | 0 | 7 | 18 |
| 22 | 0 | 19 | 14 |
| 26 | 15 | 21 | 33 |
| 27 | 14 | 21 | 38 |
| 28 | 0 | 8 | 39 |
| 29 | 0 | 17 | 49 |
| 32 | 12 | 30 | 33 |
| 33 | 59 | 73 | 69 |
| 34 | 27 | 86 | 252 |
| 36 | 0 | 9 | 17 |
| 37 | 31 | 65 | 36 |
| 38 | 43 | 51 | 25 |
| 39 | 26 | 149 | 147 |
| 42 | 20 | 25 | 4 |
| 43 | 0 | 12 | 5 |
| 44 | 31 | 56 | 77 |
| 45 | 19 | 9 | 4 |
| 46 | 28 | 47 | 34 |
| 47 | 17 | 189 | 383 |
| 48 | 0 | 142 | 234 |
| 49 | 11 | 45 | 74 |
| 51 | 11 | 79 | 131 |
| 53 | 4 | 80 | 129 |
| 55 | 82 | 320 | 287 |
| 56 | 55 | 344 | 257 |
| 57 | 6 | 32 | 53 |
| 58 | 25 | 21 | 43 |
| 59 | 0 | 0 | 37 |
| 60 | 50 | 7 | 0 |
| 61 | 13 | 32 | 37 |
| 62 | 18 | 10 | 17 |
| 63 | 12 | 18 | 0 |
| 64 | 0 | 13 | 6 |
| 65 | 0 | 9 | 66 |
| 66 | 0 | 24 | 159 |
| 67 | 26 | 27 | 11 |
| 68 | 0 | 2 | 51 |
| 69 | 0 | 0 | 76 |
| 70 | 0 | 26 | 14 |
| 73 | 0 | 8 | 0 |
| 74 | 34 | 42 | 100 |
| 75 | 20 | 38 | 27 |
| 76 | 10 | 98 | 108 |
| 77 | 0 | 13 | 37 |
| 84 | 6 | 13 | 17 |
| 86 | 2 | 33 | 55 |
| 87 | 77 | 115 | 94 |
| 89 | 0 | 5 | 159 |
| 90 | 3 | 39 | 74 |
| 92 | −2 | −8 | 64 |
| 93 | 0 | 90 | 144 |
| 95 | 19 | 127 | 226 |
| 96 | 0 | 28 | 75 |
| 97 | 0 | 0 | 24 |
| 98 | 0 | 124 | 3 |
| 99 | 50 | 0 | 0 |
| 100 | 29 | 0 | 0 |
| 102 | 4 | 31 | 25 |
| 104 | 1 | 30 | 18 |
| 105 | 92 | 130 | 101 |
| 107 | 27 | 0 | 0 |
| 108 | 9 | 26 | 32 |
| 109 | 5 | 29 | 31 |
| 116 | 7 | 35 | 98 |
| 121 | 0 | 22 | 18 |
| 122 | 0 | 16 | 12 |
| 123 | 0 | 14 | 30 |
| 130 | −19 | 20 | 74 |
| 131 | −5 | 57 | −1 |
| 133 | 22 | 28 | 32 |
| 134 | 3 | 28 | 43 |
| 135 | 5 | 17 | 47 |
| 136 | 11 | 12 | 14 |
| 137 | 0 | 66 | 116 |
| 141 | 0 | 0 | 28 |
| 142 | 0 | 77 | 16 |
| 143 | 0 | 41 | 28 |
| 144 | 1 | 16 | 77 |
| 145 | 2 | 70 | 0 |
| 146 | 53 | 212 | 29 |
| 148 | 20 | 3 | 11 |
| 149 | 0 | 30 | 9 |
| 150 | 0 | 5 | 25 |
| 151 | 34 | 0 | 0 |
| 152 | 0 | 0 | 31 |
| 153 | 0 | 47 | 46 |
| 155 | 0 | 0 | 58 |
| 158 | 0 | 42 | 0 |
| 161 | 0 | 0 | 16 |
| 175 | 17 | 0 | 0 |
| 176 | 0 | 12 | 68 |
| 178 | 0 | 51 | 47 |
| 181 | 3 | 19 | 26 |
| 182 | 42 | 37 | 60 |
| 183 | 32 | 15 | 88 |
| 184 | 19 | 117 | 250 |
| 185 | 0 | 29 | 109 |
| 186 | 17 | 73 | 31 |
| 188 | 0 | 152 | 136 |
| 189 | 0 | 116 | 19 |
| 190 | 0 | 90 | 62 |
| 198 | −8 | 25 | 3 |
| 201 | 0 | 0 | 73 |
| 202 | 16 | 0 | 36 |
| 206 | 0 | 81 | 127 |
| 208 | 56 | 14 | 189 |
| 209 | 20 | 137 | 21 |
| 210 | 0 | 107 | 0 |
| 212 | 8 | 4 | 48 |
| 213 | 0 | 0 | 37 |
| 215 | 0 | 90 | 140 |
| 216 | 0 | 64 | 189 |
| 217 | 0 | 5 | 128 |
| 218 | 0 | 26 | 85 |
| 219 | 4 | 41 | 15 |
| 220 | 8 | 31 | 56 |
| 221 | 0 | 28 | 66 |

LDL-C Lowering Activity—in High Fat Diet C57 Mice

The in-vivo LDL-c lowering for test compound was tested in C57 mice which were kept on high fat diet for 4 weeks and the blood was collected by retro-orbital sinus puncture method under light ether anesthesia on day 0 (pretreatment). Animal are grouped based on LDL-c levels, after that 7 days treatment with vehicle or test compound orally at a dose of 30 mpk dose once a day was given. On completion of treatment on day 7 of the treatment the blood was collected for LDL-c and TC levels measurement. The percent change in LDL-c and TC in test compound group Vs Vehicle group was calculated and presented in the table 2 below

TABLE 2

| | Dose | % Change Vs Vehicle |
|---|---|---|
| Example No | (mg/Kg) | Control in LDLc |
| 1 | 30 | −52 ± 4 |
| 2 | 30 | −45 ± 5 |
| 6 | 30 | 0.6 ± 9 |
| 8 | 30 | −30 ± 8 |
| 10 | 30 | −15 ± 8 |
| 11 | 30 | −48 ± 5 |
| 34 | 30 | −31 ± 8 |
| 39 | 30 | −29 ± 5 |
| 43 | 30 | −27 ± 10 |
| 47 | 30 | −5 ± 7 |
| 53 | 30 | −15 ± 6 |
| 56 | 30 | −5 ± 8 |
| 57 | 30 | −43 ± 9 |
| 58 | 30 | −44 ± 3 |
| 66 | 30 | −24.0 ± 7.1 |
| 76 | 30 | −28 ± 7 |
| 86 | 30 | −28 ± 4 |
| 87 | 30 | −36 ± 5 |
| 90 | 30 | −5.5 ± 4.5 |
| 95 | 30 | −16 ± 9 |
| 105 | 30 | −7 ± 7 |
| 134 | 30 | −38 ± 7 |
| 142 | 30 | −26 ± 10 |
| 144 | 30 | −66 ± 10 |
| 182 | 30 | −5 ± 11 |
| 184 | 30 | −7 ± 7 |
| 206 | 30 | −18.6 ± 5.7 |
| 215 | 30 | −29 ± 5 |
| 216 | 30 | −40 ± 6 |
| 220 | 30 | −16 ± 6.4 |

The novel compounds of the present invention can be formulated into suitable pharmaceutically acceptable compositions by combining with suitable excipients by techniques and processes and concentrations as are well known. The pharmaceutical composition comprising the compounds of present invention in combination with suitable pharmaceutically acceptable excipients such as diluents, binders, bulking agents or any other necessary pharmaceutical excipients.

The pharmaceutical composition is provided by employing conventional techniques. Preferably the composition is in unit dosage form containing an effective amount of the active component, that is, the compounds of formula (I) according to this invention.

The quantity of active component, that is, the compounds of formula (I) according to this invention, in the pharmaceutical composition and unit dosage form thereof may be varied or adjusted widely depending upon the particular application method, the potency of the particular compound and the desired concentration. Generally, the quantity of active component will range between 0.5% to 90% by weight of the composition.

In another embodiment, the compound of the present invention may be used alone or in combination with a second medicament as may be necessary depending on the condition of the patient, the severity of the disease and such other conditions which are well known to a skilled practitioner. Such second medicament when required may be selected from a HMG-Co-A reductase inhibitor, angiotension converting enzyme (ACE) inhibitor, calcium channel blocker, aldosterone synthase inhibitor, aldoasterone antagonist, dual angiotension converting enzyme/neutral endopeptidase (ACE/NEP) inhibitor, endothelin antagonist, angiotension II receptor blocker (ARB) including their pharmaceutically acceptable salts as well as a combination of one or more medicines from any of these classes along with the compound of formula (I) of the present invention.

In certain instances, it may be appropriate to administer at least one of the compounds described herein or a pharmaceutically acceptable salt, ester, or prodrug thereof in combination with another therapeutic agent. Several reasons can be attributed for using a combination therapy depending on the need of the patient. As an example, if one of the side effects experienced by a patient upon receiving one of the compounds herein is hypertension, then it may be appropriate to administer an anti-hypertensive agent in combination with the initial therapeutic agent. Or, by way of example only, the benefit experienced by a patient may be increased by administering one of the compounds described herein with another therapeutic agent (which also includes a therapeutic regimen) that also has therapeutic benefit. Several such instances are well known to a skilled person and the use of combination therapy may be envisaged for all such situations. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of certain compounds disclosed herein with agents found in the following pharmacotherapeutic classifications as indicated below. These lists should not be construed to be closed, but should instead serve as illustrative examples common to the relevant therapeutic area at present. Moreover, combination regimens may include a variety of routes of administration and should include oral, intravenous, intraocular, subcutaneous, dermal, and inhaled topical.

For the treatment of metabolic disorders, compounds disclosed herein may be administered with an agent selected from the group comprising: insulin, insulin derivatives and mimetics, insulin secretagogues, insulin sensitizers, biguanide agents, alpha-glucosidase inhibitors, insulinotropic sulfonylurea receptor ligands, meglitinides, GLP-1 (glucagon like peptide-1), GLP-1 analogs, dipeptidyl peptidase IV (DPPIV) inhibitors, GPR-119 inhibitors, sodium-dependent glucose co-transporter (SGLT2) inhibitors, PPAR modulators, non-glitazone type PPAR.delta. agonist, HMG-CoA reductase inhibitors, cholesterol-lowering drugs, rennin inhibitors, anti-thrombotic and anti-platelet agents and anti-obesity agents.

For the treatment of metabolic disorders, compounds disclosed herein may be administered with an agent selected from the group comprising: insulin, metformin, Glipizide, glyburide, Amaryl, gliclazide, meglitinides, nateglinide, repaglinide, amylin mimetics (for example, pramlintide), acarbose, miglitol, voglibose, Exendin-4, vildagliptin, Liraglutide, naliglutide, saxagliptin, pioglitazone, rosiglitazone, HMG-CoA reductase inhibitors (for example, rosuvastatin, atrovastatin, simvastatin, lovastatin, pravastatin, fluvastatin, cerivastatin, rosuvastatin, pitavastatin and like), cholesterol-lowering drugs (for example, fibrates which include: fenofibrate, benzafibrate, clofibrate, gemfibrozil and like; cholesterol absorption inhibitors such as Ezetimibe, eflucimibe etc.

In another embodiment, method of treating hyperlipidemia and disorders related to/caused by hyperlipidemia comprising the administering to a patient in need thereof an effective amount of a compound of formula (I) or pharmaceutical composition thereof.

In another embodiment of the present invention, use of compound of general formula (I), their tautomeric form, their pharmaceutically acceptable salts, or their pharmaceutical composition in a medicament for treating hyperlipidemia and related disease. Compounds have also beneficial effect cholesterol lowering.

From the foregoing description, one skilled in the art can easily ascertain the essential characteristics of this invention, and without departing from the spirit and scope thereof, can make various changes and modifications of the invention to adapt it to various usages and conditions. Such different embodiments are also to be considered to be within the scope of the present invention.

1. Compounds of general formula (I) 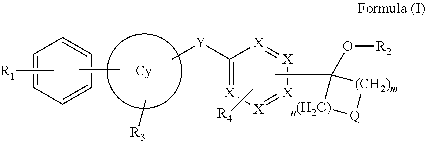
their tautomer forms, their stereoisomers, their pharmaceutically acceptable salts and pharmaceutical compositions containing them wherein
‘Cy’ is selected from heterocyclic groups selected from saturated or partially unsaturated or unsaturated, monocyclic or bicyclic or spirocyclic groups containing 0-4 heteroatoms selected from O, N or S;
‘Y’ is selected from either a bond, or O, S(O)ₒ, CO, (C₁-C₃)alkyl, C(O)NR₅, NR₅ or SO₂NR₅;
wherein R₅ represents H, (C₁-C₆)alkyl, (C₃-C₆)cycloalkyl;
‘Q’ selected from O, S(O)ₒ or NR₇ wherein R₇ is selected from H, (C₁-C₆)alkyl, (C₃-C₆)cycloalkyl, acyl, —C(O)OR₆, wherein R₆ represents (C₁-C₆) linear or branched alkyl;
‘o’ selected from integers from 0-2;
‘m’ and ‘n’ selected from integers from 1-4
‘p’ selected from integers from 1-4
‘X’ at each occurrence independently selected from either C or N;
R₁ is selected from hydrogen, halo or substituted or unsubstituted groups selected from alkyl, haloalkyl, perhaloalkyl, alkoxy, haloalkoxy, perhaloalkoxy, cycloalkyl, alkoxy, cycloalkoxy, aryl, aryloxy, aralkyl, aralkoxy, heterocyclyl, heterocyclylalkyl, heterocyclooxy, heterocyclylalkoxy, heterocyclylalkoxyacyl, carboxylic acid and its derivatives such as esters and amides, carbonylamino, hydroxyalkyl, alkoxyalkyl, aryloxyalkyl, aralkoxyalkyl, alkylthio, thioalkyl, alkylsulfonyl, hydroxyl, sulfonic acid and its derivatives;
R₂ is selected from hydrogen, or substituted or unsubstituted groups selected from alkyl, haloalkyl, perhaloalkyl, cycloalkyl, deuterated alkyl, alkynyl, alkenyl, aryl, aralkyl, heterocyclyl, heterocyclylalkyl, heterocyclylalkoxyacyl, acyl, carbonylamino, hydroxyalkyl, alkoxyalkyl, aryloxyalkyl, aralkoxyalkyl, alkylsulfonyl, alkylthioalkyl, alkylsulfinylalkyl, alkylsulfonylalkyl, alkylcarboxylic acid;
R₃ and R₄ independently selected from hydrogen, halo, cyano, hydroxy or substituted or unsubstituted groups selected from alkyl, haloalkyl, perhaloalkyl, alkoxy, haloalkoxy, perhaloalkoxy, cycloalkyl, alkoxy, cycloalkoxy, aryl, aryloxy, aralkyl, aralkoxy, heterocyclyl, heterocyclylalkyl, heterocyclooxy, heterocyclylalkoxy, heterocyclylalkoxyacyl, carboxylic acid and its derivatives such as esters and amides, carbonylamino, hydroxyalkyl, alkoxyalkyl, aryloxyalkyl, aralkoxyalkyl, alkylthio, thioalkyl, alkylsulfonyl, hydroxyl, sulfonic acid and its derivatives.

2. Compound of formula (I) as claimed in claim 1, wherein ‘Cy’ is selected from pyrrolidinyl, piperidinyl, piperazinyl, diazepinyl, oxazolyl, oxadiazolyl, indolinyl, pyridothienyl, hexahydrocyclopenta[c]pyrrol, hexahydropyrrolo[3,4-c]pyrrol, dihydropyrrolo[3,4-c]pyrazol, 5H-imidazo[4,5-c]pyridine, 5,6-dihydropyridin-, hexahydrocyclopenta[c]pyrrol, 3,9-diazaspiro[5.5]undecan, tetrahydropyrrolo[3,4-c]pyrrol, 3,4-dihydroisoquinolin-3,3a,6,6a-tetrahydrocyclopenta[c]pyrrol-, 3,6-diazabicyclo[3.1.1]heptan, dihydrothieno[3,2-c]pyridine, dihydrothiazolo[5,4-c]pyridine, diazaspiro[4.5]decan heterocycles.

3. Compound of general formula (I) as claimed in claim 1, wherein ‘Y’ is selected form a bond, O, S(O)ₒ, CO, C(O)NR₅, wherein R₅ represents H.

4. Compound of general formula (I) as claimed in claim 1, wherein compound is selected from:
(4-(3-hydroxyoxetan-3-yl)phenyl)(4-(4-(trifluoromethyl)phenyl)piperidin-1-yl)methanone;
(4-(3-methoxyoxetan-3-yl)phenyl)(4-(4-(trifluoromethyl)phenyl)piperidin-1-yl)methanone;
N-(4-(3-hydroxyoxetan-3-yl)phenyl)-1-(4-(trifluoromethyl)phenyl)piperidine-4-carboxamide;
N-(4-(3-hydroxyoxetan-3-yl)phenyl)-4-(4-(trifluoromethyl)phenyl)piperidine-1-carboxamide;
N-(4-(3-methoxyoxetan-3-yl)phenyl)-4-(4-(trifluoromethyl)phenyl)piperidine-1-carboxamide;
(4-(3-hydroxyoxetan-3-yl)phenyl)(4-(4-(trifluoromethyl)phenyl)piperazin-1-yl)methanone;
3-(4-((4-(4-(trifluoromethyl)phenyl)piperazin-1-yl)sulfonyl)phenyl)oxetan-3-ol;
3-(4-(4-(4-(trifluoromethyl)phenyl)piperidin-1-yl)phenyl)oxetan-3-ol;
1-(4-(3-methoxyoxetan-3-yl)phenyl)-4-(4-(trifluoromethyl)phenyl)piperidine;
(4-(3-hydroxyoxetan-3-yl)phenyl)(5-(4-(trifluoromethyl)phenyl)hexahydrocyclopenta[c]pyrrol-2(1H)-yl)methanone;
(4-(3-hydroxyoxetan-3-yl)phenyl)(5-(4-(trifluoromethyl)phenyl)hexahydropyrrolo[3,4-c]pyrrol-2(1H)-yl)methanone;
(4-(3-(2-hydroxyethoxy)oxetan-3-yl)phenyl)(5-(4-(trifluoromethyl)phenyl)hexahydropyrrolo[3,4-c]pyrrol-2(1H)-yl)methanone;
2-((3-(4-(5-(4-(trifluoromethyl)phenyl)octahydropyrrolo[3,4-c]pyrrole-2-carbonyl)phenyl)oxetan-3-yl)oxy)acetic acid;
(4-(3-fluoro-5-(trifluoromethyl)phenyl)piperidin-1-yl)(4-(3-hydroxyoxetan-3-yl)phenyl)methanone;
(4-(3-hydroxyoxetan-3-yl)phenyl)(4-(3-(4-methyl-1H-imidazol-1-yl)-5-(trifluoromethyl)phenyl)piperidin-1-yl)methanone;
3-(4-(3-(3-fluoro-4-(trifluoromethyl)phenyl)-1,2,4-oxadiazol-5-yl)phenyl)oxetan-3-ol;
3-(4-((1-(4-(trifluoromethyl)phenyl)piperidin-4-yl)oxy)phenyl)oxetan-3-ol;
(4-(3-hydroxyoxetan-3-yl)phenyl)(2-(4-(trifluoromethyl)phenyl)-6,7-dihydrothieno [3,2-c]pyridin-5(4H)-yl)methanone;
(1-benzyl-3-(4-(trifluoromethyl)phenyl)-4,6-dihydropyrrolo[3,4-c]pyrazol-5 (1H)-yl)(4-(3-hydroxyoxetan-3-yl)phenyl)methanone;
(4-(3-hydroxyoxetan-3-yl)phenyl)(1-methyl-3-(4-(trifluoromethyl)phenyl)-4,6-dihydropyrrolo[3,4-c]pyrazol-5 (1H)-yl)methanone;
(4-(3-hydroxyoxetan-3-yl)phenyl)(2-methyl-3-(4-(trifluoromethyl)phenyl)-2,6-dihydropyrrolo[3,4-c]pyrazol-5 (4H)-yl)methanone;
(4-(3-hydroxyoxetan-3-yl)phenyl)(4-(4-(trifluoromethoxy)phenyl)piperidin-1-yl)methanone;
(4-(3-hydroxyoxetan-3-yl)phenyl)(4-(p-tolyl)piperidin-1-yl)methanone;
(4-(3-hydroxyoxetan-3-yl)phenyl)(4-(3-(trifluoromethyl)phenyl)piperidin-1-yl)methanone;
(3-(3-hydroxyoxetan-3-yl)phenyl)(4-(p-tolyl)piperidin-1-yl)methanone;
(4-(3-chloro-4-methoxyphenyl)piperidin-1-yl)(4-(3-hydroxyoxetan-3-yl)phenyl)methanone;
(4-(3-hydroxyoxetan-3-yl)phenyl)(4-(4-methoxy phenyl)piperidin-1-yl)methanone;
(4-(3-fluoro-4-methoxy phenyl)piperidin-1-yl)(4-(3-hydroxyoxetan-3-yl)phenyl)methanone;
(4-(3-chloro-4-methylphenyl)piperidin-1-yl)(4-(3-hydroxyoxetan-3-yl)phenyl)methanone;
(4-(3-hydroxyoxetan-3-yl)phenyl)(4-(3-methoxy-4-(trifluoromethyl)phenyl)piperidin-1-yl)methanone;
(4-(3-fluoro-4-(trifluoromethyl)phenyl)piperidin-1-yl)(4-(3-hydroxyoxetan-3-yl)phenyl)methanone;
(4-(3-chloro-4-(trifluoromethyl)phenyl)piperidin-1-yl)(4-(3-hydroxyoxetan-3-yl)phenyl)methanone;
(4-(3-hydroxyoxetan-3-yl)phenyl)(4-(2-(trifluoromethyl)phenyl)piperidin-1-yl)methanone;
(4-(3-hydroxyoxetan-3-yl)phenyl)(3-(4-(trifluoromethyl)phenyl)piperidin-1-yl)methanone;
2-((3-(4-(4-(4-(trifluoromethyl)phenyl)piperidine-1-carbonyl)phenyl)oxetan-3-yl)oxy)acetic acid;
3-(4-(4-(4-(trifluoromethyl)phenyl)piperidine-1-carbonyl)phenyl)oxetan-3-yl acetate;
(4-(3-methoxyoxetan-3-yl)phenyl)(4-(3-(trifluoromethyl)phenyl)piperidin-1-yl)methanone;
(4-(3-fluoro-4-(trifluoromethyl)phenyl)piperidin-1-yl)(4-(3-methoxyoxetan-3-yl)phenyl)methanone;
(4-(3-methoxyoxetan-3-yl)phenyl)(3-(4-(trifluoromethyl)phenyl)piperidin-1-yl)methanone;
(4-(3-ethoxyoxetan-3-yl)phenyl)(4-(3-(trifluoromethyl)phenyl)piperidin-1-yl)methanone;
(4-(3-methoxyoxetan-3-yl)phenyl)(4-(2-(trifluoromethyl)phenyl)piperidin-1-yl)methanone;
(4-(3-ethoxyoxetan-3-yl)phenyl)(4-(3-fluoro-4-(trifluoromethyl)phenyl) piperidin-1-yl)methanone;
(4-(3-ethoxyoxetan-3-yl)phenyl)(4-(4-(trifluoromethyl)phenyl)piperidin-1-yl)methanone;
(4-(3-isopropoxyoxetan-3-yl)phenyl)(4-(4-(trifluoromethyl)phenyl)piperidin-1-yl)methanone;
(4-(3-isobutoxyoxetan-3-yl)phenyl)(4-(4-(trifluoromethyl)phenyl)piperidin-1-yl)methanone;
(4-(3-(cyclopropylmethoxy)oxetan-3-yl)phenyl)(4-(4-(trifluoromethyl)phenyl)piperidin-1-yl)methanone;
(3-(3-hydroxyoxetan-3-yl)phenyl)(4-(4-(trifluoromethyl)phenyl)piperidin-1-yl)methanone;
(3-(3-hydroxyoxetan-3-yl)phenyl)(3-(4-(trifluoromethyl)phenyl)piperidin-1-yl)methanone;
(5-(3-hydroxyoxetan-3-yl)pyridin-3-yl)(4-(4-(trifluoromethyl)phenyl)piperidin-1-yl)methanone;
(3-(3-methoxyoxetan-3-yl)phenyl)(4-(4-(trifluoromethyl)phenyl)piperidin-1-yl)methanone;
(3-(3-methoxyoxetan-3-yl)phenyl)(3-(4-(trifluoromethyl)phenyl)piperidin-1-yl)methanone;
N-(3-(3-hydroxyoxetan-3-yl)phenyl)-4-(4-(trifluoromethyl)phenyl)piperidine-1-carboxamide;
(4-(3-methoxyoxetan-3-yl)phenyl)(4-(4-(trifluoromethyl)phenyl)piperazin-1-yl)methanone;
3-(4-((4-(4-(trifluoromethyl)phenyl)piperidin-1-yl)sulfonyl)phenyl)oxetan-3-ol;
(3-(3-hydroxyoxetan-3-yl)phenyl)(5-(4-(trifluoromethyl)phenyl)hexahydropyrrolo[3,4-c]pyrrol-2(1H)-yl)methanone;
(3-(3-methoxyoxetan-3-yl)phenyl)(5-(4-(trifluoromethyl)phenyl)hexahydropyrrolo[3,4-c]pyrrol-2(1H)-yl)methanone;
(4-(3-methoxyoxetan-3-yl)phenyl)(5-(4-(trifluoromethyl)phenyl)hexahydropyrrolo[3,4-c]pyrrol-2(1H)-yl)methanone;
(4-(3-ethoxyoxetan-3-yl)phenyl)(5-(4-(trifluoromethyl)phenyl)hexahydropyrrolo[3,4-c]pyrrol-2(1H)-yl)methanone;
(4-(4-(difluoromethyl) phenyl) piperidin-1-yl)(3-(3-(methoxy-d3) oxetan-3-yl) phenyl) methanone;
N-(4-(3-methoxyoxetan-3-yl) phenyl)-1-(4-(trifluoromethyl) phenyl) piperidine-4-carboxamide;
(4-(3-hydroxy-1,1-dioxidothietan-3-yl) phenyl)(5-(4-(trifluoromethyl) phenyl)hexahydropyrrolo[3,4-c]pyrrol-2(1H)-yl)methanone;
(4-(3-hydroxy-1,1-dioxidothietan-3-yl)phenyl)(4-(4-(trifluoromethyl)phenyl)piperidin-1-yl)methanone;
(4-(3-hydroxyoxetan-3-yl) phenyl)(4-(4-hydroxyphenyl) piperidin-1-yl)methanone;
(4-(3-hydroxyoxetan-3-yl)phenyl)(4-phenylpiperidin-1-yl)methanone;
(4-(4-(difluoromethyl)phenyl)piperidin-1-yl)(3-(3-methoxyoxetan-3-yl)phenyl)methanone;
(4-(4-(difluoromethyl)phenyl)piperidin-1-yl)(3-(3-hydroxyoxetan-3-yl)phenyl)methanone;
(4-(3-hydroxyoxetan-3-yl)phenyl)(2-(2-methoxy pyridin-4-yl)-4-(4-(trifluoromethyl)phenyl)piperazin-1-yl)methanone;
(4-(4-(difluoromethyl)phenyl)piperidin-1-yl)(4-(3-methoxyoxetan-3-yl)phenyl)methanone;
(4-(4-(difluoromethyl)phenyl)piperidin-1-yl)(4-(3-hydroxyoxetan-3-yl)phenyl)methanone;
(4-(3-(but-2-yn-1-yloxy)oxetan-3-yl)phenyl)(4-(4-(trifluoromethyl)phenyl)piperidin-1-yl)methanone;
N-(4-(3-hydroxyoxetan-3-yl)phenyl)-5-phenyl-2-(4-(trifluoromethyl)phenyl)oxazole-4-carboxamide;
(4-(3-(prop-2-yn-1-yloxy)oxetan-3-yl)phenyl)(4-(4-(trifluoromethyl)phenyl)piperidin-1-yl)methanone;
4-(4-(3-methoxyoxetan-3-yl)phenyl)-1-(4-(trifluoromethyl)phenyl)piperidin-4-ol;
N-(4-(3-methoxyoxetan-3-yl)phenyl)-5-(4-(trifluoromethyl)phenyl)hexahydropyrrolo[3,4-c]pyrrole-2(1H)-carboxamide;
N-(4-(3-hydroxyoxetan-3-yl)phenyl)-5-(4-(trifluoromethyl)phenyl)hexahydropyrrolo[3,4-c]pyrrole-2(1H)-carboxamide;
(4-(3-(methoxy-d3)oxetan-3-yl)phenyl)(4-(4-(trifluoromethyl)phenyl)piperidin-1-yl)methanone;
5-(3-hydroxyoxetan-3-yl)-2-(4-(4-(trifluoromethyl) phenyl) piperidin-1-yl) benzoic acid;
3-(3-hydroxyoxetan-3-yl)-5-(4-((4-(trifluoromethyl)phenyl)piperidin-1-yl)benzoic acid;
(3-(3-hydroxyoxetan-3-yl)phenyl)(2-(4-(trifluoromethyl)phenyl)-1,4,6,7-tetrahydro-5H-imidazo[4,5-c]pyridin-5-yl)methanone;
(4-(3-hydroxyoxetan-3-yl)phenyl)(2-(4-(trifluoromethyl)phenyl)-1,4,6,7-tetrahydro-5H-imidazo[4,5-c]pyridin-5-yl)methanone;
(4-(3-hydroxyoxetan-3-yl)-2-morpholinophenyl)(4-(4-(trifluoromethyl)phenyl)piperidin-1-yl)methanone;
(4-(3-hydroxyoxetan-3-yl)-2-(piperazin-1-yl)phenyl)(4-(4-(trifluoromethyl)phenyl)piperidin-1-yl)methanone;
1-(4-(3-ethoxyoxetan-3-yl)phenyl)-4-(4-(trifluoromethyl)phenyl)piperidine;
3-(3-morpholino-5-(4-(4-(trifluoromethyl)phenyl)piperidin-1-yl)phenyl)oxetan-3-ol;
tert-butyl 4-(5-(3-hydroxyoxetan-3-yl)-2-(4-(4-(trifluoromethyl)phenyl)piperidine-1-carbonyl)phenyl)piperazine-1-carboxylate;
(4-hydroxy-4-(4-(trifluoromethyl)phenyl)piperidin-1-yl)(4-(3-hydroxyoxetan-3-yl)phenyl)methanone;
(4-(3-hydroxyoxetan-3-yl)phenyl)(4-(4-(trifluoromethyl)phenyl)-5,6-dihydropyridin-1 (2H)-yl)methanone;
(4-(3-hydroxyoxetan-3-yl) phenyl)(1-(4-(trifluoromethyl)phenyl)-4,6-dihydropyrrolo[3,4-c]pyrazol-5(1H)-yl)methanone;
(4-(3-hydroxyoxetan-3-yl)phenyl)(2-(4-(trifluoromethyl)phenyl)-2,6-dihydropyrrolo[3,4-c]pyrazol-5 (4H)-yl)methanone;
(4-(3-methoxyoxetan-3-yl)phenyl)(2-(4-(trifluoromethyl)phenyl)-2,6-dihydropyrrolo[3,4-c]pyrazol-5 (4H)-yl)methanone;
(4-(3-hydroxyoxetan-3-yl)phenyl)(5-methoxy-5-(4-(trifluoromethyl)phenyl) hexahydrocyclopenta[c]pyrrol-2(1H)-yl)methanone;
(5-methoxy-5-(4-(trifluoromethyl)phenyl)hexahydrocyclopenta[c]pyrrol-2(1H)-yl)(4-(3-methoxyoxetan-3-yl)phenyl)methanone;
(4-(3-hydroxyoxetan-3-yl)phenyl)(5-(4-(trifluoromethyl)phenyl)-3,5-dihydropyrrolo[3,4-c]pyrrol-2(1H)-yl)methanone;
(4-(3-methoxyoxetan-3-yl)phenyl)(5-(4-(trifluoromethyl)phenyl)-3,5-dihydropyrrolo[3,4-c]pyrrol-2(1H)-yl)methanone;
(4-(3-hydroxyoxetan-3-yl)phenyl)(5-(4-(trifluoromethyl)phenyl)-3,4,5,6-tetrahydropyrrolo[3,4-c]pyrrol-2(1H)-yl)methanone;
(3-(3-hydroxyoxetan-3-yl)phenyl)(5-(4-(trifluoromethyl)phenyl)-3,4,5,6-tetrahydropyrrolo[3,4-c]pyrrol-2(1H)-yl)methanone;
(3-(3-methoxyoxetan-3-yl)phenyl)(5-(4-(trifluoromethyl)phenyl)-3,4,5,6-tetrahydropyrrolo[3,4-c]pyrrol-2(1H)-yl)methanone;
3-(4-((5-(4-(trifluoromethyl)phenyl)hexahydropyrrolo[3,4-c]pyrrol-2(1H)-yl)sulfonyl)phenyl)oxetan-3-ol;
(4-(3-hydroxyoxetan-3-yl)phenyl)(9-(4-(trifluoromethyl)phenyl)-3,9-diazaspiro[5.5]undecan-3-yl)methanone;
(3-(3-hydroxyoxetan-3-yl)phenyl)(9-(4-(trifluoromethyl)phenyl)-3,9-diazaspiro[5.5]undecan-3-yl)methanone;
(4-(3-methoxyoxetan-3-yl)phenyl)(9-(4-(trifluoromethyl)phenyl)-3,9-diazaspiro[5.5]undecan-3-yl)methanone;
(4-(3-hydroxyoxetan-3-yl)phenyl)(3-(4-(trifluoromethyl)phenyl)pyrrolidin-1-yl)methanone;
((5-(4-fluorophenyl)-3,3a,6,6a-tetrahydrocyclopenta[c]pyrrol-2(1H)-yl)(4-(3-hydroxyoxetan-3-yl)phenyl)methanone;
(4-(3-ethoxy oxetan-3-yl)phenyl)(5-(4-(trifluoromethyl)phenyl)-3,4,5,6-tetrahydropyrrolo[3,4-c]pyrrol-2(1H)-yl)methanone;
(5-hydroxy-5-(4-(trifluoromethyl)phenyl)hexahydrocyclopenta[c]pyrrol-2(1H)-yl)(4-(3-hydroxyoxetan-3-yl)phenyl)methanone;
(4-(3-hydroxyoxetan-3-yl)phenyl)(6-(4-(trifluoromethyl)phenyl)-3,4-dihydroisoquinolin-2(1H)-yl)methanone;
(4-(3-hydroxyoxetan-3-yl)phenyl)(5-(4-(trifluoromethyl)phenyl)indolin-1-yl)methanone;
(4-(3-methoxyoxetan-3-yl)phenyl)(4-(3-(4-methyl-1H-imidazol-1-yl)-4-(trifluoromethyl)phenyl)piperidin-1-yl)methanone;
2,2-dimethyl-7-((3-(4-(4-(4-(trifluoromethyl)phenyl)piperidine-1-carbonyl)phenyl)oxetan-3-yl)oxy)heptanoic acid;
(4-(methoxymethyl)-4-(4-(trifluoromethyl)phenyl)piperidin-1-yl)(4-(3-methoxyoxetan-3-yl)phenyl)methanone;
(4-(hydroxymethyl)-4-(4-(trifluoromethyl)phenyl)piperidin-1-yl)(4-(3-hydroxyoxetan-3-yl)phenyl)methanone;
1-(4-(3-methoxyoxetan-3-yl)benzoyl)-4-(4-(trifluoromethyl)phenyl)piperidine-4-carboxylic acid;
methyl1-(4-(3-hydroxyoxetan-3-yl)benzoyl)-4-(4-(trifluoromethyl)phenyl) piperidine-4-carboxylate;
1-(4-(3-hydroxyoxetan-3-yl)benzoyl)-4-(4-(trifluoromethyl)phenyl) piperidine-4-carboxy c acid;
(4-(3-chloro-4-(trifluoromethyl)phenyl)piperidin-1-yl)(4-(3-methoxyoxetan-3-yl)phenyl)methanone;
1-(4-(3-hydroxyoxetan-3-yl)benzoyl)-4-(4-(trifluoromethyl)phenyl)piperidine-4-carbonitrile;
1-(4-(3-methoxyoxetan-3-yl)benzoyl)-4-(4-(trifluoromethyl)phenyl)piperidine-4-carbonitrile;
(4-(3-hydroxyoxetan-3-yl)phenyl)(4-methoxy-4-(4-(trifluoromethyl)phenyl)piperidin-1-yl)methanone;
(4-methoxy-4-(4-(trifluoromethyl)phenyl)piperidin-1-yl)(4-(3-methoxyoxetan-3-yl)phenyl)methanone;
(3-(3-hydroxyoxetan-3-yl)phenyl)(4-(3-(trifluoromethyl)phenyl)piperidin-1-yl)methanone;
(4-(3-fluoro-4-(trifluoromethyl)phenyl)piperidin-1-yl)(3-(3-hydroxyoxetan-3-yl)phenyl)methanone;
(3-(3-hydroxyoxetan-3-yl)phenyl)(4-(2-(trifluoromethyl)phenyl)piperidin-1-yl)methanone;
(4-(3-ethoxy oxetan-3-yl)phenyl)(3-(4-(trifluoromethyl)phenyl)piperidin-1-yl)methanone;
3-(3-(3-(4-(trifluoromethyl)phenyl)-1,2,4-oxadiazol-5-yl)phenyl)oxetan-3-ol;
5-(3-(3-methoxyoxetan-3-yl)phenyl)-3-(4-(trifluoromethyl)phenyl)-1,2,4-oxadiazole;
3-(4-(3-(4-(trifluoromethyl)phenyl)-1,2,4-oxadiazol-5-yl)phenyl)oxetan-3-ol;
5-(4-(3-methoxyoxetan-3-yl)phenyl)-3-(4-(trifluoromethyl)phenyl)-1,2,4-oxadiazole;
(4-(3-hydroxyoxetan-3-yl)phenyl)(4-(4-((trifluoromethyl)thio)phenyl)piperidin-1-yl)methanone;
(4-(3-methoxyoxetan-3-yl)phenyl)(4-(4-((trifluoromethyl)thio)phenyl)piperidin-1-yl)methanone;
(4-(3-hydroxyoxetan-3-yl)phenyl)(5-(p-tolyl)-3,3a,6,6a-tetrahydrocyclopenta[c]pyrrol-2(1H)-yl)methanone;
(4-(3-hydroxyoxetan-3-yl)phenyl)(5-(4-(methylthio)phenyl)-3,3a,4,6a-tetrahydrocyclopenta[c]pyrrol-2(1H)-yl)methanone;
(4-(3-hydroxyoxetan-3-yl)phenyl)(6-(4-(trifluoromethyl)phenyl)-3,6-diazabicyclo[3.1.1]heptan-3-yl)methanone;
(4-(3-(cyclopropylmethoxy)oxetan-3-yl)phenyl)(5-(4-(trifluoromethyl)phenyl)hexahydropyrrolo[3,4-c]pyrrol-2(1H)-yl)methanone;
(4-(3-(methoxy-d3)oxetan-3-yl)phenyl)(5-(4-(trifluoromethyl)phenyl)hexahydropyrrolo[3,4-c]pyrrol-2(1H)-yl)methanone;
(4-(3-isobutoxyoxetan-3-yl)phenyl)(5-(4-(trifluoromethyl)phenyl)hexahydropyrrolo[3,4-c]pyrrol-2(1H)-yl)methanone;
(4-(3-isopropoxyoxetan-3-yl)phenyl)(5-(4-(trifluoromethyl)phenyl)hexahydropyrrolo[3,4-c]pyrrol-2(1H)-yl)methanone;
(4-(3-(2-methoxyethoxy)oxetan-3-yl)phenyl)(5-(4-(trifluoromethyl)phenyl)hexahydropyrrolo[3,4-c]pyrrol-2(1H)-yl)methanone;
3-(4-(5-(4-(trifluoromethyl)phenyl)octahydropyrrolo[3,4-c]pyrrole-2-carbonyl)phenyl)oxetan-3-yl acetate;
3-(4-(5-(4-(trifluoromethyl)phenyl)octahydropyrrolo[3,4-c]pyrrole-2-carbonyl)phenyl)oxetan-3-yl pivalate;
tert-butyl 4-hydroxy-4-(4-(4-(4-(trifluoromethyl)phenyl)piperidine-1-carbonyl)phenyl)piperidine-1-carboxylate;
(4-(4-hydroxypiperidin-4-yl)phenyl)(4-(4-(trifluoromethyl)phenyl)piperidin-1-yl)methanone;
(4-(4-hydroxytetrahydro-2H-pyran-4-yl)phenyl)(4-(4-(trifluoromethyl)phenyl)piperidin-1-yl)methanone;
(4-(4-methoxytetrahydro-2H-pyran-4-yl)phenyl)(4-(4-(trifluoromethyl)phenyl)piperidin-1-yl)methanone;
(4-(4-methoxy piperidin-4-yl)phenyl)(4-(4-(trifluoromethyl)phenyl)piperidin-1-yl)methanone;
(4-(3-hydroxythietan-3-yl)phenyl)(4-(4-(trifluoromethyl)phenyl)piperidin-1-yl)methanone;
(4-(3-hydroxythietan-3-yl)phenyl)(5-(4-(trifluoromethyl)phenyl)hexahydropyrrolo[3,4-c]pyrrol-2(1H)-yl)methanone;
(4-(3-methoxythietan-3-yl)phenyl)(4-(4-(trifluoromethyl)phenyl)piperidin-1-yl)methanone;
(4-(3-ethoxythietan-3-yl)phenyl)(4-(4-(trifluoromethyl)phenyl)piperidin-1-yl)methanone;
(4-(3-methoxythietan-3-yl)phenyl)(5-(4-(trifluoromethyl)phenyl)hexahydropyrrolo[3,4-c]pyrrol-2(1H)-yl)methanone;
(4-(3-ethoxythietan-3-yl)phenyl)(5-(4-(trifluoromethyl)phenyl)hexahydropyrrolo[3,4-c]pyrrol-2(1H)-yl)methanone;
tert-butyl 3-hydroxy-3-(4-(4-(4-(trifluoromethyl)phenyl)piperidine-1-carbonyl)phenyl)pyrrolidine-1-carboxylate;
(4-(3-hydroxypyrrolidin-3-yl)phenyl)(4-(4-(trifluoromethyl)phenyl)piperidin-1-yl)methanone;
(4-(3-methoxypyrrolidin-3-yl)phenyl)(4-(4-(trifluoromethyl)phenyl)piperidin-1-yl)methanone;
tert-butyl 3-hydroxy-3-(4-(4-(4-(trifluoromethyl)phenyl)piperidine-1-carbonyl)phenyl)azetidine-1-carboxylate;
(4-(3-hydroxyazetidin-3-yl)phenyl)(4-(4-(trifluoromethyl)phenyl)piperidin-1-yl)methanone;
(4-(4-(methoxy-d3)piperidin-4-yl)phenyl)(4-(4-(trifluoromethyl)phenyl)piperidin-1-yl)methanone;
tert-butyl 4-methoxy-4-(4-(4-(4-(trifluoromethyl)phenyl)piperidine-1-carbonyl)phenyl)piperidine-1-carboxylate;
1-(4-methoxy-4-(4-(4-(4-(trifluoromethyl)phenyl)piperidine-1-carbonyl) phenyl)piperidin-1-yl)ethan-1-one;
ethyl 4-methoxy-4-(4-(4-(4-(trifluoromethyl)phenyl)piperidine-1-carbonyl) phenyl)piperidine-1-carboxylate;
(4-(4-methoxy-1-methylpiperidin-4-yl)phenyl)(4-(4-(trifluoromethyl)phenyl) piperidin-1-yl)methanone;
tert-butyl 4-ethoxy-4-(4-(4-(4-(trifluoromethyl)phenyl)piperidine-1-carbonyl) phenyl)piperidine-1-carboxylate;
(4-(4-ethoxypiperidin-4-yl)phenyl)(4-(4-(trifluoromethyl)phenyl)piperidin-1-yl)methanone;
1-(4-ethoxy-4-(4-(4-(4-(trifluoromethyl)phenyl)piperidine-1-carbonyl)phenyl)piperidin-1-yl)ethan-1-one;
ethyl 4-ethoxy-4-(4-(4-(4-(trifluoromethyl)phenyl)piperidine-1-carbonyl)phenyl)piperidine-1-carboxylate;
(4-(4-ethoxy-1-methylpiperidin-4-yl)phenyl)(4-(4-(trifluoromethyl)phenyl)piperidin-1-yl)methanone;
tert-butyl 4-hydroxy-4-(4-(5-(4-(trifluoromethyl)phenyl)octahydropyrrolo[3,4-c]pyrrole-2-carbonyl)phenyl)piperidine-1-carboxylate;
tert-butyl 4-methoxy-4-(4-(5-(4-(trifluoromethyl)phenyl)octahydropyrrolo[3,4-c]pyrrole-2-carbonyl)phenyl)piperidine-1-carboxylate;
(4-(4-methoxy piperidin-4-yl)phenyl)(5-(4-(trifluoromethyl)phenyl)hexahydropyrrolo[3,4-c]pyrrol-2(1H)-yl)methanone;
(4-(4-methoxy-1-(2,2,2-trifluoro ethyl)piperidin-4-yl)phenyl)(4-(4-(trifluoromethyl)phenyl)piperidin-1-yl)methanone;
(4-(4-ethoxy-1-(2,2,2-trifluoroethyl)piperidin-4-yl)phenyl)(4-(4-(trifluoromethyl)phenyl)piperidin-1-yl)methanone;
(3-(3-hydroxyoxetan-3-yl)phenyl)(2-(4-(trifluoromethyl)phenyl)-6,7-dihydrothieno [3,2-c]pyridin-5(4H)-yl)methanone;
(4-(3-hydroxyoxetan-3-yl)phenyl)(3-(4-(trifluoromethyl)phenyl)-6,7-dihydrothieno [3,2-c]pyridin-5(4H)-yl)methanone;
(3-(3-hydroxyoxetan-3-yl)phenyl)(2-(4-(trifluoromethyl)phenyl)-6,7-dihydrothiazolo[5,4-c]pyridin-5(4H)-yl)methanone;
N-(4-(3-hydroxyoxetan-3-yl)phenyl)-2-(4-(trifluoromethyl)phenyl)-6,7-dihydrothiazolo[5,4-c]pyridine-5(4H)-carboxamide;
(4-(3-hydroxyoxetan-3-yl)phenyl)(2-(4-(trifluoromethyl)phenyl)-6,7-dihydrothiazolo[5,4-c]pyridin-5(4H)-yl)methanone;
(3-(3-hydroxyoxetan-3-yl)phenyl)(3-(4-(trifluoromethyl)phenyl)-6,7-dihydrothieno [3,2-c]pyridin-5(4H)-yl)methanone;
(1-benzyl-3-(4-(trifluoromethyl)phenyl)-4,6-dihydropyrrolo[3,4-c]pyrazol-5 (1H)-yl)(3-(3-hydroxyoxetan-3-yl)phenyl)methanone;
(3-(3-ethoxyoxetan-3-yl)phenyl)(5-(4-(trifluoromethyl)phenyl)hexahydropyrrolo[3,4-c]pyrrol-2(1H)-yl)methanone;
ethyl 2-((3-(3-(5-(4-(trifluoromethyl)phenyl)octahydropyrrolo[3,4-c]pyrrole-2-carbonyl)phenyl)oxetan-3-yl)oxy)acetate;
(3-(3-isobutoxyoxetan-3-yl)phenyl)(5-(4-(trifluoromethyl)phenyl) hexahydropyrrolo[3,4-c]pyrrol-2(1H)-yl)methanone;
(3-(3-(cyclopropylmethoxy)oxetan-3-yl)phenyl)(5-(4-(trifluoromethyl)phenyl)hexahydropyrrolo[3,4-c]pyrrol-2(1H)-yl)methanone;
N-(3-(3-hydroxyoxetan-3-yl)phenyl)-5-(4-(trifluoromethyl)phenyl) hexahydropyrrolo[3,4-c]pyrrole-2(1H)-carboxamide;
N-(3-(3-methoxyoxetan-3-yl)phenyl)-5-(4-(trifluoromethyl)phenyl) hexahydropyrrolo[3,4-c]pyrrole-2(1H)-carboxamide;
(4-(3-hydroxyoxetan-3-yl)phenyl)(4-(4-(trifluoromethyl)phenyl)-1,4-diazepan-1-yl)methanone;
(4-(3-methoxyoxetan-3-yl)phenyl)(4-(4-(trifluoromethyl)phenyl)-1,4-diazepan-1-yl)methanone;
(4-(3-hydroxyoxetan-3-yl)phenyl)(8-(4-(trifluoromethyl)phenyl)-2,8-diazaspiro[4.5]decan-2-yl)methanone;
4-(3-hydroxyoxetan-3-yl)-N-(1-(4-(trifluoromethyl)phenyl)pyrrolidin-3-yl)benzamide;
(4-(3-(2-hydroxyethoxy)oxetan-3-yl)phenyl)(4-(4-(trifluoromethyl)phenyl)piperidin-1-yl)methanone;
(4-(3-(2-(ethylthio)ethoxy)oxetan-3-yl)phenyl)(4-(4-(trifluoromethyl)phenyl)piperidin-1-yl)methanone;
(4-(3-(2-(ethyl sulfonyl)ethoxy)oxetan-3-yl)phenyl)(4-(4-(trifluoromethyl)phenyl)piperidin-1-yl)methanone;
(4-(3-hydroxyoxetan-3-yl)phenyl)(4-(4-(methylthio)phenyl)piperidin-1-yl)methanone;
(4-(3-methoxyoxetan-3-yl)phenyl)(4-(4-(methylthio)phenyl)piperidin-1-yl)methanone;
(4-(4-hydroxypiperidin-4-yl)phenyl)(4-(4-(methylthio)phenyl)piperidin-1-yl)methanone;
(4-(4-methoxy piperidin-4-yl)phenyl)(4-(4-(methylthio)phenyl)piperidin-1-yl)methanone;
(3-(3-(methoxy-d3)oxetan-3-yl)phenyl)(5-(4-(trifluoromethyl)phenyl)hexahydropyrrolo[3,4-c]pyrrol-2(1H)-yl)methanone;
(3-(3-isopropoxyoxetan-3-yl)phenyl)(5-(4-(trifluoromethyl)phenyl)hexahydropyrrolo[3,4-c]pyrrol-2(1H)-yl)methanone;
N-(3-(3-hydroxyoxetan-3-yl)-5-morpholinophenyl)-1-(4-(trifluoromethyl)phenyl)piperidine-4-carboxamide;
methyl 4-(5-hydroxy-2-(4-(3-hydroxyoxetan-3-yl)benzoyl)octahydrocyclopenta[c]pyrrol-5-yl)benzoate;
(4-(3-hydroxyoxetan-3-yl)phenyl)(5-(4-methoxyphenyl)-3,3a,4,6a-tetrahydrocyclopenta[c]pyrrol-2 (1H)-yl)methanone;
(4-(3-hydroxyoxetan-3-yl)phenyl)(5-(4-(2-(methylthio)ethoxy)phenyl)-3,3a,4,6a-tetrahydrocyclopenta[c]pyrrol-2(1H)-yl)methanone;
N-(3-(3-hydroxyoxetan-3-yl)-5-(4-methylpiperazin-1-yl)phenyl)-1-(4-(trifluoromethyl)phenyl)piperidine-4-carboxamide;
(4-(3-methoxyoxetan-3-yl)phenyl)(5-(4-(trifluoromethyl)phenyl)-3,4,5,6-tetrahydropyrrolo[3,4-c]pyrrol-2(1H)-yl)methanone;
(5-hydroxy-5-(6-methoxypyridin-3-yl)hexahydrocyclopenta[c]pyrrol-2 (1H)-yl)(4-(3-hydroxyoxetan-3-yl)phenyl)methanone;
3-(4-((1-(4-(trifluoromethyl)phenyl)piperidin-4-yl)amino)phenyl)oxetan-3-ol;

5. A compound selected from:
tert-butyl 4-methoxy-4-(4-(4-(5-(trifluoromethyl)pyridin-2-yl)piperidine-1-carbonyl)phenyl)piperidine-1-carboxylate;
(4-(4-methoxypiperidin-4-yl)phenyl)(4-(5-(trifluoromethyl)pyridin-2-yl)piperidin-1-yl)methanone;
(4-(3-methoxyoxetan-3-yl)phenyl)(5-(6-(trifluoromethyl)pyridin-3-yl)hexahydropyrrolo[3,4-c]pyrrol-2(1H)-yl)methanone;
(4-(3-hydroxyoxetan-3-yl)phenyl)(5-(5-(trifluoromethyl)pyridin-2-yl)hexahydropyrrolo[3,4-c]pyrrol-2(1H)-yl)methanone;
(3-(3-methoxyoxetan-3-yl)phenyl)(5-(5-(trifluoromethyl)pyridin-2-yl)hexahydropyrrolo[3,4-c]pyrrol-2(1H)-yl)methanone;
(4-(3-methoxyoxetan-3-yl)phenyl)(5-(5-(trifluoromethyl)pyridin-2-yl)hexahydropyrrolo[3,4-c]pyrrol-2(1H)-yl)methanone;
(3-(3-hydroxyoxetan-3-yl)phenyl)(5-(5-(trifluoromethyl)pyridin-2-yl)hexahydropyrrolo[3,4-c]pyrrol-2(1H)-yl)methanone;
(4-(3-methoxyoxetan-3-yl)phenyl)(4-(5-(trifluoromethyl)pyridin-2-yl)piperidin-1-yl)methanone;
(4-(3-hydroxyoxetan-3-yl)phenyl)(4-(5-(trifluoromethyl)pyridin-2-yl)piperidin-1-yl)methanone;
(4-(3-methoxyoxetan-3-yl)phenyl)(4-(6-(trifluoromethyl)pyridin-3-yl)piperidin-1-yl)methanone;
(4-(3-hydroxyoxetan-3-yl)phenyl)(4-(6-(trifluoromethyl)pyridin-3-yl)piperidin-1-yl)methanone;
(4-(3-hydroxyoxetan-3-yl)phenyl)(5-(6-(trifluoromethyl)pyridin-3-yl)hexahydropyrrolo[3,4-c]pyrrol-2(1H)-yl)methanone;
(4-(3-hydroxyoxetan-3-yl) phenyl)(4-(5-(trifluoromethyl) pyridin-2-yl) piperazin-1-yl)methanone;
(4-(3-hydroxyoxetan-3-yl) phenyl)(4-(6-(trifluoromethyl) pyridin-3-yl) piperazin-1-yl) methanone;
(4-(3-methoxyoxetan-3-yl)phenyl)(4-(5-(trifluoromethyl)pyridin-2-yl)piperazin-1-yl)methanone;
(4-(3-ethoxyoxetan-3-yl)phenyl)(4-(5-(trifluoromethyl)pyridin-2-yl)piperazin-1-yl)methanone;
(3-(3-hydroxyoxetan-3-yl)phenyl)(4-(5-(trifluoromethyl)pyridin-2-yl)piperazin-1-yl)methanone;
(3-(3-methoxyoxetan-3-yl)phenyl)(4-(5-(trifluoromethyl)pyridin-2-yl)piperazin-1-yl)methanone;
(4-(3-hydroxyoxetan-3-yl)phenyl)(4-(2-(trifluoromethyl)pyridin-4-yl)piperazin-1-yl)methanone;
(4-(3-hydroxyoxetan-3-yl)phenyl)(4-(2-(trifluoromethyl)pyridin-4-yl)piperidin-1-yl)methanone.

6. A pharmaceutical composition comprising a therapeutically effective amount of compound of formula (I) as claimed in claim 1 and together with one or more suitable pharmaceutically acceptable excipients.

7. A pharmaceutical composition comprising a compound of formula (I) as claimed in claim 1 in combination with one or more pharmaceutically active agents selected from group comprising insulin, insulin derivatives and mimetics, insulin secretagogues, insulin sensitizers, biguanide agents, alpha-glucosidase inhibitors, insulinotropic sulfonylurea receptor ligands, meglitinides, GLP-1 (glucagon like peptide-1), GLP-1 analogs, dipeptidyl peptidase IV (DPP-IV) inhibitors, GPR-1 19 activators, sodium-dependent glucose co-transporter (SGLT2) inhibitors, PPAR modulators, non-glitazone type PPAR.delta. agonist, HMG-CoA reductase inhibitors, cholesterol-lowering drugs, rennin inhibitors, anti-thrombotic and anti-platelet agents and anti-obesity agents or pharmaceutically acceptable salts thereof.

8. (canceled)

9. A method of treating hyperlipidemia and disorders related to hyperlipidemia comprising the administering to a patient in need thereof an effective amount of a compound as claimed in claim 1 or pharmaceutical composition thereof.

10. A pharmaceutical composition comprising a therapeutically effective amount of compound of claim 4 together with one or more suitable pharmaceutically acceptable excipients.

11. A pharmaceutical composition comprising a compound of claim 4 in combination with one or more pharmaceutically active agents selected from group comprising insulin, insulin derivatives and mimetics, insulin secretagogues, insulin sensitizers, biguanide agents, alpha-glucosidase inhibitors, insulinotropic sulfonylurea receptor ligands, meglitinides, GLP-1 (glucagon like peptide-1), GLP-1 analogs, dipeptidyl peptidase IV (DPP-IV) inhibitors, GPR-1 19 activators, sodium-dependent glucose co-transporter (SGLT2) inhibitors, PPAR modulators, non-glitazone type PPAR.delta. agonist, HMG-CoA reductase inhibitors, cholesterol-lowering drugs, rennin inhibitors, anti-thrombotic and anti-platelet agents and anti-obesity agents or pharmaceutically acceptable salts thereof.

12. A method of treating hyperlipidemia and disorders related to hyperlipidemia comprising the administering to a patient in need thereof an effective amount of a compound of claim 4 or pharmaceutical composition thereof.

13. A pharmaceutical composition comprising a therapeutically effective amount of compound of claim 5 together with one or more suitable pharmaceutically acceptable excipients.

14. A pharmaceutical composition comprising a compound of claim 5 in combination with one or more pharmaceutically active agents selected from group comprising insulin, insulin derivatives and mimetics, insulin secretagogues, insulin sensitizers, biguanide agents, alpha-glucosidase inhibitors, insulinotropic sulfonylurea receptor ligands, meglitinides, GLP-1 (glucagon like peptide-1), GLP-1 analogs, dipeptidyl peptidase IV (DPP-IV) inhibitors, GPR-1 19 activators, sodium-dependent glucose co-transporter (SGLT2) inhibitors, PPAR modulators, non-glitazone type PPAR.delta. agonist, HMG-CoA reductase inhibitors, cholesterol-lowering drugs, rennin inhibitors, anti-thrombotic and anti-platelet agents and anti-obesity agents or pharmaceutically acceptable salts thereof.

15. A method of treating hyperlipidemia and disorders related to hyperlipidemia comprising the administering to a patient in need thereof an effective amount of a compound of claim 5 or pharmaceutical composition thereof.
